SEQUENCE LISTING (24 sequences)

SEQ ID NO: 1 - DNA, 22 bp, Artificial sequence
  Synthetic oligonucleotide primer.
ctacgcaccacagtcaaggaag

SEQ ID NO: 2 - DNA, 19 bp, Artificial sequence
  Synthetic oligonucleotide primer.
tgcatccatattcccccca

SEQ ID NO: 3 - DNA, 20 bp, Artificial sequence
  Synthetic oligonucleotide primer.
ggccagcaatacaacatcac

SEQ ID NO: 4 - DNA, 19 bp, Artificial sequence
  Synthetic oligonucleotide primer.
cccagaaacaccttacacg

SEQ ID NO: 5 - DNA, 21 bp, Artificial sequence
  Synthetic oligonucleotide primer.
tagcagcaatcaaaggcatgc

SEQ ID NO: 6 - DNA, 21 bp, Artificial sequence
  Synthetic oligonucleotide primer.
tagcagcaatcaaaggcatgc

SEQ ID NO: 7 - DNA, 19 bp, Artificial sequence
  Synthetic oligonucleotide primer.
acaggtcgctcatcccgtc

SEQ ID NO: 8 - DNA, 18 bp, Artificial sequence
  Synthetic oligonucleotide primer.
cccaaggatcccgactgc

SEQ ID NO: 9 - DNA, 21 bp, Artificial sequence
  Synthetic oligonucleotide primer.
aaatcccatgagtttcacgcc

SEQ ID NO: 10 - DNA, 22 bp, Artificial sequence
  Synthetic oligonucleotide primer.
cagacccctaactggtgctgtt

SEQ ID NO: 11 - DNA, 21 bp, Artificial sequence
  Synthetic oligonucleotide primer.
cggctttgagacagagtgcaa

SEQ ID NO: 12 - DNA, 21 bp, Artificial sequence
  Synthetic oligonucleotide primer.
cgcaccagctctttgaattcc

SEQ ID NO: 13 - DNA, 22 bp, Artificial sequence
  Synthetic oligonucleotide primer.
tgtggatggtgagctgtactcg

SEQ ID NO: 14 - DNA, 21 bp, Artificial sequence
  Synthetic oligonucleotide primer.
gttgagccaaaaggccaggta

SEQ ID NO: 15 - DNA, 22 bp, Artificial sequence
  Synthetic oligonucleotide primer.
tgccagatatcgaggaggtgaa

SEQ ID NO: 16 - DNA, 21 bp, Artificial sequence
  Synthetic oligonucleotide primer.
tgacttcctcttggccttgct

SEQ ID NO: 17 - DNA, 21 bp, Artificial sequence
  Synthetic oligonucleotide primer.
tgaccaccttctacaattccc

SEQ ID NO: 18 - DNA, 21 bp, Artificial sequence
  Synthetic oligonucleotide primer.
aactcttcaagtccttcctcc

SEQ ID NO: 19 - DNA, 21 bp, Artificial sequence
  Synthetic oligonucleotide primer.
ttccccacccaggtttacatc

SEQ ID NO: 20 - DNA, 21 bp, Artificial sequence
  Synthetic oligonucleotide primer.
aaagttcatgacgcctggagc

SEQ ID NO: 21 - DNA, 21 bp, Artificial sequence
  Synthetic oligonucleotide primer.
gaatgtggacaccaaccagga

SEQ ID NO: 22 - DNA, 21 bp, Artificial sequence
  Synthetic oligonucleotide primer.
ttcaaagcgcctcagctcttc

SEQ ID NO: 23 - DNA, 20 bp, Artificial sequence
  Synthetic oligonucleotide primer.
cgcaacgcagtgtgcatatt

SEQ ID NO: 24 - DNA, 21 bp, Artificial sequence
  Synthetic oligonucleotide primer.
accgccagtctggttggtaaa

(19) United States
(12) Patent Application Publication
(10) Pub. No.: US 2010/0086481 A1
(43) Pub. Date: Apr. 8, 2010

(54) DIFFERENTIAL EXPRESSION OF MOLECULES ASSOCIATED WITH INTRA-CEREBRAL HEMORRHAGE

(76) Inventors: Alison E. Baird, Brooklyn, NY (US); David F. Moore, Rockville, MD (US); Ehud Goldin, Rockville, MD (US); Kory Johnson, Rockville, MD (US)

Correspondence Address: KLARQUIST SPARKMAN, LLP, 121 S.W. SALMON STREET, SUITE #1600, PORTLAND, OR, 97204-2988, US

(21) Appl. No.: 12/307,910

(22) PCT Filed: Jul. 11, 2007
(86) PCT No.: PCT/US07/73272

§ 371 Date: Jul. 6, 2009

Related U.S. Application Data
(60) Provisional application No. 60/807,027, filed on Jul. 11, 2006.

(51) Int. Cl. C12Q 1/68; C40B 40/06; C40B 40/10; A61K 49/00

(52) U.S. Cl. 424/9.1; 435/6; 506/16; 506/18

(57) ABSTRACT

Methods are provided for evaluating a stroke, for example for determining whether a subject has had a hemorrhagic stroke, determining the severity or likely neurological recovery of a subject who has had a hemorrhagic stroke, and determining a treatment regimen for a subject who has had a hemorrhagic stroke, as are arrays and kits that can be used to practice the methods. In particular examples, the method includes screening for expression of hemorrhagic stroke related genes (or proteins), such as genes (or proteins) involved in suppression of the immune response, genes (or proteins) involved in vascular repair, genes (or proteins) involved in the acute inflammatory response, genes (or proteins) involved in cell adhesion, genes (or proteins) involved in hypoxia, genes (or proteins) involved in signal transduction, and genes (or proteins) involved in the response to the altered cerebral microenvironment. Arrays and kits are provided that can be used in the disclosed methods. Also provided are methods of identifying one or more agents that alter the activity (such as the expression) of a hemorrhagic stroke-related molecule.

### CROSS-REFERENCE TO RELATED APPLICATION

This application claims the benefit of U.S. Provisional Application No. 60/807,027 filed Jul. 11, 2006.

### FIELD

This application relates to methods of evaluating a stroke, methods of identifying a treatment modality for a subject who has had a hemorrhagic stroke, methods of identifying compounds that alter the activity of a hemorrhagic stroke-related molecule, as well as arrays and kits that can be used to practice the disclosed methods.

### BACKGROUND

Stroke is the third leading cause of death and the leading cause of adult disability in developed countries (Simons et al., Stroke 29:1341-6, 1998; Adams et al., Ischemic Cerebrovascular Disease. New York: Oxford, 2001). Strokes are caused by an interruption of blood flow to the brain, by either an intravascular occlusion (such as an arterial thrombus) or a hemorrhage. A hemorrhagic stroke occurs when a blood vessel ruptures and leaks blood into (intracerebral hemorrhage) or around the brain (subarachnoid hemorrhage), and accounts for about 10-15% of strokes. The American Heart Association estimates that there are approximately three million stroke survivors in the United States, most of whom are disabled. Despite the prevalence and burden of this disease, stroke precipitants and pathophysiological mechanisms in individual patients are often unknown. It is also difficult to accurately predict whether a stroke will lead to only minor neurological sequalae or more serious medical consequences.

Gene expression profiling involves the study of mRNA levels in a tissue sample to determine the expression levels of genes that are expressed or transcribed from genomic DNA. Following a stroke, released brain antigens can be detected in the blood. Such antigens include S100B, neuron specific enolase (NSE), and glial fibrillary acid protein (GFAP), although S100B and GFAP are of low sensitivity for early stroke diagnosis, and NSE and myelin basic protein (MBP) MBP are non-specific (Lamers et al., Brain. Res. Bull. 61:261-4, 2003). Four soluble factors that have demonstrated moderate sensitivity and specificity for the diagnosis of stroke include two markers of inflammation (matrix metalloproteinase-9 and vascular cell adhesion molecule), one marker of glial activation (S100beta) and one thrombosis marker (von Willebrand factor) (Lynch et al., Stroke 35:57-63, 2004).

### SUMMARY

Although stroke is one of the leading causes of morbidity and mortality in developed countries, methods for rapidly and accurately determining whether a subject has had a stroke are expensive and invasive. Therefore, new methods are needed for evaluating a stroke, for example for determining whether the subject has suffered a stroke, and determine what type of stroke the subject had (e.g. ischemic or hemorrhagic). For example, methods are needed to determine whether a hemorrhagic stroke has occurred, for determining the severity of the stroke or the likely neurological recovery of the subject who had a hemorrhagic stroke, or combinations thereof. In some examples, the hemorrhagic stroke is an intracerebral hemorrhagic (ICH) stroke. In particular examples, the disclosed methods offer a potentially lower cost alternative to expensive imaging modalities (such as MRI and CT scans), can be used in instances where those imaging modalities are not available (such as in field hospitals), and can be more convenient than placing individuals in scanners (for example for subjects who can not be subjected to MRI, such as those having certain types of metallic implants in their bodies).

Using these methods, appropriate therapy protocols for subjects who have had a hemorrhagic stroke can be identified and administered. For example, because the results of the disclosed methods are highly reliable predictors of the hemorrhagic nature of the stroke, the results can also be used (alone or in combination with other clinical evidence and brain scans) to determine whether surgery to evacuate the blood clot, administration of an anti-hypertensive agent, administration of a coagulant, management of increased intracranial pressure, prophylaxis of seizures, or combinations thereof, should be used to treat the subject. In certain examples, antihypertensives or blood clotting therapy (or both) is given to the subject once the results of the differential expression assay are known if the assay provides an indication that the stroke is hemorrhagic in nature.

The inventors have identified changes in gene expression in peripheral blood mononuclear cells (PBMCs) that allow one to evaluate a stroke, for example to determine whether a subject has had a hemorrhagic or ischemic stroke, to determine the severity of a hemorrhagic stroke, to determine the likely neurological recovery of the subject, or combinations thereof. For example, such methods can be used to determine if the subject has had an intracerebral hemorrhagic stroke, and not an ischemic stroke. The disclosed methods allow one to screen many genes simultaneously and serially and only a relatively small amount of cell or tissue sample is needed. Changes in gene expression were observed in at least 25 genes, at least 30 genes, at least 119 genes, at least 316 genes, at least 446 genes, or even at least 1263 genes as detected by 37-1500 gene probes depending on sensitivity and specificity of the analysis used and the comparative sample (whether control or ischemic stroke). In particular examples, subjects who had an intracerebral hemorrhagic stroke showed altered gene expression in IL1R2 and amphiphysin (and in some examples also CD163, TAP2, granzyme M and haptoglobin) or any combinations thereof, such as a change in expression in at least 1, at least 2, at least 3, at least 4, at least 5, or all 6 of these genes. In some examples, subjects who had a hemorrhagic stroke showed altered gene expression in at least four of the following seven classes of genes: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction. In some examples, subjects who had a hemorrhagic stroke showed increased gene expression in at least these seven classes of genes.

The disclosed gene expression fingerprint of hemorrhagic stroke (such as intracerebral hemorrhagic stroke) enables methods of evaluating a stroke. The disclosed methods are the first that permit accurate diagnosis of a hemorrhagic stroke (such as an intracerebral hemorrhagic stroke) using PBMCs with high sensitivity and specificity. In some examples, the disclosed methods are at least 75% sensitive (such as at least 80% sensitive or at least 90% sensitive) and at least 80% specific (such as at least 85% specific, at least 95% specific, or 100% specific) for identifying those subjects who have suffered an intracerebral hemorrhagic stroke, for example within the past 72 hours. In particular examples, the disclosed methods are at least 75% sensitive and 100% specific for predicting the likelihood of neurological recovery of a subject who has had an intracerebral hemorrhagic stroke.

In some examples, the method involves detecting patterns of increased protein expression, decreased protein expression, or both. Such patterns of expression can be detected either at the nucleic acid level (such as quantitation of mRNAs associated with protein expression) or the protein level (such as quantitative spectroscopic detection of proteins). Certain methods involve not only detection of patterns of expression, but detection of the magnitude of expression (increased, decreased, or both), wherein such patterns are associated with the subject having had a hemorrhagic stroke, or is associated with predicted clinical sequalae, such as neurological recovery following a hemorrhagic stroke.

The disclosed methods can be performed on a subject who is suspected of having had a stroke, for example prior to radiographic investigation. For example, the disclosed methods can be used to distinguish subjects having an ICH from subjects having an ischemic stroke. In another example, the method is performed on a subject known to have had a hemorrhagic stroke, as the disclosed assays permit early and accurate stratification of risk of long-lasting neurological impairment.

In one example, the method of evaluating a stroke includes determining whether a subject has changes in expression in four or more hemorrhagic stroke-associated molecules that comprise, consist essentially of, or consist of, sequences (such as a DNA, RNA or protein sequence) involved in acute inflammatory response, cell adhesion, suppression of the immune response, hypoxia, hematoma formation or vascular repair, response to the altered cerebral microenvironment, and signal transduction.

In other examples, hemorrhagic stroke-associated molecules comprise, consist essentially of, or consist of, IL1R2, amphiphysin, TAP2, CD163, granzyme M, and haptoglobin, or any 1, 2, 3, 4, 5, or 6 of these molecules (such as IL1R2, amphiphysin, and TAP2). For example, hemorrhagic stroke-associated molecules can comprise, consist essentially of, or consist of, 4 or more, such as 5 or more, 10 or more, 15 or more, 20 or more, 25 or more, 30 or more, 60 or more, 100 or more, 110 or more, 119 or more, 316 or more, 446 or more, 500 or more, 1000 or more, 1200 or more, or 1263 or more of the nucleic acid or protein sequences listed in Tables 2-8 and 15-16. Any of the identified sequences can be used in combination with such sets or subsets of sequences.

In a particular example, evaluating a stroke includes detecting differential expression in at least four hemorrhagic stroke-related molecules of the subject, such as any combination of at least four genes (or the corresponding proteins) listed in any of Tables 2-8 and 15-16, wherein the presence of differential expression of at least four hemorrhagic-stroke related molecules indicates that the subject has had a hemorrhagic stroke, such as an intracerebral hemorrhagic stroke. Therefore, such methods can be used to diagnose a hemorrhagic stroke, such as an ICH stroke. In particular examples, the at least four hemorrhagic-stroke related molecules include at least one of IL1R2, amphiphysin, TAP2, CD163, granzyme M, and haptoglobin, such as at least 2, at least 3, at least 4, at least 5 or at least 6 of such molecules. For example, the method can include determining if the subject has increased gene (or protein) expression of at least one of IL1R2, haptoglobin, amphiphysin, or CD163, optionally in combination with determining if the subject has altered gene (or protein) expression of any other combination of other hemorrhagic stroke-associated molecules, such as any combination of at least 2 other genes (for example any combination of at least 3, at least 5, at least 10, at least 20, at least 50, at least 100, at least 200, or even at least 500 genes) listed in Tables 2-8 and 15-16, such as decreased expression of TAP2 and granzyme M.

In a particular example, differential expression is detected by determining if the subject has increased gene (or protein) expression of at least one of IL1R2, haptoglobin, amphiphysin, or CD163, and determining if the subject has decreased gene (or protein) expression of at least one of TAP2 or granzyme M. For example, differential expression can be detected by determining if the subject has increased gene (or protein) expression of IL1R2, haptoglobin, amphiphysin, and CD163, and determining if the subject has decreased gene (or protein) expression of TAP2 and granzyme M, wherein the presence of differential expression of at least four of these molecules indicates that the subject has had a hemorrhagic stroke.

In one example, the method includes determining if the subject has an increase or decrease in gene expression in any combination of at least four of the genes listed in Tables 2-8 and 15-16, for example an increase in at least 5, at least 10, at least 15, at least 20, at least 25, or at least 30 of the genes listed in Tables 2-8 and 15-16. An increase or decrease in expression in any combination of four or more of the genes listed in Tables 2-8 and 15-16 (or the corresponding proteins), and particularly any combination of at least one gene (or protein) from each of these classes of genes: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, indicates that the subject has had an ICH.

In one example, the method of evaluating a stroke includes determining if the subject has a change in gene expression (such as an increase or decrease) in any combination of at least 4 of the 47 genes listed in Table 2, for example a change in expression in at least 10, at least 20, at least 30, at least 40, or at least 45 of the probes listed in Tablet. Any one of the set of genes can be identified by a single one or the genes listed in Table 2. Any one of the genes (or proteins) in Table 2 can be combined with any other combination of the genes (or proteins) in Table 2 to produce a combination or subcombination of genes. A change in expression in any combination of four or more of the genes listed in Table 2 (or the corresponding proteins) indicates that the subject has had a hemorrhagic stroke, such as an ICH.

In another example, the method of evaluating a stroke includes determining if the subject has a change in gene expression (such as an increase or decrease) in any combination of at least 4 of the genes listed in Table 5 or 8, for example an increase or decrease in any combination of at least 10, at least 15, at least 20, at least 25, at least 100, at least 200, at least 300, or at least 316 of the genes listed in Table 5 or 8. Any one of the set of genes (or proteins) can be identified by a single one or the genes (or proteins) listed in Table 5 or 8. Any one of the genes (or proteins) in Table 5 or 8 can be combined with any other combination of the genes (or proteins) in Table 5 or 8 to produce a combination or subcombination of genes. A change in expression in any combination of four or more of the genes listed in Table 5 or 8 (or the corresponding proteins) indicates that the subject has had a hemorrhagic stroke, such as an ICH.

The disclosed methods can be used in combination with methods that permit diagnosis of a stroke. Such methods can be performed before or during classification of a stroke (e.g. to determine if the stroke is ischemic or hemorrhagic). For example, the method can include determining if there is significant upregulation in at least 4 of the 15 genes/proteins listed in Table 14, wherein significant upregulation in 4 or more of the 15 genes/proteins listed in Table 14 (such as at least 5, 6, 7, 8, 9, 10, 11, 12, 13, 14, or 15) of the genes/proteins listed in Table 14, indicates that the subject has suffered a stroke. However, such genes/proteins do not classify the stroke as ischemic or hemorrhagic. Therefore, using the methods provided herein, use of at least four (such as at least 10 or at least 30) of the genes/proteins listed in Tables 2-8 and 15-16 can be used to classify a stroke as hemorrhagic while use of at least four (such as at least 10 or at least 25) the genes/proteins listed in Tables 15 and 17-18 can be used to classify a stroke as ischemic.

In some examples, the amount of gene (or protein) expression in the subject is compared to a control, such as the gene (or protein) expression of a subject who has not had a hemorrhagic stroke, wherein an increase or decrease in expression in any combination of four or more hemorrhagic stroke related genes listed in Tables 2-8 and 15-16 compared to the control indicates that the subject has experienced an hemorrhagic stroke. For example, an increase or decrease in expression in any combination of at least one gene (or the corresponding protein) from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, compared to the control indicates that the subject has experienced a hemorrhagic stroke, such as an ICH.

In some examples, the amount of gene (or protein) expression in the subject is compared to a control, such as the gene (or protein) expression of a subject who has had an ischemic stroke or a subject who has not had a stroke, wherein an increase or decrease in expression in any combination of four or more hemorrhagic stroke related genes listed in Tables 2-8 and 15-16 compared to the control indicates that the subject has experienced an hemorrhagic stroke.

In particular examples evaluating the stroke includes predicting a likelihood of severity of neurological sequalae of the hemorrhagic stroke, such as an intracerebral hemorrhagic stroke. In some examples, evaluating the stroke includes predicting a likelihood of neurological recovery of the subject. For example, if there is differential expression (such as increased expression) in at least four of the hemorrhagic-stroke related molecules listed in Tables 2-8 and 15-16 (such as differential expression of IL1R2, haptoglobin, amphiphysin, and TAP2), indicates that the subject has a higher risk of long-term adverse neurological sequalae and therefore a lower likelihood of neurological recovery. In another example, detecting a change in expression in any combination of 10 or more of the genes listed in Tables 2-8 and 15-16 (or the corresponding proteins) indicates that the subject has a higher risk of long-term adverse neurological sequalae and therefore a lower likelihood of neurological recovery. In yet another example, detecting a change in expression in any combination of at least 10 of the 47 of the genes listed in Table 2, at least 10 of the 1263 of the genes listed in Table 3, at least 10 of the 119 of the genes listed in Table 4, at least 10 of the 30 of the genes listed in Table 5, at least 10 of the 446 of the genes listed in Table 6, at least 10 of the 25 of the genes listed in Table 7, at least 4 of the 5 of the genes listed in Table 15, or at least 10 of the 18 of the genes listed in Table 16, for example an increase or decrease in any combination of at least 20, at least 50, at least 100, at least 200, at least 300, or at least 500 of the genes listed in Tables 2-8 and 15-16 indicates that the subject has a higher risk of long-term adverse neurological sequalae and therefore a lower likelihood of neurological recovery. In some examples, differential expression in the subject is compared to differential expression of a subject who has not had an hemorrhagic stroke, wherein a change in expression in at least four the hemorrhagic-stroke related molecules listed in Tables 2-8 and 15-16, such as any combination of 10 or more of the genes listed in Tables 2-8 and 15-16 (or the corresponding proteins) compared to the control indicates that the subject has a higher risk of long-term adverse neurological sequalae and therefore a lower likelihood of neurological recovery. In some examples, the amount of expression is quantitated, wherein a greater change in expression in at least four the hemorrhagic-stroke related molecules listed in Tables 2-8 and 15-16 compared to the control indicates that the subject has a higher risk of long-term adverse neurological sequalae and therefore a lower likelihood of neurological recovery.

The disclosed methods can further include administering to a subject a treatment to avoid or reduce hemorrhagic injury if the presence of differential expression indicates that the subject has had a hemorrhagic stroke. For example, a change in expression in at least four hemorrhagic stroke related molecules, such as a combination that includes at least four of the molecules listed in Tables 2-8 and 15-16, indicates that the subject has had a hemorrhagic stroke (and not an ischemic stroke) and is in need of the appropriate therapy, such as surgery to evacuate the blood clot, monitoring and treatment of intracranial pressure, brain swelling, and seizures, administration of a blood coagulant, administration of an anti-hypertensive (for example to treat high blood pressure), or combinations thereof. Therefore, the disclosed methods differentiate hemorrhagic (such as intracerebral hemorrhage) from ischemic stroke, and allow one to administer the appropriate therapy to the subject. In some examples, the amount of differential expression in the subject is compared to the expression of a subject who has not had a hemorrhagic stroke, wherein a change in expression in at least four hemorrhagic stroke related molecules listed in Tables 2-8 and 15-16 (or the corresponding proteins) compared to the control indicates that the subject would benefit from one or more of the therapies described above. In some examples, the amount of differential expression in the subject is compared to the expression of a subject who has had an ischemic stroke, wherein a change in expression in at least four hemorrhagic stroke related molecules listed in Tables 2-8 and 15-16 (or the corresponding proteins) compared to the control indicates that the subject would benefit from one or more of the therapies described above.

Differential expression can be detected at any time following the onset of clinical signs and symptoms that indicate a potential stroke, such as within 24 hours, within 72 hours, within 2-11 days, within 7-14 days, or within 90 days of onset of clinical signs and symptoms that indicate a potential stroke. Examples of such signs and symptoms include, but are not limited to: headache, sensory loss (such as numbness, particularly confined to one side of the body or face), paralysis (such as hemiparesis), pupillary changes, blindness (including bilateral blindness), ataxia, memory impairment, dysarthria, somnolence, and other effects on the central nervous system recognized by those of skill in the art.

In particular examples, the disclosed methods include isolating nucleic acid molecules (such as mRNA molecules) or proteins from PBMCs of a subject suspected of having had a hemorrhagic stroke (or known to have had a hemorrhagic stroke), for example an intracerebral hemorrhagic stroke. The isolated nucleic acid or protein molecules can be contacted with or applied to an array, for example an array that includes oligonucleotide probes (or probes that can bind proteins, such as an antibody) capable of hybridizing to hemorrhagic stroke-associated genes (or proteins). In one example, proteins isolated from a biological sample are quantitated, for instance by quantitative mass spectroscopy, to determine whether proteins associated with hemorrhagic stroke or prognosis of hemorrhagic stroke are upregulated, downregulated, or both. In some examples, PBMCs are obtained within at least the previous 72 hours of a time when the stroke is suspected of occurring, such as within the previous 24 hours.

Also provided herein are arrays that include molecules (such as oligonucleotide probes or antibody probes that specifically hybridize or bind to at least four hemorrhagic stroke-related sequences) that permit evaluation of a stroke. For example, the array can include or consist of probes (such as an oligonucleotide probes or antibodies) specific for the hemorrhagic-stroke related molecules provided in Tables 2-8 and 15-16, such as probes capable of hybridizing or binding to genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction. Such arrays can permit quantitation of hemorrhagic stroke-related nucleic acid or protein sequences present in a sample, such as a sample that includes PBMC nucleic acid molecules or proteins. Kits including such arrays are also disclosed. Such arrays can further include probes that are specific for the molecules listed in Table 14, 17, 18, or combinations thereof.

Also provided in the present disclosure are methods of identifying one or more agents that alter the activity (such as the expression) of a hemorrhagic stroke-related molecule (for example a gene or protein), such as one or more of those listed in Tables 2-8 and 15-16. If desired, multiple test agents and multiple hemorrhagic stroke-related molecules can be screened at the same time. In one example, the method is used to screen the effect of one test agent on multiple hemorrhagic stroke-related molecules simultaneously (such as all of the hemorrhagic stroke-related molecules listed in any of Tables 2-8 and 15-16). In another example, the method is used to screen the effect of multiple test agents on one hemorrhagic stroke-related molecule, such as one of the molecules listed in Tables 2-8 and 15-16. In particular examples, the identified agent alters the activity of a hemorrhagic stroke-related molecule that is upregulated or downregulated following a hemorrhagic stroke. For example, the agent can normalize activity of a hemorrhagic stroke-related molecule that is upregulated or downregulated following a hemorrhagic stroke, such as by increasing the activity of a hemorrhagic stroke-related molecule that is down-regulated following a hemorrhagic stroke, or decreasing activity of a hemorrhagic stroke-related molecule that is upregulated following a hemorrhagic stroke. The disclosed methods can be performed in vitro (for example in a cell culture) or in vivo (such as in a mammal).

In one example, the test agent is an agent in pre-clinical or clinical trials or approved by a regulatory agency (such as the Food and Drug Administration, FDA), to treat hemorrhagic stroke. For example, the method can be used to determine if the agent alters the activity of one or more hemorrhagic stroke-related molecules that modifies response to treatment and can predict the best responders.

The disclosed methods can also be used in toxicogenomics, for example to identify genes or proteins whose expression is altered in response to medication-induced toxicity and side-effects. In one example, the disclosed hemorrhagic stroke-related molecules are screened to identify those whose activity is altered in response to an agent. For example, the disclosed hemorrhagic stroke-related molecules can be used determine if an agent promotes or induces an intracerebral hemorrhagic stroke. If the agent promotes or induces differential expression of at least four of the disclosed hemorrhagic stroke-related molecules (such as those listed in Tables 2-8 and 15-16) in an otherwise normal cell or mammal (for example as compared to a similar mammal not administered the test agent), this indicates that the agent may cause or promote an hemorrhagic stroke in vivo. Such a result may indicate that further studies of the agent are needed. In another example, cells from a subject who is to receive a pharmaceutical agent are obtained (such as PBMCs), and the pharmaceutical agent incubated with the cells as described above, to determine if the pharmaceutical agent causes or promotes differential expression of one or more hemorrhagic stroke-related molecules. Such a result would indicate that the subject may react adversely to the agent, or that a lower dose of the agent should be administered.

The disclosure also provides brain imaging tracers or white blood cell tracers for molecular imaging, such as imaging to determine if a subject has had a hemorrhagic stroke. Briefly, a labeled antibody that recognizes a hemorrhagic stroke-related molecule, such as one or more of those listed in Tables 2-8 and 15-16. In one example, the label is a fluorophore, radioisotope, or other compound that can be used in diagnostic imaging, such as a nuclear medicine radio-isotope (for example ^{99m}Technetium for use with single photon emission computed tomography, ¹⁸Fluorodeoxyglucose (¹⁸FDG) for use with positron emission tomography, or a paramagnetic contrast agent for magnetic resonance imaging). The labeled antibody can be administered to the subject, for example intravenously, and the subject imaged using standard methods.

The foregoing and other features of the disclosure will become more apparent from the following detailed description of a several embodiments.

### BRIEF DESCRIPTION OF THE DRAWINGS

FIGS. 1A and 1B are graphs showing the relative amount of (A) IL1R2 and (B) amphiphysin expression in normal subjects and subjects who suffered a hemorrhagic stroke.

FIG. 2 is a bar graph showing the relative amount of amphiphysin expression in normal referent subjects and in subjects who suffered a hemorrhagic stroke 2-11 days before.

### SEQUENCE LISTING

The nucleic acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NOS: 1-2 are oligonucleotide sequences (forward and reverse, respectively) used to perform real-time PCR to determine expression levels of interleukin-1 receptor, type II (IL1R2).

SEQ ID NOS: 3-4 are oligonucleotide sequences (forward and reverse, respectively) used to perform real-time PCR to determine expression levels of IL1R2.

SEQ ID NOS: 5-6 are oligonucleotide sequences (forward and reverse, respectively) used to perform real-time PCR to determine expression levels of amphiphysin.

SEQ ID NOS: 7-8 are oligonucleotide sequences (forward and reverse, respectively) used to perform real-time PCR to determine expression levels of CD163.

SEQ ID NOS: 9-10 are oligonucleotide sequences (forward and reverse, respectively) used to perform real-time PCR to determine expression levels of F5.

SEQ ID NOS: 11-12 are oligonucleotide sequences (forward and reverse, respectively) used to perform real-time PCR to determine expression levels of S100A9.

SEQ ID NOS: 13-14 are oligonucleotide sequences (forward and reverse, respectively) used to perform real-time PCR to determine expression levels of SEMA4C.

SEQ ID NOS: 15-16 are oligonucleotide sequences (forward and reverse, respectively) used to perform real-time PCR to determine expression levels of IRF1.

SEQ ID NOS: 17-18 are oligonucleotide sequences (forward and reverse, respectively) used to perform real-time PCR to determine expression levels of CD6.

SEQ ID NOS: 19-20 are oligonucleotide sequences (forward and reverse, respectively) used to perform real-time PCR to determine expression levels of CASC3.

SEQ ID NOS: 21-22 are oligonucleotide sequences (forward and reverse, respectively) used to perform real-time PCR to determine expression levels of NUCB1.

SEQ ID NOS: 23-24 are oligonucleotide sequences (forward and reverse, respectively) used to perform real-time PCR to determine expression levels of FDFT1.

### DETAILED DESCRIPTION

### Abbreviations and Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank Accession Nos. referred to herein are the sequences available at least as early as Jul. 11, 2006.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Amph: amphiphysin

FC: fold change

ICH: intracerebral hemorrhage

IL1R2: interleukin-1 receptor, type II

IS: ischemic stroke

PBMC: peripheral blood mononuclear cell

Real time PCR: real time polymerase chain reaction

TAP2: Transporter associated with antigen processing

Administration: To provide or give a subject an agent, such as an anti-hypertensive or a blood coagulation factor, by any effective route. Exemplary routes of administration include, but are not limited to, oral, injection (such as subcutaneous, intramuscular, intradermal, intraperitoneal, and intravenous), sublingual, rectal, transdermal, intranasal, vaginal and inhalation routes.

Amphiphysin (Amph): A src homology 3 domain-containing protein that links endocytic proteins to the clathrin-mediated endocytic sites. The presence of Amph antibodies in a subject has been associated with the paraneoplastic disorder stiff-person syndrome. The term amphiphysin includes any amphiphysin gene, cDNA, mRNA, or protein from any organism and that is an amphiphysin that can function in endocytosis. Amphiphysin sequences are publicly available. For example, GenBank Accession Nos: U07616 and AAA21865 disclose human amphiphysin nucleic acid and protein sequences, respectively and GenBank Accession Nos: Y13381 and CAA73808 disclose rat amphiphysin nucleic acid and proteins sequences, respectively.

In one example, an amphiphysin sequence includes a full-length wild-type (or native) sequence, as well as amphiphysin allelic variants, variants, fragments, homologs or fusion sequences that retain the ability to function in endocytosis. In certain examples, amphiphysin has at least 80% sequence identity, for example at least 85%, at least 90%, at least 95%, or at least 98% sequence identity to a native amphiphysin and retains amphiphysin biological activity. In other examples, amphiphysin has a sequence that hybridizes under very high stringency conditions to a sequence set forth in GenBank Accession No. U07616 or Y13381, and retains the ability to encode a protein having amphiphysin biological activity.

Amplifying a nucleic acid molecule: To increase the number of copies of a nucleic acid molecule, such as a gene or fragment of a gene, for example a region of a hemorrhagic stroke-associated gene. The resulting products are called amplification products or amplicons.

An example of in vitro amplification is the polymerase chain reaction (PCR), in which a biological sample obtained from a subject (such as a sample containing PBMCs) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Quantitative real-time PCR is another form of in vitro amplifying nucleic acid molecules, enabled by Applied Biosystems (TaqMan PCR). The 5′ nuclease assay provides a real-time method for detecting only specific amplification products. During amplification, annealing of the probe to its target sequence generates a substrate that is cleaved by the 5′ nuclease activity of Taq DNA polymerase when the enzyme extends from an upstream primer into the region of the probe. This dependence on polymerization ensures that cleavage of the probe occurs only if the target sequence is being amplified. The use of fluorogenic probes makes it possible to eliminate post-PCR processing for the analysis of probe degradation. The probe is an oligonucleotide with both a reporter fluorescent dye and a quencher dye attached. While the probe is intact, the proximity of the quencher greatly reduces the fluorescence emitted by the reporter dye by Förster resonance energy transfer (FRET) through space. Probe design and synthesis has been simplified by the finding that adequate quenching is observed for probes with the reporter at the 5′ end and the quencher at the 3′ end.

Anti-hypertensive: An agent that can reduce or control hypertension (high blood pressure) in a mammal, such as a human. There are several classes of antihypertensives, each of which lowers blood pressure by a different means. Examples of such classes include diuretics (such as a thiazide diuretic), angiotensin-converting enzyme (ACE)-inhibitors, anti-adrenergics, calcium channel blockers, angiotensin II receptor antagonists, aldosterone antagonists, vasodilators, centrally acting adrenergic drugs, adrenergic neuron blockers, and herbals that provoke hypotension. Particular examples of thiazide or thiazide like diuretics include chlortalidone, epitizide, hydrochlorothiazide, chlorothiazide, indapamide and metolazone. Such agents can be administered to a subject to treat or prevent hemorrhagic stroke, such as an intracerebral hemorrhagic stroke.

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called DNA chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from at least four, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array consists essentially of oligonucleotide probes or primers which can be used to detect hemorrhagic stroke-associated sequences, such as any combination of at least four of those listed in Tables 5 or 8, such as at least 10, at least 20, at least 50, at least 100, at least 150, at least 160, at least 170, at least 175, at least 180, at least 185, at least 200, at least 400, at least 500, at least 700, at least 1000, at least 1100, or at least 1200 of the sequences listed in any of Tables 2-8 and 15-16. In some examples, an array includes oligonucleotide probes or primers which can be used to detect at least one gene from each of the following gene classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 2, at least 3, at least 5, or even at least 10 genes from each of the classes of genes.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including nucleic acids to which proteins are bound, or vice versa. In some examples, an array consists essentially of antibodies to hemorrhagic stroke-associated proteins, such as any combination of at least four of those listed in Tables 5 or 8, such as at least 10, at least 20, at least 50, at least 100, at least 150, at least 160, at least 170, at least 175, at least 180, at least 185, at least 200, at least 400, at least 500, at least 700, at least 1000, at least 1100, or at least 1200 of the sequences listed in any of Tables 2-8 and 15-16. In particular examples, an array includes antibodies or proteins that can detect at least one protein from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 2, at least 3, at least 5, or even at least 10 genes from each class.

Binding or stable binding: An association between two substances or molecules, such as the hybridization of one nucleic acid molecule to another (or itself), the association of an antibody with a peptide, or the association of a protein with another protein or nucleic acid molecule. An oligonucleotide molecule binds or stably binds to a target nucleic acid molecule if a sufficient amount of the oligonucleotide molecule forms base pairs or is hybridized to its target nucleic acid molecule, to permit detection of that binding. For example a probe or primer specific for a hemorrhagic stroke-associated nucleic acid molecule can stably bind to the hemorrhagic stroke-associated nucleic acid molecule.

Binding can be detected by any procedure known to one skilled in the art, such as by physical or functional properties of the target: oligonucleotide complex. For example, binding can be detected functionally by determining whether binding has an observable effect upon a biosynthetic process such as expression of a gene, DNA replication, transcription, translation, and the like.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt. In another example, the method involves detecting a signal, such as a detectable label, present on one or both nucleic acid molecules (or antibody or protein as appropriate).

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

CD163: A hemoglobin scavenger receptor. The term CD163 includes any CD163 gene, cDNA, mRNA, or protein from any organism and that is a CD163 that can function as a hemoglobin scavenger receptor. CD163 sequences are publicly available. For example, GenBank Accession Nos: Y18388 and CAB45233 disclose human CD163 nucleic acid and protein sequences, respectively and GenBank Accession Nos: NM_{—}053094 and NP_{—}444324 disclose mouse CD163 nucleic acid and proteins sequences, respectively.

In one example, a CD163 sequence includes a full-length wild-type (or native) sequence, as well as CD163 allelic variants, variants, fragments, homologs or fusion sequences that retain the ability to function as a hemoglobin scavenger receptor. In certain examples, CD163 has at least 80% sequence identity, for example at least 85%, 90%, 95%, or 98% sequence identity to a native CD163. In other examples, CD163 has a sequence that hybridizes under very high stringency conditions to a sequence set forth in GenBank Accession No. Y18388 or NM_{—}053094, and retains CD163 activity.

cDNA (complementary DNA): A piece of DNA lacking internal, non-coding segments (introns) and regulatory sequences which determine transcription. cDNA can be synthesized by reverse transcription from messenger RNA extracted from cells.

Clinical indications of stroke: One or more signs or symptoms that are associated with a subject having (or had) a stroke, such as a hemorrhagic stroke. Particular examples include, but are not limited to: severe headache, sensory loss (such as numbness, particularly confined to one side of the body or face), paralysis (such as hemiparesis), pupillary changes, blindness (including bilateral blindness), ataxia, memory impairment, dysarthria, somnolence, and other effects on the central nervous system recognized by those of skill in the art.

Intracerebral hemorrhagic strokes begin abruptly, and symptoms worsen as the hemorrhage expands. Nausea, vomiting, seizures, and loss of consciousness are common and can occur within seconds to minutes.

Coagulants: Agents that increase blood clotting. Coagulants can promote the formation of new clots, and stimulate existing clots to grow, for example by increasing the production of proteins necessary for blood to clot. Examples include, but are not limited to anti-thrombin, protein C, fresh frozen plasma, cryoprecipitate, and platelets. Administration of coagulants is one treatment for hemorrhagic stroke (such as an intracerebral hemorrhagic stroke), for example to prevent further strokes.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as a stroke-related sequence, for example any of the sequences listed in Tables 2-8 and 14-18) to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

DNA (deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Differential expression: A difference, such as an increase or decrease, in the conversion of the information encoded in a gene (such as a hemorrhagic stroke related gene) into messenger RNA, the conversion of mRNA to a protein, or both. In some examples, the difference is relative to a control or reference value, such as an amount of gene expression that is expected in a subject who has not had a hemorrhagic stroke, an amount expected in a subject who has had an ischemic stroke, or an amount expected in a subject who has had a hemorrhagic stroke. Detecting differential expression can include measuring a change in gene or protein expression, such as a change in expression of one or more hemorrhagic stroke-related genes or proteins.

Downregulated or inactivation: When used in reference to the expression of a nucleic acid molecule (such as a hemorrhagic stroke-associated nucleic acid molecule), such as a gene, refers to any process which results in a decrease in production of a gene product. A gene product can be RNA (such as mRNA, rRNA, tRNA, and structural RNA) or protein. Therefore, gene downregulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a normal cell). For example these genes listed in Tables 2-4 and 6-7 having a negative t-statistic value and the genes listed in Table 16 with a negative FC value are downregulated in subjects who have had an intracerebral hemorrhagic stroke. In one example, a control is a relative amount of gene expression or protein expression in a PBMC in a subject who has not suffered a hemorrhagic stroke or in a subject who has had an ischemic stroke.

Evaluating a stroke: To determine whether a hemorrhagic stroke has occurred in a subject, to determine the severity of a hemorrhagic stroke, to determine the likely neurological recovery of a subject who has had a hemorrhagic stroke, or combinations thereof. In a particular example, includes determining whether the subject has had an ICH, for example and not an ischemic stroke.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule (such as a hemorrhagic stroke-associated nucleic acid molecule) can be altered relative to a normal (wild type) nucleic acid molecule. Alterations in gene expression, such as differential expression, includes but is not limited to: (1) overexpression; (2) underexpression; or (3) suppression of expression. Alternations in the expression of a nucleic acid molecule can be associated with, and in fact cause, a change in expression of the corresponding protein.

Protein expression (such as expression of a hemorrhagic stroke-associated protein) can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard. Controls or standards for comparison to a sample, for the determination of differential expression, include samples believed to be normal (in that they are not altered for the desired characteristic, for example a sample from a subject who has not had an hemorrhagic stroke) as well as reference values, even though possibly arbitrarily set, keeping in mind that such values can vary from laboratory to laboratory.

Reference standards and values may be set based on a known or determined population value and can be supplied in the format of a graph or table that permits comparison of measured, experimentally determined values.

Gene expression profile (or fingerprint): Differential or altered gene expression can be detected by changes in the detectable amount of gene expression (such as cDNA or mRNA) or by changes in the detectable amount of proteins expressed by those genes. A distinct or identifiable pattern of gene expression, for instance a pattern of high and low expression of a defined set of genes or gene-indicative nucleic acids such as ESTs; in some examples, as few as one or two genes provides a profile, but more genes can be used in a profile, for example at least 3, at least 4, at least 5, at least 10, at least 20, at least 25, at least 50, at least 80, at least 100, at least 190, at least 200, at least 300, at least 400, at least 500, at least 700, or at least 1000 or more. A gene expression profile (also referred to as a fingerprint) can be linked to a tissue or cell type (such as PBMCs), to a particular stage of normal tissue growth or disease progression (such as hemorrhagic stroke), or to any other distinct or identifiable condition that influences gene expression in a predictable way. Gene expression profiles can include relative as well as absolute expression levels of specific genes, and can be viewed in the context of a test sample compared to a baseline or control sample profile (such as a sample from a subject who has not had a hemorrhagic stroke). In one example, a gene expression profile in a subject is read on an array (such as a nucleic acid or protein array).

Granzyme M (GM): A trypsin-fold serine protease that participates in target cell death initiated by cytotoxic lymphocytes. Also referred to as (lymphocyte met-ase 1). Granzyme M sequences are publicly available. For example, GenBank Accession Nos: BC025701 and CH471242.1 disclose human granzyme M nucleic acid sequences and GenBank Accession Nos: AAH25701.1 and EAW61189 disclose human granzyme M protein sequences.

In one example, a granzyme M sequence includes a full-length wild-type (or native) sequence, as well as granzyme M allelic variants, variants, fragments, homologs or fusion sequences that retain the ability to participate in target cell death initiated by cytotoxic lymphocytes. In certain examples, granzyme M has at least 80% sequence identity, for example at least 85%, 90%, 95%, or 98% sequence identity to a native granzyme M and retains granzyme M biological activity. In other examples, granzyme M has a sequence that hybridizes under very high stringency conditions to a sequence set forth in GenBank Accession No. BC025701 and CH471242.1, and encodes a protein having granzyme M activity.

Haptoglobin (Hp): A hemoglobin (Hb) binding plasma protein that functions as an antioxidant and a vascular endothelial protector. Hp exists in two major allelic variants: Hp1 and Hp2. Hp forms complexes with free Hb that are rapidly cleared by the liver and by macrophages. The term haptoglobin includes any haptoglobin gene, cDNA, mRNA, or protein from any organism and that is a haptoglobin that can complex with hemoglobin. Haptoglobin sequences are publicly available. For example, GenBank Accession Nos: NM_{—}005143 and NP_{—}005134 disclose human haptoglobin nucleic acid and protein sequences, respectively and GenBank Accession Nos: NP_{—}059066 and NP_{—}444324 disclose mouse haptoglobin nucleic acid and protein sequences, respectively.

In one example, a haptoglobin sequence includes a full-length wild-type (or native) sequence, as well as haptoglobin allelic variants, variants, fragments, homologs or fusion sequences that retain the ability to complex with hemoglobin. In certain examples, haptoglobin has at least 80% sequence identity, for example at least 85%, 90%, 95%, or 98% sequence identity to a native haptoglobin and retains haptoglobin biological activity. In other examples, haptoglobin has a sequence that hybridizes under very high stringency conditions to a sequence set forth in GenBank Accession No. NM_{—}005143 or NM_{—}017370, and encodes a protein having haptoglobin activity.

Hemorrhagic stroke: A hemorrhagic stroke occurs when an artery in the brain leaks or ruptures and causes bleeding inside the brain tissue or near the surface of the brain (as contrasted with an ischemic stroke which develops when a blood vessel that supplies blood to the brain is blocked or narrowed). There are two primary types of hemorrhagic strokes: intracerebral hemorrhage (ICH) and subarachnoid hemorrhage. ICHs occur within the brain, while subarachnoid hemorrhages occur between the pia mater and the arachnoid mater of the meninges. In particular examples, the present disclosure is limited to diagnosis and treatment of an ICH stroke.

About 10% of all strokes are ICHs, such hemorrhages account for a much higher percentage of deaths due to stroke. Among those older than 60, ICH is more common than subarachnoid hemorrhage. Causes of intracerebral hemorrhage include high blood pressure and, in the elderly, fragile blood vessels.

Hemorrhagic Stroke-related (or associated) molecule: A molecule whose expression is affected by a hemorrhagic stroke, such as an ICH stroke. Such molecules include, for instance, nucleic acid sequences (such as DNA, cDNA, or mRNAs) and proteins. Specific examples include those listed in Tables 2-8 and 15-16, as well as fragments of the full-length genes, cDNAs, or mRNAs (and proteins encoded thereby) whose expression is altered (such as upregulated or downregulated) in response to a hemorrhagic stroke.

Examples of hemorrhagic stroke-related molecules whose expression is upregulated following a hemorrhagic stroke include genes involved in acute inflammatory response, genes involved in cell adhesion, genes involved in hypoxia, genes involved in hematoma formation or vascular repair, and genes involved in the response to the altered cerebral microenvironment. Specific examples of hemorrhagic stroke-related molecules whose expression is upregulated following a hemorrhagic stroke include IL1R2, haptoglobin, amphiphysin, and CD163, or any one of these, and specific examples of hemorrhagic stroke-related molecules whose expression is downregulated following a hemorrhagic stroke include B-cell CLL/lymphoma 6 and granzyme M.

Hemorrhagic stroke-related molecules can be involved in or influenced by a hemorrhagic stroke in different ways, including causative (in that a change in a hemorrhagic stroke-related molecule leads to development of or progression to hemorrhagic stroke) or resultive (in that development of or progression to hemorrhagic stroke causes or results in a change in the hemorrhagic stroke-related molecule).

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

In particular examples, an array includes probes or primers that can hybridize to hemorrhagic stroke-related nucleic acid molecules (such as mRNA or cDNA molecules), for example under very high or high stringency conditions.

The following is an exemplary set of hybridization conditions and is not limiting:

Very High Stringency (Detects Sequences that Share at Least 90% Identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share at Least 80% Identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share at Least 50% Identity)

Hybridization: 6×SSC at RT to 55° C. for 16-20 hours

Wash at least twice: 2×-3×SSC at RT to 55° C. for 20-30 minutes each.

Interleukin-1 receptor, type II (IL1R2): Receptor for interleukin 1 family member 9 (IL1F9), which can function as a scavenger receptor for IL-1 thereby reducing binding of IL-1 to its receptor. The term IL1R2 includes any IL1R2 gene, cDNA, mRNA, or protein from any organism and that is an IL1R2 that can function as a receptor for IL1F9. IL1R2 sequences are publicly available. For example, GenBank Accession Nos: NM_{—}003854 and AAZ38712 disclose human IL1R2 nucleic acid and protein sequences, respectively and GenBank Accession Nos: NM_{—}133575 and NP_{—}598259 disclose rat IL1R2 nucleic acid and protein sequences, respectively.

In one example, a IL1R2 sequence includes a full-length wild-type (or native) sequence, as well as IL1R2 allelic variants, variants, fragments, homologs or fusion sequences that retain the ability to function as a receptor for IL1F9. In certain examples, IL1R2 has at least 80% sequence identity, for example at least 85%, at least 90%, at least 95%, or at least 98% sequence identity to a native IL1R2. In other examples, IL1R2 has a sequence that hybridizes under very high stringency conditions to a sequence set forth in GenBank Accession No. NM_{—}003854 or NM_{—}133575, and retains IL1R2 activity.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include hemorrhagic stroke-associated nucleic acid molecules (such as DNA or RNA) and proteins purified by standard purification methods. The term also embraces nucleic acid molecules and proteins prepared by recombinant expression in a host cell as well as chemically synthesized nucleic acid molecules and proteins. For example, an isolated cell, such as an isolated PBMC is one that is substantially separated from other cells, such as other blood cells.

Label: An agent capable of detection, for example by ELISA, spectrophotometry, flow cytometry, or microscopy. For example, a label can be attached to a nucleic acid molecule or protein, thereby permitting detection of the nucleic acid molecule or protein. For example a nucleic acid molecule or an antibody that specifically binds to a hemorrhagic stroke-associated molecule can include a label. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Neurological sequalae: Any abnormality of the nervous system (such as the central nervous system) following or resulting from a disease or injury or treatment, for example following a hemorrhagic stroke.

Nucleic acid array: An arrangement of nucleic acids (such as DNA or RNA) in assigned locations on a matrix, such as that found in cDNA arrays, or oligonucleotide arrays. In a particular example, a nucleic acid array includes probes or primers that can hybridize under high or very high stringency conditions to hemorrhagic stroke-related nucleic acid molecules, such as at least four of such molecules.

Nucleic acid molecules representing genes: Any nucleic acid, for example DNA (intron or exon or both), cDNA, or RNA (such as mRNA), of any length suitable for use as a probe or other indicator molecule, and that is informative about the corresponding gene (such as a hemorrhagic stroke-associated gene).

Nucleic acid molecules: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, nucleic acid molecule can be circular or linear.

The disclosure includes isolated nucleic acid molecules that include specified lengths of a hemorrhagic stroke-related nucleotide sequence, for example those listed in Tables 2-8 and 15-16. Such molecules can include at least 10, at least 15, at least 20, at least 25, at least 30, at least 35, at least 40, at least 45 or at least 50 consecutive nucleotides of these sequences or more, and can be obtained from any region of an hemorrhagic stroke-related nucleic acid molecule.

Nucleotide: Includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Oligonucleotide: A plurality of joined nucleotides joined by native phosphodiester bonds, between about 6 and about 300 nucleotides in length, for example about 6 to 300 contiguous nucleotides of a hemorrhagic stroke-associated nucleic acid molecule. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides. In particular examples, an oligonucleotide includes these numbers of contiguous nucleotides of a hemorrhagic stroke-related nucleic acid molecule. Such an oligonucleotide can be used on a nucleic acid array to detect the presence of the hemorrhagic stroke-related nucleic acid molecule.

Oligonucleotide probe: A short sequence of nucleotides, such as at least 8, at least 10, at least 15, at least 20, at least 21, at least 25, or at least 30 nucleotides in length, used to detect the presence of a complementary sequence (such as a hemorrhagic stroke-associated nucleic acid sequence) by molecular hybridization. In particular examples, oligonucleotide probes include a label that permits detection of oligonucleotide probe:target sequence hybridization complexes. For example, an oligonucleotide probe can include these numbers of contiguous nucleotides of a hemorrhagic stroke-related nucleic acid molecule, along with a detectable label. Such an oligonucleotide probe can be used on a nucleic acid array to detect the presence of the hemorrhagic stroke-related nucleic acid molecule.

Peripheral blood mononuclear cells (PBMCs): Cells present in the blood that have one round nucleus. Examples include lymphocytes, monocytes, and natural killer cells. PBMCs do not include neutrophils, eosinophils or basophils.

Primers: Short nucleic acid molecules, for instance DNA oligonucleotides 10-100 nucleotides in length, such as about 15, 20, 25, 30 or 50 nucleotides or more in length, such as this number of contiguous nucleotides of a hemorrhagic stroke-associated nucleic acid molecule. Primers can be annealed to a complementary target DNA strand by nucleic acid hybridization to form a hybrid between the primer and the target DNA strand. Primer pairs can be used for amplification of a nucleic acid sequence, such as by PCR or other nucleic acid amplification methods known in the art.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5,© 1991, Whitehead Institute for Biomedical Research, Cambridge, Mass.). One of ordinary skill in the art will appreciate that the specificity of a particular primer increases with its length.

In one example, a primer includes at least 15 consecutive nucleotides of a hemorrhagic stroke-related nucleotide molecule, such as at least 18 consecutive nucleotides, at least 20, at least 25, at least 30, at least 35, at least 40, at least 45, at least 50 or more consecutive nucleotides of a hemorrhagic stroke-related nucleotide sequence. Such primers can be used to amplify a hemorrhagic stroke-related nucleotide sequence, for example using PCR.

Purified: The term “purified” does not require absolute purity; rather, it is intended as a relative term. Thus, for example, a purified protein preparation is one in which the protein referred to is more pure than the protein in its natural environment within a cell. For example, a preparation of a protein (such as a hemorrhagic stroke-associated protein) is purified such that the protein represents at least 50% of the total protein content of the preparation. Similarly, a purified oligonucleotide preparation is one in which the oligonucleotide is more pure than in an environment including a complex mixture of oligonucleotides. In addition, a purified cell, such as a purified PBMC, is one that is substantially separated from other cells, such as other blood cells. In one example, purified PBMCs are at least 90% pure, such as at least 95% pure, or even at least 99% pure.

Sample: A biological specimen containing genomic DNA, RNA (including mRNA), protein, or combinations thereof, obtained from a subject. Examples include, but are not limited to, peripheral blood, urine, saliva, tissue biopsy, surgical specimen, amniocentesis samples and autopsy material. In one example, a sample includes PBMCs.

Semaphorin 4C (Sema4C): A group 4 transmembrane semaphorin that interacts with SFAP75 and may play a role in neural function in brain. Sema4C sequences are publicly available. For example, GenBank Accession Nos: NM_{—}017789.3 and NP_{—}060259.3 disclose human Sema4C nucleic acid and protein sequences, respectively and GenBank Accession Nos: AF461179.1 and AAL67573.1 disclose Xenopus Sema4C nucleic acid and protein sequences, respectively.

In one example, a Sema4C sequence includes a full-length wild-type (or native) sequence, as well as Sema4C allelic variants, variants, fragments, homologs or fusion sequences that retain the ability to interact with SFAP75. In certain examples, Sema4C has at least 80% sequence identity, for example at least 85%, 90%, 95%, or 98% sequence identity to a native Sema4C and retains the ability to interact with SFAP75. In other examples, Sema4C has a sequence that hybridizes under very high stringency conditions to a sequence set forth in GenBank Accession No. NM_{—}017789.3 or AF461179.1 and encodes a protein having Sema4C activity.

Sequences involved in (or related to) acute inflammatory response: Nucleic acid molecules (such as genes, cDNA, and mRNA) and the corresponding protein, whose expression when altered (such as upregulated or downregulated) initiates or promotes an acute inflammatory response (such as promoting or enhancing the exudation of plasma proteins and leukocytes into the surrounding tissue), for example in response to an ICH. Particular examples include CD163 and maltase-glucoamylase.

Sequences involved in (or related to) altered cerebral microenvironment: Nucleic acid molecules (such as genes, cDNA, and mRNA) and the corresponding protein, whose expression is altered (such as upregulated or downregulated) in PBMCs in response to changes in the brain microenvironment, for example to enhance synaptic vesicle recycling in the brain, or to increase neuronal recovery and repair. Particular examples include amphiphysin and GAS7.

Sequences involved in (or related to) cell adhesion: Nucleic acid molecules (such as genes, cDNA, and mRNA) and the corresponding protein, whose expression when altered (such as upregulated or downregulated) promotes or enhances cell adhesion, such as the binding of one cell to another cell, or the binding of a cell or to a surface or matrix, for example in response to an ICH. A particular example includes acyl CoA synthase.

Sequences involved in (or related to) hematoma formation/vascular repair: Nucleic acid molecules (such as mRNA, cDNA, genes) and the corresponding protein, whose expression is altered (such as upregulated or downregulated) in response to injury to a blood vessel. Modification of expression of such molecules (such as up- or downregulation) can result in hematoma degradation, coagulation, repair of the vascular system, or combinations thereof, for example in response to an ICH. Such genes may promote healing of damaged blood vessels, such as those that have hemorrhaged, for example resulting in the formation of a hematoma. Particular examples include, but are not limited to, haptoglobin, factor 5, and two genes related to induction of megakaryocyte formation, v-maf musculoaopneurotic fibrosarcoma oncogene homolog B and HIV-1 Rev binding protein.

Sequences involved in (or related to) hypoxia: Nucleic acid molecules (such as genes, cDNA, and mRNA) and the corresponding protein, whose expression is altered (such as upregulated or downregulated) in response to decreased available oxygen in the blood and tissues. For example, the brain is hypoxic following a stroke. A particular example includes solute carrier family 2, member 3.

Sequences involved in (or related to) signal transduction: Nucleic acid molecules (such as genes, cDNA, and mRNA) and the corresponding protein, whose expression when altered (such as upregulated or downregulated) converts one signal into another type of signal, for example to increases signal transmission between cells or with a cell, for example in response to an ICH. Particular examples include centaurin, alpha 2 and cytochrome P450.

Sequences involved in (or related to) suppression of the immune response: Nucleic acid molecules (such as genes, cDNA, and mRNA) and the corresponding protein, which can reduce or inhibit an immune response, such as reducing or inhibiting white blood cell proliferation. In a specific example, expression of one or more of such genes is altered (such as upregulated or downregulated) in response to injury to a blood vessel, for example in response to an ICH. A particular example includes, but is not limited to, IL1R2.

Subject: Living multi-cellular vertebrate organisms, a category that includes human and non-human mammals, such as veterinary subjects. In a particular example, a subject is one who had or is suspected of having had a stroke, such as an intracerebral hemorrhagic stroke.

Target sequence: A sequence of nucleotides located in a particular region in the human genome that corresponds to a desired sequence, such as a hemorrhagic stroke-related sequence. The target can be for instance a coding sequence; it can also be the non-coding strand that corresponds to a coding sequence. Examples of target sequences include those sequences associated with stroke, such as any of those listed in Tables 2-8 and 14-18.

Test agent: Any substance, including, but not limited to, a protein (such as an antibody), nucleic acid molecule, organic compound, inorganic compound, or other molecule of interest. In particular examples, a test agent can permeate a cell membrane (alone or in the presence of a carrier). In particular examples, a test agent is one whose effect on hemorrhagic stroke is to be determined.

Therapeutically effective amount: An amount of a pharmaceutical preparation that alone, or together with a pharmaceutically acceptable carrier or one or more additional therapeutic agents, induces the desired response. A therapeutic agent, such as a coagulant or an anti-hypertensive, is administered in therapeutically effective amounts.

Therapeutic agents can be administered in a single dose, or in several doses, for example daily, during a course of treatment. However, the effective amount of can be dependent on the source applied, the subject being treated, the severity and type of the condition being treated, and the manner of administration. Effective amounts a therapeutic agent can be determined in many different ways, such as assaying for a reduction in blood pressure, reduction in intracranial pressure, reduction in brain swelling, reduction in seizures, increased blood clotting, improvement of physiological condition of a subject having hypertension or having had a hemorrhagic stroke, or combinations thereof. Effective amounts also can be determined through various in vitro, in vivo or in situ assays.

In one example, it is an amount sufficient to partially or completely alleviate symptoms of hemorrhagic stroke within a subject. Treatment can involve only slowing the progression of the hemorrhagic stroke temporarily, but can also include halting or reversing the progression of the hemorrhagic stroke permanently. For example, a pharmaceutical preparation can decrease one or more symptoms of hemorrhagic stroke, for example decrease a symptom by at least 20%, at least 50%, at least 70%, at least 90%, at least 98%, or even at least 100%, as compared to an amount in the absence of the pharmaceutical preparation.

Transporter associated with antigen processing (TAP2): Forms a heterodimer with TAP1, and the heterodimer binds antigenic peptides (such as MHC class I molecules) and transports them from the cytosol into the lumen of the endoplasmic reticulum (ER) in an ATP-dependent manner. The term TAP2 includes any TAP2 gene, cDNA, mRNA, or protein from any organism and that is a TAP2 that can transport antigenic peptides into the ER. TAP2 sequences are publicly available. For example, GenBank Accession Nos: NT_{—}007592 and NP_{—}061313 disclose human TAP2 nucleic acid and protein sequences, respectively and GenBank Accession Nos: NM_{—}032056 and NP_{—}114445 disclose rat TAP2 nucleic acid and protein sequences, respectively.

In one example, a TAP2 sequence includes a full-length wild-type (or native) sequence, as well as TAP2 allelic variants, variants, fragments, homologs or fusion sequences that retain the ability to transport antigenic peptides into the ER. In certain examples, TAP2 has at least 80% sequence identity, for example at least 85%, 90%, 95%, or 98% sequence identity to a native TAP2 and retains the ability to transport antigenic peptides into the ER. In other examples, TAP2 has a sequence that hybridizes under very high stringency conditions to a sequence set forth in GenBank Accession No. NT_{—}007592 or NM_{—}032056 and encodes a protein having TAP2 activity.

Treating a disease: “Treatment” refers to a therapeutic intervention that ameliorates a sign or symptom of a disease or pathological condition, such a sign or symptom of intracerebral hemorrhagic stroke. Treatment can also induce remission or cure of a condition, such as a hemorrhagic stroke. In particular examples, treatment includes preventing a disease, for example by inhibiting the full development of a disease, such as preventing development of a disease or disorder that results from a hemorrhagic stroke. Prevention of a disease does not require a total absence of disease. For example, a decrease of at least 50% can be sufficient.

Under conditions sufficient for: A phrase that is used to describe any environment that permits the desired activity.

In one example, includes administering a test agent to a subject sufficient to allow the desired activity. In particular examples, the desired activity is altering the activity (such as the expression) of a hemorrhagic stroke-related molecule, for example normalizing such activity to control levels (such as a level found in a subject not having had a stroke).

Upregulated or activation: When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene upregulation or activation includes processes that increase transcription of a gene or translation of mRNA, such as a hemorrhagic stroke-associated gene or other nucleic acid molecule.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product, such as a hemorrhagic stroke-associated gene product. In certain examples, production of a gene product increases by at least 2-fold, for example at least 3-fold or at least 4-fold, as compared to a control (such an amount of gene expression in a normal cell). For example these genes listed in Tables 2-4 or 6-7 having a positive t-statistic value and genes listed in Tables 15 and 16 with a positive FC value are upregulated in subjects who have had an ICH stroke. In one example, a control is a relative amount of gene expression in a PBMC in a subject who has not suffered a hemorrhagic stroke, or in a subject who has had an ischemic stroke, or combinations thereof.

### Hemorrhagic Stroke-Related Molecules

The inventors have identified at least 25 genes whose expression is altered (such as upregulated or downregulated) following a hemorrhagic stroke, such as an intracerebral hemorrhagic stroke (ICH). The number of genes identified depended on the specificity and sensitivity of the algorithm used, as well as which subjects were compared. For example, using the Holm dataset, 50 hemorrhagic stroke-related probes were identified when comparing intracerebral hemorrhagic stroke, ischemic stroke and control subjects (Table 2), using the false discovery rate (fdr) dataset, the Holm dataset, or the PAM dataset, 1263, 119, or 30 hemorrhagic stroke-related genes were identified respectively, when comparing intracerebral hemorrhagic stroke and control subjects, (Tables 3-5, respectively), and using the fdr dataset, the Holm dataset, or the PAM dataset, 446, 25, or 316 hemorrhagic stroke-related genes were identified respectively, when comparing intracerebral hemorrhagic stroke and ischemic stroke subjects (Tables 6-8, respectively). Using other algorithms, 15 genes were found to be significantly upregulated in subjects who had suffered a stroke (whether IS or ICH) compared to normal subjects (Table 14), 5 genes were significantly unregulated in ICH subjects relative to IS subjects (Table 15), 18 genes were significantly differentially expressed in ICH subjects relative to normal subjects (Table 16), and 1 gene was significantly upregulated in IS subjects relative to normal subjects (Table 17). One skilled in the art will appreciate that changes in protein expression can be detected as an alternative to detecting gene expression.

Several genes not previously associated with hemorrhagic stroke were identified, such as at least IL1R2, haptoglobin, amphiphysin, and TAP2. In particular examples, some genes were upregulated (IL1R2, haptoglobin, amphiphysin) and some genes were downregulated (TAP2 and granzyme M) following a hemorrhagic stroke. In one example, classes of genes whose expression was altered following a hemorrhagic stroke were identified: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction.

Based on the identification of these hemorrhagic stroke-related molecules, methods were developed to evaluate a stroke. For example, the disclosed methods can be used to diagnose a hemorrhagic stroke, determine the severity of a hemorrhagic stroke, determine the likely neurological recovery of a subject who had a hemorrhagic stroke, or combinations thereof. In particular examples, the hemorrhagic stroke is an intracerebral hemorrhagic stroke. The method can further include determining an appropriate therapy for a subject found to have experienced hemorrhagic stroke using the disclosed assays.

The disclosed methods provide a rapid, straightforward, and accurate genetic screening method performed in one assay for evaluating hemorrhagic stroke, such as intracerebral hemorrhagic stroke. It allows identification of subjects who may require coagulant or anti-hypertensive therapy (or other appropriate therapy) following a hemorrhagic stroke. For example, by establishing that an individual has had a hemorrhagic stroke, effective therapeutic measures, such as the emergent administration of a coagulant or anti-hypertensive to treat the stroke or to prevent such hemorrhagic stroke recurrence and extension, can be instituted.

### Evaluation of a Hemorrhagic Stroke

Provided herein are methods of evaluating a stroke. Particular examples of evaluating a stroke include determining whether a subject, such as an otherwise healthy subject, or a subject suspected or at risk of having a hemorrhagic stroke, has had hemorrhagic stroke, assessing the severity of a hemorrhagic stroke, predicting the likelihood of neurological recovery of a subject who has had a hemorrhagic stroke, or combinations thereof. The identification of a subject who has had a hemorrhagic stroke (such as an intracerebral hemorrhagic stroke) can help to evaluate other clinical data (such as neurological impairment or brain imaging information) to determine whether a hemorrhagic stroke (and not an ischemic stroke) has occurred. In particular examples, the method can determine with a reasonable amount of sensitivity and specificity whether a subject has suffered a hemorrhagic stroke (such as an ICH) within the previous 5 days, such as within the previous 72 hours, the previous 48 hours, previous 24 hours, or previous 12 hours. In some examples, isolated or purified PBMCs obtained from the subject are used to determine whether a subject has had a hemorrhagic stroke, such as an ICH.

In particular examples, the method also includes administering an appropriate treatment therapy to subjects who have had a hemorrhagic stroke. For example, subjects identified or evaluated as having had a hemorrhagic stroke can then be provided with appropriate treatments, such as anti-hypertensive agents or agents that promote blood clotting or combinations thereof, that would be appropriate for a subject identified as having had a hemorrhagic stroke but not as appropriate for a subject who has had an ischemic stroke. It is helpful to be able to classify a subject as having had a hemorrhagic stroke, because the treatments for hemorrhagic stroke are often distinct from the treatments for ischemic stroke. In fact, treating a hemorrhagic stroke with a therapy designed for an ischemic stroke (such as a thrombolytic agent) can have devastating clinical consequences. Hence using the results of the disclosed assays to help distinguish ischemic from hemorrhagic stroke offers a substantial clinical benefit, and allows subjects to be selected for treatments appropriate to hemorrhagic stroke but not ischemic stroke.

In particular examples, methods of evaluating a stroke involve detecting differential expression (such as an increase or decrease in gene or protein expression) in any combination of at least four hemorrhagic stroke-related molecules of the subject, such as any combination of at least four of the genes (or proteins) listed in any of Tables 2-8 and 15-16. In one example, the method includes screening expression of one or more of IL1R2, CD163, amphiphysin, or TAP2, or a combination of hemorrhagic stroke-related molecules that includes at least 1, at least 2, at least 3, or at least 4 of these molecules. For example, the method can include screening expression of IL1R2, along with other hemorrhagic stroke-related molecules (such as any combination that includes at least 3 additional molecules listed in Tables 2-8 and 15-16, for example haptoglobin, amphiphysin, TAP2, CD163, and granzyme M).

Differential expression can be represented by increased or decreased expression in the at least one hemorrhagic stroke-related molecule (for instance, a nucleic acid or a protein). For example, differential expression includes, but is not limited to, an increase or decrease in an amount of a nucleic acid molecule or protein, the stability of a nucleic acid molecule or protein, the localization of a nucleic acid molecule or protein, or the biological activity of a nucleic acid molecule or protein. Specific examples include evaluative methods in which changes in gene expression in at least four hemorrhagic stroke-related nucleic acid molecules (or corresponding protein) are detected (for example nucleic acids or proteins obtained from a subject thought to have had or known to have had a hemorrhagic stroke), such as changes in gene (or protein) expression in any combination of at least 5, at least 10, at least 15, at least 20, at least 25, at least 50, at least 100, at least 150, at least 160, at least 170, at least 175, at least 180, at least 185, at least 200, at least 250, at least 300, at least 400, at least 500, at least 700, at least 1000, at least 1100, or at least 1263 hemorrhagic stroke-related molecules. Exemplary hemorrhagic stroke-related molecules are provided in Tables 2-8 and 15-16.

In particular examples a change in expression is detected in a subset of hemorrhagic stroke-related molecules (such as nucleic acid sequences or protein sequences) that selectively evaluate a stroke, for example to determine if a subject has had a hemorrhagic stroke. In a particular example, the subset of molecules can include a set of any combination of four hemorrhagic stroke-related genes listed in Table 5 or 8. In a particular example, the subset of molecules includes any combination of at least one gene (or protein) from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 2, at least 3, at least 5, or at least 10 genes from each class.

In a particular example, differential expression is detected in hemorrhagic stroke-related molecules that are both upregulated and down regulated. For example, increased expression of one or more of (such as 2, 3, or 4 of) IL1R2, haptoglobin, amphiphysin, and CD163 and decreased gene (or protein) expression of one or more of TAP2, Sema4C, or granzyme M, indicates that the subject has had a hemorrhagic stroke, has had a severe hemorrhagic stroke, has a lower likelihood of neurological recovery, or combinations thereof. For example, differential expression can be detected by determining if the subject has increased gene (or protein) expression of IL1R2, CD163, and amphiphysin, and determining if the subject has decreased gene (or protein) expression of TAP2 or granzyme M, wherein detection of such increased and decreased expression indicates that the subject has suffered a hemorrhagic stroke.

In particular examples, the number of hemorrhagic stroke-related genes screened is at least 5, at least 10, at least 15, at least 20, at least 25, at least 30, at least 60, at least 70, at least 100, at least 110, at least 130, at least 140, at least 150, at least 200, at least 250, at least 300, at least 400, at least 500, at least 1000, or at least 1263 hemorrhagic stroke-related molecules. In other examples, the methods employ screening no more than 1263, no more than 1000, no more than 500, no more than 446, no more than 316, no more than 250, no more than 200, no more than 150, no more than 119, no more than 100, no more than 63, no more than 50, no more than 30, no more than 25, no more than 20, no more than 15, no more than 10, no more than 5, or no more than 4 hemorrhagic stroke-related genes. Examples of particular hemorrhagic stroke-related genes are shown in Tables 2-8 and 15-16. In one example, the number of hemorrhagic stroke-related genes screened includes at least one gene from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 2, at least 3, at least 5, or at least 10 genes from each class. In some examples, detection of differential expression of at least four molecules listed in Tables 2-8 and 15-16 indicates that the subject has had a hemorrhagic stroke, has had a severe hemorrhagic stroke, has a lower likelihood of neurological recovery, or combinations thereof, while detection of differential expression of in no more than two molecules listed in Tables 2-8 and 15-16 indicates that the subject has not had a hemorrhagic stroke, has had a mild hemorrhagic stroke, has a greater likelihood of neurological recovery, or combinations thereof.

In certain methods, differential expression includes over- or under-expression of a hemorrhagic stroke-related molecule. In some examples the presence of differential expression is evaluated by determining a t-statistic value that indicates whether a gene or protein is up- or down-regulated. For example, an absolute t-statistic value can be determined. In some examples, a negative t-statistic indicates that the gene or protein is downregulated, while a positive t-statistic indicates that the gene or protein is upregulated. In particular examples, a t-statistic less than −3 indicates that the gene or protein is downregulated, such as less than −3.5, less than −4.0, less than −5.0, less than −6.0, less than −7.0 or even less than −8.0, while a t-statistic of at least 3, such as at least 3.5, at least 4.0, at least 5.0, at least 6.0, at least 7.0, at least 8.0, at least 9.0, at least 10, or at least 15, indicates that the gene or protein is upregulated.

For instance, differential expression can include overexpression, for instance overexpression of any combination of at least 4 molecules (such at least 10 or at least 20 molecules) shown in Tables 2-4 or 6-7 with a positive t-statistic value (such as a t-statistic value of at least 3, such as at least 4, at least 6 or even at least 8) or shown in Tables 15 and 16 with a positive FC value (such as an FC value of at least 1.2). In a particular example, differential expression includes differential expression of any combination of at least one gene from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 2, at least 3, at least 5, or at least 10 genes from each of the classes. In another particular example, differential expression includes differential expression of any combination of at least one gene from at least three of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 4, at least 5, or all of the classes. In another example, differential expression includes underexpression, for instance underexpression of any combination of at least four molecules (such at least 50 or at least 150 molecules) shown in Tables 2-4 or 6-7 with a negative t-statistic value (such as a t-statistic value of less than −3, such as less than −4, less than −6 or even less than −7 or Table 16 with a negative FC value (such as a value less than −1.3). In a specific example, differential expression includes any combination of increased expression or decreased expression of at least 4 hemorrhagic stroke-related molecules shown in Tables 2-4, 6-7 or 16, such as upregulation of at least 3 hemorrhagic stroke-related molecules shown in Tables 2-4 or 6-7 with a positive t-statistic value or Tables 15-16 with a positive FC value and downregulation of at least one hemorrhagic stroke related molecule shown in Tables 2-4 or 6-7 with a negative t-statistic value or Table 16 with a negative FC value, or for example upregulation of at least 4 hemorrhagic stroke-related molecules shown in Tables 2-4 or 6-7 with a positive t-statistic value or Tables 15-16 with a positive FC value, or for example, upregulation of at least 2 hemorrhagic stroke-related molecules shown in Tables 2-4 or 6-7 with a positive t-statistic value or Tables 15-16 with a positive FC value and downregulation of at least 2 hemorrhagic stroke related molecules shown in Tables 2-4 or 6-7 with a negative t-statistic value or Table 16 with a negative FC value.

In some examples, differential expression of proteins that are associated with hemorrhagic stroke includes detecting patterns of such expression, such as detecting upregulation of IL1R2, haptoglobin, amphiphysin, and CD163, and detecting downregulation of TAP2, granzyme M or Sema4C. For example, detecting upregulation or downregulation can include a magnitude of change of at least 25%, at least 50%, at least 100%, or even at least 200%, such as a magnitude of change of at least 25% for CD163; at least 25% for IL1R2; at least 25% for haptoglobin; at least 25% for amphiphysin; at least 25% for TAP2; at least 25% for Sema4C; and at least 25% for granzyme M. Alternatively, upregulation is detected by a level having a t-value of at least 4 and downregulation is detected by a level having a t-value value of no more than −4.

In particular examples, the disclosed method of evaluating a stroke is at least 75% sensitive (such as at least 80% sensitive, at least 85% sensitive, at least 90% sensitive, or at least 95% sensitive) and at least 80% specific (such as at least 85% specific, at least 90% specific, at least 95% specific, or 100% specific) for determining whether a subject has had a hemorrhagic stroke, such as an ICH.

As used herein, the term “hemorrhagic stroke-related molecule” includes hemorrhagic stroke-related nucleic acid molecules (such as DNA, RNA, for example cDNA or mRNA) and hemorrhagic stroke-related proteins. The term is not limited to those molecules listed in Tables 2-8 and 15-16 (and molecules that correspond to those listed), but also includes other nucleic acid molecules and proteins that are influenced (such as to level, activity, localization) by or during a hemorrhagic stroke (such as an intracerebral hemorrhagic stroke), including all of such molecules listed herein. Examples of particular hemorrhagic stroke-related genes are listed in Tables 2-8 and 15-16, such as IL1R2, haptoglobin, amphiphysin, TAP2, CD163, and granzyme M. In examples where the hemorrhagic-related molecule is a hemorrhagic stroke-related nucleic acid sequence, exemplary methods of detecting differential expression include in vitro nucleic acid amplification, nucleic acid hybridization (which can include quantified hybridization), RT-PCR, real time PCR, or combinations thereof. In examples where the hemorrhagic stroke-related molecule is an hemorrhagic-related protein sequence, exemplary methods of detecting differential expression include in vitro hybridization (which can include quantified hybridization) such as hybridization to a protein-specific binding agent for example an antibody, quantitative spectroscopic methods (for example mass spectrometry, such as surface-enhanced laser desorption/ionization (SELDI)-based mass spectrometry) or combinations thereof. However, one skilled in the art will recognize that other nucleic acid or protein detection methods can be used.

In particular examples, methods of evaluating a subject who has had or is thought to have had an hemorrhagic stroke includes determining a level of expression (for example in a PBMC) of any combination of at least 4 of the genes (or proteins) listed in Tables 2-8 and 15-16, such as at least 10, at least 15, at least 20, or at least 30 of the genes listed in Tables 5 or 8, such as at least 20, at least 30, at least 50, at least 100, at least 200, or at least 500 of the genes listed in Tables 2-8 and 15-16. In one example, the method includes determining a level of expression of at least IL1R2, amphiphysin, TAP2, and CD163, or any combination of hemorrhagic stroke-related molecules that includes 1, 2, 3, or 4 of these molecules. In one example, the method includes determining a level of expression of at least one gene from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 2, at least 3, at least 5, or at least 10 genes from each class.

Methods of evaluating a stroke can include diagnosing a stroke, stratifying the seriousness of an intracerebral hemorrhagic event, and predicting neurological recovery. Similarly, methods of evaluating a stroke can include determining the severity of a hemorrhagic stroke, predicting neurological recovery, or combinations thereof. For example, a change in expression in any combination of at least four of the genes listed in Tables 2-8 and 15-16 indicates that the subject has had a hemorrhagic stroke. For example, an increase in expression in one or more of IL1R2, haptoglobin, amphiphysin, or CD163, and a decrease in expression of one or more of TAP2, granzyme M and Sema4C, in particular examples indicates that the subject has had a hemorrhagic stroke, such as an ICH.

The disclosed methods of evaluating a stroke can include a diagnosis of a stroke. For example, a diagnosis of stroke (whether IS or ICH) can be made, as well as classification of the stroke as ischemic or hemorrhagic. Diagnosis of stroke can be performed before or during classification of a stroke (e.g. to determine if the stroke is ischemic or hemorrhagic). For example, it can first be determined whether the subject has suffered a stroke, then determined if the stroke is ischemic or hemorrhagic. Alternatively, such diagnosis and classification can be done simultaneously (or near simultaneously), for example by using one or more arrays with the appropriate probes. For example, the method can include determining if there is significant upregulation in at least 4 of the 15 genes/proteins listed in Table 14, wherein significant upregulation in 4 or more of the 15 genes/proteins listed in Table 14 (such as at least 5, 6, 7, 8, 9, 10, 11, 12, 13, 14, or 15) of the genes/proteins listed in Table 14, indicates that the subject has suffered a stroke. However, such genes/proteins do not classify the stroke as ischemic or hemorrhagic. To classify the stroke as hemorrhagic, at least four (such as at least 10 or at least 30) of the genes/proteins listed in Tables 2-8 and 15-16 can be used, and to classify the stroke as ischemic at least four (such as at least 10 or at least 25) the genes/proteins listed in Tables 15 and 17-18 can be used. Methods of using the genes/proteins listed in Tables 2-8 and 14-18 to classify a stroke as hemorrhagic or ischemic are provided herein.

Determining the level of expression can involve measuring an amount of the hemorrhagic stroke-related molecules in a sample derived from the subject, such as a purified PBMC sample. Such an amount can be compared to that present in a control sample (such as a sample derived from a subject who has not had a hemorrhagic stroke or a standard hemorrhagic stroke-related molecule level in analogous samples from a subject not having had a hemorrhagic stroke or not having a predisposition developing hemorrhagic stroke), wherein a difference (such as an increase or a decrease reflecting an upregulation or downregulation, respectively) in the level of any combination of at least four hemorrhagic stroke-related molecules listed in Tables 2-8 and 15-16, such as any combination of at least four hemorrhagic stroke-related molecules listed in Table 5, in the subject relative to the control sample is diagnostic for hemorrhagic stroke, such as an intracerebral hemorrhagic stroke.

In other examples, the method includes determining a level of expression of any combination of at least four sequences listed in Table 5, such as at least 10 or at least 50 of the sequences listed in Table 8, for example at least 40 of the genes listed in Table 2, such as at least 50 of the genes listed in Table 3, such as at least 50 of the genes listed in Table 4, such as at least 50 of the genes listed in Table 6, at least 10 of the hemorrhagic stroke-related molecules listed in Table 7, at least 4 of the hemorrhagic stroke-related molecules listed in Table 15, or at least 10 of the hemorrhagic stroke-related molecules listed in Table 16. In one example, a change in expression detected in at least four genes listed in Table 5 or 8 (or the corresponding proteins), such as at least 10 of the genes (or the corresponding proteins) listed in Table 5 or 8, such as 50 or more of the genes listed in Table 2, 3, 4, 6, 7, 15 or 16 (or the corresponding proteins), such as 500 or more of the genes listed in Table 2, 3, 4, 6, 7, 15 or 16 (or the corresponding proteins, indicates that the subject has had a more severe hemorrhagic stroke, has a higher risk of long term adverse neurological sequalae, or combinations thereof, than a subject having a change in expression in less than 50, such as less than 10 or less than three of the molecules listed in Tables 2-8 and 15-16. Determining the level of expression can involve measuring an amount of the hemorrhagic stroke-related molecules in a sample derived from the subject. Such an amount can be compared to that present in a control sample (such as a sample derived from a subject who has not had a hemorrhagic stroke or a sample derived from the subject at an earlier time), wherein a difference (such as an increase or a decrease reflecting an upregulation or downregulation, respectively) in the level of at least four of the hemorrhagic stroke-related molecules listed in Tables 2-8 and 15-16 (such as at least 25 or at least 50 of the hemorrhagic stroke-related molecules listed in Tables 2-8 and 15-16) in the subject relative to the control sample indicates that the subject has had a more severe hemorrhagic stroke, has a higher risk of long term adverse neurological sequalae, or both.

The disclosed methods can further include administering to the subject an appropriate treatment to avoid or reduce hemorrhagic injury, if the presence of differential expression indicates that the subject has had a hemorrhagic stroke. Since the results of the disclosed assays are reliable predictors of the hemorrhagic nature of the stroke, the results of the assay can be used (alone or in combination with other clinical evidence and brain scans) to determine whether blood clotting therapy designed to clot a neurovascular hemorrhage should be administered to the subject. In certain example, coagulant or anti-hypertensive therapy (or both) is given to the subject once the results of the differential gene assay are known if the assay provides an indication that the stroke is hemorrhagic in nature. Such methods can reduce brain damage following a hemorrhagic stroke.

In particular examples, the method includes determining if there is an alteration in the expression of at least four sequences listed in Table 5, such as at least 10 or at least 50 of the sequences listed in Table 8, such as at least 10 or at least 50 of the sequences listed in Table 8, for example at least 40 of the genes listed in Table 2, such as at least 50 of the genes listed in Table 3, such as at least 50 of the genes listed in Table 4, such as at least 50 of the genes listed in Table 6, at least 10 of the hemorrhagic stroke-related molecules listed in Table 7, at least 4 of the hemorrhagic stroke-related molecules listed in Table 15, or at least 10 of the hemorrhagic stroke-related molecules listed in Table 16. In some examples, detecting differential expression of at least four hemorrhagic stroke-related molecules involves quantitatively or qualitatively analyzing a DNA, mRNA, cDNA, protein, or combinations thereof.

If differential expression is detected in at least four, at least 5, at least 18, at least 25, at least 30, at least 119, at least 316, at least 446, or at least 1263 hemorrhagic stroke-related molecules is identified, this indicates that the subject has experienced a hemorrhagic stroke (and not an ischemic stroke), and a treatment is selected to prevent or reduce brain damage or to provide protection from the onset of brain damage. Examples of such treatment include administration of a coagulant, an anti-hypertensive, an anti-seizure agent, or combinations thereof. A particular example includes administration of a coagulant to increase clotting of blood at the hemorrhage, alone or in combination with one or more agents that prevent further strokes, such as anti-hypertensive agents or anti-seizure agents. In particular examples, the level of expression of a protein in a subject can be appropriately increased or decreased by expressing in the subject a recombinant genetic construct that includes a promoter operably linked to a nucleic acid molecule, wherein the nucleic acid molecule includes at least 10 (such as at least 15, at least 20, or at least 25) consecutive nucleotides of a hemorrhagic stroke-related nucleic acid sequence (such as any of the sequences listed in Tables 2-8 and 15-16). Expression of the nucleic acid molecule will change expression of the hemorrhagic stroke-related protein. The nucleic acid molecule can be in an antisense orientation relative to the promoter (for example to decrease expression of a gene that is undesirably upregulated) or in sense orientation relative to the promoter (for example to increase expression of a gene that is undesirably downregulated). In some examples, the recombinant genetic construct expresses an ssRNA corresponding to a hemorrhagic stroke-related nucleic acid sequence, such as an siRNA (or other inhibitory RNA molecule that can be used to decrease expression of a hemorrhagic stroke-related molecule whose expression is undesirably increased).

In examples of the methods described herein, detecting differential expression of at least four hemorrhagic stroke-related molecules involves determining whether a gene expression profile from the subject indicates development or progression of brain injury.

In particular examples, the disclosed methods are performed following the onset of signs and symptoms associated with hemorrhagic stroke. Examples of such symptoms include, but are not limited to headache, sensory loss (such as numbness, particularly confined to one side of the body or face), paralysis (such as hemiparesis), pupillary changes, blindness (including bilateral blindness), ataxia, memory impairment, dysarthria, somnolence, and other effects on the central nervous system recognized by those of skill in the art. In particular examples, the method of evaluating a stroke is performed after a sufficient period of time for the differential regulation of the genes (or proteins) to occur, for example at least 24 hours after onset of the symptom or constellation of symptoms that have indicated a potential intracerebral hemorrhagic event. In other examples, the method is performed prior to performing any diagnostics imaging tests (such as those that can find anatomic evidence of hemorrhagic stroke). For example, it can be difficult to quickly obtain a brain scan of a subject using imaging modalities (such as CT and MRI) to detect hemorrhagic strokes. Hence the assay described herein is able to detect the stroke even before definitive brain imaging evidence of the stroke is known.

The neurological sequalae of a hemorrhagic event in the central nervous system can have consequences that range from the insignificant to devastating, and the disclosed assays permit early and accurate stratification of risk of long-lasting neurological impairment. For example, a test performed as early as within the first 24 hours of onset of signs and symptoms of a stroke, and even as late as 2-11 or 7-14 days or even as late as 90 days or more after the event can provide clinical data that is highly predictive of the eventual care needs of the subject.

The disclosed assay is also able to identify subjects who have had a hemorrhagic stroke in the past, for example more than 2 weeks ago or even more than 90 days ago. The identification of such subjects helps evaluate other clinical data (such as neurological impairment or brain imaging information) to determine whether a hemorrhagic stroke has occurred.

In particular examples, the disclosed methods provide a lower cost alternative to expensive imaging modalities (such as MRI and CT scans), can be used in instances where those imaging modalities are not available (such as in field hospitals), can be more convenient than placing people in scanners (especially considering that some people are not able to fit in the scanner, or can not be subjected to MRI if they have certain types of metallic implants in their bodies), or combinations thereof.

### Clinical Specimens

Appropriate specimens for use with the current disclosure in diagnosing and prognosing hemorrhagic stroke include any conventional clinical samples, for instance blood or blood-fractions (such as serum). Techniques for acquisition of such samples are well known in the art (for example see Schluger et al. J. Exp. Med. 176:1327-33, 1992, for the collection of serum samples). Serum or other blood fractions can be prepared in the conventional manner. For example, about 200 μL of serum can be used for the extraction of DNA for use in amplification reactions. However, if DNA is not amplified, larger amounts of blood can be collected. For example, if at least 5 μg of mRNA is desired, about 20-30 mls of blood can be collected.

In one example, PBMCs are used as a source of isolated nucleic acid molecules or proteins. Substantially purified or isolated PBMCs are those that have been separated, for example, from other leukocytes in the blood. One advantage of using blood (for example instead of brain tissue) is that it is easily available can be drawn serially. In a particular example, PBMCs are isolated from a subject suspected of having had a hemorrhagic stroke, or known to have had a hemorrhagic stroke, such as an intracerebral hemorrhagic stroke. If needed, control PBMCs can be obtained from a subject who has not had a stroke, or has had an ischemic stroke.

Once a sample has been obtained, the sample can be used directly, concentrated (for example by centrifugation or filtration), purified, amplified, or combinations thereof. For example, rapid DNA preparation can be performed using a commercially available kit (such as the InstaGene Matrix, BioRad, Hercules, Calif.; the NucliSens isolation kit, Organon Teknika, Netherlands. In one example, the DNA preparation method yields a nucleotide preparation that is accessible to, and amenable to, nucleic acid amplification. Similarly, RNA can be prepared using a commercially available kit (such as the RNeasy Mini Kit, Qiagen, Valencia, Calif.).

In particular examples, proteins or nucleic acid molecules isolated from PBMCs are contacted with or applied to a hemorrhagic stroke detection array.

### Arrays for Detecting Nucleic Acid and Protein Sequences

In particular examples, methods for detecting a change in expression in the disclosed hemorrhagic stroke-related molecules listed in Tables 2-8 and 15-16 use the arrays disclosed herein. Arrays can be used to detect the presence of sequences whose expression is upregulated or downregulated in response to a hemorrhagic stroke, such as sequences listed in Tables 2-8 and 15-16, for example using specific oligonucleotide probes or antibody probes. The arrays herein termed “hemorrhagic stroke detection arrays,” are used to evaluate a stroke, for example to determine whether a subject has had a hemorrhagic stroke (such as an intracerebral hemorrhagic stroke), determine the severity of the stroke, predict the likelihood of neurological recovery of a subject who has had a hemorrhagic stroke, to identify an appropriate therapy for a subject who has had a hemorrhagic stroke, or combinations thereof. In particular examples, the disclosed arrays can include nucleic acid molecules, such as DNA or RNA molecules, or antibodies.

### Nucleic Acid Arrays

In one example, the array includes nucleic acid oligonucleotide probes that can hybridize to nucleic acid molecules (such as gene, cDNA or mRNA sequences). For example, the array can consist or consist essentially of any combination of probes that specifically bind to or hybridize to at least four of the hemorrhagic stroke-related sequences listed in Tables 2-8 and 15-16, such as at least 10, at least 20, at least 25, at least 30, at least 50, at least 100, at least 119, at least 140, at least 180, at least 200, at least 300, at least 316, at least 446, at least 500, at least 1000, or at least 1263 of the genes listed in any of Tables 2-8 and 15-16, such as at least 25 of the hemorrhagic stroke-related gene sequences listed in Table 2, at least 100 of the genes listed in Table 3, at least 20 of the genes listed in Table 4, at least 10 of the genes listed in Table 5, at least 50 of the genes listed in Table 6, at least 10 of the genes listed in Table 7, at least 4 of the genes listed in Table 15, or at least 10 of the genes listed in Table 16. In particular examples, an array comprises, consists essentially of, or consists of, oligonucleotides that can recognize all 47 hemorrhagic stroke-associated genes listed in Table 2, all 1263 of the hemorrhagic stroke-related genes listed in Table 3, all 119 of the hemorrhagic stroke-related genes listed in Table 4, all 30 of the hemorrhagic stroke-related genes listed in Table 5, all 446 of the hemorrhagic stroke-related genes listed in Table 6, all 25 of the hemorrhagic stroke-related genes listed in Table 7, all 316 of the hemorrhagic stroke-related genes listed in Table 8, all 5 of the hemorrhagic stroke-related genes listed in Table 15, all 18 of the hemorrhagic stroke-related genes listed in Table 16, or combinations thereof. Certain of such arrays (as well as the methods described herein) can include hemorrhagic stroke-related molecules that are not listed in Tables 2-8 and 15-16. In some examples, the array includes one or more probes that serve as controls. An array that consists essentially of probes that can hybridize to the listed hemorrhagic stroke-related genes includes control probes, such as 1-50 control probes (for example 1-20 or 1-10 control probes), ischemic stroke probes (such as at least four of those in Tables 17-18, for example probes that recognize all molecules listed in Tables 17-18), stroke diagnostic probes (such as at least 4 of those listed in Table 14, for example probes that recognize all molecules listed in Table 14), or combinations thereof.

In a specific example, an array includes, consists essentially of, or consists of oligonucleotide probes that can recognize at least IL1R2, haptoglobin, amphiphysin, TAP2, CD163, and granzyme M. For example, the array can include, consist essentially of, or consist of oligonucleotide probes that can recognize at least 1, at least 2, at least 3, at least 4, at least 5 or at least 6 of the following: IL1R2, haptoglobin, amphiphysin, TAP2, CD163, and granzyme M. For example, if the array includes probes that recognize 1-6 of these, in particular examples the array only further includes other hemorrhagic stroke-related sequences, and in some examples the array only further includes other hemorrhagic stroke-related sequences and probes that serve as controls.

In another specific example, an array includes, consists essentially of, or consists of oligonucleotide probes that can recognize at least one gene involved in the acute inflammatory response, at least one gene involved in cell adhesion, at least one gene involved in suppression of the immune response, at least one gene involved in hypoxia, at least one gene involved in vascular repair, at least one gene involved in the response to the altered cerebral microenvironment, and at least one gene involved in signal transduction, or at least 2, at least 3, at least 5, or at least 10 genes from each of these families.

In one example, a set of oligonucleotide probes is attached to the surface of a solid support for use in detection of hemorrhagic stroke-associated sequences, such as those nucleic acid sequences (such as cDNA or mRNA) obtained from the subject. Additionally, if an internal control nucleic acid sequence is used (such as a nucleic acid sequence obtained from a PBMC from a subject who has not had a hemorrhagic stroke or a nucleic acid sequence obtained from a PBMC from a subject who has had an ischemic stroke) an oligonucleotide probe can be included to detect the presence of this control nucleic acid molecule.

The oligonucleotide probes bound to the array can specifically bind sequences obtained from the subject, or amplified from the subject (such as under high stringency conditions). Thus, sequences of use with the method are oligonucleotide probes that recognize hemorrhagic stroke-related sequences, such as gene sequences (or corresponding proteins) listed in Tables 2-8 and 15-16. Such sequences can be determined by examining the hemorrhagic stroke-related sequences, and choosing oligonucleotide sequences that specifically anneal to a particular hemorrhagic stroke-related sequence (such as those listed in Tables 2-8 and 15-16 or represented by those listed in Tables 2-8 and 15-16), but not others. One of skill in the art can identify other hemorrhagic stroke-associated oligonucleotide molecules that can be attached to the surface of a solid support for the detection of other hemorrhagic stroke-associated nucleic acid sequences.

The methods and apparatus in accordance with the present disclosure takes advantage of the fact that under appropriate conditions oligonucleotides form base-paired duplexes with nucleic acid molecules that have a complementary base sequence. The stability of the duplex is dependent on a number of factors, including the length of the oligonucleotides, the base composition, and the composition of the solution in which hybridization is effected. The effects of base composition on duplex stability can be reduced by carrying out the hybridization in particular solutions, for example in the presence of high concentrations of tertiary or quaternary amines.

The thermal stability of the duplex is also dependent on the degree of sequence similarity between the sequences. By carrying out the hybridization at temperatures close to the anticipated Tₘ's of the type of duplexes expected to be formed between the target sequences and the oligonucleotides bound to the array, the rate of formation of mis-matched duplexes may be substantially reduced.

The length of each oligonucleotide sequence employed in the array can be selected to optimize binding of target hemorrhagic stroke-associated nucleic acid sequences. An optimum length for use with a particular hemorrhagic stroke-associated nucleic acid sequence under specific screening conditions can be determined empirically. Thus, the length for each individual element of the set of oligonucleotide sequences including in the array can be optimized for screening. In one example, oligonucleotide probes are from about 20 to about 35 nucleotides in length or about 25 to about 40 nucleotides in length.

The oligonucleotide probe sequences forming the array can be directly linked to the support. Alternatively, the oligonucleotide probes can be attached to the support by non-hemorrhagic stroke-associated sequences such as oligonucleotides or other molecules that serve as spacers or linkers to the solid support.

### Protein Arrays

In another example, an array includes, consists essentially of, or consists of protein sequences (or a fragment of such proteins, or antibodies specific to such proteins or protein fragments) that can specifically bind to at least four of the hemorrhagic stroke-related protein sequences listed in 2-8 and 15-16, such as at least 25 of the hemorrhagic stroke-related protein sequences listed in Table 2, at least 100 of the proteins listed in Table 3, at least 20 of the proteins listed in Table 4, at least 10 of the proteins listed in Table 5, at least 50 of the proteins listed in Table 6, at least 10 of the proteins listed in Table 7, at least 4 of the proteins listed in Table 15, or at least 10 of the proteins listed in Table 16. In particular examples, an array comprises, consists essentially of, or consists of, proteins that can recognize all 47 hemorrhagic stroke-associated proteins listed in Table 2, all 1263 of the hemorrhagic stroke-related proteins listed in Table 3, all 119 of the hemorrhagic stroke-related proteins listed in Table 4, all 30 of the hemorrhagic stroke-related proteins listed in Table 5, all 446 of the hemorrhagic stroke-related proteins listed in Table 6, all 25 of the hemorrhagic stroke-related proteins listed in Table 7, all 316 of the hemorrhagic stroke-related proteins listed in Table 8, all 5 of the hemorrhagic stroke-related proteins listed in Table 15, all 18 of the hemorrhagic stroke-related proteins listed in Table 16, or combinations thereof. Such arrays can also comprise, consist essentially of, or consist of any particular subset of the proteins listed in Tables 2-8 and 15-16. For example, an array can include probes that can recognize at least one protein involved in the acute inflammatory response, at least one protein involved in cell adhesion, at least one protein involved in suppression of the immune response, at least one protein involved in hypoxia, at least one protein involved in vascular repair, at least one gene involved in the response to the altered cerebral microenvironment, and at least one gene involved in signal transduction, or at least 2, at least 3, at least 5, or at least 10 proteins from each of these families. In another specific example, the array includes protein probes that recognize one or more of the following proteins: IL1R2, haptoglobin, amphiphysin, TAP2, CD163, Sema4C, or granzyme M. For example, the array can include a protein probe that recognizes IL1R2 and additional probes that recognize other hemorrhagic stroke-related proteins (such as any combination of at least 3 or at least 25 of those listed in Tables 2-8 and 15-16). For example, if the array includes probes that recognize these, in particular examples the array only further includes other hemorrhagic stroke-related proteins, and in some examples the array only further includes other hemorrhagic stroke-related proteins and probes that serve as controls. An array that consists essentially of probes that can detect the listed hemorrhagic stroke-related proteins, further includes control probes, such as 1-50 control probes (for example 1-20 or 1-10 control probes).

The proteins or antibodies forming the array can be directly linked to the support. Alternatively, the proteins or antibodies can be attached to the support by spacers or linkers to the solid support.

Changes in expression of hemorrhagic stroke-related proteins can be detected using, for instance, a hemorrhagic stroke protein-specific binding agent, which in some instances is labeled with an agent that can be detected. In certain examples, detecting a change in protein expression includes contacting a protein sample obtained from the PBMCs of a subject with a hemorrhagic stroke protein-specific binding agent (which can be for example present on an array); and detecting whether the binding agent is bound by the sample and thereby measuring the levels of the hemorrhagic stroke-related protein present in the sample. A difference in the level of at least four hemorrhagic stroke-related proteins in the sample, relative to the level of the hemorrhagic stroke-related proteins found an analogous sample from a subject who has not had a hemorrhagic stroke, in particular examples indicates that the subject has suffered a hemorrhagic stroke.

### Array Substrate

The solid support can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluoride, polyfluoroethylene-propylene, polyethylenevinyl alcohol, polymethylpentene, polychlorotrifluoroethylene, polysulformes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567).

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides or proteins (such as antibodies) are not amenable to non-specific binding, or when non-specific binding occurs, such materials can be readily removed from the surface without removing the oligonucleotides or proteins (such as antibodies).

In one example, the solid support surface is polypropylene. Polypropylene is chemically inert and hydrophobic. Non-specific binding is generally avoidable, and detection sensitivity is improved. Polypropylene has good chemical resistance to a variety of organic acids (such as formic acid), organic agents (such as acetone or ethanol), bases (such as sodium hydroxide), salts (such as sodium chloride), oxidizing agents (such as peracetic acid), and mineral acids (such as hydrochloric acid). Polypropylene also provides a low fluorescence background, which minimizes background interference and increases the sensitivity of the signal of interest.

In another example, a surface activated organic polymer is used as the solid support surface. One example of a surface activated organic polymer is a polypropylene material aminated via radio frequency plasma discharge. Such materials are easily utilized for the attachment of nucleotide molecules. The amine groups on the activated organic polymers are reactive with nucleotide molecules such that the nucleotide molecules can be bound to the polymers. Other reactive groups can also be used, such as carboxylated, hydroxylated, thiolated, or active ester groups.

### Array Formats

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit a low background fluorescence.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as see PCT applications WO 85/01051 and WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second (2° set of rows that are now perpendicular to the first set. This process creates a multiple-channel array whose intersection generates a plurality of discrete cells.

The oligonucleotides can be bound to the polypropylene support by either the 3′ end of the oligonucleotide or by the 5′ end of the oligonucleotide. In one example, the oligonucleotides are bound to the solid support by the 3′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support.

In particular examples, the oligonucleotide probes on the array include one or more labels, that permit detection of oligonucleotide probe:target sequence hybridization complexes.

### Detection of Nucleic Acid and Protein Molecules

The nucleic acid molecules and proteins obtained from the subject (for example from PBMCs) can contain altered levels of one or more genes associated with hemorrhagic stroke, such as those listed in Tables 2-8 and 15-16. Changes in expression can be detected to evaluate a stroke, or example to determine if the subject has had a hemorrhagic stroke, to determine the severity of the stroke, to determine the likelihood of neurological recovery of a subject who has had a hemorrhagic stroke, to determine the appropriate therapy for a subject who has had a hemorrhagic stroke, or combinations thereof. The present disclosure is not limited to particular methods of detection. Any method of detecting a nucleic acid molecule or protein can be used, such as physical or functional assays. For example, the level of gene activation can be quantitated utilizing methods well known in the art and those disclosed herein, such as Northern-Blots, RNase protection assays, nucleic acid or antibody probe arrays, quantitative PCR (such as TaqMan assays), dot blot assays, in-situ hybridization, or combinations thereof. In addition, proteins can be quantitated using antibody probe arrays, quantitative spectroscopic methods (for example mass spectrometry, such as surface-enhanced laser desorption/ionization (SELDI)-based mass spectrometry), or combinations thereof.

Methods for labeling nucleic acid molecules and proteins so that they can be detected are well known. Examples of such labels include non-radiolabels and radiolabels. Non-radiolabels include, but are not limited to enzymes, chemiluminescent compounds, fluorophores, metal complexes, haptens, colorimetric agents, dyes, or combinations thereof. Radiolabels include, but are not limited to, ³H, ¹²⁵I and ³⁵S. Radioactive and fluorescent labeling methods, as well as other methods known in the art, are suitable for use with the present disclosure. In one example, the primers used to amplify the subject's nucleic acids are labeled (such as with biotin, a radiolabel, or a fluorophore). In another example, the amplified nucleic acid samples are end-labeled to form labeled amplified material. For example, amplified nucleic acid molecules can be labeled by including labeled nucleotides in the amplification reactions. In another example, nucleic acid molecules obtained from a subject are labeled, and applied to an array containing oligonucleotides. In a particular example, proteins obtained from a subject are labeled and subsequently analyzed, for example by applying them to an array.

In one example, nucleic acid molecules obtained from the subject that include those molecules associated with hemorrhagic stroke are applied to an hemorrhagic stroke detection array for time sufficient and under conditions (such as very high stringency or high stringency hybridization conditions) sufficient to allow hybridization between the isolated nucleic acid molecules and the probes on the array, thereby forming a hybridization complex of isolated nucleic acid molecule:oligonucleotide probe. In particular examples, the isolated nucleic acid molecules or the oligonucleotide probes (or both) include a label. In one example, a pre-treatment solution of organic compounds, solutions that include organic compounds, or hot water, can be applied before hybridization (see U.S. Pat. No. 5,985,567).

Hybridization conditions for a given combination of array and target material can be optimized routinely in an empirical manner close to the Tₘ of the expected duplexes, thereby maximizing the discriminating power of the method. Identification of the location in the array, such as a cell, in which binding occurs, permits a rapid and accurate identification of sequences associated with hemorrhagic stroke present in the amplified material (see below).

The hybridization conditions are selected to permit discrimination between matched and mismatched oligonucleotides. Hybridization conditions can be chosen to correspond to those known to be suitable in standard procedures for hybridization to filters and then optimized for use with the arrays of the disclosure. For example, conditions suitable for hybridization of one type of target would be adjusted for the use of other targets for the array. In particular, temperature is controlled to substantially eliminate formation of duplexes between sequences other than exactly complementary hemorrhagic stroke-associated wild-type of mutant sequences. A variety of known hybridization solvents can be employed, the choice being dependent on considerations known to one of skill in the art (see U.S. Pat. No. 5,981,185).

Once the nucleic acid molecules associated with hemorrhagic stroke from the subject have been hybridized with the oligonucleotides present in the hemorrhagic stroke detection array, the presence of the hybridization complex can be analyzed, for example by detecting the complexes. For example the complexes can be detected to determine if there are changes in gene expression (such as increases or decreases), such as changes in expression of any combination of four or more of the genes listed in Tables 2-8 and 15-16, such as 20 or more of the genes listed in Tables 2-8 and 15-16, or such as 150 or more of the genes listed in Tables 2-8 and 15-16. In particular examples, changes in gene expression are quantitated, for instance by determining the amount of hybridization. In particular examples, the hybridization complexes formed are compared to hybridization complexes formed by a control, such as complexes formed between nucleic acid molecules isolated from a subject who has had an ischemic stroke, has had no stroke, or both, and the probes on the hemorrhagic stroke detection array.

The presence of increased expression of four or more genes listed in Tables 2-8 and 15-16 with a positive t-statistic value (such as a t-statistic value of at least 3) or positive FC value (such as at least 1.2), or decreased expression of four or more genes listed in Tables 2-8 and 16 with a negative t-statistic value (such as a t-statistic value of no more than −3) or negative FC value (such as less than −1.2), or any combination thereof, such as decreased expression of at least one gene and increased expression of at least 3 genes listed in Tables 2-8 or 15-16, after multiple comparison correction, indicates that the subject has had a hemorrhagic stroke (such as an ICH). In particular examples, the intensity of the t-value can indicate the severity of the hemorrhagic stroke. For example, detection of a t-statistic of 19 for IL1R2 as compared to detection of a t-statistic of 3 for IL1R2 indicates a more severe stroke.

Detecting a hybridized complex in an array of oligonucleotide probes has been previously described (see U.S. Pat. No. 5,985,567). In one example, detection includes detecting one or more labels present on the oligonucleotides, the sequences obtained from the subject, or both. In particular examples, developing includes applying a buffer. In one example, the buffer is sodium saline citrate, sodium saline phosphate, tetramethylammonium chloride, sodium saline citrate in ethylenediaminetetra-acetic, sodium saline citrate in sodium dodecyl sulfate, sodium saline phosphate in ethylenediaminetetra-acetic, sodium saline phosphate in sodium dodecyl sulfate, tetramethylammonium chloride in ethylenediaminetetra-acetic, tetramethylammonium chloride in sodium dodecyl sulfate, or combinations thereof. However, other suitable buffer solutions can also be used.

Detection can further include treating the hybridized complex with a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or calorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera (manufactured by Photometrics, Inc. of Tucson, Ariz.). In particular examples, these steps are not performed when fluorophores or radiolabels are used.

Similar methods can be used to detect and analyze complexes formed between antibodies on an array and hemorrhagic stroke proteins. Hemorrhagic stroke proteins obtained from the subject (for example from PBMCs) are applied to an hemorrhagic stroke detection array for time sufficient and under conditions sufficient to allow specific binding between the isolated proteins and the antibody probes on the array, thereby forming a complex of isolated protein:antibody probe. In particular examples, the isolated proteins or the probes (or both) include a label. In one example, a pre-treatment solution of organic compounds, solutions that include organic compounds, or hot water, can be applied before hybridization (see U.S. Pat. No. 5,985,567). Identification of the location in the array, such as a cell, in which binding occurs, permits a rapid and accurate identification of sequences associated with hemorrhagic stroke present in the amplified material.

Once the proteins associated with hemorrhagic stroke from the subject bind to the antibody (or other probe) present in the hemorrhagic stroke detection array, the presence of the complex can be analyzed, for example by detecting the complexes. For example the complexes can be detected to determine if there are changes in gene expression (such as increases or decreases), such as changes in expression of any combination of four or more of the proteins listed in Tables 2-8 and 15-16, such as 20 or more of the proteins listed in Tables 2-8 and 15-16, or such as 150 or more of the proteins listed in Tables 2-8 and 15-16. In particular examples, changes in protein expression are quantitated, for instance by determining the amount of binding. In particular examples, the complexes formed are compared to complexes formed by a control, such as complexes formed between proteins isolated from a subject who has had an ischemic stroke, has had no stroke, or both, and the probes on the hemorrhagic stroke detection array.

The presence of increased expression of four or more proteins listed in Tables 2-4 or 6-7 with a positive t-statistic value (such as a t-statistic value of at least 3 or at least 6) or listed in Table 15 or 16 with a positive FC value, or decreased expression of four or more genes listed in Tables 2-4 or 6-7 with a negative t-statistic value (such as a t-statistic value of no more than −3 such as no more than −6) or listed in Table 16 with a negative FC value, or any combination thereof such as decreased expression of at least one gene and increased expression of at least 3 genes listed in Tables 2-4, 6-7 or 15-16, after multiple comparison correction, indicates that the subject has had a hemorrhagic stroke (such as an ICH). In particular examples, the intensity of the T-value can indicate the severity of the hemorrhagic stroke. For example, detection of a t-statistic of 15 for IL1R2 as compared to detection of a t-statistic of 5 for IL1R2, indicates a more severe stroke.

Detecting a hybridized complex in an array of antibody probes has been previously described (for example see Sanchez-Carbayo, Antibody Arrays: Technical Considerations And Clinical Applications in Cancer, Clin. Chem. 2006 Jun. 29). In one example, detection includes detecting one or more labels present on the antibodies, the proteins obtained from the subject, or both. In particular examples, developing includes applying a buffer. In one example, the buffer is sodium saline citrate, sodium saline phosphate, tetramethylammonium chloride, sodium saline citrate in ethylenediaminetetra-acetic, sodium saline citrate in sodium dodecyl sulfate, sodium saline phosphate in ethylenediaminetetra-acetic, sodium saline phosphate in sodium dodecyl sulfate, tetramethylammonium chloride in ethylenediaminetetra-acetic, tetramethylammonium chloride in sodium dodecyl sulfate, or combinations thereof. However, other suitable buffer solutions can also be used.

### Kits

The present disclosure provides for kits that can be used to evaluate a stroke, for example to determine if a subject has had a hemorrhagic stroke (such as an intracerebral hemorrhagic stroke), to determine the severity of the stroke, to determine the likelihood of neurological recovery of a subject who has had a hemorrhagic stroke, to determine the appropriate therapy for a subject who has had a hemorrhagic stroke, or combinations thereof. Such kits allow one to determine if a subject has a differential expression in hemorrhagic stroke-related genes, such as any combination of four or more of those listed in Tables 2-8 and 15-16, such as any combination of 10 or more of those listed in Tables 2-8 and 15-16, or any combination of 50 or more of those listed in Tables 2-8 and 15-16, for example any combination of at least one gene from each of the following classes of genes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction (such as at least 2 or at least 3 genes from each gene class).

In particular examples, the disclosed kits include one or more of the disclosed arrays. For example, the kits can include a binding molecule, such as an oligonucleotide probe that selectively hybridizes to a hemorrhagic stroke-related molecule that is the target of the kit. In particular examples, the oligonucleotides probes are attached to an array. In one example, the kit includes oligonucleotide probes or primers (or antibodies) that recognize any combination of at least four of the molecules in Table 5 or 8, such as at least 5, at least 10, at least 15, at least 20, at least 50, at least 60, at least 100, at least 119, at least 150, at least 170, at least 175, at least 180, at least 185, at least 200, at least 316, at least 446, at least 500, at least 525, at least 550, at least 1000, or at least 1263 of the sequences listed in any of Tables 2-8 and 15-16. In particular examples, the kit includes oligonucleotide probes or primers (or antibodies) that recognize at least one gene (or protein) from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction, such as at least 2, at least 3, at least 5, or at least 10 genes from each class.

In one particular example, the kit includes oligonucleotide probes or primers (or antibodies) that recognize at least IL1R2, CD163, amphiphysin, and TAP2. In one particular example, the kit includes oligonucleotide probes or primers (or antibodies) that recognize at least 1, at least 2, at least 3, or at least 4, of IL1R2, CD163, amphiphysin, and TAP2, and can further include oligonucleotide probes or primers (or antibodies) that recognize haptoglobin, granzyme M or Sema4C. In another particular example, the kit includes oligonucleotide probes or primers (or antibodies) that recognize IL1R2, for example in combination with oligonucleotide probes or primers (or antibodies) that recognize any combination of at least three hemorrhagic stroke related molecules listed in Tables 2-8 and 15-16.

In a particular example, kits include antibodies capable of binding to hemorrhagic stroke-related proteins. Such antibodies can be present on an array.

In particular examples, the kit further includes an array for diagnosis of stroke, such as an array that consists essentially of or consists of at least four probes specific for the molecules listed in Table 14 (such as all the molecules listed in Table 14). In some examples, the kit further includes an array for classification of ischemic stroke, such as an array that consists essentially of or consists of at least 4 probes specific for the molecules listed in Tables 17 and 18 (such as all the molecules listed in Tables 17 and 18). An array that “consists essentially of” particular probes can further include control probes (such as 1-10 or 1-50 control probes), but not other probes.

The kit can further include one or more of a buffer solution, a conjugating solution for developing the signal of interest, or a detection reagent for detecting the signal of interest, each in separate packaging, such as a container. In another example, the kit includes a plurality of hemorrhagic stroke-related target nucleic acid sequences for hybridization with a hemorrhagic stroke detection array to serve as positive control. The target nucleic acid sequences can include oligonucleotides such as DNA, RNA, and peptide-nucleic acid, or can include PCR fragments.

### Hemorrhagic Stroke Therapy

The present disclosure also provides methods of reducing brain injury in a subject determined to have suffered a hemorrhagic stroke, such as an intracerebral hemorrhagic stroke. For example, if using the assays described above a change in expression in at least four of the hemorrhagic stroke-related molecules listed in Tables 2-8 and 15-16 is detected in the subject, for example at least five of the hemorrhagic stroke-related molecules listed in Tables 5 or 8 is detected in the subject, a treatment is selected to avoid or reduce brain injury or to delay the onset of brain injury. In another example, if using the screening methods described above a change in expression in at least 50 of the hemorrhagic stroke-related molecules listed in any of Tables 2-8 and 15-16 is detected in the subject, a treatment is selected to avoid or reduce brain injury or to delay the onset of brain injury. The subject then can be treated in accordance with this selection, for example by administration of agents that increase blood clotting, reduce blood pressure, reduce intracerebral pressure, reduce brain swelling, reduce seizures, or combinations thereof. Particular examples of such agents include one or more coagulants, one or more anti-hypertensives, or combinations thereof. In some examples, the treatment selected is specific and tailored for the subject, based on the analysis of that subject's profile for one or more hemorrhagic stroke-related molecules.

### Screening Test Agents

Based on the identification of multiple hemorrhagic stroke-related molecules whose expression is altered following a hemorrhagic stroke (such as those listed in Tables 2-8 and 15-16), the disclosure provides methods for identifying agents that can enhance, normalize, or reverse these effects. For example, the method permits identification of agents that normalize activity of a hemorrhagic stroke-related molecule, such as a gene (or its corresponding protein) involved in suppression of the immune response, anaerobic metabolism, vascular repair, calcium-binding proteins, and ubiquitin-related genes, or combinations thereof. Normalizing activity (such as the expression) of a hemorrhagic stroke-related molecule can include decreasing activity of a hemorrhagic stroke-related molecule whose activity is increased following a hemorrhagic stroke, or increasing activity of a hemorrhagic stroke-related molecule whose activity is decreased following a hemorrhagic stroke. In another example, the method permits identification of agents that enhance the activity of a hemorrhagic stroke-related molecule, such as a hemorrhagic stroke-related molecule whose activity provides a protective effect to the subject following a hemorrhagic stroke. For example, the method permits identification of agonists. In yet another example, the method permits identification of agents that decrease the activity of a hemorrhagic stroke-related molecule, such as a hemorrhagic stroke-related molecule whose activity results in one or more negative symptoms of hemorrhagic stroke. For example, the method permits identification of antagonists.

In particular examples the identified agents can be used to treat a subject who has had a hemorrhagic stroke (such as an intracerebral hemorrhagic stroke), for example to alleviate or prevent one or more symptoms of a hemorrhagic stroke, such as paralysis or memory loss.

The disclosed methods can be performed in vitro, for example by adding the test agent to cells in culture, or in vivo, for example by administering the test agent to a mammal (such as a human or a laboratory animal, for example a mouse, rat, dog, or rabbit). In particular examples, the method includes exposing the cell or mammal to conditions sufficient for mimicking a hemorrhagic stroke. The one or more test agents are added to the cell culture or administered to the mammal under conditions sufficient to alter the activity of one or more hemorrhagic stroke-related molecules, such as at least one of the molecules listed in Tables 2-8 and 15-16. Subsequently, the activity of the hemorrhagic stroke-related molecule is determined, for example by measuring expression of one or more hemorrhagic stroke-related molecules or by measuring an amount of biological activity of one or more hemorrhagic stroke-related proteins. A change in the activity one or more hemorrhagic stroke-related molecule indicates that the test agent alters the activity of a hemorrhagic stroke-related molecule listed in Tables 2-8 and 15-16. In particular examples, the change in activity is determined by a comparison to a standard, such as an amount of activity present when no hemorrhagic stroke has occurred, or an amount of activity present when a hemorrhagic stroke has occurred, or to a control.

Any suitable compound or composition can be used as a test agent, such as organic or inorganic chemicals, including aromatics, fatty acids, and carbohydrates; peptides, including monoclonal antibodies, polyclonal antibodies, and other specific binding agents; phosphopeptides; or nucleic acid molecules. In a particular example, the test agent includes a random peptide library (for example see Lam et al., Nature 354:82-4, 1991), random or partially degenerate, directed phosphopeptide libraries (for example see Songyang et al., Cell 72:767-78, 1993). A test agent can also include a complex mixture or “cocktail” of molecules.

Therapeutic agents identified with the disclosed approaches can be used as lead compounds to identify other agents having even greater desired activity. In addition, chemical analogs of identified chemical entities, or variants, fragments, or fusions of peptide test agents, can be tested for their ability to alter activity of a hemorrhagic stroke-related molecule using the disclosed assays. Candidate agents can be tested for safety in animals and then used for clinical trials in animals or humans.

### In Vivo Assays

In one example, the method is an in vivo assay. For example, agents identified as candidates in an in vitro assay can be tested in vivo for their ability to alter (such as normalize) the activity of a hemorrhagic stroke-related molecule (such as one or more of those listed in Tables 2-8 and 15-16). In particular examples, the mammal has had a hemorrhagic stroke or has been exposed to conditions that induce a hemorrhagic stroke. Simultaneously or at a time thereafter, one or more test agents are administered to the subject under conditions sufficient for the test agent to have the desired effect on the subject, for example to alter (such as normalize) the activity of a hemorrhagic stroke-related molecule or a pattern of hemorrhagic stroke-related molecules. In particular examples, the test agent has the desired effect on more than one hemorrhagic stroke-related molecule.

Methods of providing conditions sufficient for inducing an ischemic stroke in vivo are known in the art. For example, hemorrhagic stroke can be induced in a mammal by administration of autologous blood or other agents (such as type IV bacterial collagenase), for example administration to the basal ganglia (such as the striatum).

One or more test agents are administered to the subject under conditions sufficient for the test agent to have the desired effect on the subject. Any appropriate method of administration can be used, such as intravenous, intramuscular, intraperitoneal, or transdermal. The agent can be administered at a time subsequent to the hemorrhagic stroke, or at substantially the same time as the hemorrhagic stroke. In one example, the agent is added at least 30 minutes after the hemorrhagic stroke, such as at least 1 hour, at least 2 hours, at least 6 hours, at least 24 hours, at least 72 hours, at least 7 days, at least 14 days, at least 30 days, at least 60 days or even at least 90 days after the hemorrhagic stroke.

### Detecting Expression

The effect on the one or more test agents on the activity of one or more hemorrhagic stroke-related molecules can be determined using methods known in the art. For example, the effect on expression of one or more hemorrhagic stroke-related genes can be determined using the arrays and methods disclosed herein. For example, RNA can be isolated from cells obtained from a subject (such as PBMCs) administered the test agent. The isolated RNA can be labeled and exposed to an array containing one or more nucleic acid molecules (such as a primer or probe) that can specifically hybridize to one or more pre-selected hemorrhagic stroke-related genes, such at least 1, at least 2, or at least 3 of those listed in Tables 2-8 and 15-16, or to a pre-selected pattern of such genes that is associated with hemorrhagic stroke. In a particular example, the one or more pre-selected hemorrhagic stroke-related genes include at least one gene involved in acute inflammatory response, at least one gene involved in cell adhesion, at least one gene involved in suppression of the immune response, at least one gene involved in hypoxia, at least one gene involved in hematoma/vascular repair, at least one gene involved in the response to altered cerebral microenvironment and at least one gene involved in signal transduction, or combinations thereof. In another example, proteins are isolated from the cultured cells exposed to the test agent, or from cells obtained from a subject (such as PBMCs) administered the test agent. The isolated proteins can be analyzed to determine amounts of expression or biological activity of one or more hemorrhagic stroke-related proteins, such at least 1, at least 2, or at least 3 of those listed in Tables 2-8 and 15-16, or a pattern of upregulation or downregulation of pre-identified or pre-selected proteins. In a particular example, the one or more pre-selected hemorrhagic stroke-related proteins include at least one involved in acute inflammatory response, at least one protein involved in cell adhesion, at least one protein involved in suppression of the immune response, at least one protein involved in hypoxia, at least one protein involved in hematoma/vascular repair, at least one protein involved in the response to altered cerebral microenvironment and at least one protein involved in signal transduction, or combinations thereof. In a particular example, mass spectrometry is used to analyze the proteins.

In particular examples, differential expression of a hemorrhagic stroke-related molecule is compared to a standard or a control. One example of a control includes the amount of activity of a hemorrhagic stroke-related molecule present or expected in a subject who has not had a hemorrhagic stroke, wherein an increase or decrease in activity in a test sample of a hemorrhagic stroke-related molecule (such as those listed in Tables 2-8 and 15-16) compared to the control indicates that the test agent alters the activity of at least one hemorrhagic stroke-related molecule. Another example of a control includes the amount of activity of a hemorrhagic stroke-related molecule present or expected in a subject who has had a hemorrhagic stroke, wherein an increase or decrease in activity in a test sample (such as gene expression, amount of protein, or biological activity of a protein) of a hemorrhagic stroke-related molecule (such as those listed in Tables 2-8 and 15-16) compared to the control indicates that the test agent alters the activity of at least one hemorrhagic stroke-related molecule. Detecting differential expression can include measuring a change in gene expression, measuring an amount of protein, or determining an amount of the biological activity of a protein present.

In particular examples, test agents that altered the activity of a hemorrhagic stroke-related molecule are selected.

The disclosure is further illustrated by the following non-limiting Examples.

### Example 1

### Isolation of Samples

This example describes methods used to obtain RNA from PBMCs. Subjects included eight who had an acute intracerebral hemorrhage within the previous 72 hours and up to 5 days (confirmed ICH on neuroimaging studies), 19 who had an acute ischemic stroke (IS) within the previous 72 hours, and 20 control subjects (subjects who had not previously had a stroke). The subjects were reasonably comparable in terms of age, sex and pre-morbid risk factors consistent with a community based stroke population.

Eight patients with ICH were recruited from Suburban Hospital, Bethesda, Md. Inclusion criteria were age >21 years and willingness to participate in the study after informed consent was given. Exclusion criteria were cardiovascular instability, severe anemia (hemoglobin <8.0 g/dL), current infection and current severe allergic disorders. ICH was confirmed by neuroimaging studies, including computed tomography (CT) and/or magnetic resonance imaging (MRI) using gradient recalled echo (GRE) sequences. Included patients with ICH had confluent intracerebral hematomas on neuroimaging studies; those patients with hemorrhagic transformation of a cerebral infarct, traumatic ICH, microbleeds and non-acute ICH were excluded, which greatly reduced our number of ICH patients. Stroke severity was determined by serial neurological examinations and by the NIH Stroke Scale (NIHSS) score (see Brott et al., Stroke 20:871-5, 1989). Prior risk of stroke was estimated from the Framingham Stroke Profile (Wolf et al., Stroke 22:312-8, 1991), a composite score of age, history of hypertension, systolic blood pressure, smoking, cardiovascular disease, diabetes, atrial fibrillation, and left ventricular hypertrophy.

These 20 “normal” subjects were as similar in age and vascular risk factor profiles to the ICH patients as was feasibly possible. Subjects were >21 years of age and willing to participate in the study after informed consent was obtained. Exclusion criteria were active medical problems, current symptomatic infection, and current severe allergic disorders. Stroke risk factors were recorded according to the Framingham risk profile, as described above for the ICH patients.

The clinical and demographic details of the 8 patients with confirmed ICH on neuroimaging studies and the 18 referent subjects in the index cohort are shown in Table 1 (2 of the 20 referent subjects were not included due to poor signal from the array; discussed below). Continuous data are presented as means±SD. Categorical data are presented as numbers (%).

The causes of the ICHs were hypertension (n=4), amyloid angiopathy (n=2), dural arterio-venous fistula (n=1) and uncertain (n=1). The referent subjects were older than the patients with ICH, but not significantly. The groups had similar Framingham stroke risk scores. The referent subjects had a higher rate of statin use than the ICH patients (p=0.03). The two external test cohorts together consisted of 7 ICH patients and 10 referent control subjects.

TABLE 1
Demographics of test subjects

| | | | | | Test Cohort 1 | | | | | | | |
|---|---|---|---|---|---|---|---|---|---|---|---|---|
| | | | | | Classification of | | | | Test Cohort 2 | | | |
| | Index Cohort | | | | PAM list | | | | Real time PCR | | | |
| Factor | ICH | | Referent | | ICH | | Referent | | ICH | | Referent | |
| N | 8 | | 18 | | 4** | | 6 | | 5** | | 4 | |
| Age (years) | 69.1 | | 75.1 | | 79.3 | | 49.8 | | \hat{ }84.5 | | 49.5 | |
| Age range | 50-84 | | 62-84 | | 70-86 | | 33-58 | | 83-86 | | 32-58 | |
| Sex | 5 F, 3 M | | 10 F, 8 M | | 2 M, 2 F | | 3 M, 3 F | | 2 F, 3 M | | 2 F, 2 M | |
| Race | | | | | | | | | | | | |
| White | 7 | (88) | 13 | (72) | 4 | (100) | 5 | (83) | 5 | (100) | 3 | (75) |
| African American | 1 | (12) | 4 | (22) | 0 | (0) | 0 | (0) | 0 | (0) | 0 | (0) |
| Hispanic | 0 | (0) | 1 | (6) | 0 | (0) | 0 | (0) | 0 | (0) | 1 | (25) |
| Asian | 0 | (0) | 0 | (0) | 0 | (0) | 1 | (17) | 0 | (0) | 0 | (0) |
| Risk Factors | | | | | | | | | | | | |
| HT | 5 | (63) | 10 | | 4 | (100) | 1 | (17) | 2 | (100) | 3 | (75) |
| DM | 0 | (0) | 2 | | 0 | (0) | 0 | (0) | 0 | (0) | 2 | (50) |
| Smoking history | 3 | (38) | 11 | | 2 | (50) | 0 | (0) | 0 | (0) | 4 | (100) |
| CAD | 2 | (25) | 2 | | 1 | (25) | 0 | (0) | 1 | (50) | 0 | (0) |
| Hyperlipidemia | 4 | (50) | 13 | | 2 | (50) | 1 | (17) | 2 | (100) | 1 | (25) |
| Framingham | 10 | | 11.9 | | 16 | | 2 | | 17 | | 10 | |
| Score | | | | | | | | | | | | |
| Medications | | | | | | | | | | | | |
| Antiplatelet | \hat{ }\hat{ }\hat{ }3 | (60) | 12 | (67) | 2 | (50) | 0 | (0) | 0 | (0) | 2 | (50) |
| Coumadin | 0 | (0) | 0 | (0) | 1 | (25) | 0 | (0) | 0 | (0) | 0 | (0) |

| | | | | | | | | | | | |
|---|---|---|---|---|---|---|---|---|---|---|---|
| Statins | 1 | (20) | 11* | 2 | (50) | 1 | (17) | 1 | (50) | 0 | (0) |
| Antihypertensive | 3 | (60) | 11 | 3 | (75) | 1 | (17) | 2 | (100) | 2 | (50) |
| Stroke-Related | | | | | | | | | | | |

| | | | | | | |
|---|---|---|---|---|---|---|
| Time (hours) | 35.9 | N/A | 52.3 | N/A | \hat{ }\hat{ }52.8 | N/A |
| NIHSS | 12 | N/A | 7.5 | N/A | \hat{ }4 | N/A |
| Differential White | | | | | | |
| Blood Cell Counts | | | | | | |
| (Thou/μL) | | | | | | |
| Total | 10.0 | 5.7* | 6.6 | 4.6 | 6.8 | 5.8 |
| Neutrophils | 8.2 | 3.1* | 4.3 | 2.5 | 4.7 | 3.1 |
| Monocytes | 0.8 | 0.5 | 0.7 | 0.3 | 0.7 | 0.5 |
| Lymphoctyes | 1.4 | 1.8 | 1.5 | 1.7 | 1.4 | 2.1 |

Medications refer to medications taken prior to the stroke
*p < 0.05
\hat{ }incomplete clinical data in 3 subjects
\hat{ }\hat{ }in 2^{nd} test cohort, there were 8 time points tested after stroke in the 5 ICH subjects, ranging from 2 days until 11 days, time is for first blood draw in the 5 patients
\hat{ }\hat{ }\hat{ }medication data not available in 3 subjects
**two of these patients (ICH) were in the first test cohort and in test cohort 2

Approximately 30 milliliters of blood was drawn via aseptic antecubital fossa venipuncture into four yellow top ACD A tubes (ACD Acid citrate dextrose A, 22.0 g/L trisodium citrate, 8.0 g/L aitric acid, 24.5 g/L dextrose, BD Franklin Lakes, N.J.) by aseptic antecubital fossa venipuncture. In the ICH patients blood was drawn as early as possible after onset (depending on the patient's medical stability and after full and informed consent had been obtained); the times of blood draws were <24 hours (n=2), 24 to 48 hours (n=5), and >48 hours (n=1). Acute stroke patients underwent aseptic antebrachial venipuncture followed by withdrawal of 30 ml of blood as described above, within 5 days of stroke onset.

Total RNA (5 to 15 μg) was isolated from PBMCs within two hours of bloodcollection. PBMCs were separated from whole blood with a density gradient tube (Uni-Sep, Novamed, Jerusalem, Israel) as follows: 20 to 30 mL ACD anticoagulated blood was diluted with an equal volume of phosphate buffer solution (PBS) and added to the density gradient tube, followed by centrifugation at 1000 g for 30 minutes. At the end of centrifugation, the PBMC layer was carefully removed. The PBMC proportions obtained were ˜<60% T-cell lymphocytes, ˜15% monocytes/macrophages, ˜10% B-cell lymphocytes, and ˜15% natural killer cells.

RNA was extracted with the RNeasy Mini Kit (Qiagen, Valencia, Calif.) according to the manufacturer's protocol. Briefly, harvested PBMCs are diluted 1:1 with PBS and centrifuged for 10 minutes at 4000 rpm. The resulting supernatant was discarded and the pellet resuspended in 600 μl RLT buffer (1 ml buffer+10 μl 2-β-mercaptoethanol). The sample was homogenized by passing the lysate 5-10 times through 20-G (French) needle fitted to a syringe. Cells were resuspended in 600 μl of DEPC-H₂O diluted in 70% EtOH and was loaded onto an RNeasy mini spin column fitted with a 2-ml collection tube. The sample was twice centrifuged at 14,000 rpm for 15 seconds. The RNeasy column was transferred to a new 2 ml collection tube and 500 μl of RPE buffer added followed by centrifugation at 14,000 rpm for 15 seconds. RPE buffer (500 μl) was added and the sample centrifuged at 10,000 rpm for 2 minutes. The RNeasy column was then transferred into a new 1.5 ml collection tube and RNA free water (30 μl) directly added to the RNase membrane followed by further centrifugation at 10,000 rpm for 1 minute. This was repeated and the extracted RNA stored at −80° C.

### Example 2

### RNA Labeling

This example describes methods used to label the RNA obtained in Example 1. However, one skilled in the art will appreciate that other labels and methods can be used.

RNA obtained from PBMCs was biotin-labeled and cleaned according to Affymetrix guidelines for Human Genome 133A arrays. Briefly, the Enzo BioArray HighYield RNA Transcript Labeling Kit3 (Affymetrix, P/N 900182) was used for generating labeled cRNA target. Template cDNA and the other reaction components were added to RNase-free microfuge tubes. To avoid precipitation of DTT, reactions were at room temperature while additions were made. After adding all reagents, the tube was incubated are a 37° C. for 4 to 5 hours, gently mixing the contents of the tube every 30-45 minutes during the incubation.

To ensure the quality of the initial isolated total RNA, DNase was used to remove contaminant DNA from the sample. In addition, Northern blot followed by optical density analysis was used to determine the concentration of the RNA band.

If the total RNA concentration was >5 μg, the RNA was used for subsequent gene chip hybridization as per the manufacturer's protocol.

### Example 3

### Microarray Hybridization

Coded mRNA samples were analyzed using the Affymetrix GeneChipR Human Genome U133A chips that include 22,283 gene probes (around 19,000 genes) of the best characterized human genes. All samples were hybridized in an interleaved fashion so that systematic errors resulting from chip lot variation, laboratory reagent preparation, and machine drift between ICH patients and referents were minimized. Microarrays were scanned (Axon scanner, Axon Instruments Inc, CA), and images were analyzed using GenePix image analysis software (Axon Instruments Inc, CA) allowing for gene spot fluorescent quantification following subtraction of the surrounding background fluorescent signal within the Affymetrix MASS gene chip analysis suite with production of .CEL, and .DAT output files. The .CDF file or annotation file for the Affymetrix HU133A array and the .CEL files, containing the scanned gene expression information, were the only data files used in all subsequent analyses. Data sets in which the Affymetrix-derived parameter percent present was <30% and/or the array background intensity was >100 fluorescence counts were not used in further data analysis (2 referent subjects). The average percent present call for the arrays was 45%.

### Example 4

### Data Normalization and Statistical Analysis

After exclusion of samples with unsatisfactory hybridization (see Example 3), the CEL files of 8 patients with confirmed ICH, 19 ischemic stroke subjects and 18 referent control subjects were used in the data analyses. The technique of Irizarry et al. (The Analysis of Gene Expression Data. New York: Springer, 2003) was used for analyzing gene expression data. The analysis was completed using the Bioconductor applications of the R programming language and implemented on a 64-bit operating system (SGI Prism dual Itanium CPU, Linux OS) due to the large dataset for analysis (Moore et al., 32 bit architecture—a severe bio-informatics limitation. NHLBI Symposium From Genome to Disease. 2003, Bethesda, Md.: 64). Sample RNA degradation during processing was tightly distributed and uniform across all chips.

Quantile normalization was performed on the CEL data sets from the combined stroke cohort and control subjects. After normalization, expression levels for each gene were calculated with the perfect-match array probes and a robust median polish technique after background correction and log 2 transformation. The gene expression signal was considered to be proportional to the product probe avidity and the gene abundance so, after log transformation, the model fits the probe signal to gene expression and microarray chip effects together with an error term with the assumption of a constant avidity for a particular probe. The estimated gene expression is then log-linearly dependent on the amount of the particular gene expressed in the tissue and is used in all subsequent comparative analyses as a relative measure of the level of gene expression.

The resulting expression set was compared in a pair-wise manner between the ICH patients and referent group, between ICH and ischemic stroke (IS) patients, and between IS and the referent control group, using a robust linear model in the linear models for microarray (LIMMA) R package. This R based package allows application of robust (M-estimator) linear model estimation on a gene-by-gene basis with subsequent multiple comparison corrections (MCCs) using a false discovery correction technique (FDR, Benjamin and Yekutieli, The Annals of Statistics 29:1165-88, 2001) and the more stringent Holm correction (Symth G. Limma: linear models for microarray data. In: Gentleman R, Carey V, Dudoit S, Irizarry R, Huber W, ed. Bioinformatics and Computational Biology Solutions using R and Bioconductor, R. New York: Springer, 2005: 397-420). The MCC corrected p value was <0.05 with values below this threshold accepted as statistically significant gene expression levels (three-way HCI list, Table 2). Subsequently pair-wise comparisons were done between the ICH group and control group (HC) and the ischemic (HI) to create the HC and HI lists, respectively.

Further statistical analysis used the PAM methodology (Prediction Analysis for Microarrays; Tibshirani et al., Proc. Natl. Acad. Sci. 90:6567-72, 2002) to classify samples of unknown type (prospectively obtained samples from 9 stroke patients and 18 controls). This classification method uses the shrunken centroid method to distinguish between ICH and the referent group (either normal subjects or IS subjects). To develop a classification model on a data set, the algorithm essentially uses a threshold to select a subset of genes that show differential expression above the threshold. The algorithm then classifies an unknown case as the type that has average values most similar to the unknown sample for the subset of genes. The threshold (and hence subset of genes) is chosen by cross-validation accuracy in the data set (threshold, 3.8). The classification accuracy obtained through leave-1-out cross validation of the training (i.e., index) set and the accuracy of the PAM model applied to the first independent test set cohort of 4 ICH patients and 6 referent subjects was determined (see below).

Gene annotation and ontology were determined with the Affymetrix online NetAffix suite, together with subsequent literature searches and searches of Online Mendelian Inheritance in Man and LocusLink; this allowed classification of the genes on the lists into molecular function, cellular localization, and biological function (reported, where information is available, in the gene lists in the Appendixes). Genes in the ICH PAM list were also classified into putated pathophysiological class, bearing in mind that not all gene functions (physiological and pathological) are known at the present time; some of these gene classes appear to be consistent with our current knowledge of the pathophysiology of ICH. A hierarchical cluster analysis was also performed.

Correlational graph networks from the Holm corrected differentially expressed gene list between the ICH and the referent groups were derived according to the method of Schafer and Strimmer (Schafer and Strimmer, Stat. Appl. Genet. Mol. Biol. 4:Article32. Epub 2005 Nov. 14, 2005; Schafer and Strimmer, Bioinformatics 2:754-64, 2005). Correlation graphs between the Holm multiple comparison corrected ICH and control graphs were firstly obtained. The nodes were then identified along with the correlation coefficients of the connecting edges, with red lines indicating negative correlations and blue lines indicating positive correlations. The putative pathophysiological mechanisms of the networks were examined.

Table 2 shows the results of the three-way comparison (HCI list) using Holm correction. As shown in Table 2, there are at least 50 gene probes (representing 47 genes) whose expression is significantly different between hemorrhage, control, and ischemic stroke subjects. As shown in Table 2, several genes were upregulated (positive T-statistic, such as a value that is at least 5.3) or downregulated (negative t-statistic, such as a value that is less than −5.2) following an ICH stroke.

TABLE 2
Hemorrhagic stroke related-genes using Holm correction and three-way
comparison.

| Probe Set | | | | |
|---|---|---|---|---|
| ID\hat{ } | Gene Name | t-statistic* | P Value^{$} | B^{@} |
| 200919_at | polyhomeotic homolog 2 | 5.42781316 | 0.04276157 | 4.81900166 |
| | (Drosophila) | | | |
| 201361_at | transmembrane protein 109 | −5.9592879 | 0.00676152 | 6.5916683 |
| 202499_s_at | solute carrier family 2 (facilitated | 7.47492493 | 3.35E−05 | 11.3591858 |
| | glucose transporter), member 3 | | | |
| 202880_s_at | pleckstrin homology, Sec7 and | −5.9658959 | 0.00660849 | 6.60879843 |
| | coiled-coil domains 1(cytohesin 1) | | | |
| 204116_at | interleukin 2 receptor, gamma | −5.4307607 | 0.04233302 | 4.89640009 |
| | (severe combined | | | |
| | immunodeficiency) | | | |
| 205257_s_at | amphiphysin (Stiff-Man syndrome | 9.08325007 | 1.36E−07 | 14.5512864 |
| | with breast cancer 128 kDa | | | |
| | autoantigen) | | | |
| 205403_at | interleukin 1 receptor, type II | 9.20308564 | 9.14E−08 | 16.4898638 |
| 205425_at | huntingtin interacting protein 1 | 5.85978126 | 0.00956716 | 6.21333698 |
| 205456_at | CD3e molecule, epsilon (CD3-TCR | −5.4282032 | 0.04270603 | 4.89345549 |
| | complex) | | | |
| 206025_s_at | tumor necrosis factor, alpha-induced | 7.03185944 | 0.00015776 | 9.72407584 |
| | protein 6 | | | |
| 206026_s_at | tumor necrosis factor, alpha-induced | 5.41824885 | 0.04419288 | 4.79348993 |
| | protein 6 | | | |
| 206028_s_at | c-mer proto-oncogene tyrosine | 6.85623545 | 0.00029192 | 9.37558047 |
| | kinase | | | |
| 206220_s_at | RAS p21 protein activator 3 | −5.3656035 | 0.05296938 | 4.6907174 |
| 206674_at | fms-related tyrosine kinase 3 | 6.06664176 | 0.00464887 | 6.91038013 |
| 207485_x_at | butyrophilin, subfamily 3, member | −5.5223753 | 0.03085995 | 5.19300486 |
| | A1 | | | |
| 208611_s_at | spectrin, alpha, non-erythrocytic 1 | −5.9636524 | 0.00665987 | 6.61736594 |
| | (alpha-fodrin) | | | |
| 208686_s_at | bromodomain containing 2 | −5.760929 | 0.01349446 | 5.96899025 |
| 208842_s_at | golgi reassembly stacking protein 2, | −5.3208606 | 0.06177128 | 4.54677447 |
| | 55 kDa | | | |
| 209154_at | Tax1 (human T-cell leukemia virus | 6.22313512 | 0.0026884 | 7.44743247 |
| | type I) binding protein 3 | | | |
| 209409_at | growth factor receptor-bound protein | 5.7450244 | 0.0142613 | 5.83981346 |
| | 10 | | | |
| 210039_s_at | protein kinase C, theta | −5.3584599 | 0.05428473 | 4.65762338 |
| 210915_x_at | T cell receptor beta variable 19 /// T | −5.8304721 | 0.01059491 | 6.1210925 |
| | cell receptor beta constant 1 | | | |
| 210972_x_at | T cell receptor alpha locus /// T cell | −5.9748089 | 0.00640626 | 6.62958339 |
| | receptor delta variable 2 /// T cell | | | |
| | receptor alpha variable 20 /// T cell | | | |
| | receptor alpha joining 17 /// T cell | | | |
| | receptor alpha constant | | | |
| 211372_s_at | interleukin 1 receptor, type II | 9.19422102 | 9.42E−08 | 15.9398259 |
| 211893_x_at | CD6 molecule | −5.7983325 | 0.01184804 | 6.09290686 |
| 211936_at | heat shock 70 kDa protein 5 (glucose- | 6.02882336 | 0.00530551 | 6.79700294 |
| | regulated protein, 78 kDa) | | | |
| 212017_at | hypothetical protein LOC130074 | −5.297862 | 0.06684395 | 4.47330108 |
| 212259_s_at | pre-B-cell leukemia transcription | −5.8324394 | 0.01052302 | 6.20033235 |
| | factor interacting protein 1 | | | |
| 213193_x_at | T cell receptor beta variable 19 /// T | −6.0301869 | 0.00528052 | 6.74610453 |
| | cell receptor beta constant 1 | | | |
| 213275_x_at | cathepsin B | 6.33989301 | 0.00178581 | 7.80979381 |
| 213805_at | abhydrolase domain containing 5 | 5.98488755 | 0.00618524 | 6.68548269 |
| 214255_at | ATPase, Class V, type 10A | −5.6812689 | 0.01779647 | 5.6882829 |
| 214535_s_at | ADAM metallopeptidase with | 7.51927212 | 2.87E−05 | 11.0169451 |
| | thrombospondin type 1 motif, 2 | | | |
| 216233_at | CD163 molecule | 6.33939279 | 0.00178887 | 7.50164896 |
| 217119_s_at | chemokine (C—X—C motif) receptor 3 | −5.4324543 | 0.04208843 | 4.90305326 |
| 217891_at | chromosome 16 open reading frame | −5.4983059 | 0.03353411 | 5.11364196 |
| | 58 | | | |
| 218328_at | coenzyme Q4 homolog (S. cerevisiae) | −5.4673796 | 0.03731234 | 5.0213153 |
| 218600_at | LIM domain containing 2 | −5.8304404 | 0.01059561 | 6.18995787 |
| 218615_s_at | transmembrane protein 39A | 5.96140673 | 0.00671199 | 6.50928808 |
| 218685_s_at | single-strand-selective | 5.3873204 | 0.04915901 | 4.76216135 |
| | monofunctional uracil-DNA | | | |
| | glycosylase 1 | | | |
| 218689_at | Fanconi anemia, complementation | −5.5094855 | 0.0322647 | 5.14366045 |
| | group F | | | |
| 218805_at | GTPase, IMAP family member 5 | −5.9652232 | 0.00662374 | 6.60914421 |
| 218813_s_at | SH3-domain GRB2-like endophilin | −7.5684784 | 2.42E−05 | 11.826446 |
| | B2 | | | |
| 218871_x_at | chondroitin sulfate GalNAcT-2 | 5.63179622 | 0.0211287 | 5.54223801 |
| 219988_s_at | chromosome 1 open reading frame | −5.3692537 | 0.05231064 | 4.7069779 |
| | 164 | | | |
| 221011_s_at | limb bud and heart development | −6.024406 | 0.00538781 | 6.77353102 |
| | homolog (mouse) | | | |
| 221249_s_at | family with sequence similarity 117, | −5.5397297 | 0.02906352 | 5.24056972 |
| | member A /// family with sequence | | | |
| | similarity 117, member A | | | |
| 221688_s_at | IMP3, U3 small nucleolar | −5.9790832 | 0.00631162 | 6.6280554 |
| | ribonucleoprotein, homolog (yeast) | | | |
| 37652_at | calcineurin binding protein 1 | −5.8006752 | 0.01175233 | 6.09992517 |
| 64064_at | GTPase, IMAP family member 5 | −5.7183538 | 0.01564608 | 5.82028689 |

\hat{ }Probe set ID number is the Affymetrix ID number on the HU133A array.
*Moderated t-statistic. Same interpretation as an ordinary t-statistic except that the standard errors have been moderated across genes, i.e., shrunk towards a common value, using a simple Bayesian model. Positive t-statistic indicates that the gene is upregulated following hemorrhagic stroke. Negative t-statistic indicates that the gene is downregulated following hemorrhagic stroke.
^{$}P-value uncorrected p value
^{@}The B-statistic (lods or B) is the log-odds that the gene is differentially expressed.

When the ICH and the referent groups were compared, 1500 gene probes (1263 genes) were differentially expressed on the FDR list (Table 3), while there were 139 gene probes (119 genes) after the more conservative Holm multiple comparison correction (Table 4). On the FDR list of 1500 gene probes, 719 probes were up-regulated (positive T-statistic, such as a value that is at least 3.2) and 781 gene probes were down-regulated (negative t-statistic, such as a value that is less than −3.2) following a hemorrhagic stroke. Of the 139 gene probes on the Holm listing, 88 were up-regulated (positive T-statistic, such as a value that is at least 5.9) and 51 were down-regulated (negative t-statistic, such as a value that is less than −5.9) following a hemorrhagic stroke. The ICH PAM panel consisted of 30 genes (37 probes) and classified 7/8 ICH patients and 17/18 referents correctly (threshold 3.82, overall correct classification rate of 92.4%, Table 5).

TABLE 3
ICH related-genes using FDR correction and comparison to non-stroke
subjects.

| Probe Set | | | | Adjusted | |
|---|---|---|---|---|---|
| ID\hat{ } | Gene Name | t-statistic* | P Value^{$} | P Value^{#} | B^{@} |
| 219574_at | membrane-associated ring | 6.2140115 | 1.19E−006 | 0.0002407 | 5.496195004 |
| | finger (C3HC4) 1 | | | | |
| 217521_at | Transcribed locus | 5.9641109 | 2.31E−006 | 0.0003593 | 4.876392255 |
| 222303_at | — | 5.5337713 | 7.23E−006 | 0.0008252 | 3.761734456 |
| 213817_at | CDNA FLJ13601 fis, clone | 5.3185262 | 1.29E−005 | 0.001247 | 3.201717309 |
| | PLACE1010069 | | | | |
| c | — | 4.9252208 | 3.70E−005 | 0.0025118 | 2.16919705 |
| 215397_x_at | CDNA FLJ12379 fis, clone | 4.6054579 | 8.75E−005 | 0.0046709 | 1.339093748 |
| | MAMMA1002554 | | | | |
| 209473_at | — | 4.5447313 | 0.000103 | 0.0052906 | 1.193606034 |
| 215796_at | T-cell receptor active alpha- | −4.302835 | 0.0001971 | 0.0081768 | 0.565043001 |
| | chain V-region (V-J-C) | | | | |
| | mRNA, partial cds, clone | | | | |
| | AG212 | | | | |
| 203704_s_at | — | 4.2290916 | 0.00024 | 0.0093056 | 0.3664493 |
| 215191_at | CDNA FLJ14085 fis, clone | 3.9770229 | 0.0004689 | 0.014292 | −0.235246064 |
| | HEMBB1002534 | | | | |
| 206082_at | — | −3.922638 | 0.0005413 | 0.0157053 | −0.400779592 |
| 202969_at | MRNA; cDNA | −3.893127 | 0.0005851 | 0.0164826 | −0.432320508 |
| | DKFZp667B0924 (from | | | | |
| | clone DKFZp667B0924) | | | | |
| 221725_at | CDNA clone | 3.8639169 | 0.0006319 | 0.0171291 | −0.529287784 |
| | IMAGE: 3030163 | | | | |
| 221937_at | CDNA FLJ34482 fis, clone | −3.66971 | 0.0010505 | 0.0239197 | −0.972426526 |
| | HLUNG2004067 | | | | |
| 202377_at | — | 3.6382031 | 0.0011402 | 0.0253027 | −1.104046824 |
| 217412_at | Rearranged T-cell receptor | −3.42662 | 0.0019677 | 0.0358275 | −1.625720669 |
| | alpha chain mRNA, variable | | | | |
| | region | | | | |
| 211632_at | Ig rearranged gamma-chain, | −3.404062 | 0.0020845 | 0.0371886 | −1.676080903 |
| | V-DXP′1-JH4b /// Ig | | | | |
| | rearranged gamma-chain, | | | | |
| | V-DXP′1-JH4b | | | | |
| 214807_at | MRNA; cDNA | 3.2212673 | 0.0033122 | 0.0495345 | −2.085789593 |
| | DKFZp564O0862 (from | | | | |
| | clone DKFZp564O0862) | | | | |
| 203504_s_at | ATP-binding cassette, sub- | 4.0002682 | 0.0004409 | 0.0137594 | −0.17705479 |
| | family A (ABC1), member 1 | | | | |
| 203505_at | ATP-binding cassette, sub- | 3.638904 | 0.0011382 | 0.0252858 | −1.071918781 |
| | family A (ABC1), member 1 | | | | |
| 209993_at | ATP-binding cassette, sub- | −3.338788 | 0.0024615 | 0.0411468 | −1.772670572 |
| | family B (MDR/TAP), | | | | |
| | member 1 | | | | |
| 209641_s_at | ATP-binding cassette, sub- | 5.7543558 | 4.02E−006 | 0.0005396 | 4.343179644 |
| | family C (CFTR/MRP), | | | | |
| | member 3 | | | | |
| 208161_s_at | ATP-binding cassette, sub- | 4.0191798 | 0.0004193 | 0.013292 | −0.161814061 |
| | family C (CFTR/MRP), | | | | |
| | member 3 | | | | |
| 207583_at | ATP-binding cassette, sub- | −3.317981 | 0.0025949 | 0.0424462 | −1.8916331 |
| | family D (ALD), member 2 | | | | |
| 200045_at | ATP-binding cassette, sub- | −3.68465 | 0.0010105 | 0.023375 | −0.975150833 |
| | family F (GCN20), member | | | | |
| | 1 /// ATP-binding cassette, | | | | |
| | sub-family F (GCN20), | | | | |
| | member 1 | | | | |
| 210006_at | abhydrolase domain | −4.783568 | 5.42E−005 | 0.0033259 | 1.809367609 |
| | containing 14A | | | | |
| 213805_at | abhydrolase domain | 4.212729 | 0.0002507 | 0.009499 | 0.339069402 |
| | containing 5 | | | | |
| 218405_at | activator of basal | −5.472837 | 8.51E−006 | 0.00093 | 3.606351047 |
| | transcription 1 | | | | |
| 209600_s_at | acyl-Coenzyme A oxidase | 3.9167198 | 0.0005498 | 0.015911 | −0.396195462 |
| | 1, palmitoyl | | | | |
| 204393_s_at | acid phosphatase, prostate | 5.9204784 | 2.59E−006 | 0.0003896 | 4.744902719 |
| 207275_s_at | acyl-CoA synthetase long- | 7.7487799 | 2.44E−008 | 1.84E−005 | 9.200907246 |
| | chain family member 1 | | | | |
| 201963_at | acyl-CoA synthetase long- | 7.2748901 | 7.87E−008 | 3.99E−005 | 8.113119287 |
| | chain family member 1 | | | | |
| 201661_s_at | acyl-CoA synthetase long- | 5.1015539 | 2.30E−005 | 0.0018408 | 2.632200898 |
| | chain family member 3 | | | | |
| 208636_at | actinin, alpha 1 | 4.2501786 | 0.0002268 | 0.0089432 | 0.452695719 |
| 211160_x_at | actinin, alpha 1 | 3.2123168 | 0.0033875 | 0.0502891 | −2.086756292 |
| 213102_at | ARP3 actin-related protein | 3.5725247 | 0.0013519 | 0.0280881 | −1.251465939 |
| | 3 homolog (yeast) | | | | |
| 222147_s_at | ARP5 actin-related protein | −3.766166 | 0.0008167 | 0.0203333 | |
| | 5 homolog (yeast) | | | | |
| 205209_at | activin A receptor, type IB | 4.6701558 | 7.36E−005 | 0.0041602 | 1.557012572 |
| 213198_at | activin A receptor, type IB | 3.6862217 | 0.0010063 | 0.0233097 | −0.963330725 |
| 216705_s_at | adenosine deaminase | −5.858102 | 3.05E−006 | 0.0004411 | 4.60034505 |
| 204639_at | adenosine deaminase | −4.646322 | 7.84E−005 | 0.0043151 | 1.503048874 |
| 205745_x_at | ADAM metallopeptidase | 5.9660251 | 2.29E−006 | 0.0003593 | 4.862827437 |
| | domain 17 (tumor necrosis | | | | |
| | factor, alpha, converting | | | | |
| | enzyme) | | | | |
| 202381_at | ADAM metallopeptidase | 4.1830621 | 0.0002713 | 0.0100251 | 0.275017999 |
| | domain 9 (meltrin gamma) | | | | |
| 214535_s_at | ADAM metallopeptidase | 13.353671 | 2.00E−013 | 7.43E−010 | 19.55417663 |
| | with thrombospondin type 1 | | | | |
| | motif, 2 | | | | |
| 214454_at | ADAM metallopeptidase | 3.8573237 | 0.0006429 | 0.0173489 | −0.517394546 |
| | with thrombospondin type 1 | | | | |
| | motif, 2 | | | | |
| 202912_at | adrenomedullin | 4.9990521 | 3.04E−005 | 0.0022038 | 2.401148229 |
| 204184_s_at | adrenergic, beta, receptor | 3.6428635 | 0.0011265 | 0.0251089 | −1.080930339 |
| | kinase 2 | | | | |
| 217729_s_at | amino-terminal enhancer of | −3.482649 | 0.0017044 | 0.0328251 | −1.47940264 |
| | split | | | | |
| 202486_at | AFG3 ATPase family gene | −3.867135 | 0.0006265 | 0.0170886 | −0.497499361 |
| | 3-like 2 (yeast) | | | | |
| 218096_at | 1-acylglycerol-3-phosphate | −3.909411 | 0.0005605 | 0.0160953 | −0.304176054 |
| | O-acyltransferase 5 | | | | |
| | (lysophosphatidic acid | | | | |
| | acyltransferase, epsilon) | | | | |
| 200849_s_at | S-adenosylhomocysteine | 3.7580003 | 0.0008343 | 0.0205657 | −0.800942379 |
| | hydrolase-like 1 | | | | |
| 200850_s_at | S-adenosylhomocysteine | 3.4393866 | 0.0019044 | 0.0351878 | −1.589406868 |
| | hydrolase-like 1 | | | | |
| 202820_at | aryl hydrocarbon receptor | 6.0756321 | 1.72E−006 | 0.0003031 | 5.158254442 |
| 201782_s_at | aryl hydrocarbon receptor | −5.385064 | 1.08E−005 | 0.0011161 | 3.364624315 |
| | interacting protein | | | | |
| 210517_s_at | A kinase (PRKA) anchor | −3.260737 | 0.002999 | 0.0465465 | −2.029738842 |
| | protein (gravin) 12 | | | | |
| 221718_s_at | A kinase (PRKA) anchor | 4.0902665 | 0.0003473 | 0.011778 | 0.022122652 |
| | protein 13 | | | | |
| 205771_s_at | A kinase (PRKA) anchor | −5.132534 | 2.12E−005 | 0.0017567 | 2.745493369 |
| | protein 7 | | | | |
| 211653_x_at | aldo-keto reductase family | −3.391699 | 0.0021513 | 0.0378356 | −1.705243646 |
| | 1, member C2 (dihydrodiol | | | | |
| | dehydrogenase 2; bile acid | | | | |
| | binding protein; 3-alpha | | | | |
| | hydroxysteroid | | | | |
| | dehydrogenase, type III) | | | | |
| 209160_at | aldo-keto reductase family | −5.303392 | 1.34E−005 | 0.0012764 | 3.170581717 |
| | 1, member C3 (3-alpha | | | | |
| | hydroxysteroid | | | | |
| | dehydrogenase, type II) | | | | |
| 212607_at | v-akt murine thymoma viral | −4.135078 | 0.0003083 | 0.0109442 | 0.159674429 |
| | oncogene homolog 3 | | | | |
| | (protein kinase B, gamma) | | | | |
| 201951_at | activated leukocyte cell | 5.8234522 | 3.35E−006 | 0.0004749 | 4.500591957 |
| | adhesion molecule | | | | |
| 201952_at | activated leukocyte cell | 4.9114956 | 3.84E−005 | 0.0025638 | 2.165273885 |
| | adhesion molecule | | | | |
| 217791_s_at | aldehyde dehydrogenase 18 | −4.115641 | 0.0003246 | 0.0112496 | 0.087005997 |
| | family, member A1 | | | | |
| 202022_at | aldolase C, fructose- | −4.450957 | 0.0001325 | 0.0062436 | 0.954962693 |
| | bisphosphate | | | | |
| 207206_s_at | arachidonate 12- | 3.5982628 | 0.0012647 | 0.0268656 | −1.207124821 |
| | lipoxygenase | | | | |
| 206714_at | arachidonate 15- | 4.0173153 | 0.0004214 | 0.013301 | −0.109355597 |
| | lipoxygenase, type B | | | | |
| 204446_s_at | arachidonate 5-lipoxygenase | 6.8751354 | 2.16E−007 | 7.06E−005 | 7.143490943 |
| 214366_s_at | arachidonate 5-lipoxygenase | 4.398668 | 0.0001525 | 0.0068192 | 0.864966387 |
| 204445_s_at | arachidonate 5-lipoxygenase | 3.5809859 | 0.0013226 | 0.0277674 | −1.190527294 |
| 209424_s_at | alpha-methylacyl-CoA | −3.592109 | 0.0012851 | 0.0271678 | −1.145489707 |
| | racemase | | | | |
| 207992_s_at | adenosine monophosphate | 3.3249268 | 0.0025496 | 0.0420212 | −1.820580731 |
| | deaminase (isoform E) | | | | |
| 205257_s_at | amphiphysin (Stiff-Man | 24.606614 | 4.70E−020 | 5.24E−016 | 30.88004619 |
| | syndrome with breast cancer | | | | |
| | 128 kDa autoantigen) | | | | |
| 218575_at | anaphase promoting | −4.642249 | 7.93E−005 | 0.0043518 | 1.479482015 |
| | complex subunit 1 | | | | |
| 205141_at | angiogenin, ribonuclease, | 4.0982088 | 0.00034 | 0.0116248 | 0.073479841 |
| | RNase A family, 5 /// | | | | |
| | ribonuclease, RNase A | | | | |
| | family, 4 | | | | |
| 216195_at | Ankyrin 2, neuronal | 3.5648945 | 0.0013788 | 0.0284481 | −1.284981826 |
| 212747_at | ankyrin repeat and sterile | 4.5969814 | 8.95E−005 | 0.0047624 | 1.336044754 |
| | alpha motif domain | | | | |
| | containing 1A | | | | |
| 211241_at | annexin A2 pseudogene 3 | 3.2423469 | 0.0031412 | 0.0476847 | −1.949568122 |
| 209369_at | annexin A3 | 3.695767 | 0.0009816 | 0.0228796 | −0.929439054 |
| 212159_x_at | adaptor-related protein | 3.3918963 | 0.0021502 | 0.0378356 | −1.680557229 |
| | complex 2, alpha 2 subunit | | | | |
| 210278_s_at | adaptor-related protein | −3.246169 | 0.0031111 | 0.0475158 | −2.046209608 |
| | complex 4, sigma 1 subunit | | | | |
| 215310_at | Adenomatosis polyposis | 4.4668912 | 0.000127 | 0.0060731 | 1.012170827 |
| | coli | | | | |
| 214960_at | apoptosis inhibitor 5 | 3.3619728 | 0.0023206 | 0.0396727 | −1.760827082 |
| 209584_x_at | apolipoprotein B mRNA | −3.823951 | 0.0007019 | 0.0183997 | −0.570559962 |
| | editing enzyme, catalytic | | | | |
| | polypeptide-like 3C | | | | |
| 204205_at | apolipoprotein B mRNA | −3.545332 | 0.0014503 | 0.029498 | −1.289961839 |
| | editing enzyme, catalytic | | | | |
| | polypeptide-like 3G | | | | |
| 214995_s_at | apolipoprotein B mRNA | −3.967574 | 0.0004807 | 0.0145739 | −0.279900828 |
| | editing enzyme, catalytic | | | | |
| | polypeptide-like 3G /// | | | | |
| | apolipoprotein B mRNA | | | | |
| | editing enzyme, catalytic | | | | |
| | polypeptide-like 3F | | | | |
| 209546_s_at | apolipoprotein L, 1 | −4.061354 | 0.000375 | 0.012471 | −0.055202336 |
| 221087_s_at | apolipoprotein L, 3 | −5.013123 | 2.92E−005 | 0.0021571 | 2.415912514 |
| 219716_at | apolipoprotein L, 6 | −3.903775 | 0.0005689 | 0.0162109 | −0.433153937 |
| 222013_x_at | Amyloid beta (A4) | −3.587655 | 0.0013 | 0.027405 | −1.218513413 |
| | precursor protein (peptidase | | | | |
| | nexin-II, Alzheimer disease) | | | | |
| 203219_s_at | adenine | −5.644748 | 5.38E−006 | 0.0006841 | 4.048560866 |
| | phosphoribosyltransferase | | | | |
| 213892_s_at | adenine | −3.823386 | 0.0007029 | 0.0184054 | −0.652522358 |
| | phosphoribosyltransferase | | | | |
| 203747_at | aquaporin 3 (Gill blood | −5.081099 | 2.44E−005 | 0.0019242 | 2.580423765 |
| | group) | | | | |
| 39248_at | aquaporin 3 (Gill blood | −3.425921 | 0.0019712 | 0.0358275 | −1.560552654 |
| | group) | | | | |
| 210068_s_at | aquaporin 4 | 4.2711856 | 0.0002144 | 0.0086099 | 0.500233768 |
| 205568_at | aquaporin 9 | 4.3844739 | 0.0001584 | 0.007013 | 0.838547325 |
| 218870_at | Rho GTPase activating | −4.376266 | 0.0001619 | 0.0071157 | 0.794215713 |
| | protein 15 | | | | |
| 37577_at | Rho GTPase activating | 6.7634786 | 2.88E−007 | 8.66E−005 | 6.869000176 |
| | protein 19 | | | | |
| 212738_at | Rho GTPase activating | 4.5297599 | 0.0001073 | 0.0054247 | 1.142225267 |
| | protein 19 | | | | |
| 38149_at | Rho GTPase activating | −3.81169 | 0.0007248 | 0.0187807 | −0.667955466 |
| | protein 25 | | | | |
| 204882_at | Rho GTPase activating | −3.244861 | 0.0031214 | 0.04764 | −2.027177223 |
| | protein 25 | | | | |
| 205068_s_at | Rho GTPase activating | 5.0046517 | 2.99E−005 | 0.0021923 | 2.399528811 |
| | protein 26 | | | | |
| 203910_at | Rho GTPase activating | 3.8768619 | 0.0006107 | 0.0168005 | −0.485927245 |
| | protein 29 | | | | |
| 201659_s_at | ADP-ribosylation factor- | −4.128231 | 0.0003139 | 0.0110574 | 0.185529825 |
| | like 1 | | | | |
| 205020_s_at | ADP-ribosylation factor- | 6.5781587 | 4.63E−007 | 0.000126 | 6.412365317 |
| | like 4A | | | | |
| 202208_s_at | ADP-ribosylation factor- | −6.147552 | 1.42E−006 | 0.0002709 | 5.341709786 |
| | like 4C | | | | |
| 202207_at | ADP-ribosylation factor- | −4.29996 | 0.0001986 | 0.0082246 | 0.636583059 |
| | like 4C | | | | |
| 202206_at | ADP-ribosylation factor- | −3.616456 | 0.0012065 | 0.0261513 | −1.088678256 |
| | like 4C | | | | |
| 217817_at | actin related protein 2/3 | −3.215778 | 0.0033582 | 0.0500413 | −2.06899997 |
| | complex, subunit 4, 20 kDa | | | | |
| 203428_s_at | ASF1 anti-silencing | −7.698443 | 2.76E−008 | 1.94E−005 | 9.105468688 |
| | function 1 homolog A (S. cerevisiae) | | | | |
| 206130_s_at | asialoglycoprotein receptor 2 | 3.4369337 | 0.0019164 | 0.0353216 | −1.570677391 |
| 209135_at | aspartate beta-hydroxylase | 6.1302992 | 1.49E−006 | 0.0002811 | 5.298766404 |
| 210896_s_at | aspartate beta-hydroxylase | 5.0153778 | 2.91E−005 | 0.0021512 | 2.439027924 |
| 218908_at | alveolar soft part sarcoma | −3.640518 | 0.0011334 | 0.0252051 | −1.098684526 |
| | chromosome region, | | | | |
| | candidate 1 | | | | |
| 208033_s_at | AT-binding transcription | 4.1680066 | 0.0002824 | 0.0103326 | 0.226968168 |
| | factor 1 | | | | |
| 207774_at | ATG10 autophagy related | 3.4300496 | 0.0019505 | 0.0356839 | −1.593702284 |
| | 10 homolog (S. cerevisiae) | | | | |
| 214255_at | ATPase, Class V, type 10A | −3.950979 | 0.0005023 | 0.0149627 | −0.283496772 |
| 212361_s_at | ATPase, Ca++ transporting, | 4.6314146 | 8.16E−005 | 0.0044164 | 1.401320848 |
| | cardiac muscle, slow twitch 2 | | | | |
| 212362_at | ATPase, Ca++ transporting, | 3.9993183 | 0.000442 | 0.0137747 | −0.200772262 |
| | cardiac muscle, slow twitch 2 | | | | |
| 212383_at | ATPase, H+ transporting, | 3.3500538 | 0.002392 | 0.0404668 | −1.753503448 |
| | lysosomal V0 subunit a1 | | | | |
| 214150_x_at | ATPase, H+ transporting, | 3.5216114 | 0.0015418 | 0.0307844 | −1.381087313 |
| | lysosomal 9 kDa, V0 subunite | | | | |
| 213587_s_at | ATPase, H+ transporting V0 | −3.762944 | 0.0008236 | 0.0204012 | −0.774090061 |
| | subunit E2-like (rat) | | | | |
| 201971_s_at | ATPase, H+ transporting, | 4.7273604 | 6.31E−005 | 0.0037377 | 1.693025609 |
| | lysosomal 70 kDa, V1 | | | | |
| | subunit A | | | | |
| 201972_at | ATPase, H+ transporting, | 3.6652952 | 0.0010627 | 0.0240893 | −1.018232845 |
| | lysosomal 70 kDa, V1 | | | | |
| | subunit A | | | | |
| 202874_s_at | ATPase, H+ transporting, | 5.2037675 | 1.75E−005 | 0.0015425 | 2.945874363 |
| | lysosomal 42 kDa, V1 | | | | |
| | subunit C1 | | | | |
| 202872_at | ATPase, H+ transporting, | 4.9040952 | 3.92E−005 | 0.002592 | 2.155646031 |
| | lysosomal 42 kDa, V1 | | | | |
| | subunit C1 | | | | |
| 219366_at | apoptosis, caspase | −3.785851 | 0.0007756 | 0.0196854 | −0.694210254 |
| | activation inhibitor | | | | |
| 205539_at | advillin | 3.6787016 | 0.0010262 | 0.0235991 | −0.950852573 |
| 218043_s_at | 5-azacytidine induced 2 | 5.1049863 | 2.28E−005 | 0.0018304 | 2.648637789 |
| 213589_s_at | UDP-GlcNAc:betaGal beta- | 3.7398179 | 0.000875 | 0.021299 | −0.841903909 |
| | 1,3-N- | | | | |
| | acetylglucosaminyltransferase- | | | | |
| | like 1 | | | | |
| 204194_at | BTB and CNC homology 1, | 3.8620114 | 0.0006351 | 0.0171734 | −0.501838173 |
| | basic leucine zipper | | | | |
| | transcription factor 1 | | | | |
| 217986_s_at | bromodomain adjacent to | 3.5257013 | 0.0015256 | 0.0304889 | −1.374607205 |
| | zinc finger domain, 1A | | | | |
| 220588_at | breast carcinoma amplified | −3.479353 | 0.0017189 | 0.0330187 | −1.490301402 |
| | sequence 4 | | | | |
| 214390_s_at | branched chain | 4.6722167 | 7.32E−005 | 0.0041477 | 1.563412382 |
| | aminotransferase 1, | | | | |
| | cytosolic | | | | |
| 214452_at | branched chain | 4.3707838 | 0.0001643 | 0.0071839 | 0.818473701 |
| | aminotransferase 1, | | | | |
| | cytosolic | | | | |
| 219528_s_at | B-cell CLL/lymphoma 11B | −3.600251 | 0.0012582 | 0.0268042 | −1.122516509 |
| | (zinc finger protein) | | | | |
| 205681_at | BCL2-related protein A1 | 5.0859526 | 2.40E−005 | 0.001906 | 2.619911083 |
| 209311_at | BCL2-like 2 | 5.9716622 | 2.26E−006 | 0.0003578 | 4.877103649 |
| 204908_s_at | B-cell CLL/lymphoma 3 | 4.3839069 | 0.0001586 | 0.007013 | 0.765318372 |
| 203140_at | B-cell CLL/lymphoma 6 | 6.4484079 | 6.48E−007 | 0.0001605 | 6.082317265 |
| | (zinc finger protein 51 | | | | |
| 215990_s_at | B-cell CLL/lymphoma 6 | 3.9633932 | 0.0004861 | 0.014642 | −0.248848619 |
| | (zinc finger protein 51) | | | | |
| 219072_at | B-cell CLL/lymphoma 7C | −3.364243 | 0.0023072 | 0.0395937 | −1.779304828 |
| 214643_x_at | bridging integrator 1 | −6.752528 | 2.96E−007 | 8.79E−005 | 6.842410212 |
| 210202_s_at | bridging integrator 1 | −4.796617 | 5.23E−005 | 0.0032605 | 1.863742627 |
| 210201_x_at | bridging integrator 1 | −3.29496 | 0.0027507 | 0.0439378 | −1.937894606 |
| 202931_x_at | bridging integrator 1 | −3.241899 | 0.0031448 | 0.0477024 | −2.051991096 |
| 204860_s_at | baculoviral IAP repeat- | 4.733394 | 6.21E−005 | 0.0036873 | 1.685454114 |
| | containing 1 /// similar to | | | | |
| | Baculoviral IAP repeat- | | | | |
| | containing protein 1 | | | | |
| | (Neuronal apoptosis | | | | |
| | inhibitory protein) /// similar | | | | |
| | to Baculoviral IAP repeat- | | | | |
| | containing protein 1 | | | | |
| | (Neuronal apoptosis | | | | |
| | inhibitory protein) | | | | |
| 204861_s_at | baculoviral IAP repeat- | 5.272023 | 1.46E−005 | 0.0013482 | 3.075216041 |
| | containing 1 /// similar to | | | | |
| | Baculoviral IAP repeat- | | | | |
| | containing protein 1 | | | | |
| | (Neuronal apoptosis | | | | |
| | inhibitory protein) | | | | |
| 210538_s_at | baculoviral IAP repeat- | −3.537025 | 0.0014817 | 0.0299608 | −1.280020969 |
| | containing 3 | | | | |
| 206464_at | BMX non-receptor tyrosine | 4.2966792 | 0.0002003 | 0.0082533 | 0.631448027 |
| | kinase | | | | |
| 209308_s_at | BCL2/adenovirus E1B | 4.683964 | 7.09E−005 | 0.0040916 | 1.561267901 |
| | 19 kDa interacting protein 2 | | | | |
| 202946_s_at | BTB (POZ) domain | 4.5294512 | 0.0001074 | 0.0054247 | 1.151993508 |
| | containing 3 | | | | |
| 220297_at | BTB (POZ) domain | 3.5807554 | 0.0013234 | 0.0277674 | −1.214745709 |
| | containing 7 | | | | |
| 214117_s_at | biotinidase | −3.266783 | 0.0029536 | 0.0461215 | −2.017100546 |
| 207485_x_at | butyrophilin, subfamily 3, | −6.870456 | 2.19E−007 | 7.06E−005 | 7.132260801 |
| | member A1 | | | | |
| 209770_at | butyrophilin, subfamily 3, | −4.67677 | 7.23E−005 | 0.0041288 | 1.607602252 |
| | member A1 | | | | |
| 209846_s_at | butyrophilin, subfamily 3, | −4.119178 | 0.0003216 | 0.0111965 | 0.104419317 |
| | member A2 | | | | |
| 38241_at | butyrophilin, subfamily 3, | −3.499997 | 0.00163 | 0.0318003 | −1.421916429 |
| | member A3 | | | | |
| 204820_s_at | butyrophilin, subfamily 3, | −3.692046 | 0.0009912 | 0.0230064 | −0.967713537 |
| | member A3 /// butyrophilin, | | | | |
| | subfamily 3, member A2 | | | | |
| 201457_x_at | BUB3 budding uninhibited | −4.017883 | 0.0004208 | 0.0132999 | −0.089230155 |
| | by benzimidazoles 3 | | | | |
| | homolog (yeast) | | | | |
| 212121_at | chromosome 10 open | −3.568339 | 0.0013666 | 0.0282744 | −1.250064484 |
| | reading frame 61 | | | | |
| 211376_s_at | chromosome 10 open | −3.407426 | 0.0020667 | 0.0369951 | −1.659499482 |
| | reading frame 86 | | | | |
| 220987_s_at | chromosome 11 open | −3.219017 | 0.003331 | 0.0497486 | −2.048987662 |
| | reading frame 17 /// | | | | |
| | chromosome 11 open | | | | |
| | reading frame 17 /// NUAK | | | | |
| | family, SNF1-like kinase, 2 | | | | |
| | /// NUAK family, SNF1-like | | | | |
| | kinase, 2 | | | | |
| 217969_at | chromosome 11 open | −3.556491 | 0.0014091 | 0.0288324 | −1.29769529 |
| | reading frame2 | | | | |
| 206438_x_at | chromosome 12 open | −3.239458 | 0.0031642 | 0.0478336 | −2.039190862 |
| | reading frame 38 | | | | |
| 217188_s_at | chromosome 14 open | −5.269476 | 1.47E−005 | 0.0013518 | 3.079256062 |
| | reading frame 1 | | | | |
| 202562_s_at | chromosome 14 open | −3.805795 | 0.0007361 | 0.0189632 | −0.675617112 |
| | reading frame 1 | | | | |
| 219526_at | chromosome 14 open | −3.430842 | 0.0019466 | 0.0356839 | −1.596339315 |
| | reading frame 169 | | | | |
| 215087_at | chromosome 15 open | 4.2471265 | 0.0002287 | 0.0089711 | 0.436323039 |
| | reading frame 39 | | | | |
| 219315_s_at | chromosome 16 open | −3.957249 | 0.000494 | 0.0148205 | −0.312256745 |
| | reading frame 30 | | | | |
| 204676_at | chromosome 16 open | −3.341578 | 0.0024441 | 0.0410411 | −1.794230891 |
| | reading frame 51 | | | | |
| 217891_at | chromosome 16 open | −3.322754 | 0.0025637 | 0.0421599 | −1.853415417 |
| | reading frame 58 | | | | |
| 218945_at | chromosome 16 open | 3.6936484 | 0.000987 | 0.0229583 | −0.94405567 |
| | reading frame 68 | | | | |
| 205781_at | chromosome 16 open | 3.7158412 | 0.0009315 | 0.0220336 | −0.849203521 |
| | reading frame 7 | | | | |
| 217957_at | chromosome 16 open | −4.967644 | 3.30E−005 | 0.0023297 | 2.294063294 |
| | reading frame 80 | | | | |
| 209092_s_at | chromosome 17 open | −3.501058 | 0.0016256 | 0.0317731 | −1.406170981 |
| | reading frame 25 | | | | |
| 219417_s_at | chromosome 17 open | −3.592994 | 0.0012821 | 0.0271506 | −1.192486122 |
| | reading frame 59 | | | | |
| 213617_s_at | chromosome 18 open | −4.681348 | 7.14E−005 | 0.0041099 | 1.564460353 |
| | reading frame 10 | | | | |
| 217926_at | chromosome 19 open | −3.257394 | 0.0030244 | 0.0467678 | −2.009985042 |
| | reading frame 53 | | | | |
| 204699_s_at | chromosome 1 open reading | −4.370502 | 0.0001644 | 0.0071839 | 0.758851552 |
| | frame 107 | | | | |
| 204700_x_at | chromosome 1 open reading | −4.276309 | 0.0002115 | 0.0085082 | 0.532021393 |
| | frame 107 | | | | |
| 218165_at | chromosome 1 open reading | −3.624748 | 0.0011808 | 0.0258461 | −1.143368214 |
| | frame 149 | | | | |
| 219988_s_at | chromosome 1 open reading | −4.117339 | 0.0003232 | 0.0112339 | 0.107860676 |
| | frame 164 | | | | |
| 220476_s_at | chromosome 1 open reading | 4.2932023 | 0.0002022 | 0.0082821 | 0.584561217 |
| | frame 183 | | | | |
| 217966_s_at | chromosome 1 open reading | 4.2589556 | 0.0002216 | 0.0087963 | 0.463871896 |
| | frame 24 | | | | |
| 202953_at | complement component 1, q | 3.7859774 | 0.0007754 | 0.0196854 | −0.712502494 |
| | subcomponent, B chain | | | | |
| 219004_s_at | chromosome 21 open | −5.162874 | 1.95E−005 | 0.0016545 | 2.79967566 |
| | reading frame 45 | | | | |
| 220941_s_at | chromosome 21 open | −3.304135 | 0.0026875 | 0.0432705 | −1.917202487 |
| | reading frame 91 | | | | |
| 212421_at | chromosome 22 open | 4.1451572 | 0.0003001 | 0.0107685 | 0.188156292 |
| | reading frame 9 | | | | |
| 209906_at | complement component 3a | 6.1809183 | 1.30E−006 | 0.0002525 | 5.413796702 |
| | receptor 1 | | | | |
| 220942_x_at | chromsome 3 open reading | −4.853153 | 4.50E−005 | 0.0028944 | 2.001659499 |
| | frame 28 | | | | |
| 201677_at | Chromosome 3 open | −5.356111 | 1.16E−005 | 0.0011734 | 3.295627658 |
| | reading frame 37 | | | | |
| 201678_s_at | chromosome 3 open reading | −4.203306 | 0.000257 | 0.0097242 | 0.336461793 |
| | frame 37 | | | | |
| 208247_at | chromosome 3 open reading | 3.3372711 | 0.002471 | 0.0412129 | −1.788100374 |
| | frame 51 | | | | |
| 219872_at | chromosome 4 open reading | 3.873291 | 0.0006165 | 0.0168966 | −0.467872947 |
| | frame 18 | | | | |
| 218449_at | chromosome 4 open reading | −3.451693 | 0.0018453 | 0.0343232 | −1.532007933 |
| | frame 20 | | | | |
| 220088_at | complement component 5a | 5.1976547 | 1.78E−005 | 0.0015618 | 2.87559802 |
| | receptor 1 | | | | |
| 204238_s_at | chromosome 6 open reading | −3.43219 | 0.0019398 | 0.0356353 | −1.575503074 |
| | frame 108 | | | | |
| 218561_s_at | chromosome 6 open reading | −6.367632 | 8.00E−007 | 0.0001896 | 5.890852776 |
| | frame 149 | | | | |
| 204215_at | chromosome 7 open reading | −4.364781 | 0.000167 | 0.0072521 | 0.743422444 |
| | frame 23 | | | | |
| 209446_s_at | chromosome 7 open reading | 3.4997198 | 0.0016312 | 0.0318003 | −1.431396609 |
| | frame 44 | | | | |
| 218500_at | chromosome 8 open reading | −3.958172 | 0.0004928 | 0.0148196 | −0.305022489 |
| | frame 55 | | | | |
| 220712_at | chromosome 8 open reading | 4.6650801 | 7.46E−005 | 0.004175 | 1.518394673 |
| | frame 60 | | | | |
| 218992_at | chromosome 9 open reading | −4.939579 | 3.56E−005 | 0.0024505 | 2.237866281 |
| | frame 46 | | | | |
| 204811_s_at | calcium channel, voltage- | −3.485289 | 0.0016929 | 0.0327163 | −1.439474347 |
| | dependent, alpha 2/delta | | | | |
| | subunit 2 | | | | |
| 213688_at | calmodulin 1 | −6.510788 | 5.51E−007 | 0.0001446 | 6.240465245 |
| | (phosphorylase kinase, | | | | |
| | delta) | | | | |
| 213812_s_at | calcium/calmodulin- | 5.213278 | 1.71E−005 | 0.0015095 | 2.926803598 |
| | dependent protein kinase- | | | | |
| | kinase 2, beta | | | | |
| 200625_s_at | CAP, adenylate cyclase- | 3.9952945 | 0.0004467 | 0.0138256 | −0.205588725 |
| | associated protein 1 (yeast) | | | | |
| 203357_s_at | calpain 7 | −3.845618 | 0.000663 | 0.017715 | −0.572992867 |
| 220066_at | caspase recruitment domain | 4.5183086 | 0.0001106 | 0.0055225 | 1.148843677 |
| | family, member 15 | | | | |
| 213373_s_at | caspase 8, apoptosis-related | −4.716881 | 6.49E−005 | 0.0038217 | 1.647409129 |
| | cysteine peptidase | | | | |
| 205379_at | carbonyl reductase 3 | −3.231282 | 0.0032299 | 0.048564 | −2.102146102 |
| 220175_s_at | COBW domain containing 1 | 3.422802 | 0.001987 | 0.0359971 | −1.629126784 |
| | /// COBW domain | | | | |
| | containing 2 /// COBW | | | | |
| 202048_s_at | domain containing 5 /// | −3.394545 | 0.0021357 | 0.0376896 | −1.648369734 |
| | COBW-like placental | | | | |
| | protein /// COBW domain | | | | |
| | containing 3 /// COBW | | | | |
| | domain containing 6 /// | | | | |
| | similar to COBW domain | | | | |
| | containing 3 | | | | |
| | chromobox homolog 6 | | | | |
| 204610_s_at | coiled-coil domain | −4.403981 | 0.0001503 | 0.0067472 | 0.815062002 |
| | containing 85B | | | | |
| 204335_at | coiled-coil domain | −3.892108 | 0.0005867 | 0.0164871 | −0.475140377 |
| | containing 94 | | | | |
| 204103_at | chemokine (C-C motif) | −3.68227 | 0.0010167 | 0.0234534 | −1.00608852 |
| | ligand 4 | | | | |
| 200953_s_at | cyclin D2 | −6.234765 | 1.13E−006 | 0.0002379 | 5.558570668 |
| 200952_s_at | cyclin D2 | −6.106075 | 1.59E−006 | 0.0002921 | 5.218632002 |
| 208796_s_at | cyclin G1 | −3.716767 | 0.0009293 | 0.0220336 | −0.908419649 |
| 221156_x_at | cell cycle progression 1 | 3.428333 | 0.0019591 | 0.0358116 | −1.557048491 |
| 205098_at | chemokine (C-C motif) | 3.5304313 | 0.0015071 | 0.0302551 | −1.348157061 |
| | receptor 1 | | | | |
| 206978_at | chemokine (C-C motif) | 3.8787507 | 0.0006077 | 0.0167586 | −0.497079247 |
| | receptor 2 | | | | |
| 207794_at | chemokine (C-C motif) | 3.5199822 | 0.0015483 | 0.0308311 | −1.350504247 |
| | receptor 2 | | | | |
| 208304_at | chemokine (C-C motif) | −3.411181 | 0.0020469 | 0.0367442 | −1.675334498 |
| | receptor 3 | | | | |
| 201946_s_at | chaperonin containing | −5.228948 | 1.64E−005 | 0.0014589 | 2.981866109 |
| | TCP1, subunit 2 (beta) | | | | |
| 201743_at | CD14 molecule | 3.5616738 | 0.0013903 | 0.0285361 | −1.259800117 |
| 216233_at | CD163 molecule | 18.497494 | 6.95E−017 | 3.87E−013 | 25.43080936 |
| 215049_x_at | CD163 molecule | 9.7258472 | 2.51E−010 | 6.22E−007 | 13.52665406 |
| 203645_s_at | CD163 molecule | 9.529413 | 3.87E−010 | 7.84E−007 | 13.11769962 |
| 205831_at | CD2 molecule | −5.139809 | 2.08E−005 | 0.0017291 | 2.770681136 |
| 210031_at | CD247 molecule | −4.465239 | 0.0001276 | 0.0060731 | 1.056716817 |
| 211856_x_at | CD28 molecule | −4.796364 | 5.24E−005 | 0.0032605 | 1.83280249 |
| 211861_x_at | CD28 molecule | −4.545051 | 0.000103 | 0.0052906 | 1.183081083 |
| 206545_at | CD28 molecule | −4.061393 | 0.0003749 | 0.012471 | −0.00770044 |
| 206120_at | CD33 molecule | 3.8340141 | 0.0006836 | 0.0181115 | −0.568137157 |
| 209555_s_at | CD36 molecule | 3.8015446 | 0.0007444 | 0.0191095 | −0.695026645 |
| | (thrombospondin receptor) | | | | |
| 213539_at | CD3d molecule, delta | −5.81472 | 3.42E−006 | 0.0004828 | 4.496982373 |
| | (CD3-TCR complex) | | | | |
| 205456_at | CD3e molecule, epsilon | −5.070342 | 2.51E−005 | 0.0019651 | 2.55370924 |
| | (CD3-TCR complex) | | | | |
| 206804_at | CD3g molecule, gamma | −3.530893 | 0.0015053 | 0.0302551 | −1.355587079 |
| | (CD3-TCR complex) | | | | |
| 203547_at | CD4 molecule | −3.464217 | 0.001787 | 0.0337739 | −1.500532943 |
| 215346_at | CD40 molecule, TNF | −3.424798 | 0.0019769 | 0.0358721 | −1.62296154 |
| | receptor superfamily | | | | |
| | member 5 | | | | |
| 204118_at | CD48 molecule | −3.470732 | 0.0017574 | 0.0334519 | −1.488468249 |
| 206485_at | CD5 molecule | −3.349801 | 0.0023935 | 0.0404668 | −1.805774335 |
| 208602_x_at | CD6 molecule | −4.497593 | 0.0001169 | 0.0057022 | 1.079832745 |
| 211900_x_at | CD6 molecule | −3.94658 | 0.0005081 | 0.015057 | −0.340650267 |
| 213958_at | CD6 molecule | −3.774492 | 0.0007991 | 0.0199959 | −0.771212592 |
| 211893_x_at | CD6 molecule | −3.69607 | 0.0009808 | 0.0228796 | −0.973005527 |
| 200663_at | CD63 molecule | 3.584994 | 0.0013089 | 0.0275423 | −1.183405001 |
| 214049_x_at | CD7 molecule | −3.784239 | 0.0007789 | 0.0197462 | −0.757309831 |
| 209619_at | CD74 molecule, major | −3.838426 | 0.0006757 | 0.0179669 | −0.608700768 |
| | histocompatibility complex, | | | | |
| | class II invariant chain | | | | |
| 215332_s_at | CD8b molecule | −3.364868 | 0.0023035 | 0.0395937 | −1.778361369 |
| 202878_s_at | CD93 molecule | 4.5187978 | 0.0001105 | 0.0055225 | 1.14977715 |
| 202877_s_at | CD93 molecule | 4.0522554 | 0.0003841 | 0.0126796 | −0.066413622 |
| 206761_at | CD96 molecule | −4.428809 | 0.0001406 | 0.0064487 | 0.94610521 |
| 205627_at | cytidine deaminase | 3.4628567 | 0.0017932 | 0.0338007 | −1.518453849 |
| 210440_s_at | CDC14 cell division cycle | −4.773031 | 5.58E−005 | 0.0033866 | 1.771675491 |
| | 14 homolog A (S. cerevisiae) | | | | |
| 201853_s_at | cell division cycle 25B | −3.333859 | 0.0024925 | 0.0414783 | −1.810878375 |
| | CDC42 effector protein | | | | |
| 209286_at | (Rho GTPase binding) 3 | 8.7039359 | 2.51E−009 | 2.94E−006 | 11.36556799 |
| 202246_s_at | cyclin-dependent kinase 4 | −4.708561 | 6.63E−005 | 0.0038597 | 1.628852392 |
| 214877_at | CDK5 regulatory subunit | −7.380157 | 6.05E−008 | 3.46E−005 | 8.358822206 |
| | associated protein 1-like 1 | | | | |
| 213348_at | Cyclin-dependent kinase | −4.743712 | 6.04E−005 | 0.0036153 | 1.743650125 |
| | inhibitor 1C (p57, Kip2) | | | | |
| 219534_x_at | cyclin-dependent kinase | −4.428251 | 0.0001409 | 0.0064487 | 0.897382098 |
| | inhibitor 1C (p57, Kip2) | | | | |
| 213182_x_at | cyclin-dependent kinase | −3.62215 | 0.0011888 | 0.0259445 | −1.143747021 |
| | inhibitor 1C (p57, Kip2) | | | | |
| 216894_x_at | cyclin-dependent kinase | −3.347564 | 0.0024072 | 0.0406668 | −1.819994946 |
| | inhibitor 1C (p57, Kip2) | | | | |
| 209501_at | cerebellar degeneration- | −3.759072 | 0.000832 | 0.0205308 | −0.759864252 |
| | related protein 2, 62 kDa | | | | |
| 216751_at | CMT1A duplicated region | 3.5451725 | 0.0014509 | 0.029498 | −1.312151053 |
| | transcript 4 | | | | |
| 212864_at | CDP-diacylglycerol | 3.5161782 | 0.0015635 | 0.03095 | −1.379363177 |
| | synthase (phosphatidate | | | | |
| | cytidylyltransferase) 2 | | | | |
| 207205_at | carcinoembryonic antigen- | 3.539995 | 0.0014704 | 0.0297861 | −1.303164426 |
| | related cell adhesion | | | | |
| | molecule 4 | | | | |
| 212501_at | CCAAT/enhancer binding | 3.2834811 | 0.0028317 | 0.0448458 | −1.965771632 |
| | protein (C/EBP), beta | | | | |
| 213006_at | CCAAT/enhancer binding | 7.8208216 | 2.04E−008 | 1.69E−005 | 9.380113378 |
| | protein (C/EBP), delta | | | | |
| 203973_s_at | CCAAT/enhancer binding | 5.3247709 | 1.27E−005 | 0.001237 | 3.217998047 |
| | protein (C/EBP), delta | | | | |
| 204739_at | centromere protein C 1 | −3.252694 | 0.0030604 | 0.0470963 | −2.047662823 |
| 219358_s_at | centaurin, alpha 2 | 8.8817081 | 1.66E−009 | 2.06E−006 | 11.79433186 |
| 214102_at | centaurin, delta 1 | 3.9393625 | 0.0005179 | 0.0151633 | −0.299908034 |
| 207719_x_at | centrosomal protein 170 kDa | 3.3045567 | 0.0026847 | 0.0432556 | −1.887355887 |
| 52285_f_at | centrosomal protein 76 kDa | −3.801666 | 0.0007441 | 0.0191095 | −0.695765147 |
| 209616_s_at | carboxylesterase 1 | 3.8098736 | 0.0007283 | 0.0188048 | −0.67821622 |
| | (monocyte/macrophage | | | | |
| | serine esterase 1) | | | | |
| 213509_x_at | carboxylesterase 2 | 3.4871954 | 0.0016846 | 0.0325846 | −1.479771807 |
| | (intestine, liver) | | | | |
| 215388_s_at | complement factor H /// | −3.468439 | 0.0017677 | 0.0335696 | −1.507291615 |
| | complement factor H- | | | | |
| | related 1 | | | | |
| 209508_x_at | CASP8 and FADD-like | 6.8867892 | 2.10E−007 | 7.06E−005 | 7.171950728 |
| | apoptosis regulator | | | | |
| 211316_x_at | CASP8 and FADD-like | 5.691143 | 4.76E−006 | 0.0006233 | 4.151462203 |
| | apoptosis regulator | | | | |
| 214486_x_at | CASP8 and FADD-like | 4.2296001 | 0.0002396 | 0.0093056 | 0.390119623 |
| | apoptosis regulator | | | | |
| 214906_x_at | hypothetical gene CG018 | −3.247195 | 0.0031031 | 0.0474828 | −2.026211762 |
| 220647_s_at | coiled-coil-helix-coiled- | −3.242317 | 0.0031415 | 0.0476847 | −2.072237994 |
| | coil-helix domain | | | | |
| | containing 8 | | | | |
| 218927_s_at | carbohydrate (chondroitin 4) | −3.828828 | 0.0006929 | 0.0182515 | −0.633744679 |
| | sulfotransferase 12 | | | | |
| 206756_at | carbohydrate (N- | −3.262227 | 0.0029878 | 0.0464917 | −2.009673908 |
| | acetylglucosamine 6-O) | | | | |
| | sulfotransferase 7 | | | | |
| 201953_at | calcium and integrin | −3.533793 | 0.0014941 | 0.0301569 | −1.373049343 |
| | binding 1 (calmyrin) | | | | |
| 211759_x_at | cytoskeleton associated | −3.765175 | 0.0008188 | 0.0203634 | −0.768144821 |
| | protein 1 | | | | |
| 200998_s_at | cytoskeleton-associated | 4.8040396 | 5.13E−005 | 0.0032122 | 1.88949702 |
| | protein 4 | | | | |
| 201897_s_at | CDC28 protein kinase | −3.737894 | 0.0008794 | 0.021299 | −0.835184346 |
| | regulatory subunit 1B | | | | |
| 204170_s_at | CDC28 protein kinase | −4.024008 | 0.000414 | 0.0132384 | −0.117323306 |
| | regulatory subunit 2 | | | | |
| 206207_at | Charcot-Leyden crystal | −3.373056 | 0.002256 | 0.0391517 | −1.742060366 |
| | protein /// Charcot-Leyden | | | | |
| | crystal protein | | | | |
| 220132_s_at | C-type lectin domain family | −3.281114 | 0.0028487 | 0.0449592 | −1.931100866 |
| | 2, member D | | | | |
| 205200_at | C-type lectin domain family | 3.6002946 | 0.0012581 | 0.0268042 | −1.169519553 |
| | 3, member B | | | | |
| 219890_at | C-type lectin domain family | 3.3503678 | 0.0023901 | 0.0404668 | −1.773779056 |
| | 5, member A | | | | |
| 201560_at | chloride intracellular | 4.2349587 | 0.0002362 | 0.0092348 | 0.400098978 |
| | channel 4 | | | | |
| 213317_at | chloride intracellular | −4.295299 | 0.0002011 | 0.0082533 | 0.553245716 |
| | channel 5 /// similar to | | | | |
| | chloride intracellular | | | | |
| | channel 5 | | | | |
| 200614_at | clathrin, heavy polypeptide | 3.4660033 | 0.0017788 | 0.033648 | −1.541415555 |
| | (Hc) | | | | |
| 205944_s_at | clathrin, heavy polypeptide- | 5.2563008 | 1.52E−005 | 0.0013909 | 3.03933127 |
| | like 1 | | | | |
| 222043_at | clusterin | 3.282672 | 0.0028375 | 0.0448738 | −1.970857824 |
| 204576_s_at | clusterin associated protein 1 | −3.71827 | 0.0009256 | 0.0219904 | −0.909965491 |
| 218728_s_at | cornichon homolog 4 | 3.7392849 | 0.0008762 | 0.021299 | −0.844199093 |
| | (Drosophila) | | | | |
| 218250_s_at | CCR4-NOT transcription | −5.842978 | 3.18E−006 | 0.0004538 | 4.559729094 |
| | complex, subunit 7 | | | | |
| 219400_at | contactin associated protein 1 | −3.390617 | 0.0021573 | 0.0378505 | −1.692951413 |
| 205229_s_at | coagulation factor C | 3.2711293 | 0.0029214 | 0.0456867 | −1.974753283 |
| | homolog, cochlin (Limulus | | | | |
| | polyphemus) | | | | |
| 203630_s_at | component of oligomeric | −3.31867 | 0.0025904 | 0.0424462 | −1.870753786 |
| | golgi complex 5 | | | | |
| 211011_at | collagen, type XIX, alpha 1 | 3.4106536 | 0.0020497 | 0.0367442 | −1.585023799 |
| 209156_s_at | collagen, type VI, alpha 2 | −3.482818 | 0.0017036 | 0.0328251 | −1.487885827 |
| 209132_s_at | COMM domain containing 4 | −3.57659 | 0.0013377 | 0.027963 | −1.232127592 |
| 208684_at | coatomer protein complex, | 3.6333038 | 0.0011548 | 0.0254961 | −1.112624827 |
| | subunit alpha | | | | |
| 202141_s_at | COP9 constitutive | −3.321304 | 0.0025731 | 0.0422529 | −1.823820898 |
| | photomorphogenic homolog | | | | |
| | subunit 8 (Arabidopsis) | | | | |
| 218328_at | coenzyme Q4 homolog (S. cerevisiae) | −4.569756 | 9.63E−005 | 0.0050396 | 1.258022498 |
| 218760_at | coenzyme Q6 homolog, | −3.259626 | 0.0030074 | 0.0466025 | −2.036860755 |
| | monooxygenase (S. cerevisiae) | | | | |
| 201941_at | carboxypeptidase D | 4.0181842 | 0.0004205 | 0.0132999 | −0.124240091 |
| 201940_at | carboxypeptidase D | 3.8974598 | 0.0005785 | 0.0163756 | −0.452804899 |
| 201943_s_at | carboxypeptidase D | 3.401865 | 0.0020962 | 0.0372865 | −1.684510653 |
| 206100_at | carboxypeptidase M | 5.2370624 | 1.60E−005 | 0.0014332 | 3.011103109 |
| 206918_s_at | copine I | −3.926643 | 0.0005356 | 0.0155808 | −0.345418637 |
| 217552_x_at | complement component | 6.5779604 | 4.64E−007 | 0.000126 | 6.412072722 |
| | (3b/4b) receptor 1 (Knops | | | | |
| | blood group) | | | | |
| 205931_s_at | cAMP responsive element | 6.8831696 | 2.12E−007 | 7.06E−005 | 7.144372934 |
| | binding protein 5 | | | | |
| 202160_at | CREB binding protein | 3.5119738 | 0.0015805 | 0.0310987 | −1.412212357 |
| | (Rubinstein-Taybi | | | | |
| | syndrome) | | | | |
| 201989_s_at | cAMP responsive element | −4.027119 | 0.0004106 | 0.013184 | −0.136267875 |
| | binding protein-like 2 | | | | |
| 201988_s_at | cAMP responsive element | −3.303195 | 0.0026939 | 0.043311 | −1.893960546 |
| | binding protein-like 2 | | | | |
| 206914_at | cytotoxic and regulatory T | −4.308777 | 0.0001939 | 0.0080931 | 0.609962124 |
| | cell molecule | | | | |
| 207085_x_at | colony stimulating factor 2 | 4.4039557 | 0.0001503 | 0.0067472 | 0.882785549 |
| | receptor, alpha, low-affinity | | | | |
| | (granulocyte-macrophage) | | | | |
| 210340_s_at | colony stimulating factor 2 | 3.4562119 | 0.0018241 | 0.0340701 | −1.479901768 |
| | receptor, alpha, low-affinity | | | | |
| | (granulocyte-macrophage) | | | | |
| 203591_s_at | colony stimulating factor 3 | 3.9414177 | 0.0005151 | 0.0151481 | −0.353176878 |
| | receptor (granulocyte) | | | | |
| 208866_at | casein kinase 1, alpha 1 | 3.3974592 | 0.0021199 | 0.0375801 | −1.659325793 |
| 211571_s_at | chondroitin sulfate | 7.0973548 | 1.23E−007 | 5.26E−005 | 7.681872527 |
| | proteoglycan 2 (versican) | | | | |
| 215646_s_at | chondroitin sulfate | 6.3339611 | 8.73E−007 | 0.0002028 | 5.797392458 |
| | proteoglycan 2 (versican) /// | | | | |
| | chondroitin sulfate | | | | |
| | proteoglycan 2 (versican) | | | | |
| 204619_s_at | chondroitin sulfate | 6.0890716 | 1.66E−006 | 0.000303 | 5.177048193 |
| | proteoglycan 2 (versican) | | | | |
| 204620_s_at | chondroitin sulfate | 6.0654142 | 1.77E−006 | 0.0003031 | 5.120383055 |
| | proteoglycan 2 (versican) | | | | |
| 221731_x_at | chondroitin sulfate | 5.8762902 | 2.91E−006 | 0.0004292 | 4.635102698 |
| | proteoglycan 2 (versican) | | | | |
| 204971_at | cystatin A (stefin A) | 5.1248468 | 2.16E−005 | 0.0017801 | 2.701041657 |
| 201220_x_at | C-terminal binding protein 2 | 4.3195857 | 0.0001884 | 0.0079219 | 0.61387662 |
| 210554_s_at | C-terminal binding protein 2 | 3.4303186 | 0.0019492 | 0.0356839 | −1.618943249 |
| 218923_at | chitobiase, di-N-acetyl- | 3.7964034 | 0.0007545 | 0.0193307 | −0.700824882 |
| 210844_x_at | catenin (cadherin-associated | 5.2511975 | 1.54E−005 | 0.0014024 | 3.027675667 |
| | protein), alpha 1, 102 kDa | | | | |
| 200765_x_at | catenin (cadherin-associated | 4.4035529 | 0.0001505 | 0.0067472 | 0.827567487 |
| | protein), alpha 1, 102 kDa | | | | |
| 200764_s_at | catenin (cadherin-associated | 3.7090172 | 0.0009483 | 0.0222975 | −0.898490034 |
| | protein), alpha 1, 102 kDa | | | | |
| 213275_x_at | cathepsin B | 5.9916022 | 2.14E−006 | 0.0003463 | 4.931225528 |
| 200839_s_at | cathepsin B | 4.0521749 | 0.0003842 | 0.0126796 | −0.030845835 |
| 200838_at | cathepsin B | 3.9776434 | 0.0004681 | 0.0142881 | −0.224314512 |
| 200766_at | cathepsin D (lysosomal | 5.0468915 | 2.67E−005 | 0.0020584 | 2.521325146 |
| | aspartyl peptidase) | | | | |
| 203758_at | cathepsin O | −4.315004 | 0.0001907 | 0.0079894 | 0.607939657 |
| 214743_at | cut-like 1, CCAAT | 5.6999558 | 4.65E−006 | 0.0006125 | 4.187520279 |
| | displacement protein | | | | |
| | (Drosophila) | | | | |
| 209774_x_at | chemokine (C—X—C motif) | 3.3540163 | 0.002368 | 0.0402799 | −1.728037064 |
| | ligand 2 | | | | |
| 217119_s_at | chemokine (C—X—C motif) | −6.212669 | 1.20E−006 | 0.0002407 | 5.495676727 |
| | receptor 3 | | | | |
| 207681_at | chemokine (C—X—C motif) | −3.332628 | 0.0025003 | 0.0415771 | −1.856254039 |
| | receptor 3 | | | | |
| 213315_x_at | chromosome X open | −3.306847 | 0.0026691 | 0.0431302 | −1.897096836 |
| | reading frame 40A | | | | |
| 212961_x_at | chromosome X open | −3.47027 | 0.0017594 | 0.0334519 | −1.506477356 |
| | reading frame 40B | | | | |
| 209163_at | cytochrome b-561 | −7.083343 | 1.27E−007 | 5.26E−005 | 7.652027367 |
| 201633_s_at | cytochrome b5 type B (outer | −5.031851 | 2.78E−005 | 0.0021061 | 2.481093287 |
| | mitochondrial membrane) | | | | |
| 202263_at | cytochrome b5 reductase 1 | 3.5714815 | 0.0013555 | 0.0281204 | −1.251624017 |
| 208923_at | cytoplasmic FMR1 | 3.2536396 | 0.0030531 | 0.0470787 | −2.039925523 |
| | interacting protein 1 | | | | |
| 213295_at | cylindromatosis (turban | −3.41317 | 0.0020365 | 0.0366857 | −1.596737576 |
| | tumor syndrome) | | | | |
| 202436_s_at | cytochrome P450, family 1, | 7.1543503 | 1.07E−007 | 4.75E−005 | 7.822723722 |
| | subfamily B, polypeptide 1 | | | | |
| 202435_s_at | cytochrome P450, family 1, | 6.8642415 | 2.22E−007 | 7.07E−005 | 7.116902873 |
| | subfamily B, polypeptide 1 | | | | |
| 202437_s_at | cytochrome P450, family 1, | 4.6356941 | 8.07E−005 | 0.0043967 | 1.422390127 |
| | subfamily B, polypeptide 1 | | | | |
| 202434_s_at | cytochrome P450, family 1, | 3.3047641 | 0.0026833 | 0.0432556 | −1.824421763 |
| | subfamily B, polypeptide 1 | | | | |
| 209569_x_at | DNA segment on | −3.283804 | 0.0028294 | 0.0448412 | −1.963655545 |
| | chromosome 4 (unique) 234 | | | | |
| | expressed sequence | | | | |
| 201280_s_at | disabled homolog 2, | 6.3730121 | 7.89E−007 | 0.000189 | 5.892530411 |
| | mitogen-responsive | | | | |
| | phosphoprotein | | | | |
| | (Drosophila) | | | | |
| 201279_s_at | disabled homolog 2, | 5.6555385 | 5.23E−006 | 0.000671 | 4.067400945 |
| | mitogen-responsive | | | | |
| | phosphoprotein | | | | |
| | (Drosophila) | | | | |
| 210757_x_at | disabled homolog 2, | 4.9450304 | 3.51E−005 | 0.0024299 | 2.230857636 |
| | mitogen-responsive | | | | |
| | phosphoprotein | | | | |
| | (Drosophila) | | | | |
| 201278_at | Disabled homolog 2, | 3.4578984 | 0.0018162 | 0.03398 | −1.563783579 |
| | mitogen-responsive | | | | |
| | phosphoprotein | | | | |
| | (Drosophila) | | | | |
| 205471_s_at | dachshund homolog 1 | 4.0081925 | 0.0004317 | 0.0135115 | −0.115378248 |
| | (Drosophila) | | | | |
| 203139_at | death-associated protein | 4.1353447 | 0.000308 | 0.0109442 | 0.149012444 |
| | kinase 1 | | | | |
| 201763_s_at | death-associated protein 6 | −4.129981 | 0.0003125 | 0.0110346 | 0.144263044 |
| 209782_s_at | D site of albumin promoter | −4.166334 | 0.0002837 | 0.0103617 | 0.207534971 |
| | (albumin D-box) binding | | | | |
| | protein | | | | |
| 205371_s_at | dihydrolipoamide branched | −4.192267 | 0.0002647 | 0.0098805 | 0.320799932 |
| | chain transacylase E2 | | | | |
| 201571_s_at | dCMP deaminase | −5.147641 | 2.04E−005 | 0.0016994 | 2.780531075 |
| 202262_x_at | dimethylarginine | 3.3160347 | 0.0026077 | 0.0425668 | −1.804789011 |
| | dimethylaminohydrolase 2 | | | | |
| 203409_at | damage-specific DNA | −5.289072 | 1.39E−005 | 0.0013052 | 3.150577707 |
| | binding protein 2, 48 kDa | | | | |
| 221039_s_at | development and | 7.0328086 | 1.45E−007 | 5.64E−005 | 7.52906031 |
| | differentiation enhancing | | | | |
| | factor 1 | | | | |
| 205763_s_at | DEAD (Asp-Glu-Ala-Asp) | −3.543673 | 0.0014565 | 0.0295315 | −1.296325793 |
| | box polypeptide 18 | | | | |
| 202578_s_at | DEAD (Asp-Glu-Ala-As) | −3.641141 | 0.0011316 | 0.0251896 | −1.060434834 |
| | box polypeptide 19A | | | | |
| 200702_s_at | DEAD (Asp-Glu-Ala-Asp) | −3.62553 | 0.0011784 | 0.0258445 | −1.129980661 |
| | box polypeptide 24 | | | | |
| 221699_s_at | DEAD (Asp-Glu-Ala-Asp) | −3.331055 | 0.0025103 | 0.0416502 | −1.805485634 |
| | box polypeptide 50 /// | | | | |
| | DEAD (Asp-Glu-Ala-Asp) | | | | |
| | box polypeptide 50 | | | | |
| 221081_s_at | DENN/MADD domain | −7.06017 | 1.35E−007 | 5.47E−005 | 7.573105401 |
| | containing 2D | | | | |
| 218102_at | 2-deoxyribose-5-phosphate | 5.0635265 | 2.55E−005 | 0.0019821 | 2.549225552 |
| | aldolase homolog (C. elegans) | | | | |
| 211558_s_at | deoxyhypusine synthase | −5.01955 | 2.87E−005 | 0.0021433 | 2.437676851 |
| 202481_at | dehydrogenase/reductase | −3.608352 | 0.0012321 | 0.0264526 | −1.156000373 |
| | (SDR family) member 3 | | | | |
| 205603_s_at | diaphanous homolog 2 | 5.3410534 | 1.21E−005 | 0.0011938 | 3.262418645 |
| | (Drosophila) | | | | |
| 212888_at | Dicer1, Dcr-1 homolog | 6.2936145 | 9.70E−007 | 0.0002131 | 5.693791253 |
| | (Drosophila) | | | | |
| 206061_s_at | Dicer1, Dcr-1 homolog | 5.3574322 | 1.16E−005 | 0.0011734 | 3.297311755 |
| | (Drosophila) | | | | |
| 213229_at | Dicer1, Dcr-1 homolog | 3.8200061 | 0.0007092 | 0.0185259 | −0.663504205 |
| | (Drosophila) | | | | |
| 204405_x_at | DIM1 dimethyladenosine | −3.515379 | 0.0015667 | 0.03095 | −1.379202772 |
| | transferase 1-like (S. cerevisiae) | | | | |
| 216870_x_at | deleted in lymphocytic | 3.6089402 | 0.0012302 | 0.0264526 | −1.157430128 |
| | leukemia, 2 | | | | |
| | discs, large (Drosophila) | | | | |
| 202570_s_at | homolog-associated protein 4 | 3.2471517 | 0.0031035 | 0.0474828 | −2.026350124 |
| 203791_at | Dmx-like 1 | 3.7891417 | 0.000769 | 0.0196056 | −0.701683976 |
| 215761_at | Dmx-like 2 | 4.3980768 | 0.0001527 | 0.0068192 | 0.87930287 |
| 212820_at | Dmx-like 2 | 3.5007329 | 0.0016269 | 0.0317731 | −1.435634663 |
| 205963_s_at | DnaJ (Hsp40) homolog, | −3.526575 | 0.0015222 | 0.0304477 | −1.387021788 |
| | subfamily A, member 3 | | | | |
| 212467_at | DnaJ (Hsp40) homolog, | 5.1781615 | 1.88E−005 | 0.001614 | 2.829811318 |
| | subfamily C, member 13 | | | | |
| 218435_at | DnaJ (Hsp40) homolog, | 3.4743292 | 0.0017412 | 0.0332469 | −1.477791792 |
| | subfamily C, member 15 | | | | |
| 203716_s_at | dipeptidyl-peptidase 4 | −3.746211 | 0.0008605 | 0.0210468 | −0.853590542 |
| | (CD26, adenosine | | | | |
| | deaminase complexing | | | | |
| | protein 2) | | | | |
| 218627_at | damage-regulated | 3.2827191 | 0.0028371 | 0.0448738 | −1.924923337 |
| | autophagy modulator | | | | |
| 203258_at | DR1-associated protein 1 | −5.164812 | 1.94E−005 | 0.0016538 | 2.790561585 |
| | (negative cofactor 2 alpha) | | | | |
| 201021_s_at | destrin (actin | −3.617546 | 0.0012031 | 0.0261284 | −1.147150686 |
| | depolymerizing factor) | | | | |
| 201041_s_at | dual specificity phosphatase 1 | 4.7094687 | 6.62E−005 | 0.0038597 | 1.609517757 |
| 201044_x_at | dual specificity phosphatase 1 | 4.0764145 | 0.0003603 | 0.0121088 | 0.023352009 |
| 209457_at | dual specificity phosphatase 5 | −5.617437 | 5.79E−006 | 0.0007164 | 3.995646934 |
| 218660_at | dysferlin, limb girdle | 6.9457601 | 1.81E−007 | 6.60E−005 | 7.308255352 |
| | muscular dystrophy 2B | | | | |
| | (autosomal recessive) | | | | |
| 203692_s_at | E2F transcription factor 3 | 3.8566266 | 0.0006441 | 0.0173552 | −0.540192125 |
| 203693_s_at | E2F transcription factor 3 | 3.5677866 | 0.0013685 | 0.0282886 | −1.290562077 |
| 204278_s_at | estrogen receptor binding | −3.574152 | 0.0013462 | 0.0280347 | −1.260917141 |
| | site associated, antigen, 9 | | | | |
| 213787_s_at | emopamil binding protein | −4.08811 | 0.0003493 | 0.0118097 | 0.019054972 |
| | (sterol isomerase) | | | | |
| 202735_at | emopamil binding protein | −3.345293 | 0.0024211 | 0.0407891 | −1.788041599 |
| | (sterol isomerase) | | | | |
| 208091_s_at | EGFR-coamplified and | −3.88912 | 0.0005913 | 0.0165114 | −0.452383673 |
| | overexpressed protein /// | | | | |
| | EGFR-coamplified and | | | | |
| | overexpressed protein | | | | |
| 204642_at | endothelial differentiation, | −4.038768 | 0.0003981 | 0.0129796 | −0.11096426 |
| | sphingolipid G-protein- | | | | |
| | coupled receptor, 1 | | | | |
| 221417_x_at | endothelial differentiation, | −3.462217 | 0.0017962 | 0.0338007 | −1.546970897 |
| | sphingolipid G-protein- | | | | |
| | coupled receptor, 8 | | | | |
| 214394_x_at | eukaryotic translation | −3.303622 | 0.002691 | 0.0432955 | −1.889520518 |
| | elongation factor 1 delta | | | | |
| | (guanine nucleotide | | | | |
| | exchange protein) /// similar | | | | |
| | to Elongation factor 1-delta | | | | |
| | (EF-1-delta) (Antigen NY- | | | | |
| | CO-4) /// similar to | | | | |
| | Elongation factor 1-delta | | | | |
| | (EF-1-delta) (Antigen NY- | | | | |
| | CO-4) | | | | |
| 201694_s_at | early growth response 1 | 3.5166839 | 0.0015615 | 0.03095 | −1.289341908 |
| 201693_s_at | early growth response 1 | 3.5118003 | 0.0015812 | 0.0310987 | −1.369298363 |
| 212653_s_at | EH domain binding protein 1 | −4.185726 | 0.0002694 | 0.0099707 | 0.28398139 |
| 210501_x_at | eukaryotic translation | −4.552227 | 0.000101 | 0.0052213 | 1.25268058 |
| | initiation factor 3, subunit | | | | |
| | 12 | | | | |
| 221494_x_at | eukaryotic translation | −3.75473 | 0.0008415 | 0.0207195 | −0.764888451 |
| | initiation factor 3, subunit | | | | |
| | 12 | | | | |
| 200023_s_at | eukaryotic translation | −3.532563 | 0.0014989 | 0.0302253 | −1.329606182 |
| | initiation factor 3, subunit 5 | | | | |
| | epsilon, 47 kDa | | | | |
| 201530_x_at | eukaryotic translation | 3.5580178 | 0.0014035 | 0.0287717 | −1.236761356 |
| | initiation factor 4A, isoform 1 | | | | |
| 211937_at | eukaryotic translation | −5.371809 | 1.12E−005 | 0.0011405 | 3.353043436 |
| | initiation factor 4B | | | | |
| 200004_at | eukaryotic translation | 4.2222696 | 0.0002444 | 0.009383 | 0.356527453 |
| | initiation factor 4 gamma, 2 | | | | |
| 201935_s_at | eukaryotic translation | 4.2961702 | 0.0002006 | 0.0082533 | 0.551465223 |
| | initiation factor 4 gamma, 3 | | | | |
| 201936_s_at | eukaryotic translation | 3.6334688 | 0.0011543 | 0.0254961 | −1.13063218 |
| | initiation factor 4 gamma, 3 | | | | |
| 208707_at | eukaryotic translation | 4.4472996 | 0.0001338 | 0.0062624 | 0.93694154 |
| | initiation factor 5 | | | | |
| 201123_s_at | eukaryotic translation | −5.801158 | 3.55E−006 | 0.0004944 | 4.464733997 |
| | initiation factor 5A | | | | |
| 201122_x_at | eukaryotic translation | −3.809947 | 0.0007282 | 0.0188048 | −0.671071201 |
| | initiation factor 5A | | | | |
| 214831_at | ELK4, ETS-domain protein | −3.462772 | 0.0017936 | 0.0338007 | −1.527188055 |
| | (SRF accessory protein 1) | | | | |
| 210868_s_at | ELOVL family member 6, | −3.79181 | 0.0007636 | 0.0194913 | −0.727144732 |
| | elongation of long chain | | | | |
| | fatty acids (FEN1/Elo2, | | | | |
| | SUR4/Elo3-like, yeast) | | | | |
| 201313_at | enolase 2 (gamma, | −3.223775 | 0.0032914 | 0.0492896 | −2.076776403 |
| | neuronal) | | | | |
| 201718_s_at | erythrocyte membrane | −4.055973 | 0.0003804 | 0.0126315 | −0.043303546 |
| | protein band 4.1-like 2 | | | | |
| 207347_at | excision repair cross- | −3.840298 | 0.0006724 | 0.0179215 | −0.616669739 |
| | complementing rodent | | | | |
| | repair deficiency, | | | | |
| | complementation group 6 | | | | |
| 218135_at | ERGIC and golgi 2 | −4.464176 | 0.0001279 | 0.0060775 | 0.99047641 |
| 205530_at | electron-transferring- | 3.9159589 | 0.0005509 | 0.0159223 | −0.395064569 |
| | flavoprotein dehydrogenase | | | | |
| 212627_s_at | exosome component 7 | −3.31496 | 0.0026148 | 0.0426086 | −1.893669394 |
| 205061_s_at | exosome component 9 | −3.692575 | 0.0009898 | 0.0229986 | −0.965140672 |
| 201995_at | exostoses (multiple) 1 | 3.4801097 | 0.0017155 | 0.0330115 | −1.488858072 |
| 204713_s_at | coagulation factor V | 7.8705559 | 1.81E−008 | 1.55E−005 | 9.39995824 |
| | (proaccelerin, labile factor) | | | | |
| 204714_s_at | coagulation factor V | 6.9047733 | 2.00E−007 | 7.06E−005 | 7.202864473 |
| | (proaccelerin, labile factor) | | | | |
| 212400_at | family with sequence | −3.253637 | 0.0030532 | 0.0470787 | −2.015754576 |
| | similarity 102, member A | | | | |
| 219694_at | family with sequence | 3.4017798 | 0.0020967 | 0.0372865 | −1.670962443 |
| | similarity 105, member A | | | | |
| 221249_s_at | family with sequence | −3.862552 | 0.0006341 | 0.0171698 | −0.540686253 |
| | similarity 117, member A /// | | | | |
| | family with sequence | | | | |
| | similarity 117, member A | | | | |
| 221804_s_at | family with sequence | 4.1804885 | 0.0002732 | 0.0100774 | 0.255914546 |
| | similarity 45, member B /// | | | | |
| | family with sequence | | | | |
| | similarity 45, member A | | | | |
| 218023_s_at | family with sequence | 5.7174035 | 4.43E−006 | 0.0005917 | 4.251355639 |
| | similarity 53, member C | | | | |
| 221856_s_at | family with sequence | 3.439632 | 0.0019032 | 0.0351878 | −1.580973955 |
| | similarity 63, member A | | | | |
| 218126_at | family with sequence | −3.911519 | 0.0005574 | 0.0160474 | −0.419630655 |
| | similarity 82, member C | | | | |
| 218689_at | Fanconi anemia, | −6.820166 | 2.49E−007 | 7.59E−005 | 7.008995717 |
| | complementation group F | | | | |
| 204282_s_at | phenylalanine-tRNA | −3.612701 | 0.0012183 | 0.0263005 | −1.157771585 |
| | synthetase 2 (mitochondrial) | | | | |
| 210865_at | Fas ligand (TNF | −4.361034 | 0.0001686 | 0.0073109 | 0.717257398 |
| | superfamily, member 6) | | | | |
| 211623_s_at | fibrillarin /// fibrillarin | −3.326498 | 0.0025395 | 0.0419858 | −1.819337447 |
| 203088_at | fibulin 5 | −4.67838 | 7.20E−005 | 0.0041288 | 1.543435915 |
| 203184_at | fibrillin 2 (congenital | 5.5614222 | 6.72E−006 | 0.0007799 | 3.823371594 |
| | contractural arachnodactyly) | | | | |
| 209004_s_at | F-box and leucine-rich | 3.5644958 | 0.0013802 | 0.0284511 | −1.285420779 |
| | repeat protein 5 | | | | |
| 212231_at | F-box protein 21 | −4.221394 | 0.0002449 | 0.009383 | 0.41096576 |
| 205310_at | F-box protein 46 | −4.123814 | 0.0003176 | 0.0111289 | 0.165309811 |
| 212987_at | F-box protein 9 | 5.3148623 | 1.30E−005 | 0.0012539 | 3.218957986 |
| 211306_s_at | Fc fragment of IgA, | 4.0480165 | 0.0003885 | 0.0127232 | −0.049070311 |
| | receptor for | | | | |
| 211816_x_at | Fc fragment of IgA, | 3.4189853 | 0.0020065 | 0.0362752 | −1.558474604 |
| | receptor for | | | | |
| 211734_s_at | Fc fragment of IgE, high | −4.494507 | 0.0001179 | 0.0057245 | 1.092010728 |
| | affinity I, receptor for; alpha | | | | |
| | polypeptide /// Fc fragment | | | | |
| | of IgE, high affinity I, | | | | |
| | receptor for; alpha | | | | |
| | polypeptide | | | | |
| 204006_s_at | Fc fragment of IgG, low | −3.624804 | 0.0011806 | 0.0258461 | −1.058038116 |
| | affinity IIIa, receptor | | | | |
| | (CD16a) /// Fc fragment of | | | | |
| | IgG, low affinity IIIb, | | | | |
| | receptor (CD16b) | | | | |
| 201798_s_at | fer-1-like 3, myoferlin (C. elegans) | 4.4912012 | 0.000119 | 0.005763 | 1.049913681 |
| 205418_at | feline sarcoma oncogene | 5.9270565 | 2.54E−006 | 0.0003896 | 4.78529014 |
| 221345_at | free fatty acid receptor 2 | 3.7742912 | 0.0007995 | 0.0199959 | −0.754985333 |
| 208438_s_at | Gardner-Rasheed feline | 4.6336206 | 8.12E−005 | 0.0044105 | 1.468854007 |
| | sarcoma viral (v-fgr) | | | | |
| | oncogene homolog | | | | |
| 218530_at | formin homology 2 domain | 3.9468708 | 0.0005078 | 0.015057 | −0.321820355 |
| | containing 1 | | | | |
| 218034_at | fission 1 (mitochondrial | −4.295923 | 0.0002007 | 0.0082533 | 0.547291185 |
| | outer membrane) homolog | | | | |
| | (S. cerevisiae) | | | | |
| 204560_at | FK506 binding protein 5 | 4.107492 | 0.0003317 | 0.0114603 | 0.139428193 |
| 220326_s_at | hypothetical protein | 5.1295006 | 2.14E−005 | 0.0017645 | 2.726367378 |
| | FLJ10357 | | | | |
| 58780_s_at | hypothetical protein | 4.2198917 | 0.0002459 | 0.0093959 | 0.358106904 |
| | FLJ10357 | | | | |
| 207489_at | hypothetical protein | 4.0338341 | 0.0004034 | 0.0130454 | −0.109808126 |
| | FLJ12331 | | | | |
| 218798_at | hypothetical protein | −3.539188 | 0.0014735 | 0.0298211 | −1.339420785 |
| | FLJ12949 | | | | |
| 219383_at | hypothetical protein | −3.337828 | 0.0024675 | 0.0411856 | −1.852406633 |
| | FLJ14213 | | | | |
| 212995_x_at | hypothetical protein | −3.747357 | 0.0008579 | 0.0210069 | −0.844742283 |
| | FLJ14346 | | | | |
| 218035_s_at | RNA-binding protein | 5.4428778 | 9.22E−006 | 0.000993 | 3.5426128 |
| 218844_at | hypothetical protein | −3.672072 | 0.0010441 | 0.0238623 | −1.018592493 |
| | FLJ20920 | | | | |
| 218454_at | hypothetical protein | 5.8561059 | 3.07E−006 | 0.0004411 | 4.585888994 |
| | FLJ22662 | | | | |
| 206674_at | fms-related tyrosine kinase 3 | 8.9158813 | 1.54E−009 | 2.06E−006 | 11.78456326 |
| 206980_s_at | fms-related tyrosine kinase | −5.706615 | 4.56E−006 | 0.0006053 | 4.21788194 |
| | 3 ligand | | | | |
| 210607_at | fms-related tyrosine kinase | −4.119996 | 0.0003209 | 0.0111897 | 0.158338196 |
| | 3 ligand | | | | |
| 210495_x_at | fibronectin 1 | 5.0922513 | 2.36E−005 | 0.0018807 | 2.626974991 |
| 218618_s_at | fibronectin type III domain | 5.1939187 | 1.80E−005 | 0.0015652 | 2.874308796 |
| | containing 3B | | | | |
| 209471_s_at | farnesyltransferase, CAAX | −6.417811 | 7.02E−007 | 0.00017 | 6.013878869 |
| | box, alpha | | | | |
| 200090_at | farnesyltransferase, CAAX | −5.001284 | 3.02E−005 | 0.0021978 | 2.409299438 |
| | box, alpha /// | | | | |
| | farnesyltransferase, CAAX | | | | |
| | box, alpha | | | | |
| 209189_at | v-fos FBJ murine | 4.0088762 | 0.0004309 | 0.0135115 | −0.143277902 |
| | osteosarcoma viral | | | | |
| | oncogene homolog | | | | |
| 202768_at | FBJ murine osteosarcoma | 3.7634392 | 0.0008225 | 0.0204012 | −0.711235189 |
| | viral oncogene homolog B | | | | |
| 204131_s_at | forkhead box O3A | 3.5286133 | 0.0015142 | 0.0303425 | −1.375339602 |
| 205118_at | formyl peptide receptor 1 /// | 5.9215304 | 2.58E−006 | 0.0003896 | 4.772959009 |
| | formyl peptide receptor 1 | | | | |
| 205119_s_at | formyl peptide receptor 1 /// | 5.0307434 | 2.79E−005 | 0.0021061 | 2.466325313 |
| | formyl peptide receptor 1 | | | | |
| 219889_at | frequently rearranged in | 6.25963 | 1.06E−006 | 0.0002272 | 5.619733039 |
| | advanced T-cell lymphomas | | | | |
| 209864_at | frequently rearranged in | 4.318654 | 0.0001889 | 0.0079267 | 0.640077595 |
| | advanced T-cell lymphomas 2 | | | | |
| 217370_x_at | fusion (involved in t(12; 16) | −4.05063 | 0.0003858 | 0.0126796 | −0.046999583 |
| | in malignant liposarcoma) | | | | |
| 203172_at | fragile X mental retardation, | −4.152206 | 0.0002945 | 0.0106 | 0.200605037 |
| | autosomal homolog 2 | | | | |
| 211794_at | FYN binding protein (FYB- | 4.3307103 | 0.0001829 | 0.0077777 | 0.642007649 |
| | 120/130) | | | | |
| 221245_s_at | frizzled homolog 5 | 4.694694 | 6.89E−005 | 0.0039856 | 1.608281324 |
| | (Drosophila) /// frizzled | | | | |
| | homolog 5 (Drosophila) | | | | |
| 201514_s_at | Ras-GTPase-activating | −4.295195 | 0.0002011 | 0.0082533 | 0.56604055 |
| | protein SH3-domain- | | | | |
| | binding protein | | | | |
| 203853_s_at | GRB2-associated binding | 3.2714613 | 0.0029189 | 0.0456867 | −1.967358997 |
| | protein 2 | | | | |
| 203725_at | growth arrest and DNA- | 4.2667591 | 0.000217 | 0.0086937 | 0.520267264 |
| | damage-inducible, alpha | | | | |
| 212891_s_at | growth arrest and DNA- | −3.246634 | 0.0031075 | 0.0474928 | −2.052471661 |
| | damage-inducible, gamma | | | | |
| | interacting protein 1 | | | | |
| 203066_at | B cell RAG associated | 3.9528999 | 0.0004997 | 0.0149201 | −0.31680701 |
| | protein | | | | |
| 218871_x_at | chondroitin sulfate | 4.6544425 | 7.67E−005 | 0.0042428 | 1.472480579 |
| | GalNAcT-2 | | | | |
| 222235_s_at | chondroitin sulfate | 4.5727008 | 9.56E−005 | 0.0050126 | 1.248946807 |
| | GalNAcT-2 /// similar to | | | | |
| | chondroitin beta1,4 N- | | | | |
| | acetylgalactosaminyltransferase 2 | | | | |
| 206335_at | galactosamine (N-acetyl)-6- | 6.3239096 | 8.96E−007 | 0.0002059 | 5.779394683 |
| | sulfate sulfatase (Morquio | | | | |
| | syndrome, | | | | |
| | mucopolysaccharidosis type | | | | |
| | IVA) | | | | |
| 213123_at | UDP-N-acetyl-alpha-D- | 3.590157 | 0.0012916 | 0.0272538 | −1.16392073 |
| | galactosamine:polypeptide | | | | |
| | N- | | | | |
| | acetylgalactosaminyltransferase | | | | |
| | 10 (GalNAc-T10) | | | | |
| 219013_at | UDP-N-acetyl-alpha-D- | −3.6284 | 0.0011696 | 0.0257286 | −1.029403369 |
| | galactosamine:polypeptide | | | | |
| | N- | | | | |
| | acetylgalactosaminyltransferase | | | | |
| | 11 (GalNAc-T11) | | | | |
| 218885_s_at | UDP-N-acetyl-alpha-D- | −3.63636 | 0.0011457 | 0.0253776 | −1.097243728 |
| | galactosamine:polypeptide | | | | |
| | N- | | | | |
| | acetylgalactosaminyltransferase | | | | |
| | 12 (GalNAc-T12) | | | | |
| 219271_at | UDP-N-acetyl-alpha-D- | 3.3232187 | 0.0025607 | 0.0421413 | −1.852845209 |
| | galactosamine:polypeptide | | | | |
| | N- | | | | |
| | acetylgalactosaminyltransferase | | | | |
| | 14 (GalNAc-T14) | | | | |
| 212802_s_at | GTPase activating protein | 5.0536126 | 2.62E−005 | 0.0020286 | 2.511271364 |
| | and VPS9 domains 1 | | | | |
| 31874_at | growth arrest-specific 2 like 1 | 4.4243454 | 0.0001423 | 0.0064859 | 0.884627296 |
| 202192_s_at | growth arrest-specific 7 | 5.9982522 | 2.11E−006 | 0.0003463 | 4.960463643 |
| 202191_s_at | growth arrest-specific 7 | 5.308446 | 1.32E−005 | 0.0012701 | 3.178627359 |
| 211067_s_at | growth arrest-specific 7 /// | 4.2187391 | 0.0002467 | 0.0093959 | 0.359407526 |
| | growth arrest-specific 7 | | | | |
| 210872_x_at | growth arrest-specific 7 | 3.8805892 | 0.0006047 | 0.0167115 | −0.476479009 |
| 209602_s_at | GATA binding protein 3 | −5.026144 | 2.82E−005 | 0.0021203 | 2.494288905 |
| 209604_s_at | GATA binding protein 3 | −4.78928 | 5.34E−005 | 0.0033048 | 1.915617587 |
| 209603_at | GATA binding protein 3 | −3.604179 | 0.0012455 | 0.0266858 | −1.202176556 |
| 210589_s_at | glucosidase, beta; acid | 3.6710052 | 0.001047 | 0.0239041 | −0.941374007 |
| | (includes | | | | |
| | glucosylceramidase) /// | | | | |
| | glucosidase, beta; acid, | | | | |
| | pseudogene | | | | |
| 202270_at | guanylate binding protein 1, | −5.322769 | 1.27E−005 | 0.0012383 | 3.209826749 |
| | interferon-inducible, 67 kDa | | | | |
| | /// guanylate binding protein | | | | |
| | 1, interferon-inducible, | | | | |
| | 67 kDa | | | | |
| 202269_x_at | guanylate binding protein 1, | −3.95713 | 0.0004942 | 0.0148205 | −0.327860613 |
| | interferon-inducible, 67 kDa | | | | |
| | /// guanylate binding protein | | | | |
| | 1, interferon-inducible, | | | | |
| | 67 kDa | | | | |
| 203765_at | grancalcin, EF-hand | 5.170551 | 1.91E−005 | 0.0016347 | 2.846947326 |
| | calcium binding protein /// | | | | |
| | grancalcin, EF-hand | | | | |
| | calcium binding protein | | | | |
| 64064_at | GTPase, IMAP family | −6.980471 | 1.65E−007 | 6.14E−005 | 7.401416652 |
| | member 5 | | | | |
| 218805_at | GTPase, IMAP family | −6.740512 | 3.05E−007 | 8.94E−005 | 6.81182532 |
| | member 5 /// GTPase, | | | | |
| | IMAP family member 5 | | | | |
| 219777_at | GTPase, IMAP family | −5.118 | 2.21E−005 | 0.0017933 | 2.694826934 |
| | member 6 | | | | |
| 203157_s_at | glutaminase | −4.501795 | 0.0001156 | 0.005663 | 1.137989862 |
| 203158_s_at | glutaminase | −3.6841 | 0.0010119 | 0.023375 | −0.994282181 |
| 218146_at | glycosyltransferase 8 | −4.803997 | 5.13E−005 | 0.0032122 | 1.883820709 |
| | domain containing 1 | | | | |
| 215001_s_at | glutamate-ammonia ligase | 6.9032827 | 2.01E−007 | 7.06E−005 | 7.211931747 |
| | (glutamine synthetase) | | | | |
| 205349_at | guanine nucleotide binding | 4.9193864 | 3.76E−005 | 0.0025327 | 2.184476093 |
| | protein (G protein), alpha 15 | | | | |
| | (Gq class) | | | | |
| 202615_at | Guanine nucleotide binding | 4.9510738 | 3.45E−005 | 0.0024056 | 2.25862776 |
| | protein (G protein), q | | | | |
| | polypeptide | | | | |
| 201921_at | guanine nucleotide binding | 4.4835854 | 0.0001214 | 0.0058615 | 1.044237137 |
| | protein (G protein), gamma | | | | |
| | 10 /// hypothetical protein | | | | |
| | LOC552891 /// GNG10 | | | | |
| | pseudogene | | | | |
| 217629_at | Guanine nucleotide binding | −4.022362 | 0.0004158 | 0.0132558 | −0.129572667 |
| | protein (G protein), gamma | | | | |
| | transducing activity | | | | |
| | polypeptide 2 | | | | |
| 37145_at | granulysin | −3.725681 | 0.0009079 | 0.0217998 | −0.854629872 |
| 205495_s_at | granulysin /// granulysin | −3.311004 | 0.0026412 | 0.0428338 | −1.874727886 |
| 212335_at | glucosamine (N-acetyl)-6- | 3.2533911 | 0.0030551 | 0.0470787 | −2.005598687 |
| | sulfatase (Sanfilippo disease | | | | |
| | IIID) | | | | |
| 208842_s_at | golgi reassembly stacking | −3.364738 | 0.0023043 | 0.0395937 | −1.759729792 |
| | protein 2, 55 kDa | | | | |
| 208524_at | G protein-coupled receptor | −3.778418 | 0.0007909 | 0.0199363 | −0.759793442 |
| | 15 | | | | |
| 207651_at | G protein-coupled receptor | −4.483268 | 0.0001215 | 0.0058615 | 1.051725756 |
| | 171 | | | | |
| 210279_at | G protein-coupled receptor | −4.568339 | 9.67E−005 | 0.005047 | 1.291304293 |
| | 18 | | | | |
| 210640_s_at | G protein-coupled receptor | 6.4845034 | 5.90E−007 | 0.000153 | 6.173410568 |
| | 30 | | | | |
| 211829_s_at | G protein-coupled receptor | 6.4243249 | 6.90E−007 | 0.000169 | 5.992630343 |
| | 30 | | | | |
| 200736_s_at | glutathione peroxidase 1 | 3.2579198 | 0.0030204 | 0.0467675 | −2.017711816 |
| 213170_at | glutathione peroxidase 7 | −3.584048 | 0.0013122 | 0.0275837 | −1.221262861 |
| 209409_at | growth factor receptor- | 5.3455611 | 1.20E−005 | 0.0011938 | 3.314645486 |
| | bound protein 10 | | | | |
| 215248_at | growth factor receptor- | 3.9561994 | 0.0004954 | 0.014837 | −0.293993285 |
| | bound protein 10 | | | | |
| 210999_s_at | growth factor receptor- | 3.2985761 | 0.0027256 | 0.0437257 | −1.853963312 |
| | bound protein 10 | | | | |
| 206204_at | growth factor receptor- | 3.3617924 | 0.0023216 | 0.0396727 | −1.734798733 |
| | bound protein 14 | | | | |
| 211284_s_at | granulin | 3.510023 | 0.0015885 | 0.0311589 | −1.399242598 |
| 217751_at | glutathione S-transferase | −3.38872 | 0.0021677 | 0.0380042 | −1.68153571 |
| | kappa 1 | | | | |
| 209531_at | glutathione transferase zeta | 3.309628 | 0.0026504 | 0.0428897 | −1.880465024 |
| | 1 (maleylacetoacetate | | | | |
| | isomerase) | | | | |
| 221540_x_at | general transcription factor | 4.2378448 | 0.0002344 | 0.00918 | 0.449826484 |
| | IIH, polypeptide 2, 44 kDa | | | | |
| | /// similar to TFIIH basal | | | | |
| | transcription factor complex | | | | |
| | p44 subunit (Basic | | | | |
| | transcription factor 2 44 kDa | | | | |
| | subunit) (BTF2-p44) | | | | |
| | (General transcription factor | | | | |
| | IIH polypeptide 2) /// | | | | |
| | similar to TFIIH basal | | | | |
| | transcription factor complex | | | | |
| | p44 subunit (Basic | | | | |
| | transcription factor 2 44 kDa | | | | |
| | subunit) (BTF2-p44) | | | | |
| | (General transcription factor | | | | |
| | IIH polypeptide 2) /// | | | | |
| | similar to TFIIH basal | | | | |
| | transcription factor complex | | | | |
| | p44 subunit (Basic | | | | |
| | transcription factor 2 44 kDa | | | | |
| | subunit) (BTF2-p44) | | | | |
| | (General transcription factor | | | | |
| | IIH polypeptide 2) /// | | | | |
| | similar to TFIIH basal | | | | |
| | transcription factor complex | | | | |
| | p44 subunit (Basic | | | | |
| | transcription factor 2 44 kDa | | | | |
| | subunit) (BTF2-p44) | | | | |
| | (General transcription factor | | | | |
| | IIH polypeptide 2) | | | | |
| 206312_at | guanylate cyclase 2C (heat | 3.9198857 | 0.0005452 | 0.0157992 | −0.395403161 |
| | stable enterotoxin receptor) | | | | |
| 205488_at | granzyme A (granzyme 1, | −3.235456 | 0.0031962 | 0.0481577 | −2.066327902 |
| | cytotoxic T-lymphocyte- | | | | |
| | associated serine esterase 3) | | | | |
| | /// granzyme A (granzyme | | | | |
| | 1, cytotoxic T-lymphocyte- | | | | |
| | associated serine esterase 3) | | | | |
| 210164_at | granzyme B (granzyme 2, | −4.099023 | 0.0003393 | 0.0116248 | 0.056488099 |
| | cytotoxic T-lymphocyte- | | | | |
| | associated serine esterase 1) | | | | |
| | /// granzyme B (granzyme 2, | | | | |
| | cytotoxic T-lymphocyte- | | | | |
| | associated serine esterase 1) | | | | |
| 206666_at | granzyme K (granzyme 3; | −3.337958 | 0.0024667 | 0.0411856 | −1.850137772 |
| | tryptase II) /// granzyme K | | | | |
| | (granzyme 3; tryptase II) | | | | |
| 207460_at | granzyme M (lymphocyte | −7.750349 | 2.43E−008 | 1.84E−005 | 9.235332652 |
| | met-ase 1) | | | | |
| 211940_x_at | H3 histone, family 3A /// H3 | 4.0668453 | 0.0003696 | 0.0123274 | −0.024909632 |
| | histone, family 3A | | | | |
| | pseudogene /// similar to H3 | | | | |
| | histone, family 3B | | | | |
| 211997_x_at | H3 histone, family 3B | 6.0804473 | 1.70E−006 | 0.0003031 | 5.168767149 |
| | (H3.3B) | | | | |
| 209069_s_at | H3 histone, family 3B | 3.938916 | 0.0005185 | 0.0151633 | −0.343922952 |
| | (H3.3B) | | | | |
| 211999_at | H3 histone, family 3B | 3.6139171 | 0.0012144 | 0.0262475 | −1.149245616 |
| | (H3.3B) | | | | |
| 201007_at | hydroxyacyl-Coenzyme A | 4.5052763 | 0.0001146 | 0.0056227 | 1.119680068 |
| | dehydrogenase/3-ketoacyl- | | | | |
| | Coenzyme A thiolase/enoyl- | | | | |
| | Coenzyme A hydratase | | | | |
| | (trifunctional protein), beta | | | | |
| | subunit | | | | |
| 206643_at | histidine ammonia-lyase | 3.914753 | 0.0005527 | 0.0159523 | −0.418468154 |
| 205086_s_at | kleisin beta | −3.808789 | 0.0007304 | 0.0188366 | −0.677317191 |
| 216176_at | hepatocellular carcinoma- | 3.3388445 | 0.0024611 | 0.0411468 | −1.816395512 |
| | related HCRP1 | | | | |
| 200643_at | high density lipoprotein | 3.5783444 | 0.0013317 | 0.0278887 | −1.186427982 |
| | binding protein (vigilin) | | | | |
| 203674_at | helicase with zinc finger | 7.2191971 | 9.05E−008 | 4.20E−005 | 7.981565104 |
| 219863_at | hect domain and RLD 5 | −5.51013 | 7.71E−006 | 0.0008629 | 3.69458333 |
| 217168_s_at | homocysteine-inducible, | 3.6265069 | 0.0011754 | 0.025808 | −1.142924336 |
| | endoplasmic reticulum | | | | |
| | stress-inducible, ubiquitin- | | | | |
| | like domain member 1 | | | | |
| 209960_at | hepatocyte growth factor | 4.5101834 | 0.0001131 | 0.0055737 | 1.140038475 |
| | (hepapoietin A; scatter | | | | |
| | factor) | | | | |
| 208826_x_at | histidine triad nucleotide | −7.684964 | 2.85E−008 | 1.94E−005 | 9.064674732 |
| | binding protein 1 | | | | |
| 207721_x_at | histidine triad nucleotide | −6.078022 | 1.71E−006 | 0.0003031 | 5.159287966 |
| | binding protein 1 | | | | |
| 200093_s_at | histidine triad nucleotide | −4.572629 | 9.56E−005 | 0.0050126 | 1.275769263 |
| | binding protein 1 /// | | | | |
| | histidine triad nucleotide | | | | |
| | binding protein 1 | | | | |
| 205425_at | huntingtin interacting | 6.2228987 | 1.17E−006 | 0.000239 | 5.53011363 |
| | protein 1 | | | | |
| 212293_at | homeodomain interacting | 3.5103848 | 0.001587 | 0.0311573 | −1.418182076 |
| | protein kinase 1 | | | | |
| 214472_at | histone 1, H3d | 3.9115202 | 0.0005574 | 0.0160474 | −0.412988902 |
| 212642_s_at | human immunodeficiency | −3.648463 | 0.0011102 | 0.0249641 | −1.02658605 |
| | virus type I enhancer | | | | |
| | binding protein 2 | | | | |
| 205936_s_at | hexokinase 3 (white cell) | 4.1085693 | 0.0003308 | 0.0114453 | 0.08033075 |
| 217436_x_at | major histocompatibility | −3.872049 | 0.0006185 | 0.0169312 | −0.528414628 |
| | complex, class I, A /// major | | | | |
| | histocompatibility complex, | | | | |
| | class I, H (pseudogene) /// | | | | |
| | similar to HLA class I | | | | |
| | histocompatibility antigen, | | | | |
| | A-29 alpha chain precursor | | | | |
| | (MHC class I antigen A*29) | | | | |
| | (Aw-19) | | | | |
| 211799_x_at | major histocompatibility | −4.358409 | 0.0001698 | 0.0073482 | 0.717811791 |
| | complex, class I, C | | | | |
| 217478_s_at | major histocompatibility | −3.824157 | 0.0007015 | 0.0183997 | −0.623815331 |
| | complex, class II, DM alpha | | | | |
| 203932_at | major histocompatibility | −3.634205 | 0.0011521 | 0.0254947 | −1.056546653 |
| | complex, class II, DM beta | | | | |
| | /// major histocompatibility | | | | |
| | complex, class II, DM beta | | | | |
| 213537_at | major histocompatibility | −6.587999 | 4.52E−007 | 0.0001258 | 6.436948993 |
| | complex, class II, DP alpha 1 | | | | |
| 211991_s_at | major histocompatibility | −6.311143 | 9.27E−007 | 0.0002065 | 5.749261512 |
| | complex, class II, DP alpha 1 | | | | |
| 211990_at | major histocompatibility | −4.280165 | 0.0002094 | 0.0084515 | 0.566672642 |
| | complex, class II, DP alpha 1 | | | | |
| 201137_s_at | major histocompatibility | −3.614986 | 0.0012111 | 0.0262003 | −1.155985014 |
| | complex, class II, DP beta 1 | | | | |
| 213831_at | major histocompatibility | −3.995803 | 0.0004461 | 0.0138256 | −0.172214115 |
| | complex, class II, DQ alpha 1 | | | | |
| 212671_s_at | major histocompatibility | −4.092344 | 0.0003454 | 0.011731 | 0.046805587 |
| | complex, class II, DQ alpha | | | | |
| | 1 /// major | | | | |
| | histocompatibility complex, | | | | |
| | class II, DQ alpha 2 /// | | | | |
| | similar to HLA class II | | | | |
| | histocompatibility antigen, | | | | |
| | DQ(1) alpha chain precursor | | | | |
| | (DC-4 alpha chain) | | | | |
| 211656_x_at | major histocompatibility | −3.674571 | 0.0010373 | 0.0237807 | −1.005931013 |
| | complex, class II, DQ beta 1 | | | | |
| | /// major histocompatibility | | | | |
| | complex, class II, DQ beta 1 | | | | |
| 212998_x_at | major histocompatibility | −3.458473 | 0.0018135 | 0.03398 | −1.540082498 |
| | complex, class II, DQ beta 1 | | | | |
| | /// major histocompatibility | | | | |
| | complex, class II, DQ beta 1 | | | | |
| 208894_at | major histocompatibility | −5.239628 | 1.59E−005 | 0.0014332 | 3.025365609 |
| | complex, class II, DR alpha | | | | |
| | /// major histocompatibility | | | | |
| | complex, class II, DR alpha | | | | |
| 210982_s_at | major histocompatibility | −3.95278 | 0.0004999 | 0.0149201 | −0.288358242 |
| | complex, class II, DR alpha | | | | |
| 215193_x_at | major histocompatibility | −4.972434 | 3.26E−005 | 0.0023134 | 2.327548311 |
| | complex, class II, DR beta 1 | | | | |
| 209312_x_at | major histocompatibility | −4.658021 | 7.60E−005 | 0.0042231 | 1.511437493 |
| | complex, class II, DR beta 1 | | | | |
| | /// major histocompatibility | | | | |
| | complex, class II, DR beta 1 | | | | |
| 208306_x_at | Major histocompatibility | −3.968671 | 0.0004793 | 0.014558 | −0.219488761 |
| | complex, class II, DR beta 1 | | | | |
| 200905_x_at | major histocompatibility | −4.102606 | 0.0003361 | 0.0115515 | 0.077522628 |
| | complex, class I, E | | | | |
| 211528_x_at | HLA-G histocompatibility | −3.924786 | 0.0005382 | 0.0156369 | −0.373920882 |
| | antigen, class I, G | | | | |
| 214438_at | H2.0-like homeobox 1 | 6.1182254 | 1.54E−006 | 0.0002856 | 5.26739525 |
| | (Drosophila) | | | | |
| 210457_x_at | high mobility group AT- | −4.924712 | 3.71E−005 | 0.0025118 | 2.202290331 |
| | hook 1 | | | | |
| 208808_s_at | high-mobility group box 2 | 4.5802596 | 9.37E−005 | 0.0049464 | 1.27310201 |
| 208668_x_at | high-mobility group | −3.853289 | 0.0006498 | 0.017466 | −0.566733623 |
| | nucleosomal binding | | | | |
| | domain 2 | | | | |
| 202579_x_at | high mobility group | −3.977857 | 0.0004678 | 0.0142881 | −0.258737579 |
| | nucleosomal binding | | | | |
| | domain 4 | | | | |
| 218120_s_at | heme oxygenase (decycling) 2 | −4.263943 | 0.0002186 | 0.0087153 | 0.518902503 |
| 204112_s_at | histamine N- | 3.4363345 | 0.0019194 | 0.0353466 | −1.57843365 |
| | methyltransferase | | | | |
| 213470_s_at | heterogeneous nuclear | −4.068773 | 0.0003677 | 0.0122829 | 0.005449397 |
| | ribonucleoprotein H1 (H) | | | | |
| 204647_at | homer homolog 3 | 3.3126325 | 0.0026303 | 0.0427508 | −1.879916944 |
| | (Drosophila) | | | | |
| 206697_s_at | haptoglobin | 5.6431699 | 5.40E−006 | 0.0006841 | 4.071744431 |
| 208470_s_at | haptoglobin /// haptoglobin- | 5.1715605 | 1.91E−005 | 0.0016347 | 2.848262148 |
| | related protein | | | | |
| 213926_s_at | HIV-1 Rev binding protein | 4.7178164 | 6.47E−005 | 0.0038217 | 1.690347444 |
| 218092_s_at | HIV-1 Rev binding protein | 6.6361304 | 3.99E−007 | 0.000114 | 6.555967735 |
| | /// region containing | | | | |
| | hypothetical protein | | | | |
| | LOC285086; HIV-1 Rev | | | | |
| | binding protein | | | | |
| 218091_at | HIV-1 Rev binding protein | 6.1478546 | 1.42E−006 | 0.0002709 | 5.340512512 |
| | /// region containing | | | | |
| | hypothetical protein | | | | |
| | LOC285086; HIV-1 Rev | | | | |
| | binding protein | | | | |
| 219020_at | HCLS1 binding protein 3 | 3.3344318 | 0.0024888 | 0.041449 | −1.750447132 |
| 201413_at | hydroxysteroid (17-beta) | 3.6123932 | 0.0012192 | 0.0263005 | −1.159977129 |
| | dehydrogenase 4 | | | | |
| 213540_at | hydroxysteroid (17-beta) | −4.508218 | 0.0001137 | 0.0055908 | 1.118693495 |
| | dehydrogenase 8 | | | | |
| 200599_s_at | heat shock protein 90 kDa | 3.3817275 | 0.0022067 | 0.038506 | −1.743607161 |
| | beta (Grp94), member 1 | | | | |
| 211936_at | heat shock 70 kDa protein 5 | 7.6812023 | 2.88E−008 | 1.94E−005 | 9.061677362 |
| | (glucose-regulated protein, | | | | |
| | 78 kDa) | | | | |
| 117_at | heat shock 70 kDa protein 6 | 4.4441285 | 0.000135 | 0.0062793 | 0.962217918 |
| | (HSP70B′) /// similar to heat | | | | |
| | shock 70 kDa protein 6 | | | | |
| | (HSP70B) | | | | |
| 208687_x_at | heat shock 70 kDa protein 8 | −3.749197 | 0.0008538 | 0.020929 | −0.808618995 |
| 217774_s_at | hypothetical protein | −4.331529 | 0.0001825 | 0.0077777 | 0.636657208 |
| | HSPC152 | | | | |
| 209192_x_at | HIV-1 Tat interacting | −4.543175 | 0.0001035 | 0.0052977 | 1.206265326 |
| | protein, 60 kDa | | | | |
| 202601_s_at | HIV-1 Tat specific factor 1 | −3.570133 | 0.0013603 | 0.0281697 | −1.264175367 |
| 201185_at | HtrA serine peptidase 1 | 6.2530319 | 1.08E−006 | 0.000229 | 5.605563874 |
| 204683_at | intercellular adhesion | −4.945736 | 3.50E−005 | 0.0024299 | 2.231239329 |
| | molecule 2 | | | | |
| 213620_s_at | intercellular adhesion | −3.244089 | 0.0031275 | 0.0476847 | −2.058144012 |
| | molecule 2 | | | | |
| 207826_s_at | inhibitor of DNA binding 3, | −4.069872 | 0.0003666 | 0.0122655 | −0.025709751 |
| | dominant negative helix- | | | | |
| | loop-helix protein | | | | |
| 201193_at | isocitrate dehydrogenase 1 | 4.1280137 | 0.0003141 | 0.0110574 | 0.133788875 |
| | (NADP+), soluble | | | | |
| 210046_s_at | isocitrate dehydrogenase 2 | −3.42705 | 0.0019655 | 0.0358275 | −1.601186988 |
| | (NADP+), mitochondrial | | | | |
| 219209_at | interferon induced with | −6.843417 | 2.34E−007 | 7.25E−005 | 7.064971115 |
| | helicase C domain 1 | | | | |
| 203153_at | interferon-induced protein | −3.885196 | 0.0005975 | 0.0166033 | −0.489474551 |
| | with tetratricopeptide | | | | |
| | repeats 1 /// interferon- | | | | |
| | induced protein with | | | | |
| | tetratricopeptide repeats 1 | | | | |
| 217502_at | interferon-induced protein | −8.647679 | 2.86E−009 | 3.18E−006 | 11.27869101 |
| | with tetratricopeptide | | | | |
| | repeats 2 | | | | |
| 201601_x_at | interferon induced | −8.572445 | 3.40E−009 | 3.61E−006 | 11.07737096 |
| | transmembrane protein 1 (9- | | | | |
| | 27) | | | | |
| 214022_s_at | interferon induced | −6.053528 | 1.82E−006 | 0.0003098 | 5.105034551 |
| | transmembrane protein 1 (9- | | | | |
| | 27) | | | | |
| 202727_s_at | interferon gamma receptor 1 | 5.1904147 | 1.82E−005 | 0.0015739 | 2.865547775 |
| 211676_s_at | interferon gamma receptor 1 | 5.0382235 | 2.73E−005 | 0.0020782 | 2.491115317 |
| | /// interferon gamma | | | | |
| | receptor 1 | | | | |
| 220418_at | intraflagellar transport 52 | −3.473124 | 0.0017466 | 0.0332929 | −1.50759025 1 |
| | homolog (Chlamydomonas) | | | | |
| | /// ubiquitin associated and | | | | |
| | SH3 domain containing, A | | | | |
| 201393_s_at | insulin-like growth factor 2 | 3.7088704 | 0.0009486 | 0.0222975 | −0.916919378 |
| | receptor | | | | |
| 201392_s_at | insulin-like growth factor 2 | 3.6658659 | 0.0010611 | 0.024078 | −1.039135915 |
| | receptor | | | | |
| 210095_s_at | insulin-like growth factor | −4.61452 | 8.54E−005 | 0.0045759 | 1.376610207 |
| | binding protein 3 | | | | |
| 211648_at | Immunoglobulin heavy | 3.4148635 | 0.0020277 | 0.0365865 | −1.652226321 |
| | constant gamma 1 (G1m | | | | |
| | marker) /// Immunoglobulin | | | | |
| | heavy constant gamma 1 | | | | |
| | (G1m marker) | | | | |
| 208759_at | inhibitor of kappa light | 5.2373614 | 1.60E−005 | 0.0014332 | 2.993747875 |
| | polypeptide gene enhancer | | | | |
| | in B-cells, kinase beta /// | | | | |
| | nicastrin | | | | |
| 207433_at | interleukin 10 | 4.7467925 | 5.99E−005 | 0.0035952 | 1.759734814 |
| 205992_s_at | interleukin 15 | −3.322441 | 0.0025657 | 0.0421622 | −1.843262001 |
| 209827_s_at | interleukin 16 (lymphocyte | −3.910659 | 0.0005587 | 0.0160631 | −0.379721498 |
| | chemoattractant factor) | | | | |
| 209828_s_at | interleukin 16 (lymphocyte | −3.82923 | 0.0006922 | 0.0182515 | −0.603933622 |
| | chemoattractant factor) | | | | |
| 205707_at | interleukin 17 receptor A | 5.5310092 | 7.29E−006 | 0.0008252 | 3.760384407 |
| 215691_x_at | interleukin 17 receptor B | −3.243106 | 0.0031352 | 0.0476847 | −2.04047892 |
| 211372_s_at | interleukin 1 receptor, type | 26.109122 | 1.01E−020 | 2.24E−016 | 30.71443211 |
| | II | | | | |
| 205403_at | interleukin 1 receptor, type | 19.405844 | 2.07E−017 | 1.54E−013 | 26.80773514 |
| | II | | | | |
| 210233_at | interleukin 1 receptor | 3.7691567 | 0.0008103 | 0.0202199 | −0.754697943 |
| | accessory protein | | | | |
| 221658_s_at | interleukin 21 receptor | −5.070272 | 2.51E−005 | 0.0019651 | 2.562612895 |
| 219971_at | interleukin 21 receptor | −3.590188 | 0.0012915 | 0.0272538 | −1.238275236 |
| 220054_at | interleukin 23, alpha subunit | −3.71876 | 0.0009244 | 0.0219904 | −0.917115066 |
| | p19 | | | | |
| 211269_s_at | interleukin 2 receptor, alpha | −3.520267 | 0.0015471 | 0.0308311 | −1.373329098 |
| 205291_at | interleukin 2 receptor, beta | −4.517826 | 0.0001108 | 0.0055225 | 1.203270348 |
| | /// interleukin 2 receptor, | | | | |
| | beta | | | | |
| 204116_at | interleukin 2 receptor, | −5.991858 | 2.14E−006 | 0.0003463 | 4.934872705 |
| | gamma (severe combined | | | | |
| | immunodeficiency) | | | | |
| 203828_s_at | interleukin 32 /// interleukin | −6.070581 | 1.74E−006 | 0.0003031 | 5.138343653 |
| | 32 | | | | |
| 206148_at | interleukin 3 receptor, alpha | −3.449826 | 0.0018542 | 0.0344017 | −1.536390539 |
| | (low affinity) | | | | |
| 210624_s_at | ilvB (bacterial acetolactate | −4.219376 | 0.0002463 | 0.0093959 | 0.394490026 |
| | synthase)-like | | | | |
| 202993_at | ilvB (bacterial acetolactate | −3.779598 | 0.0007885 | 0.0199198 | −0.737006639 |
| | synthase)-like | | | | |
| 221688_s_at | IMP3, U3 small nucleolar | −7.319613 | 7.04E−008 | 3.68E−005 | 8.216503503 |
| | ribonucleoprotein, homolog | | | | |
| | (yeast) | | | | |
| 203126_at | inositol(myo)-1(or 4)- | 3.7269232 | 0.0009049 | 0.0217764 | −0.882476439 |
| | monophosphatase 2 | | | | |
| 208415_x_at | inhibitor of growth family, | −4.07692 | 0.0003598 | 0.0121088 | −0.004470983 |
| | member 1 | | | | |
| 205376_at | inositol polyphosphate-4- | −4.595824 | 8.98E−005 | 0.0047658 | 1.39109981 |
| | phosphatase, type II, | | | | |
| | 105 kDa | | | | |
| 213643_s_at | inositol polyphosphate-5- | −3.367908 | 0.0022858 | 0.0394532 | −1.731726886 |
| | phosphatase, 75 kDa | | | | |
| 200791_s_at | IQ motif containing GTPase | 3.2631617 | 0.0029807 | 0.0464454 | −2.002638127 |
| | activating protein 1 | | | | |
| 220034_at | interleukin-1 receptor- | 5.4039304 | 1.02E−005 | 0.0010761 | 3.431338998 |
| | associated kinase 3 | | | | |
| 202531_at | interferon regulatory factor 1 | −7.657986 | 3.04E−008 | 2.00E−005 | 9.022177601 |
| 204057_at | interferon regulatory factor | 3.8472981 | 0.0006601 | 0.0176581 | −0.554920139 |
| | 8 /// interferon regulatory | | | | |
| | factor 8 | | | | |
| 209185_s_at | insulin receptor substrate 2 | 5.8709077 | 2.95E−006 | 0.0004325 | 4.626342036 |
| 209184_s_at | insulin receptor substrate 2 | 4.7741093 | 5.56E−005 | 0.0033861 | 1.772541175 |
| 33304_at | interferon stimulated | −3.544404 | 0.0014538 | 0.0295296 | −1.287359346 |
| | exonuclease gene 20 kDa | | | | |
| 204698_at | interferon stimulated | −3.317685 | 0.0025968 | 0.0424462 | −1.821499233 |
| | exonuclease gene 20 kDa | | | | |
| 203882_at | interferon-stimulated | −4.085791 | 0.0003514 | 0.0118647 | 0.013470233 |
| | transcription factor 3, | | | | |
| | gamma 48 kDa | | | | |
| 215177_s_at | integrin, alpha 6 | −3.307456 | 0.002665 | 0.043095 | −1.877304459 |
| 209663_s_at | integrin, alpha 7 | 5.3396809 | 1.22E−005 | 0.0011938 | 3.290116774 |
| 205786_s_at | integrin, alpha M | 6.4795743 | 5.98E−007 | 0.0001532 | 6.164969498 |
| | (complement component 3 | | | | |
| | receptor 3 subunit) /// | | | | |
| | integrin, alpha M | | | | |
| | (complement component 3 | | | | |
| | receptor 3 subunit) | | | | |
| 205718_at | integrin, beta 7 | −3.813001 | 0.0007223 | 0.0187518 | −0.660874302 |
| 211339_s_at | IL2-inducible T-cell kinase | −4.079053 | 0.0003578 | 0.0120606 | 0.047552228 |
| 202747_s_at | integral membrane protein | −3.949054 | 0.0005048 | 0.0149988 | −0.323121402 |
| | 2A | | | | |
| 202660_at | inositol 1,4,5-triphosphate | 5.638527 | 5.47E−006 | 0.0006887 | 4.04200894 |
| | receptor, type 2 | | | | |
| 209297_at | intersectin 1 (SH3 domain | 4.4961321 | 0.0001174 | 0.0057121 | 1.09773516 |
| | protein) | | | | |
| 35776_at | intersectin 1 (SH3 domain | 4.2587077 | 0.0002217 | 0.0087963 | 0.516230272 |
| | protein) | | | | |
| 209298_s_at | intersectin 1 (SH3 domain | 3.3783574 | 0.0022257 | 0.0387169 | −1.67591757 |
| | protein) | | | | |
| 212813_at | junctional adhesion | 3.8970408 | 0.0005791 | 0.0163756 | −0.439247137 |
| | molecule 3 | | | | |
| 203298_s_at | Jumonji, AT rich interactive | 6.2222872 | 1.17E−006 | 0.000239 | 5.518414859 |
| | domain 2 | | | | |
| 203297_s_at | Jumonji, AT rich interactive | 5.5235106 | 7.44E−006 | 0.0008368 | 3.730979139 |
| | domain 2 | | | | |
| 214326_x_at | jun D proto-oncogene | −3.719533 | 0.0009226 | 0.0219904 | −0.907773953 |
| 203751_x_at | jun D proto-oncogene | −3.451799 | 0.0018448 | 0.0343232 | −1.557450877 |
| 200079_s_at | lysyl-tRNA synthetase | −3.787362 | 0.0007726 | 0.0196639 | −0.682018638 |
| 218569_s_at | kelch repeat and BTB | −3.882084 | 0.0006024 | 0.0166948 | −0.498696124 |
| | (POZ) domain containing 4 | | | | |
| 220776_at | potassium inwardly- | 4.6552087 | 7.66E−005 | 0.0042428 | 1.477866216 |
| | rectifying channel, | | | | |
| | subfamily J, member 14 | | | | |
| 210119_at | potassium inwardly- | 4.7139681 | 6.54E−005 | 0.003834 | 1.683509803 |
| | rectifying channel, | | | | |
| | subfamily J, member 15 | | | | |
| 211806_s_at | potassium inwardly- | 3.8646365 | 0.0006307 | 0.0171291 | −0.445652436 |
| | rectifying channel, | | | | |
| | subfamily J, member 15 | | | | |
| 206765_at | potassium inwardly- | 3.9638873 | 0.0004854 | 0.014642 | −0.229367315 |
| | rectifying channel, | | | | |
| | subfamily J, member 2 | | | | |
| 212188_at | potassium channel | 5.042749 | 2.70E−005 | 0.002062 | 2.488419179 |
| | tetramerisation domain | | | | |
| | containing 12 | | | | |
| 212192_at | potassium channel | 4.116171 | 0.0003242 | 0.0112496 | 0.091715946 |
| | tetramerisation domain | | | | |
| | containing 12 | | | | |
| 212441_at | KIAA0232 gene product | 3.8815269 | 0.0006033 | 0.0166985 | −0.518009001 |
| 212053_at | KIAA0251 protein | 3.6209572 | 0.0011925 | 0.0259769 | −1.128604719 |
| 81737_at | KIAA0251 protein | 3.5355572 | 0.0014873 | 0.0300472 | −1.327836238 |
| 215696_s_at | KIAA0310 | 3.8392722 | 0.0006742 | 0.0179484 | −0.614705395 |
| 217929_s_at | KIAA0319-like | 3.3258065 | 0.0025439 | 0.0420125 | −1.852460717 |
| 204308_s_at | KIAA0329 | 3.2649506 | 0.0029673 | 0.0462889 | −1.985006287 |
| 213304_at | KIAA0423 | −3.371968 | 0.0022623 | 0.0392298 | −1.707163049 |
| 204303_s_at | KIAA0427 | 3.2576722 | 0.0030223 | 0.0467675 | −2.022231461 |
| 203955_at | KIAA0649 | 3.9632469 | 0.0004862 | 0.014642 | −0.290520154 |
| 31826_at | KIAA0674 | 6.0122705 | 2.03E−006 | 0.0003402 | 4.995084148 |
| 212663_at | KIAA0674 | 5.1940716 | 1.80E−005 | 0.0015652 | 2.899158078 |
| 216913_s_at | KIAA0690 | 3.5995983 | 0.0012604 | 0.0268163 | −1.205824646 |
| 212359_s_at | KIAA0913 | 3.4107008 | 0.0020494 | 0.0367442 | −1.665599244 |
| 212453_at | KIAA1279 | −4.073467 | 0.0003631 | 0.0121856 | −0.013147389 |
| 216807_at | KIAA1751 /// hypothetical | 4.1887744 | 0.0002672 | 0.0099065 | 0.281175585 |
| | protein LOC642155 | | | | |
| 218342_s_at | KIAA1815 | −5.406299 | 1.02E−005 | 0.0010744 | 3.42641518 |
| 220368_s_at | KIAA2010 | −3.306489 | 0.0026716 | 0.0431381 | −1.884478291 |
| 220777_at | kinesin family member 13A | 4.9438695 | 3.52E−005 | 0.0024299 | 2.235643257 |
| 202962_at | kinesin family member 13B | 3.9782991 | 0.0004673 | 0.0142881 | −0.269465165 |
| 216969_s_at | kinesin family member 22 | −6.117807 | 1.54E−006 | 0.0002856 | 5.245713904 |
| 202183_s_at | kinesin family member 22 | −3.331289 | 0.0025088 | 0.0416502 | −1.855527456 |
| 201991_s_at | kinesin family member 5B | 5.5427871 | 7.06E−006 | 0.0008112 | 3.775937376 |
| | /// immediate early response 2 | | | | |
| 202393_s_at | Kruppel-like factor 10 | 3.8673313 | 0.0006262 | 0.0170886 | −0.502719574 |
| 214276_at | Kruppel-like factor 12 | −4.038491 | 0.0003984 | 0.0129796 | −0.117619646 |
| 203543_s_at | Kruppel-like factor 9 | 3.9412872 | 0.0005153 | 0.0151481 | −0.343654649 |
| 219157_at | kelch-like 2, Mayven | 10.76177 | 2.80E−011 | 8.91E−008 | 15.40078527 |
| | (Drosophila) | | | | |
| 221838_at | kelch-like 22 (Drosophila) | −4.964486 | 3.33E−005 | 0.0023349 | 2.274980975 |
| 214470_at | killer cell lectin-like | −4.849044 | 4.55E−005 | 0.0029026 | 2.009527674 |
| | receptor subfamily B, | | | | |
| | member 1 /// killer cell | | | | |
| | lectin-like receptor | | | | |
| | subfamily B, member 1 | | | | |
| 207796_x_at | killer cell lectin-like | −4.442037 | 0.0001357 | 0.0062883 | 0.93001647 |
| | receptor subfamily D, | | | | |
| | member 1 | | | | |
| 210606_x_at | killer cell lectin-like | −4.035614 | 0.0004015 | 0.0130255 | −0.071298817 |
| | receptor subfamily D, | | | | |
| | member 1 | | | | |
| 207795_s_at | killer cell lectin-like | −3.515839 | 0.0015649 | 0.03095 | −1.383222047 |
| | receptor subfamily D, | | | | |
| | member 1 | | | | |
| 204162_at | kinetochore associated 2 | −3.885946 | 0.0005963 | 0.0166033 | −0.479730301 |
| 210633_x_at | keratin 10 (epidermolytic | −3.515467 | 0.0015664 | 0.03095 | −1.381424467 |
| | hyperkeratosis; keratosis | | | | |
| | palmaris et plantaris) | | | | |
| 222060_at | keratin 8-like 2 | 3.9501502 | 0.0005034 | 0.0149754 | −0.305114408 |
| 210644_s_at | leukocyte-associated | 4.4466944 | 0.0001341 | 0.0062624 | 0.946285902 |
| | immunoglobulin-like | | | | |
| | receptor 1 | | | | |
| 203041_s_at | lysosomal-associated | 3.8916959 | 0.0005873 | 0.0164871 | −0.463307898 |
| | membrane protein 2 | | | | |
| 211005_at | linker for activation of T | −3.834931 | 0.0006819 | 0.0180895 | −0.596375078 |
| | cells | | | | |
| 208118_x_at | SLC7A5 pseudogene /// | −3.466485 | 0.0017766 | 0.033635 | −1.516995918 |
| | hypothetical protein | | | | |
| | LOC440345 /// PI-3-kinase- | | | | |
| | related kinase SMG-1 | | | | |
| | pseudogene /// PI-3-kinase- | | | | |
| | related kinase SMG-1-like | | | | |
| | locus ///hypothetical protein | | | | |
| | LOC646866 | | | | |
| 221581_s_at | linker for activation of T | 3.3516616 | 0.0023822 | 0.0404292 | −1.78658776 |
| | cells family, member 2 | | | | |
| 207734_at | lymphocyte transmembrane | −4.227441 | 0.000241 | 0.0093063 | 0.398811534 |
| | adaptor 1 | | | | |
| 221011_s_at | hypothetical protein | −7.094931 | 1.24E−007 | 5.26E−005 | 7.670411186 |
| | DKFZp566J091 | | | | |
| 204891_s_at | lymphocyte-specific protein | −5.37594 | 1.10E−005 | 0.0011342 | 3.374561148 |
| | tyrosine kinase | | | | |
| 204890_s_at | lymphocyte-specific protein | −4.891931 | 4.05E−005 | 0.0026469 | 2.113491247 |
| | tyrosine kinase | | | | |
| 202594_at | leptin receptor overlapping | −3.81272 | 0.0007229 | 0.0187518 | −0.648641381 |
| | transcript-like 1 | | | | |
| 207170_s_at | LETM1 domain containing 1 | −3.237237 | 0.0031819 | 0.0480165 | −2.054813033 |
| 212658_at | lipoma HMGIC fusion | 7.5928187 | 3.57E−008 | 2.28E−005 | 8.837614782 |
| | partner-like 2 | | | | |
| 207857_at | leukocyte immunoglobulin- | 4.2641953 | 0.0002185 | 0.0087153 | 0.476686967 |
| | like receptor, subfamily A | | | | |
| | (with TM domain), member | | | | |
| | 2 /// leukocyte | | | | |
| | immunoglobulin-like | | | | |
| | receptor, subfamily A (with | | | | |
| | TM domain), member 2 | | | | |
| 206881_s_at | leukocyte immunoglobulin- | 3.8508562 | 0.000654 | 0.0175358 | −0.570861562 |
| | like receptor, subfamily A | | | | |
| | (without TM domain), | | | | |
| | member 3 | | | | |
| 211135_x_at | leukocyte immunoglobulin- | 3.6099763 | 0.0012269 | 0.0264402 | −1.153537851 |
| | like receptor, subfamily B | | | | |
| | (with TM and ITIM | | | | |
| | domains), member 2 /// | | | | |
| | leukocyte immunoglobulin- | | | | |
| | like receptor, subfamily B | | | | |
| | (with TM and ITIM | | | | |
| | domains), member 3 | | | | |
| 211133_x_at | leukocyte immunoglobulin- | 3.3585626 | 0.0023408 | 0.0398777 | −1.768745 |
| | like receptor, subfamily B | | | | |
| | (with TM and ITIM | | | | |
| | domains), member 2 /// | | | | |
| | leukocyte immunoglobulin- | | | | |
| | like receptor, subfamily B | | | | |
| | (with TM and ITIM | | | | |
| | domains), member 3 | | | | |
| 210152_at | leukocyte immunoglobulin- | 3.291733 | 0.0027732 | 0.0441712 | −1.895798714 |
| | like receptor, subfamily B | | | | |
| | (with TM and ITIM | | | | |
| | domains), member 4 | | | | |
| 219541_at | Lck interacting | −4.665314 | 7.45E−005 | 0.004175 | 1.507473821 |
| | transmembrane adaptor 1 | | | | |
| 212687_at | LIM and senescent cell | 3.9025399 | 0.0005708 | 0.0162223 | −0.412073362 |
| | antigen-like domains 1 | | | | |
| 203713_s_at | lethal giant larvae homolog | −3.275582 | 0.0028887 | 0.0454273 | −1.971005784 |
| | 2 (Drosophila) | | | | |
| 212017_at | hypothetical protein | −4.023938 | 0.0004141 | 0.0132384 | −0.084581521 |
| | LOC130074 | | | | |
| 212934_at | hypothetical protein | 4.3279966 | 0.0001842 | 0.0078194 | 0.650018717 |
| | LOC137886 | | | | |
| 217104_at | similar to cervical cancer | 3.4384927 | 0.0019088 | 0.03521 | −1.568551282 |
| | suppressor-1 | | | | |
| 211456_x_at | metallothionein 1H-like | −3.444164 | 0.0018813 | 0.0348465 | −1.572923165 |
| | protein /// hypothetical | | | | |
| | protein LOC650610 | | | | |
| 214947_at | hypothetical protein | 3.7958411 | 0.0007556 | 0.0193307 | −0.689329398 |
| | LOC651803 | | | | |
| 221834_at | Peroxisomal LON protease | 3.6036769 | 0.0012471 | 0.0266949 | −1.194179793 |
| | like | | | | |
| 207584_at | lipoprotein, Lp(a) | 3.7227952 | 0.0009148 | 0.0218473 | −0.853280126 |
| 202651_at | lysophosphatidylglycerol | 4.0542562 | 0.0003821 | 0.0126515 | −0.056237967 |
| | acyltransferase 1 | | | | |
| 212272_at | lipin 1 | −4.846367 | 4.58E−005 | 0.0029152 | 1.992243627 |
| 202460_s_at | lipin 2 | −4.382191 | 0.0001594 | 0.0070314 | 0.794576049 |
| 203549_s_at | lipoprotein lipase | 4.9978257 | 3.05E−005 | 0.0022039 | 2.426210782 |
| 203548_s_at | lipoprotein lipase | 4.935084 | 3.61E−005 | 0.0024727 | 2.254926095 |
| 219759_at | leukocyte-derived arginine | −3.907514 | 0.0005633 | 0.0161552 | −0.376598806 |
| | aminopeptidase | | | | |
| 219346_at | leucine rich repeat and | −3.370134 | 0.0022729 | 0.0393506 | −1.775139187 |
| | fibronectin type III domain | | | | |
| | containing 3 | | | | |
| 211596_s_at | leucine-rich repeats and | −4.020256 | 0.0004182 | 0.013273 | −0.147924475 |
| | immunoglobulin-like | | | | |
| | domains 1 | | | | |
| 200785_s_at | low density lipoprotein- | 3.3594924 | 0.0023353 | 0.0398444 | −1.717967191 |
| | related protein 1 (alpha-2- | | | | |
| | macroglobulin receptor) | | | | |
| 219338_s_at | leucine rich repeat | 3.3102032 | 0.0026466 | 0.0428616 | −1.87074341 |
| | containing 49 | | | | |
| 221535_at | large subunit GTPase 1 | −3.48778 | 0.0016821 | 0.032564 | −1.44682498 |
| | homolog (S. cerevisiae) | | | | |
| 208771_s_at | leukotriene A4 hydrolase | 3.8851162 | 0.0005976 | 0.0166033 | −0.490414402 |
| 207339_s_at | lymphotoxin beta (TNF | −3.214812 | 0.0033664 | 0.0500752 | −2.089468885 |
| | superfamily, member 3) | | | | |
| 210128_s_at | leukotriene B4 receptor | 3.9453904 | 0.0005097 | 0.0150644 | −0.332213009 |
| 216388_s_at | leukotriene B4 receptor | 3.293685 | 0.0027596 | 0.0440481 | −1.902401735 |
| 203005_at | lymphotoxin beta receptor | 3.9369553 | 0.0005212 | 0.0152021 | −0.346843793 |
| | (TNFR superfamily, | | | | |
| | member 3) | | | | |
| 218729_at | latexin | 3.6428256 | 0.0011266 | 0.0251089 | −1.077492558 |
| 202145_at | lymphocyte antigen 6 | −7.366572 | 6.26E−008 | 3.49E−005 | 8.33334838 |
| | complex, locus E | | | | |
| 215967_s_at | lymphocyte antigen 9 | −3.220146 | 0.0033216 | 0.049641 | −2.089990745 |
| 210754_s_at | v-yes-1 Yamaguchi sarcoma | 3.8737514 | 0.0006157 | 0.0168966 | −0.516338522 |
| | viral related oncogene | | | | |
| | homolog | | | | |
| 202625_at | v-yes-1 Yamaguchi sarcoma | 3.5087733 | 0.0015936 | 0.0312318 | −1.422809629 |
| | viral related oncogene | | | | |
| | homolog | | | | |
| 212449_s_at | lysophospholipase I | 3.4035513 | 0.0020872 | 0.0372074 | −1.671090013 |
| 204458_at | lysophospholipase 3 | 3.2600029 | 0.0030046 | 0.0465906 | −1.993286231 |
| | (lysosomal phospholipase | | | | |
| | A2) | | | | |
| 203897_at | LYR motif containing 1 | −3.449039 | 0.0018579 | 0.0344425 | −1.543920308 |
| 209348_s_at | v-maf musculoaponeurotic | −4.371953 | 0.0001638 | 0.0071839 | 0.748060662 |
| | fibrosarcoma oncogene | | | | |
| | homolog (avian) | | | | |
| 206363_at | v-maf musculoaponeurotic | −3.273666 | 0.0029028 | 0.0455804 | −1.926365201 |
| | fibrosarcoma oncogene | | | | |
| | homolog (avian) | | | | |
| 218559_s_at | v-maf musculoaponeurotic | 6.0797981 | 1.70E−006 | 0.0003031 | 5.148454877 |
| | fibrosarcoma oncogene | | | | |
| | homolog B (avian) | | | | |
| 204970_s_at | v-maf musculoaponeurotic | 3.670063 | 0.0010496 | 0.0239197 | −1.023404582 |
| | fibrosarcoma oncogene | | | | |
| | homolog G (avian) | | | | |
| 218573_at | melanoma antigen family H, 1 | −3.723412 | 0.0009133 | 0.0218473 | −0.863646681 |
| 220302_at | male germ cell-associated | 3.2415841 | 0.0031473 | 0.0477078 | −2.047265164 |
| | kinase | | | | |
| 219999_at | mannosidase, alpha, class | 5.5456828 | 7.01E−006 | 0.0008091 | 3.779701447 |
| | 2A, member 2 | | | | |
| 202032_s_at | mannosidase, alpha, class | 3.5941545 | 0.0012783 | 0.0271014 | −1.218652337 |
| | 2A, member 2 | | | | |
| 202670_at | mitogen-activated protein | 3.4119357 | 0.002043 | 0.0367257 | −1.632702718 |
| | kinase kinase 1 | | | | |
| 203266_s_at | mitogen-activated protein | 3.467906 | 0.0017701 | 0.0335696 | −1.500104338 |
| | kinase kinase 4 | | | | |
| 205698_s_at | mitogen-activated protein | 4.1438069 | 0.0003012 | 0.0107899 | 0.181883139 |
| | kinase kinase 6 | | | | |
| 219278_at | mitogen-activated protein | 3.7627633 | 0.000824 | 0.0204012 | −0.705356626 |
| | kinase kinase kinase 6 | | | | |
| 205027_s_at | mitogen-activated protein | 5.1191426 | 2.20E−005 | 0.0017933 | 2.678768498 |
| | kinase kinase kinase 8 | | | | |
| 206296_x_at | mitogen-activated protein | −3.29514 | 0.0027494 | 0.0439378 | −1.922675559 |
| | kinase kinase kinase kinase 1 | | | | |
| 206571_s_at | mitogen-activated protein | 3.4171547 | 0.0020159 | 0.0364023 | −1.66044563 |
| | kinase kinase kinase kinase 4 | | | | |
| 213489_at | Microtubule-associated | −5.110564 | 2.25E−005 | 0.0018163 | 2.689399727 |
| | protein, RP/EB family, | | | | |
| | member 2 | | | | |
| 200644_at | MARCKS-like 1 | −7.088418 | 1.26E−007 | 5.26E−005 | 7.662378032 |
| 205819_at | macrophage receptor with | 4.1624083 | 0.0002866 | 0.0104535 | 0.216873832 |
| | collagenous structure | | | | |
| 217993_s_at | methionine | −4.054679 | 0.0003817 | 0.0126515 | −0.067611343 |
| | adenosyltransferase II, beta | | | | |
| 206267_s_at | megakaryocyte-associated | −4.435209 | 0.0001382 | 0.006378 | 0.942182356 |
| | tyrosine kinase | | | | |
| 210734_x_at | MYC associated factor X | −5.62543 | 5.67E−006 | 0.0007052 | 3.995366075 |
| 208403_x_at | MYC associated factor X | −3.284359 | 0.0028254 | 0.0448357 | −1.955189154 |
| 212064_x_at | MYC-associated zinc finger | −4.442678 | 0.0001355 | 0.0062883 | 0.936194067 |
| | protein (purine-binding | | | | |
| | transcription factor) | | | | |
| 209623_at | methylcrotonoyl-Coenzyme | −4.18912 | 0.0002669 | 0.0099065 | 0.294657014 |
| | A carboxylase 2 (beta) | | | | |
| 202107_s_at | MCM2 minichromosome | −3.369469 | 0.0022767 | 0.0393577 | −1.714141317 |
| | maintenance deficient 2, | | | | |
| | mitotin (S. cerevisiae) | | | | |
| 201555_at | MCM3 minichromosome | −3.99162 | 0.0004511 | 0.0139028 | −0.204548199 |
| | maintenance deficient 3 (S. cerevisiae) | | | | |
| 222036_s_at | MCM4 minichromosome | −3.425985 | 0.0019709 | 0.0358275 | −1.600878116 |
| | maintenance deficient 4 (S. cerevisiae) | | | | |
| 201930_at | MCM6 minichromosome | −4.193058 | 0.0002642 | 0.0098805 | 0.327924669 |
| | maintenance deficient 6 | | | | |
| | (MIS5 homolog, S. pombe) | | | | |
| | (S. cerevisiae) | | | | |
| 217599_s_at | MyoD family inhibitor | −3.521095 | 0.0015438 | 0.0307978 | −1.387591108 |
| | domain containing | | | | |
| 210153_s_at | malic enzyme 2, NAD(+)- | 3.2974068 | 0.0027337 | 0.043806 | −1.895461101 |
| | dependent, mitochondrial /// | | | | |
| | protein kinase, cAMP- | | | | |
| | dependent, regulatory, type | | | | |
| | II, beta | | | | |
| 202618_s_at | methyl CpG binding protein | −4.133561 | 0.0003095 | 0.0109647 | 0.154499067 |
| | 2 (Rett syndrome) | | | | |
| 207079_s_at | mediator of RNA | −3.462096 | 0.0017967 | 0.0338007 | −1.527608198 |
| | polymerase II transcription, | | | | |
| | subunit 6 homolog (S. cerevisiae) | | | | |
| 212535_at | MADS box transcription | 3.7335966 | 0.0008893 | 0.0214696 | −0.867987027 |
| | enhancer factor 2, | | | | |
| | polypeptide A (myocyte | | | | |
| | enhancer factor 2A) | | | | |
| 207968_s_at | MADS box transcription | −3.72812 | 0.0009021 | 0.0217318 | −0.849084828 |
| | enhancer factor 2, | | | | |
| | polypeptide C (myocyte | | | | |
| | enhancer factor 2C) | | | | |
| 212830_at | multiple EGF-like-domains 9 | 3.7165622 | 0.0009298 | 0.0220336 | −0.90938831 |
| 212831_at | multiple EGF-like-domains 9 | 3.4621648 | 0.0017964 | 0.0338007 | −1.525691707 |
| 206028_s_at | c-mer proto-oncogene | 10.469242 | 5.13E−011 | 1.43E−007 | 14.90589976 |
| | tyrosine kinase | | | | |
| 211913_s_at | c-mer proto-oncogene | 4.2805887 | 0.0002091 | 0.0084515 | 0.518779243 |
| | tyrosine kinase | | | | |
| 209703_x_at | methyltransferase like 7A | 4.2496147 | 0.0002272 | 0.0089432 | 0.432557453 |
| 217868_s_at | methyltransferase like 9 | 3.8045735 | 0.0007385 | 0.0190021 | −0.674756638 |
| 218109_s_at | major facilitator superfamily | 4.7468747 | 5.98E−005 | 0.0035952 | 1.728829723 |
| | domain containing 1 | | | | |
| 212945_s_at | MAX gene associated | −3.273408 | 0.0029046 | 0.0455804 | −1.971319233 |
| 206522_at | maltase-glucoamylase | 8.9425055 | 1.45E−009 | 2.06E−006 | 11.67816349 |
| | (alpha-glucosidase) /// | | | | |
| | similar to Maltase- | | | | |
| | glucoamylase, intestinal | | | | |
| 214696_at | hypothetical protein | 7.7417859 | 2.48E−008 | 1.84E−005 | 9.178335493 |
| | MGC14376 | | | | |
| 219812_at | hypothetical protein | −3.411796 | 0.0020437 | 0.0367257 | −1.58299562 |
| | LOC79037 | | | | |
| 220934_s_at | hypothetical protein | −3.742728 | 0.0008683 | 0.0211928 | −0.840145541 |
| | MGC3196 | | | | |
| 204880_at | O-6-methylguanine-DNA | −4.229354 | 0.0002398 | 0.0093056 | 0.379865831 |
| | methyltransferase | | | | |
| 204168_at | microsomal glutathione S- | 4.1564443 | 0.0002912 | 0.0105514 | 0.192247085 |
| | transferase 2 | | | | |
| 217871_s_at | macrophage migration | −4.21758 | 0.0002474 | 0.0094014 | 0.378863451 |
| | inhibitory factor | | | | |
| | (glycosylation-inhibiting | | | | |
| | factor) | | | | |
| 209585_s_at | multiple inositol | −3.458014 | 0.0018157 | 0.03398 | −1.507957353 |
| | polyphosphate histidine | | | | |
| | phosphatase, 1 | | | | |
| 207233_s_at | microphthalmia-associated | 3.2396083 | 0.003163 | 0.0478336 | −2.071786492 |
| | transcription factor | | | | |
| 209467_s_at | MAP kinase interacting | 4.2920628 | 0.0002028 | 0.0082921 | 0.525267109 |
| | serine/threonine kinase 1 | | | | |
| 204918_s_at | myeloid/lymphoid or | −4.306252 | 0.0001953 | 0.0081193 | 0.583823593 |
| | mixed-lineage leukemia | | | | |
| | (trithorax homolog, | | | | |
| | Drosophila); translocated to, 3 | | | | |
| 220615_s_at | male sterility domain | 4.1308743 | 0.0003117 | 0.0110259 | 0.153954894 |
| | containing 1 | | | | |
| 202519_at | MLX interacting protein | 4.1584797 | 0.0002896 | 0.0105289 | 0.204917195 |
| 207329_at | matrix metallopeptidase 8 | 3.561556 | 0.0013908 | 0.0285361 | −1.226425789 |
| | (neutrophil collagenase) | | | | |
| 203936_s_at | matrix metallopeptidase 9 | 6.2084895 | 1.21E−006 | 0.0002412 | 5.494417575 |
| | (gelatinase B, 92 kDa | | | | |
| | gelatinase, 92 kDa type IV | | | | |
| | collagenase) | | | | |
| 204959_at | myeloid cell nuclear | 3.3907661 | 0.0021564 | 0.0378505 | −1.701977779 |
| | differentiation antigen | | | | |
| 218212_s_at | molybdenum cofactor | −3.49272 | 0.0016608 | 0.0322933 | −1.448873612 |
| | synthesis 2 | | | | |
| 218865_at | MOCO sulphurase C- | 5.5875919 | 6.27E−006 | 0.0007548 | 3.914199721 |
| | terminal domain containing 1 | | | | |
| 221636_s_at | MOCO sulphurase C- | 4.2271534 | 0.0002412 | 0.0093063 | 0.423787778 |
| | terminal domain containing 2 | | | | |
| 204438_at | mannose receptor, C type 1 | 5.069328 | 2.51E−005 | 0.0019651 | 2.582052934 |
| | /// mannose receptor, C type | | | | |
| | 1-like 1 | | | | |
| 212199_at | Morf4 family associated | −4.198185 | 0.0002606 | 0.009808 | 0.330645263 |
| | protein 1-like 1 | | | | |
| 219162_s_at | mitochondrial ribosomal | −3.342168 | 0.0024404 | 0.0410104 | −1.823298715 |
| | protein L11 | | | | |
| 218558_s_at | mitochondrial ribosomal | −4.226846 | 0.0002414 | 0.0093063 | 0.379071532 |
| | protein L39 | | | | |
| 218202_x_at | mitochondrial ribosomal | −3.577656 | 0.001334 | 0.0279122 | −1.246037437 |
| | protein L44 | | | | |
| 201717_at | mitochondrial ribosomal | −5.351497 | 1.18E−005 | 0.0011826 | 3.283723955 |
| | protein L49 | | | | |
| 211594_s_at | mitochondrial ribosomal | −4.620417 | 8.41E−005 | 0.0045156 | 1.40079184 |
| | protein L9 | | | | |
| 212145_at | mitochondrial ribosomal | −3.511203 | 0.0015837 | 0.0311191 | −1.387148207 |
| | protein S27 | | | | |
| 219607_s_at | membrane-spanning 4- | 4.0363846 | 0.0004007 | 0.0130255 | −0.075575416 |
| | domains, subfamily A, | | | | |
| | member 4 | | | | |
| 211450_s_at | mutS homolog 6 (E. coli) | −5.81249 | 3.44E−006 | 0.0004828 | 4.484041843 |
| 218733_at | male-specific lethal 2-like 1 | −3.223815 | 0.0032911 | 0.0492896 | −2.105139445 |
| | (Drosophila) | | | | |
| 219451_at | methionine sulfoxide | 3.3937545 | 0.0021401 | 0.0376972 | −1.660918433 |
| | reductase B2 | | | | |
| 204745_x_at | metallothionein 1G | −3.474791 | 0.0017391 | 0.0332359 | −1.487573113 |
| 216862_s_at | mature T-cell proliferation 1 | −4.321874 | 0.0001873 | 0.0078884 | 0.716317639 |
| 210212_x_at | mature T-cell proliferation 1 | −3.518457 | 0.0015544 | 0.0308899 | −1.382137492 |
| 204871_at | mitochondrial transcription | −3.350474 | 0.0023894 | 0.0404668 | −1.800061689 |
| | termination factor | | | | |
| 205323_s_at | metal-regulatory | 4.4764912 | 0.0001238 | 0.0059433 | 1.050900118 |
| | transcription factor 1 | | | | |
| 214975_s_at | myotubularin related protein 1 | −3.393994 | 0.0021388 | 0.0376972 | −1.707666741 |
| 213511_s_at | myotubularin related protein 1 | −3.292331 | 0.002769 | 0.0441676 | −1.875326364 |
| 202197_at | myotubularin related protein 3 | 4.8644697 | 4.36E−005 | 0.002825 | 2.056382365 |
| 221369_at | melatonin receptor 1A | 3.6496035 | 0.001107 | 0.0249298 | −1.022866151 |
| 210360_s_at | metastasis suppressor 1 | −4.015339 | 0.0004236 | 0.0133407 | −0.110107379 |
| 212096_s_at | mitochondrial tumor | −3.461608 | 0.001799 | 0.0338007 | −1.52793833 |
| | suppressor 1 | | | | |
| 210386_s_at | metaxin 1 | 5.0677388 | 2.52E−005 | 0.0019666 | 2.571078366 |
| 207847_s_at | mucin 1, cell surface | 3.4839684 | 0.0016986 | 0.0327991 | −1.476671718 |
| | associated | | | | |
| 222132_s_at | multiple substrate lipid | −3.620916 | 0.0011926 | 0.0259769 | −1.140012259 |
| | kinase | | | | |
| 206877_at | MAX dimerization protein 1 | 4.2258856 | 0.000242 | 0.009314 | 0.404395855 |
| 212347_x_at | MAX dimerization protein 4 | −3.326427 | 0.0025399 | 0.0419858 | −1.84580392 |
| 209124_at | myeloid differentiation | 3.6264536 | 0.0011756 | 0.025808 | −1.141630363 |
| | primary response gene (88) | | | | |
| 205145_s_at | myosin, light polypeptide 5, | −4.151689 | 0.0002949 | 0.0106 | 0.193777662 |
| | regulatory /// similar to | | | | |
| | Superfast myosin regulatory | | | | |
| | light chain 2 (MyLC-2) | | | | |
| | (MYLC2) (Myosin | | | | |
| | regulatory light chain 5) | | | | |
| 204173_at | myosin, light polypeptide | 3.6540033 | 0.0010944 | 0.024757 | −1.07190672 |
| | 6B, alkali, smooth muscle | | | | |
| | and non-muscle | | | | |
| 59375_at | myosin XVB pseudogene | 3.3466222 | 0.002413 | 0.0407024 | −1.805912094 |
| 211916_s_at | myosin IA | 4.311803 | 0.0001924 | 0.008043 | 0.60317388 |
| 217409_at | myosin VA (heavy | 3.754011 | 0.0008431 | 0.0207356 | −0.832633693 |
| | polypeptide 12, myoxin) | | | | |
| 208189_s_at | myosin VIIA | 7.0276054 | 1.47E−007 | 5.64E−005 | 7.51220835 |
| 33197_at | myosin VIIA | 5.8568816 | 3.06E−006 | 0.0004411 | 4.595628412 |
| 201414_s_at | nucleosome assembly | −3.890535 | 0.0005891 | 0.0165114 | −0.470574993 |
| | protein 1-like 4 | | | | |
| 219217_at | asparaginyl-tRNA | −3.606698 | 0.0012374 | 0.0265376 | −1.156024517 |
| | synthetase 2 | | | | |
| | (mitochondrial)(putative) | | | | |
| 201521_s_at | nuclear cap binding protein | −4.189647 | 0.0002666 | 0.0099065 | 0.332784282 |
| | subunit 2, 20 kDa | | | | |
| 204961_s_at | neutrophil cytosolic factor | 3.4233479 | 0.0019842 | 0.0359761 | −1.609928411 |
| | 1, (chronic granulomatous | | | | |
| | disease, autosomal 1) /// | | | | |
| | similar to Neutrophil | | | | |
| | cytosol factor 1 (NCF-1) | | | | |
| | (Neutrophil NADPH | | | | |
| | oxidase factor 1) (47 kDa | | | | |
| | neutrophil oxidase factor) | | | | |
| | (p47-phox) (NCF-47K) (47 kDa | | | | |
| | autosomal chronic | | | | |
| | granulomatous disease | | | | |
| | protein) (NOXO2) | | | | |
| 209949_at | neutrophil cytosolic factor 2 | 3.5476728 | 0.0014415 | 0.0293618 | −1.321237334 |
| | (65 kDa, chronic | | | | |
| | granulomatous disease, | | | | |
| | autosomal 2) | | | | |
| 207677_s_at | neutrophil cytosolic factor | 5.396957 | 1.04E−005 | 0.0010913 | 3.421733075 |
| | 4, 40 kDa | | | | |
| 205147_x_at | neutrophil cytosolic factor | 4.8684863 | 4.31E−005 | 0.0028028 | 2.065511742 |
| | 4, 40 kDa | | | | |
| 207760_s_at | nuclear receptor co- | 4.1534278 | 0.0002936 | 0.0105852 | 0.176678889 |
| | repressor 2 | | | | |
| 211010_s_at | natural cytotoxicity | −4.021124 | 0.0004172 | 0.0132622 | −0.117397823 |
| | triggering receptor 3 | | | | |
| 211583_x_at | natural cytotoxicity | −3.86853 | 0.0006243 | 0.0170677 | −0.525152135 |
| | triggering receptor 3 | | | | |
| 214279_s_at | NDRG family member 2 | −3.852181 | 0.0006517 | 0.0174958 | −0.574329039 |
| 204125_at | NADH dehydrogenase | 3.3838511 | 0.0021948 | 0.0383883 | −1.688829265 |
| | (ubiquinone) 1 alpha | | | | |
| | subcomplex, assembly | | | | |
| | factor 1 | | | | |
| 218200_s_at | NADH dehydrogenase | −5.766288 | 3.89E−006 | 0.0005291 | 4.356053461 |
| | (ubiquinone) 1 beta | | | | |
| | subcomplex, 2, 8 kDa | | | | |
| 203371_s_at | NADH dehydrogenase | 4.1256494 | 0.0003161 | 0.0110921 | 0.144979142 |
| | (ubiquinone) 1 beta | | | | |
| | subcomplex, 3, 12 kDa | | | | |
| 221979_at | NADH dehydrogenase | −4.258472 | 0.0002219 | 0.0087963 | 0.461301854 |
| | (ubiquinone) 1 beta | | | | |
| | subcomplex, 6, 17 kDa | | | | |
| 78383_at | NADH dehydrogenase | −3.945712 | 0.0005093 | 0.0150644 | −0.3312007 |
| | (ubiquinone) 1 beta | | | | |
| | subcomplex, 6, 17 kDa | | | | |
| 208714_at | NADH dehydrogenase | −3.820058 | 0.0007091 | 0.0185259 | −0.617626644 |
| | (ubiquinone) flavoprotein 1, | | | | |
| | 51 kDa | | | | |
| 202150_s_at | neural precursor cell | −3.715459 | 0.0009324 | 0.0220336 | −0.874944296 |
| | expressed, developmentally | | | | |
| | down-regulated 9 | | | | |
| 211089_s_at | NIMA (never in mitosis | 3.2520573 | 0.0030653 | 0.0471394 | −2.025842322 |
| | gene a)-related kinase 3 | | | | |
| 218888_s_at | neuropilin (NRP) and | 4.8975403 | 3.99E−005 | 0.0026226 | 2.137562138 |
| | tolloid (TLL)-like 2 | | | | |
| 211914_x_at | neurofibromin 1 | 3.5319242 | 0.0015013 | 0.030237 | −1.377257545 |
| | (neurofibromatosis, von | | | | |
| | Recklinghausen disease, | | | | |
| | Watson disease) | | | | |
| 203574_at | nuclear factor, interleukin 3 | 5.5729076 | 6.52E−006 | 0.0007603 | 3.874026645 |
| | regulated | | | | |
| 201502_s_at | nuclear factor of kappa light | 4.560604 | 9.87E−005 | 0.0051366 | 1.277276334 |
| | polypeptide gene enhancer | | | | |
| | in B-cells inhibitor, alpha | | | | |
| 214448_x_at | nuclear factor of kappa light | −4.136872 | 0.0003068 | 0.0109381 | 0.162388308 |
| | polypeptide gene enhancer | | | | |
| | in B-cells inhibitor, beta | | | | |
| 217722_s_at | neugrin, neurite outgrowth | −7.00942 | 1.54E−007 | 5.80E−005 | 7.472039365 |
| | associated | | | | |
| 201077_s_at | NHP2 non-histone | −3.866422 | 0.0006277 | 0.0170997 | −0.543843713 |
| | chromosome protein 2-like | | | | |
| | 1 (S. cerevisiae) | | | | |
| 202008_s_at | nidogen 1 | −3.363949 | 0.0023089 | 0.0395937 | −1.728370168 |
| 218133_s_at | NIF3 NGG1 interacting | −3.774581 | 0.0007989 | 0.0199959 | −0.731791057 |
| | factor 3-like 1 (S. pombe) | | | | |
| 201709_s_at | nipsnap homolog 1 (C. elegans) | −4.85116 | 4.52E−005 | 0.0028944 | 1.989139057 |
| 201591_s_at | nischarin | 4.8616161 | 4.39E−005 | 0.0028385 | 2.005805898 |
| 213915_at | natural killer cell group 7 | −3.323884 | 0.0025564 | 0.0421014 | −1.867713513 |
| | sequence | | | | |
| 218240_at | NFKB inhibitor interacting | 5.2140562 | 1.70E−005 | 0.0015095 | 2.976553329 |
| | Ras-like 2 | | | | |
| 219553_at | non-metastatic cells 7, | −3.718363 | 0.0009254 | 0.0219904 | −0.899743492 |
| | protein expressed in | | | | |
| | (nucleoside-diphosphate | | | | |
| | kinase) | | | | |
| 209755_at | nicotinamide nucleotide | 4.3326215 | 0.000182 | 0.0077777 | 0.68125485 |
| | adenylyltransferase 2 | | | | |
| 205006_s_at | N-myristoyltransferase 2 | −3.344732 | 0.0024246 | 0.0408057 | −1.753305782 |
| 218889_at | nucleolar complex | −4.672328 | 7.31E−005 | 0.0041477 | 1.547524296 |
| | associated 3 homolog (S. cerevisiae) | | | | |
| 221970_s_at | nucleolar protein 11 | −3.451823 | 0.0018447 | 0.0343232 | −1.544529435 |
| 209104_s_at | nucleolar protein family A, | −4.925385 | 3.70E−005 | 0.0025118 | 2.199056718 |
| | member 2 (H/ACA small | | | | |
| | nucleolar RNPs) | | | | |
| 208698_s_at | non-POU domain | −4.90777 | 3.88E−005 | 0.0025742 | 2.167167105 |
| | containing, octamer-binding | | | | |
| 200057_s_at | non-POU domain | −4.031064 | 0.0004063 | 0.0131036 | −0.109405441 |
| | containing, octamer-binding | | | | |
| 202445_s_at | Notch homolog 2 | 3.6889002 | 0.0009993 | 0.0231716 | −0.925029403 |
| | (Drosophila) | | | | |
| 222115_x_at | cytokine-like nuclear factor | −3.388015 | 0.0021716 | 0.0380426 | −1.713514417 |
| | n-pac | | | | |
| 209798_at | nuclear protein, ataxia- | −3.906012 | 0.0005656 | 0.0161776 | −0.40553123 |
| | telangiectasia locus | | | | |
| 221210_s_at | N-acetylneuraminate | 4.3512407 | 0.0001731 | 0.0074472 | 0.708106598 |
| | pyruvate lyase | | | | |
| | (dihydrodipicolinate | | | | |
| | synthase) | | | | |
| 202228_s_at | neuroplastin | 4.2662134 | 0.0002173 | 0.0086937 | 0.487632067 |
| 208709_s_at | nardilysin (N-arginine | 5.0031235 | 3.00E−005 | 0.0021941 | 2.370985765 |
| | dibasic convertase) | | | | |
| 206237_s_at | neuregulin 1 | 4.1426696 | 0.0003021 | 0.0108052 | 0.157843047 |
| 206343_s_at | neuregulin 1 | 3.9722595 | 0.0004748 | 0.0144534 | −0.247145403 |
| 208241_at | neuregulin 1 | 3.6187831 | 0.0011992 | 0.02607 | −1.147964641 |
| 202600_s_at | nuclear receptor interacting | 4.6313092 | 8.17E−005 | 0.0044164 | 1.419488852 |
| | protein 1 | | | | |
| 219084_at | nuclear receptor binding | 3.4886005 | 0.0016785 | 0.0325236 | −1.44314523 |
| | SET domain protein 1 | | | | |
| 220248_x_at | NSFL1 (p97) cofactor (p47) | 4.3219254 | 0.0001872 | 0.0078884 | 0.647161773 |
| 209073_s_at | numb homolog (Drosophila) | 3.6427597 | 0.0011268 | 0.0251089 | −1.068225006 |
| 207545_s_at | numb homolog (Drosophila) | 3.2710925 | 0.0029217 | 0.0456867 | −1.957907921 |
| 208922_s_at | nuclear RNA export factor 1 | 3.9927127 | 0.0004498 | 0.0139011 | −0.232780172 |
| 218708_at | NTF2-like export factor 1 | −3.420185 | 0.0020003 | 0.0362094 | −1.584241125 |
| 206553_at | 2′-5′-oligoadenylate | −4.072051 | 0.0003645 | 0.0122131 | 0.008158459 |
| | synthetase 2, 69/71 kDa | | | | |
| 204972_at | 2′-5′-oligoadenylate | −3.905269 | 0.0005667 | 0.0161886 | −0.421009914 |
| | synthetase 2, 69/71 kDa | | | | |
| 200077_s_at | ornithine decarboxylase | 3.6431266 | 0.0011257 | 0.0251089 | −1.049914038 |
| | antizyme 1 | | | | |
| 219100_at | oligonucleotide/oligosaccharide- | −3.644466 | 0.0011218 | 0.0251089 | −1.068739421 |
| | binding fold containing 1 | | | | |
| 203446_s_at | oculocerebrorenal syndrome | 3.5190253 | 0.0015521 | 0.0308796 | −1.387721046 |
| | of Lowe | | | | |
| 221090_s_at | 2-oxoglutarate and iron- | −3.315776 | 0.0026094 | 0.0425668 | −1.884264315 |
| | dependent oxygenase | | | | |
| | domain containing 1 | | | | |
| 202074_s_at | optineurin | −4.093744 | 0.0003441 | 0.0117213 | 0.05084135 |
| 210028_s_at | origin recognition complex, | −3.279258 | 0.002862 | 0.0451018 | −1.942601343 |
| | subunit 3-like (yeast) | | | | |
| 204957_at | origin recognition complex, | −3.623062 | 0.001186 | 0.0259085 | −1.135141206 |
| | subunit 5-like (yeast) | | | | |
| 209221_s_at | oxysterol binding protein- | 3.2409524 | 0.0031523 | 0.0477512 | −2.058574469 |
| | like 2 | | | | |
| 206048_at | ovo-like 2 (Drosophila) | −3.27126 | 0.0029204 | 0.0456867 | −1.991947718 |
| 219133_at | 3-oxoacyl-ACP synthase, | −3.937976 | 0.0005198 | 0.015181 | −0.34235903 |
| | mitochondrial | | | | |
| 214615_at | purinergic receptor P2Y, G- | −3.637881 | 0.0011412 | 0.0253027 | −1.074735632 |
| | protein coupled, 10 | | | | |
| 218589_at | purinergic receptor P2Y, G- | −3.531712 | 0.0015021 | 0.030237 | −1.323330122 |
| | protein coupled, 5 | | | | |
| 220001_at | peptidyl arginine deiminase, | 8.3835748 | 5.30E−009 | 5.14E−006 | 10.63185834 |
| | type IV | | | | |
| 211413_s_at | peptidyl arginine deiminase, | 3.7336438 | 0.0008892 | 0.0214696 | −0.829771785 |
| | type IV | | | | |
| 205232_s_at | platelet-activating factor | −3.985046 | 0.000459 | 0.0140883 | −0.226566338 |
| | acetylhydrolase 2, 40 kDa | | | | |
| 205233_s_at | platelet-activating factor | −3.93962 | 0.0005176 | 0.0151633 | −0.325599823 |
| | acetylhydrolase 2, 40 kDa | | | | |
| 208878_s_at | p21 (CDKN1A)-activated | 3.4046896 | 0.0020811 | 0.0371588 | −1.657169927 |
| | kinase 2 | | | | |
| 208644_at | poly (ADP-ribose) | −3.262899 | 0.0029827 | 0.0464454 | −1.998088098 |
| | polymerase family, member 1 | | | | |
| 217738_at | pre-B-cell colony enhancing | 3.9390953 | 0.0005183 | 0.0151633 | −0.339832259 |
| | factor 1 /// pre-B cell | | | | |
| | enhancing factor 1 | | | | |
| | pseudogene | | | | |
| 217739_s_at | pre-B-cell colony enhancing | 3.5616484 | 0.0013904 | 0.0285361 | −1.306068914 |
| | factor 1 /// pre-B cell | | | | |
| | enhancing factor 1 | | | | |
| | pseudogene | | | | |
| 212259_s_at | pre-B-cell leukemia | −5.160862 | 1.97E−005 | 0.0016545 | 2.782298376 |
| | transcription factor | | | | |
| | interacting protein 1 | | | | |
| 207838_x_at | pre-B-cell leukemia | −4.510906 | 0.0001128 | 0.0055737 | 1.100787503 |
| | transcription factor | | | | |
| | interacting protein 1 | | | | |
| 214177_s_at | pre-B-cell leukemia | −3.63023 | 0.0011641 | 0.0256319 | −1.108425914 |
| | transcription factor | | | | |
| | interacting protein 1 | | | | |
| 210368_at | protocadherin gamma | 3.2606543 | 0.0029996 | 0.0465465 | −1.992435948 |
| | subfamily B, 4 /// | | | | |
| | protocadherin gamma | | | | |
| | subfamily A, 8 | | | | |
| 219940_s_at | PCI domain containing 2 | −4.172697 | 0.0002789 | 0.0102379 | 0.277504474 |
| 205559_s_at | proprotein convertase | 4.0311068 | 0.0004063 | 0.0131036 | −0.107025633 |
| | subtilisin/kexin type 5 | | | | |
| 213652_at | Proprotein convertase | 3.836898 | 0.0006784 | 0.0180177 | −0.612209043 |
| | subtilisin/kexin type 5 | | | | |
| 203118_at | proprotein convertase | −3.543875 | 0.0014557 | 0.0295315 | −1.330080983 |
| | subtilisin/kexin type 7 | | | | |
| 221918_at | PCTAIRE protein kinase 2 | −4.174838 | 0.0002773 | 0.0101965 | 0.24359603 |
| 202730_s_at | programmed cell death 4 | −7.258288 | 8.20E−008 | 4.06E−005 | 8.071670715 |
| | (neoplastic transformation | | | | |
| | inhibitor) | | | | |
| 202731_at | programmed cell death 4 | −4.994268 | 3.08E−005 | 0.0022108 | 2.384742204 |
| | (neoplastic transformation | | | | |
| | inhibitor) | | | | |
| 214582_at | phosphodiesterase 3B, | −3.275578 | 0.0028888 | 0.0454273 | −1.989595866 |
| | cGMP-inhibited | | | | |
| 203857_s_at | protein disulfide isomerase | 3.55392 | 0.0014185 | 0.0289756 | −1.313968785 |
| | family A, member 5 | | | | |
| 208638_at | protein disulfide isomerase | 3.7052882 | 0.0009575 | 0.0224641 | −0.950502419 |
| | family A, member 6 | | | | |
| 214121_x_at | PDZ and LIM domain 7 | 4.2574858 | 0.0002224 | 0.0088039 | 0.551999517 |
| | (enigma) | | | | |
| 220865_s_at | prenyl (decaprenyl) | 3.5726871 | 0.0013513 | 0.0280881 | −1.181562766 |
| | diphosphate synthase, | | | | |
| | subunit 1 | | | | |
| 200788_s_at | phosphoprotein enriched in | −4.450165 | 0.0001328 | 0.0062436 | 0.966904371 |
| | astrocytes 15 | | | | |
| 200787_s_at | phosphoprotein enriched in | −3.64938 | 0.0011076 | 0.0249298 | −1.08974448 |
| | astrocytes 15 | | | | |
| 211941_s_at | phosphatidylethanolamine | −5.971017 | 2.26E−006 | 0.0003578 | 4.894349825 |
| | binding protein 1 | | | | |
| 210825_s_at | phosphatidylethanolamine | −3.844699 | 0.0006646 | 0.0177366 | −0.549903787 |
| | binding protein 1 | | | | |
| 205353_s_at | phosphatidylethanolamine | −3.214489 | 0.0033691 | 0.050078 | −2.08853874 |
| | binding protein 1 | | | | |
| 218025_s_at | peroxisomal D3,D2-enoyl- | −6.668995 | 3.67E−007 | 0.0001061 | 6.635968187 |
| | CoA isomerase | | | | |
| 218319_at | pellino homolog 1 | 3.8557177 | 0.0006457 | 0.0173757 | −0.579288125 |
| | (Drosophila) | | | | |
| 207621_s_at | phosphatidylethanolamine | −3.996436 | 0.0004454 | 0.0138256 | −0.199619713 |
| | N-methyltransferase | | | | |
| 202861_at | period homolog 1 | 7.2015321 | 9.46E−008 | 4.30E−005 | 7.937670727 |
| | (Drosophila) | | | | |
| 36829_at | period homolog 1 | 5.4780521 | 8.40E−006 | 0.0009262 | 3.613619011 |
| | (Drosophila) | | | | |
| 221045_s_at | period homolog 3 | −3.468049 | 0.0017695 | 0.0335696 | −1.521241413 |
| | (Drosophila) | | | | |
| 221811_at | per1-like domain containing 1 | −5.576296 | 6.46E−006 | 0.0007574 | 3.872348612 |
| 206351_s_at | peroxisome biogenesis | −3.96326 | 0.0004862 | 0.014642 | −0.289905451 |
| | factor 10 | | | | |
| 205094_at | peroxisomal biogenesis | −3.563408 | 0.0013841 | 0.0285048 | −1.283829302 |
| | factor 12 | | | | |
| 211033_s_at | peroxisomal biogenesis | −3.396508 | 0.0021251 | 0.0376416 | −1.675415885 |
| | factor 7 | | | | |
| 210908_s_at | prefoldin subunit 5 | −3.476125 | 0.0017332 | 0.0332077 | −1.501781671 |
| 202464_s_at | 6-phosphofructo-2- | 5.6547545 | 5.24E−006 | 0.000671 | 4.060361204 |
| | kinase/fructose-2,6- | | | | |
| | biphosphatase 3 | | | | |
| 200886_s_at | phosphoglycerate mutase 1 | 3.3944535 | 0.0021362 | 0.0376896 | −1.593541122 |
| | (brain) /// similar to | | | | |
| | Phosphoglycerate mutase 1 | | | | |
| | (Phosphoglycerate mutase | | | | |
| | isozyme B) (PGAM-B) | | | | |
| | (BPG-dependent PGAM 1) | | | | |
| 201118_at | phosphogluconate | 3.2874178 | 0.0028036 | 0.0445602 | −1.918214819 |
| | dehydrogenase /// UDP- | | | | |
| | glucose dehydrogenase | | | | |
| 200738_s_at | phosphoglycerate kinase 1 | 4.4653902 | 0.0001275 | 0.0060731 | 1.022934428 |
| 207384_at | peptidoglycan recognition | 3.2428282 | 0.0031374 | 0.0476847 | −1.983993924 |
| | protein 1 | | | | |
| 201121_s_at | progesterone receptor | 3.6747982 | 0.0010367 | 0.0237807 | −1.005694649 |
| | membrane component 1 | | | | |
| 219394_at | phosphatidylglycerophosphate | 3.8990457 | 0.000576 | 0.0163307 | −0.441818616 |
| | synthase 1 | | | | |
| 201600_at | prohibitin 2 | −3.714089 | 0.0009358 | 0.0220657 | −0.923348887 |
| 200919_at | polyhomeotic-like 2 | 13.491127 | 1.57E−013 | 7.00E−010 | 19.73316172 |
| | (Drosophila) | | | | |
| 203278_s_at | PHD finger protein 21A | 3.8745484 | 0.0006144 | 0.0168823 | −0.517347194 |
| 202738_s_at | phosphorylase kinase, beta | 4.1961442 | 0.000262 | 0.0098448 | 0.298023798 |
| 221689_s_at | phosphatidylinositol glycan | −3.631985 | 0.0011588 | 0.0255407 | −1.114212971 |
| | anchor biosynthesis, class P | | | | |
| 217620_s_at | phosphoinositide-3-kinase, | 3.3682068 | 0.002284 | 0.0394532 | −1.719683015 |
| | catalytic, beta polypeptide | | | | |
| 202743_at | phosphoinositide-3-kinase, | −3.596907 | 0.0012692 | 0.0269344 | −1.208781096 |
| | regulatory subunit 3 (P55, | | | | |
| | gamma) | | | | |
| 204269_at | pim-2 oncogene | −4.339921 | 0.0001784 | 0.0076467 | 0.659167825 |
| 204572_s_at | protein (peptidylprolyl | −4.139537 | 0.0003046 | 0.0108782 | 0.168394561 |
| | cis/trans isomerase) NIMA- | | | | |
| | interacting, 4 (parvulin) | | | | |
| 219155_at | phosphatidylinositol transfer | −4.716141 | 6.50E−005 | 0.0038217 | 1.661137309 |
| | protein, cytoplasmic 1 | | | | |
| 218667_at | praja 1 | −3.231765 | 0.003226 | 0.0485378 | −2.000004816 |
| 204612_at | protein kinase (cAMP- | −3.261628 | 0.0029923 | 0.0464971 | −1.962525619 |
| | dependent, catalytic) | | | | |
| | inhibitor alpha | | | | |
| 216551_x_at | phospholipase C, gamma 1 | −3.700005 | 0.0009708 | 0.0226994 | −0.939677238 |
| | /// copine family member IX | | | | |
| 205203_at | phospholipase D1, | 5.1795509 | 1.87E−005 | 0.001614 | 2.869156194 |
| | phosphatidylcholine- | | | | |
| | specific | | | | |
| 219566_at | pleckstrin homology domain | −3.673771 | 0.0010395 | 0.0238058 | −1.017174025 |
| | containing, family F (with | | | | |
| | FYVE domain) member 1 | | | | |
| 218290_at | pleckstrin homology domain | −3.407099 | 0.0020684 | 0.0369951 | −1.660021054 |
| | containing, family J member 1 | | | | |
| 204958_at | polo-like kinase 3 | 3.7036125 | 0.0009617 | 0.0225341 | −0.913048166 |
| | (Drosophila) | | | | |
| 215462_at | polo-like kinase 3 | 3.2790999 | 0.0028632 | 0.0451018 | −1.938826238 |
| | (Drosophila) | | | | |
| 213241_at | plexin C1 | 4.6667879 | 7.42E−005 | 0.004175 | 1.514941223 |
| 204285_s_at | phorbol-12-myristate-13- | −3.426374 | 0.0019689 | 0.0358275 | −1.57611863 |
| | acetate-induced protein 1 | | | | |
| 206503_x_at | promyelocytic leukemia | −3.34519 | 0.0024218 | 0.0407891 | −1.827801726 |
| 209034_at | proline-rich nuclear receptor | −3.340408 | 0.0024514 | 0.0411013 | −1.844255008 |
| | coactivator 1 | | | | |
| 203616_at | polymerase (DNA directed), | −5.42464 | 9.69E−006 | 0.0010296 | 3.489870959 |
| | beta | | | | |
| 201115_at | polymerase (DNA directed), | −5.389803 | 1.06E−005 | 0.0011072 | 3.389374537 |
| | delta 2, regulatory subunit | | | | |
| | 50 kDa | | | | |
| 207515_s_at | polymerase (RNA) I | −5.342959 | 1.21E−005 | 0.0011938 | 3.285994086 |
| | polypeptide C, 30 kDa | | | | |
| 217420_s_at | polymerase (RNA) II (DNA | 4.7756456 | 5.54E−005 | 0.0033813 | 1.794438442 |
| | directed) polypeptide A, | | | | |
| | 220 kDa | | | | |
| 203664_s_at | polymerase (RNA) II (DNA | −3.370551 | 0.0022705 | 0.039341 | −1.760088633 |
| | directed) polypeptide D | | | | |
| 208361_s_at | polymerase (RNA) III | −3.787137 | 0.000773 | 0.0196639 | −0.710242225 |
| | (DNA directed) polypeptide | | | | |
| | D, 44 kDa | | | | |
| 218866_s_at | polymerase (RNA) III | −4.424747 | 0.0001422 | 0.0064859 | 0.880228392 |
| | (DNA directed) polypeptide | | | | |
| | K, 12.3 kDa | | | | |
| 203497_at | PPAR binding protein | 3.2511795 | 0.0030721 | 0.0472112 | −2.024300496 |
| 209434_s_at | phosphoribosyl | −4.281335 | 0.0002087 | 0.0084515 | 0.526113759 |
| | pyrophosphate | | | | |
| | amidotransferase | | | | |
| 202065_s_at | protein tyrosine | 3.6435674 | 0.0011245 | 0.0251089 | −1.100773385 |
| | phosphatase, receptor type, f | | | | |
| | polypeptide (PTPRF), | | | | |
| | interacting protein (liprin), | | | | |
| | alpha 1 | | | | |
| 202066_at | protein tyrosine | 3.2553223 | 0.0030402 | 0.0469801 | −2.043962733 |
| | phosphatase, receptor type, f | | | | |
| | polypeptide (PTPRF), | | | | |
| | interacting protein (liprin), | | | | |
| | alpha 1 | | | | |
| 200661_at | protective protein for beta- | 5.1153421 | 2.22E−005 | 0.0017996 | 2.684553909 |
| | galactosidase | | | | |
| | (galactosialidosis) | | | | |
| 201293_x_at | peptidylprolyl isomerase A | −3.36665 | 0.0022931 | 0.0395241 | −1.75999496 |
| | (cyclophilin A) | | | | |
| 211378_x_at | peptidylprolyl isomerase A | −3.350221 | 0.002391 | 0.0404668 | −1.803678035 |
| | (cyclophilin A) | | | | |
| 211978_x_at | peptidylprolyl isomerase A | −3.299201 | 0.0027213 | 0.043688 | −1.915402447 |
| | (cyclophilin A) | | | | |
| 210502_s_at | peptidylprolyl isomerase E | −3.669575 | 0.0010509 | 0.0239197 | −1.008073851 |
| | (cyclophilin E) | | | | |
| 207758_at | Protein phosphatase 1F | 3.3114187 | 0.0026384 | 0.04282 | −1.912439683 |
| | (PP2C domain containing) | | | | |
| 202014_at | protein phosphatase 1, | 3.2288821 | 0.0032494 | 0.048825 | −2.055247461 |
| | regulatory (inhibitor) | | | | |
| | subunit 15A | | | | |
| 212750_at | protein phosphatase 1, | −4.909646 | 3.86E−005 | 0.0025689 | 2.20013485 |
| | regulatory (inhibitor) | | | | |
| | subunit 16B | | | | |
| 41577_at | protein phosphatase 1, | −3.518204 | 0.0015554 | 0.0308899 | −1.336958672 |
| | regulatory (inhibitor) | | | | |
| | subunit 16B | | | | |
| 213849_s_at | protein phosphatase 2 | −3.284133 | 0.002827 | 0.0448357 | −1.958462839 |
| | (formerly 2A), regulatory | | | | |
| | subunit B (PR 52), beta | | | | |
| | isoform | | | | |
| 213305_s_at | protein phosphatase 2, | −3.965976 | 0.0004828 | 0.0146157 | −0.270658033 |
| | regulatory subunit B (B56), | | | | |
| | gamma isoform | | | | |
| 214083_at | Protein phosphatase 2, | −3.889946 | 0.00059 | 0.0165114 | −0.455729463 |
| | regulatory subunit B (B56), | | | | |
| | gamma isoform | | | | |
| 201594_s_at | protein phosphatase 4, | 4.0043528 | 0.0004361 | 0.0136305 | −0.181189613 |
| | regulatory subunit 1 | | | | |
| 214617_at | perforin 1 (pore forming | −4.74251 | 6.06E−005 | 0.0036173 | 1.707928613 |
| | protein) | | | | |
| 201859_at | proteoglycan 1, secretory | 3.7261571 | 0.0009068 | 0.0217964 | −0.901209187 |
| | granule | | | | |
| 201858_s_at | proteoglycan 1, secretory | 3.7248602 | 0.0009098 | 0.0217998 | −0.881645013 |
| | granule | | | | |
| 200603_at | protein kinase, cAMP- | 3.8331052 | 0.0006852 | 0.0181313 | −0.609864972 |
| | dependent, regulatory, type | | | | |
| | I, alpha (tissue specific | | | | |
| | extinguisher 1) | | | | |
| 206099_at | protein kinase C, eta | −3.903842 | 0.0005688 | 0.0162109 | −0.4380415 |
| 218764_at | protein kinase C, eta | −3.778505 | 0.0007907 | 0.0199363 | −0.707292885 |
| 210039_s_at | protein kinase C, theta | −6.853394 | 2.28E−007 | 7.17E−005 | 7.089585199 |
| 210038_at | protein kinase C, theta | −5.530647 | 7.30E−006 | 0.0008252 | 3.79220792 |
| 202178_at | protein kinase C, zeta | −3.968501 | 0.0004795 | 0.014558 | −0.275167269 |
| 206445_s_at | protein arginine | −3.942933 | 0.0005131 | 0.0151224 | −0.315594704 |
| | methyltransferase 1 | | | | |
| 203103_s_at | PRP19/PSO4 pre-mRNA | −3.738231 | 0.0008786 | 0.021299 | −0.85927654 |
| | processing factor 19 | | | | |
| | homolog (S. cerevisiae) | | | | |
| 208447_s_at | phosphoribosyl | −3.493771 | 0.0016563 | 0.0322342 | −1.436267267 |
| | pyrophosphate synthetase 1 | | | | |
| 219168_s_at | proline rich 5 (renal) | −4.699217 | 6.80E−005 | 0.0039477 | 1.59932363 |
| 47069_at | proline rich 5 (renal) | −4.455505 | 0.0001309 | 0.0061941 | 0.973083191 |
| 202879_s_at | pleckstrin homology, Sec7 | −3.470596 | 0.001758 | 0.0334519 | −1.529031395 |
| | and coiled-coil domains | | | | |
| | 1(cytohesin 1) | | | | |
| 202880_s_at | pleckstrin homology, Sec7 | −3.232715 | 0.0032183 | 0.0484547 | −2.038966644 |
| | and coiled-coil domains | | | | |
| | 1(cytohesin 1) | | | | |
| 209158_s_at | pleckstrin homology, Sec7 | −3.742987 | 0.0008677 | 0.0211928 | −0.826655529 |
| | and coiled-coil domains 2 | | | | |
| | (cytohesin-2) | | | | |
| 210758_at | PC4 and SFRS1 interacting | −3.649747 | 0.0011065 | 0.0249298 | −1.065694547 |
| | protein 1 | | | | |
| 202659_at | proteasome (prosome, | −3.382304 | 0.0022035 | 0.0384797 | −1.699580216 |
| | macropain) subunit, beta | | | | |
| | type, 10 | | | | |
| 204279_at | proteasome (prosome, | −4.917067 | 3.79E−005 | 0.0025408 | 2.161402811 |
| | macropain) subunit, beta | | | | |
| | type, 9 (large | | | | |
| | multifunctional peptidase 2) | | | | |
| 201252_at | proteasome (prosome, | −3.318592 | 0.0025909 | 0.0424462 | −1.858034479 |
| | macropain) 26S subunit, | | | | |
| | ATPase, 4 /// similar to 26S | | | | |
| | protease regulatory subunit | | | | |
| | 6B (MIP224) (MB67- | | | | |
| | interacting protein) (TAT- | | | | |
| | binding protein 7) (TBP-7) | | | | |
| 201198_s_at | proteasome (prosome, | 4.5144731 | 0.0001118 | 0.0055344 | 1.108528079 |
| | macropain) 26S subunit, | | | | |
| | non-ATPase, 1 | | | | |
| 200814_at | proteasome (prosome, | −6.067133 | 1.76E−006 | 0.0003031 | 5.126788139 |
| | macropain) activator subunit | | | | |
| | 1 (PA28 alpha) | | | | |
| 212723_at | phosphatidylserine receptor | 4.2005609 | 0.0002589 | 0.0097791 | 0.303780047 |
| 211711_s_at | phosphatase and tensin | 3.242948 | 0.0031365 | 0.0476847 | −2.066234845 |
| | homolog (mutated in | | | | |
| | multiple advanced cancers | | | | |
| | 1) | | | | |
| 206574_s_at | protein tyrosine phosphatase | −3.352979 | 0.0023743 | 0.0403305 | −1.798717241 |
| | type IVA, member 3 | | | | |
| 217777_s_at | protein tyrosine | −5.042402 | 2.70E−005 | 0.002062 | 2.492290453 |
| | phosphatase-like A domain | | | | |
| | containing 1 | | | | |
| 202006_at | protein tyrosine | 3.6813214 | 0.0010193 | 0.0234633 | −1.006965581 |
| | phosphatase, non-receptor | | | | |
| | type 12 | | | | |
| 213136_at | protein tyrosine | 3.6442504 | 0.0011225 | 0.0251089 | −1.091320927 |
| | phosphatase, non-receptor | | | | |
| | type 2 | | | | |
| 213137_s_at | protein tyrosine | 3.5486358 | 0.001438 | 0.0293157 | −1.337650355 |
| | phosphatase, non-receptor | | | | |
| | type 2 | | | | |
| 205171_at | protein tyrosine | −3.274455 | 0.002897 | 0.0455242 | −1.936046497 |
| | phosphatase, non-receptor | | | | |
| | type 4 (megakaryocyte) | | | | |
| 204852_s_at | protein tyrosine | −3.753565 | 0.0008441 | 0.0207369 | −0.81519252 |
| | phosphatase, non-receptor | | | | |
| | type 7 | | | | |
| 204960_at | protein tyrosine | −3.453943 | 0.0018347 | 0.0342115 | −1.543248457 |
| | phosphatase, receptor type, | | | | |
| | C-associated protein | | | | |
| 221840_at | protein tyrosine | 3.7346234 | 0.0008869 | 0.0214585 | −0.858583092 |
| | phosphatase, receptor type, E | | | | |
| 200677_at | pituitary tumor- | 4.1576558 | 0.0002903 | 0.0105349 | 0.239233016 |
| | transforming 1 interacting | | | | |
| | protein | | | | |
| 206157_at | pentraxin-related gene, | 3.2216752 | 0.0033088 | 0.049517 | −2.081708082 |
| | rapidly induced by IL-1 beta | | | | |
| 202990_at | phosphorylase, glycogen; | 3.831527 | 0.000688 | 0.0181655 | −0.619929851 |
| | liver (Hers disease, | | | | |
| | glycogen storage disease | | | | |
| | type VI) | | | | |
| 212263_at | quaking homolog, KH | 5.3418653 | 1.21E−005 | 0.0011938 | 3.247778353 |
| | domain RNA binding | | | | |
| | (mouse) | | | | |
| 212636_at | quaking homolog, KH | 3.716191 | 0.0009307 | 0.0220336 | −0.908636132 |
| | domain RNA binding | | | | |
| | (mouse) | | | | |
| 212262_at | quaking homolog, KH | 3.2528714 | 0.0030591 | 0.0470963 | −2.026360286 |
| | domain RNA binding | | | | |
| | (mouse) | | | | |
| 201482_at | quiescin Q6 | 3.6677766 | 0.0010558 | 0.0240075 | −0.960258387 |
| 212866_at | R3H domain and coiled-coil | −3.402653 | 0.002092 | 0.037263 | −1.633480478 |
| | containing 1 | | | | |
| 202252_at | RAB13, member RAS | 4.4781095 | 0.0001232 | 0.0059304 | 1.030029421 |
| | oncogene family | | | | |
| 217763_s_at | RAB31, member RAS | 5.5810831 | 6.38E−006 | 0.0007574 | 3.890838429 |
| | oncogene family | | | | |
| 217764_s_at | RAB31, member RAS | 4.4541374 | 0.0001314 | 0.0062037 | 0.99303238 |
| | oncogene family | | | | |
| 217762_s_at | RAB31, member RAS | 3.7228006 | 0.0009147 | 0.0218473 | −0.896149479 |
| | oncogene family | | | | |
| 206039_at | RAB33A, member RAS | −4.783528 | 5.42E−005 | 0.0033259 | 1.826241148 |
| | oncogene family | | | | |
| 209181_s_at | Rab | 4.0980711 | 0.0003401 | 0.0116248 | 0.109722119 |
| | geranylgeranyltransferase, | | | | |
| | beta subunit | | | | |
| 204460_s_at | RAD1 homolog (S. pombe) | −4.550561 | 0.0001014 | 0.0052326 | 1.211555802 |
| 210216_x_at | RAD1 homolog (S. pombe) | −3.352923 | 0.0023746 | 0.0403305 | −1.811129071 |
| 201223_s_at | RAD23 homolog B (S. cerevisiae) | 3.3947269 | 0.0021348 | 0.0376896 | −1.68684609 |
| 212646_at | raft-linking protein | −4.620352 | 8.41E−005 | 0.0045156 | 1.443543789 |
| 200750_s_at | RAN, member RAS | −3.366595 | 0.0022934 | 0.0395241 | −1.744972227 |
| | oncogene family | | | | |
| 202483_s_at | RAN binding protein 1 | −4.976076 | 3.23E−005 | 0.0022994 | 2.321294163 |
| 211955_at | RAN binding protein 5 | −3.198218 | 0.0035094 | 0.0511728 | −2.086289595 |
| 214487_s_at | RAP2A, member of RAS | −3.476998 | 0.0017293 | 0.0331904 | −1.508778336 |
| | oncogene family /// RAP2B, | | | | |
| | member of RAS oncogene | | | | |
| | family | | | | |
| 203097_s_at | Rap guanine nucleotide | 3.6240801 | 0.0011828 | 0.0258655 | −1.130227944 |
| | exchange factor (GEF) 2 | | | | |
| 215992_s_at | Rap guanine nucleotide | 4.0476699 | 0.0003888 | 0.0127232 | −0.083213358 |
| | exchange factor (GEF) 2 /// | | | | |
| | similar to Rap guanine | | | | |
| | nucleotide exchange factor | | | | |
| | 2 (PDZ domain containing | | | | |
| | guanine nucleotide | | | | |
| | exchange factor 1) (PDZ- | | | | |
| | GEF1) (RA-GEF) | | | | |
| 204070_at | retinoic acid receptor | −7.315638 | 7.11E−008 | 3.68E−005 | 8.198820117 |
| | responder (tazarotene | | | | |
| | induced) 3 | | | | |
| 206220_s_at | RAS p21 protein activator 3 | −3.990046 | 0.000453 | 0.0139223 | −0.228386854 |
| 205590_at | RAS guanyl releasing | −5.976124 | 2.23E−006 | 0.0003578 | 4.913421176 |
| | protein 1 (calcium and | | | | |
| | DAG-regulated) | | | | |
| 203185_at | Ras association | 3.5744411 | 0.0013452 | 0.0280347 | −1.257686623 |
| | (RalGDS/AF-6) domain | | | | |
| | family 2 | | | | |
| 49306_at | Ras association | 4.3680986 | 0.0001655 | 0.0072161 | 0.74649399 |
| | (RalGDS/AF-6) domain | | | | |
| | family 4 | | | | |
| 203132_at | retinoblastoma 1 (including | 3.3206482 | 0.0025774 | 0.042292 | −1.853707726 |
| | osteosarcoma) | | | | |
| 201092_at | retinoblastoma binding | −3.461426 | 0.0017998 | 0.0338007 | −1.459013192 |
| | protein 7 | | | | |
| 221827_at | RanBP-type and C3HC4- | −3.376448 | 0.0022366 | 0.0388607 | −1.728525816 |
| | type zinc finger containing 1 | | | | |
| 215127_s_at | RNA binding motif, single | 3.2508813 | 0.0030744 | 0.0472141 | −2.056864194 |
| | stranded interacting protein | | | | |
| | 1 /// chromosome 2 open | | | | |
| | reading frame 12 | | | | |
| 203748_x_at | RNA binding motif, single | 3.4254135 | 0.0019738 | 0.0358449 | −1.573315081 |
| | stranded interacting protein | | | | |
| | 1 /// region containing | | | | |
| | chromosome 2 open reading | | | | |
| | frame 12; RNA binding | | | | |
| | motif, single stranded | | | | |
| | interacting protein 1 | | | | |
| 211974_x_at | recombining binding protein | 3.995672 | 0.0004463 | 0.0138256 | −0.226610249 |
| | suppressor of hairless | | | | |
| | (Drosophila) | | | | |
| 212612_at | REST corepressor 1 | 3.3408222 | 0.0024488 | 0.041089 | −1.791580506 |
| 218777_at | receptor accessory protein 4 | 3.9027319 | 0.0005705 | 0.0162223 | −0.430708647 |
| 215201_at | RALBP1 associated Eps | 3.3806277 | 0.0022129 | 0.0385538 | −1.721122439 |
| | domain containing 1 | | | | |
| 220570_at | resistin | 4.982554 | 3.17E−005 | 0.0022669 | 2.386826391 |
| 218194_at | REX2, RNA exonuclease 2 | −4.435454 | 0.0001382 | 0.006378 | 0.936889763 |
| | homolog (S. cerevisiae) | | | | |
| 203659_s_at | ret finger protein 2 | −3.474998 | 0.0017382 | 0.0332359 | −1.464453355 |
| 202964_s_at | regulatory factor X, 5 | −6.568302 | 4.75E−007 | 0.0001267 | 6.387230752 |
| | (influences HLA class II | | | | |
| | expression) | | | | |
| 218430_s_at | regulatory factor X domain | −3.75278 | 0.0008458 | 0.0207567 | −0.833055056 |
| | containing 2 | | | | |
| 218723_s_at | response gene to | −3.355382 | 0.0023598 | 0.040171 | −1.774231113 |
| | complement 32 | | | | |
| 209568_s_at | ral guanine nucleotide | 3.8281998 | 0.0006941 | 0.01826 | −0.593999046 |
| | dissociation stimulator-like 1 | | | | |
| 209110_s_at | ral guanine nucleotide | 4.5321275 | 0.0001066 | 0.0054228 | 1.157756408 |
| | dissociation stimulator-like 2 | | | | |
| 202388_at | regulator of G-protein | 4.1592531 | 0.0002891 | 0.0105244 | 0.224364062 |
| | signalling 2, 24 kDa | | | | |
| 200059_s_at | ras homolog gene family, | 4.2518542 | 0.0002258 | 0.0089214 | 0.460974737 |
| | member A | | | | |
| 200885_at | ras homolog gene family, | −4.447853 | 0.0001336 | 0.0062624 | 0.954050093 |
| | member C | | | | |
| 219045_at | ras homolog gene family, | −3.608608 | 0.0012313 | 0.0264526 | −1.159547192 |
| | member F (in filopodia) | | | | |
| 204951_at | ras homolog gene family, | −4.895416 | 4.01E−005 | 0.0026299 | 2.143584049 |
| | member H | | | | |
| 218323_at | ras homolog gene family, | 4.9871693 | 3.13E−005 | 0.0022462 | 2.351696222 |
| | member T1 | | | | |
| 222148_s_at | ras homolog gene family, | 3.6083076 | 0.0012322 | 0.0264526 | −1.184052052 |
| | member T1 | | | | |
| 204730_at | regulating synaptic | −3.738476 | 0.000878 | 0.021299 | −0.850341898 |
| | membrane exocytosis 3 | | | | |
| 209684_at | Ras and Rab interactor 2 | 3.4795425 | 0.001718 | 0.0330187 | −1.482175086 |
| 219457_s_at | Ras and Rab interactor 3 | 4.1348632 | 0.0003084 | 0.0109442 | 0.169153984 |
| 220439_at | Ras and Rab interactor 3 | 3.7399726 | 0.0008746 | 0.021299 | −0.815042942 |
| 202130_at | RIO kinase 3 (yeast) | 3.2471073 | 0.0031038 | 0.0474828 | −2.041727026 |
| 209941_at | receptor (TNFRSF)- | −4.67766 | 7.21E−005 | 0.0041288 | 1.540113991 |
| | interacting serine-threonine | | | | |
| | kinase 1 | | | | |
| 201785_at | ribonuclease, RNase A | 7.5450772 | 4.02E−008 | 2.49E−005 | 8.69840649 |
| | family, 1 (pancreatic) | | | | |
| 216667_at | ribonuclease, RNase A | 7.9404174 | 1.53E−008 | 1.36E−005 | 9.485566694 |
| | family, 2 (liver, eosinophil- | | | | |
| | derived neurotoxin) | | | | |
| 206111_at | ribonuclease, RNase A | 6.1911281 | 1.27E−006 | 0.000249 | 5.45081304 |
| | family, 2 (liver, eosinophil- | | | | |
| | derived neurotoxin) | | | | |
| 203022_at | ribonuclease H2, subunit A | −3.776635 | 0.0007946 | 0.0199959 | −0.746667541 |
| 209565_at | ring finger protein 113A | −5.602337 | 6.02E−006 | 0.0007376 | 3.955565351 |
| 201779_s_at | ring finger protein 13 | 3.4333096 | 0.0019343 | 0.0355626 | −1.614078726 |
| 221430_s_at | ring finger protein 146 | 5.160403 | 1.97E−005 | 0.0016545 | 2.832559757 |
| 212047_s_at | ring finger protein 167 | −4.40883 | 0.0001484 | 0.0066929 | 0.848623923 |
| 216798_at | ribonuclease/angiogenin | 3.5711695 | 0.0013566 | 0.0281204 | −1.237174552 |
| | inhibitor 1 /// hypothetical | | | | |
| | protein FLJ23519 | | | | |
| 216621_at | Rho-associated, coiled-coil | 3.2789846 | 0.002864 | 0.0451018 | −1.96790588 |
| | containing protein kinase 1 | | | | |
| 218394_at | rogdi homolog (Drosophila) | 4.5169344 | 0.000111 | 0.0055225 | 1.166994701 |
| 210426_x_at | RAR-related orphan | −4.021103 | 0.0004172 | 0.0132622 | −0.161414102 |
| | receptor A | | | | |
| 210479_s_at | RAR-related orphan | −3.575557 | 0.0013413 | 0.0279853 | −1.274155948 |
| | receptor A | | | | |
| 205191_at | retinitis pigmentosa 2 (X- | 3.3317358 | 0.0025059 | 0.0416403 | −1.8475666 |
| | linked recessive) | | | | |
| 201756_at | replication protein A2, | −3.207931 | 0.003425 | 0.0506432 | −2.072247497 |
| | 32 kDa | | | | |
| 209507_at | replication protein A3, | −4.351603 | 0.000173 | 0.0074472 | 0.692123864 |
| | 14 kDa | | | | |
| 212191_x_at | ribosomal protein L13 | −3.3278 | 0.0025311 | 0.041902 | −1.856477787 |
| 214351_x_at | ribosomal protein L13 /// | −3.49646 | 0.0016449 | 0.0320401 | −1.442060332 |
| | similar to ribosomal protein | | | | |
| | L13 | | | | |
| 200715_x_at | ribosomal protein L13a | −3.99766 | 0.0004439 | 0.013816 | −0.193252593 |
| 211942_x_at | ribosomal protein L13a /// | −3.553862 | 0.0014187 | 0.0289756 | −1.259096827 |
| | similar to ribosomal protein | | | | |
| | L13a /// similar to ribosomal | | | | |
| | protein L13a; 60S ribosomal | | | | |
| | protein L13a; 23 kD highly | | | | |
| | basic protein | | | | |
| 220960_x_at | ribosomal protein L22 | −3.661276 | 0.0010739 | 0.0243178 | −1.019404571 |
| 203012_x_at | ribosomal protein L23a | −3.454963 | 0.0018299 | 0.0341507 | −1.529289261 |
| 213084_x_at | ribosomal protein L23a | −3.450528 | 0.0018508 | 0.0343685 | −1.566511701 |
| 211666_x_at | ribosomal protein L3 | −3.99006 | 0.000453 | 0.0139223 | −0.19819782 |
| 215963_x_at | ribosomal protein L3 /// | −3.297282 | 0.0027346 | 0.043806 | −1.86966255 |
| | similar to 60S ribosomal | | | | |
| | protein L3 (L4) | | | | |
| 200002_at | ribosomal protein L35 | −3.34394 | 0.0024295 | 0.0408571 | −1.752574709 |
| 219762_s_at | ribosomal protein L36 | −3.599339 | 0.0012612 | 0.0268163 | −1.141391787 |
| 202029_x_at | ribosomal protein L38 | −4.192448 | 0.0002646 | 0.0098805 | 0.322151958 |
| 210115_at | ribosomal protein L39-like | −3.413489 | 0.0020349 | 0.0366855 | −1.659796427 |
| 216215_s_at | Ribosomal protein L41 | −3.339427 | 0.0024575 | 0.0411468 | −1.823090187 |
| 211972_x_at | ribosomal protein, large, P0 | −4.171509 | 0.0002798 | 0.0102535 | 0.272186375 |
| 208856_x_at | ribosomal protein, large, P0 | −3.578695 | 0.0013305 | 0.0278887 | −1.240907845 |
| 214167_s_at | ribosomal protein, large, P0 | −4.199013 | 0.00026 | 0.0098029 | 0.324055574 |
| | /// similar to ribosomal | | | | |
| | protein P0 | | | | |
| 211542_x_at | ribosomal protein S10 | −3.237133 | 0.0031827 | 0.0480165 | −2.05026604 |
| 212578_x_at | ribosomal protein S17 | −3.24347 | 0.0031324 | 0.0476847 | −2.037558887 |
| 217753_s_at | ribosomal protein S26 /// | −5.239186 | 1.59E−005 | 0.0014332 | 3.042053812 |
| | similar to 40S ribosomal | | | | |
| | protein S26 | | | | |
| 208903_at | Ribosomal protein S28 | −3.773839 | 0.0008004 | 0.0199959 | −0.755039904 |
| 200024_at | ribosomal protein S5 | −3.384265 | 0.0021925 | 0.0383779 | −1.702994837 |
| 213801_x_at | ribosomal protein SA /// | −4.782806 | 5.43E−005 | 0.0033259 | 1.84102526 |
| | similar to 40S ribosomal | | | | |
| | protein SA (p40) (34/67 kDa | | | | |
| | laminin receptor) | | | | |
| | (Colon carcinoma laminin- | | | | |
| | binding protein) | | | | |
| | (NEM/1CHD4) (Multidrug | | | | |
| | resistance-associated protein | | | | |
| | MGr1-Ag) /// similar to | | | | |
| | Laminin receptor 1 | | | | |
| 212955_s_at | ribosomal protein SA /// | −3.328031 | 0.0025296 | 0.041902 | −1.854419924 |
| | polymerase (RNA) II (DNA | | | | |
| | directed) polypeptide I, | | | | |
| | 14.5 kDa | | | | |
| 212590_at | related RAS viral (r-ras) | −4.154538 | 0.0002927 | 0.010571 | 0.177538426 |
| | oncogene homolog 2 | | | | |
| 208456_s_at | related RAS viral (r-ras) | −3.253809 | 0.0030518 | 0.0470787 | −2.03681605 |
| | oncogene homolog 2 | | | | |
| 201476_s_at | ribonucleotide reductase M1 | −3.816359 | 0.000716 | 0.0186551 | −0.678928654 |
| | polypeptide | | | | |
| 214629_x_at | reticulon 4 | 5.4353129 | 9.41E−006 | 0.0010085 | 3.52120334 |
| 210968_s_at | reticulon 4 | 3.4612717 | 0.0018005 | 0.0338007 | −1.52848835 |
| 219684_at | receptor transporter protein 4 | −5.497652 | 7.97E−006 | 0.0008834 | 3.662251874 |
| 219957_at | RUN and FYVE domain | 3.7812485 | 0.0007851 | 0.0198789 | −0.721550596 |
| | containing 2 | | | | |
| 216976_s_at | RYK receptor-like tyrosine | −3.363059 | 0.0023142 | 0.0396362 | −1.772602373 |
| | kinase | | | | |
| 205863_at | S100 calcium binding | 5.6697411 | 5.03E−006 | 0.000656 | 4.132277826 |
| | protein A12 (calgranulin C) | | | | |
| 202917_s_at | S100 calcium binding | 6.291558 | 9.75E−007 | 0.0002131 | 5.69316671 |
| | protein A8 (calgranulin A) | | | | |
| 214370_at | S100 calcium binding | −3.399446 | 0.0021092 | 0.0374709 | −1.653296212 |
| | protein A8 (calgranulin A) | | | | |
| 203535_at | S100 calcium binding | 6.627653 | 4.08E−007 | 0.000115 | 6.534375705 |
| | protein A9 (calgranulin B) | | | | |
| 204351_at | S100 calcium binding | 4.4562535 | 0.0001307 | 0.0061941 | 0.992552415 |
| | protein P | | | | |
| 220330_s_at | SAM domain, SH3 domain | 4.4171826 | 0.0001451 | 0.0065847 | 0.889493127 |
| | and nuclear localisation | | | | |
| | signals, 1 | | | | |
| 204900_x_at | Sin3A-associated protein, | 4.804201 | 5.13E−005 | 0.0032122 | 1.871086644 |
| | 30 kDa | | | | |
| 218854_at | squamous cell carcinoma | 4.192474 | 0.0002646 | 0.0098805 | 0.301950935 |
| | antigen recognized by T | | | | |
| | cells 2 | | | | |
| 209486_at | disrupter of silencing 10 | −5.287723 | 1.40E−005 | 0.0013052 | 3.148994281 |
| 213236_at | SAM and SH3 domain | 6.3133264 | 9.22E−007 | 0.0002065 | 5.755701955 |
| | containing 1 | | | | |
| 41644_at | SAM and SH3 domain | 5.4976382 | 7.97E−006 | 0.0008834 | 3.683493911 |
| | containing 1 | | | | |
| 201771_at | secretory carrier membrane | −3.503221 | 0.0016166 | 0.0316258 | −1.400560388 |
| | protein 3 | | | | |
| 205790_at | src family associated | −4.231128 | 0.0002387 | 0.0093056 | 0.430626972 |
| | phosphoprotein 1 | | | | |
| 204362_at | src family associated | 5.3043064 | 1.34E−005 | 0.0012764 | 3.152409702 |
| | phosphoprotein 2 | | | | |
| 216899_s_at | src family associated | 3.7759379 | 0.0007961 | 0.0199959 | −0.771630871 |
| | phosphoprotein 2 | | | | |
| 215754_at | scavenger receptor class B, | 6.5663251 | 4.78E−007 | 0.0001267 | 6.383543044 |
| | member 2 | | | | |
| 205508_at | sodium channel, voltage- | 3.3957489 | 0.0021292 | 0.0376845 | −1.675658574 |
| | gated, type I, beta | | | | |
| 212589_at | Sterol carrier protein 2 | −3.681314 | 0.0010193 | 0.0234633 | −0.994205318 |
| 218217_at | serine carboxypeptidase 1 | 4.050768 | 0.0003857 | 0.0126796 | −0.06538095 |
| 200958_s_at | syndecan binding protein | 3.6157902 | 0.0012085 | 0.0261711 | −1.165750945 |
| | (syntenin) | | | | |
| 201093_x_at | succinate dehydrogenase | −4.789653 | 5.33E−005 | 0.0033048 | 1.823696837 |
| | complex, subunit A, | | | | |
| | flavoprotein (Fp) | | | | |
| 215652_at | succinate dehydrogenase | −3.572334 | 0.0013525 | 0.0280881 | −1.240547061 |
| | complex, subunit D, integral | | | | |
| | membrane protein /// similar | | | | |
| | to Succinate dehydrogenase | | | | |
| | [ubiquinone] cytochrome b | | | | |
| 202082_s_at | small subunit, mitochondrial | 3.2965339 | 0.0027397 | 0.0438575 | −1.933246382 |
| | precursor (CybS) | | | | |
| | (Succinate-ubiquinone | | | | |
| | reductase membrane anchor | | | | |
| | subunit) (QPs3) (CII-4) | | | | |
| | (Succinate dehydrogenase | | | | |
| | complex subunit D) | | | | |
| | (Succinate-ubiquinone oxi . . . | | | | |
| | SEC14-like 1 (S. cerevisiae) | | | | |
| 212887_at | Sec23 homolog A (S. cerevisiae) | 3.2478869 | 0.0030977 | 0.0474828 | −2.007581111 |
| 212902_at | SEC24 related gene family, | 4.538584 | 0.0001048 | 0.0053418 | 1.168840876 |
| | member A (S. cerevisiae) | | | | |
| 203789_s_at | sema domain, | 3.3982188 | 0.0021158 | 0.0375372 | −1.644826163 |
| | immunoglobulin domain | | | | |
| | (Ig), short basic domain, | | | | |
| | secreted, (semaphorin) 3C | | | | |
| 46665_at | sema domain, | −8.88408 | 1.65E−009 | 2.06E−006 | 11.76724808 |
| | immunoglobulin domain | | | | |
| | (Ig), transmembrane domain | | | | |
| | (TM) and short cytoplasmic | | | | |
| | domain, (semaphorin) 4C | | | | |
| 41220_at | septin 9 | −3.880289 | 0.0006052 | 0.0167115 | −0.462154044 |
| 217977_at | selenoprotein X, 1 | 3.2270697 | 0.0032643 | 0.0490149 | −1.995476502 |
| 202833_s_at | serpin peptidase inhibitor, | 3.6497865 | 0.0011064 | 0.0249298 | −1.021810954 |
| | clade A (alpha-1 | | | | |
| | antiproteinase, antitrypsin), | | | | |
| | member 1 | | | | |
| 211429_s_at | serpin peptidase inhibitor, | 3.3814158 | 0.0022085 | 0.0385065 | −1.727765762 |
| | clade A (alpha-1 | | | | |
| | antiproteinase, antitrypsin), | | | | |
| | member 1 | | | | |
| 206034_at | serpin peptidase inhibitor, | 4.0271608 | 0.0004106 | 0.013184 | −0.087261655 |
| | clade B (ovalbumin), | | | | |
| | member 8 | | | | |
| 213370_s_at | Scm-like with four mbt | −3.239883 | 0.0031608 | 0.0478336 | −2.069334589 |
| | domains 1 | | | | |
| 37004_at | surfactant, pulmonary- | −3.562882 | 0.001386 | 0.0285172 | −1.270139183 |
| | associated protein B | | | | |
| 210116_at | SH2 domain protein 1A, | −4.560044 | 9.89E−005 | 0.0051366 | 1.267219598 |
| | Duncan's disease | | | | |
| | (lymphoproliferative | | | | |
| | syndrome) | | | | |
| 211209_x_at | SH2 domain protein 1A, | −4.553623 | 0.0001006 | 0.0052138 | 1.209258708 |
| | Duncan's disease | | | | |
| | (lymphoproliferative | | | | |
| | syndrome) | | | | |
| 211211_x_at | SH2 domain protein 1A, | −4.125885 | 0.0003159 | 0.0110921 | 0.112002011 |
| | Duncan's disease | | | | |
| | (lymphoproliferative | | | | |
| | syndrome) | | | | |
| 211210_x_at | SH2 domain protein 1A, | −4.102185 | 0.0003364 | 0.0115515 | 0.045987459 |
| | Duncan's disease | | | | |
| | (lymphoproliferative | | | | |
| | syndrome) | | | | |
| 207351_s_at | SH2 domain protein 2A | −3.697451 | 0.0009773 | 0.0228272 | −0.967999281 |
| 201312_s_at | SH3 domain binding | 4.4991084 | 0.0001165 | 0.0056915 | 1.094296518 |
| | glutamic acid-rich protein | | | | |
| | like | | | | |
| 211250_s_at | SH3-domain binding protein 2 | 4.3256664 | 0.0001854 | 0.0078519 | 0.665768553 |
| 209370_s_at | SH3-domain binding protein 2 | 3.4731675 | 0.0017464 | 0.0332929 | −1.521432673 |
| 218813_s_at | SH3-domain GRB2-like | −3.515977 | 0.0015643 | 0.03095 | −1.404751905 |
| | endophilin B2 | | | | |
| 204656_at | Src homology 2 domain | 3.7052084 | 0.0009577 | 0.0224641 | −0.834417401 |
| | containing adaptor protein B | | | | |
| 52940_at | single immunoglobulin and | −4.852165 | 4.51E−005 | 0.0028944 | 1.989519335 |
| | toll-interleukin 1 receptor | | | | |
| | (TIR) domain | | | | |
| 218921_at | single immunoglobulin and | −3.488888 | 0.0016773 | 0.0325236 | −1.469916457 |
| | toll-interleukin 1 receptor | | | | |
| | (TIR) domain | | | | |
| 202897_at | signal-regulatory protein | 4.3911658 | 0.0001556 | 0.0069054 | 0.821639939 |
| | alpha | | | | |
| 222248_s_at | sirtuin (silent mating type | −3.31182 | 0.0026357 | 0.0428077 | −1.912881019 |
| | information regulation 2 | | | | |
| | homolog) 4 (S. cerevisiae) | | | | |
| 205484_at | signaling threshold | −5.594881 | 6.15E−006 | 0.0007484 | 3.913038159 |
| | regulating transmembrane | | | | |
| | adaptor 1 | | | | |
| 203489_at | CD27-binding (Siva) | −4.279118 | 0.0002099 | 0.0084598 | 0.508890348 |
| | protein | | | | |
| 206181_at | signaling lymphocytic | −4.366114 | 0.0001664 | 0.0072404 | 0.722340143 |
| | activation molecule family | | | | |
| | member 1 | | | | |
| 219159_s_at | SLAM family member 7 | −5.797399 | 3.59E−006 | 0.0004962 | 4.437017041 |
| 217507_at | solute carrier family 11 | 5.5773418 | 6.44E−006 | 0.0007574 | 3.896571346 |
| | (proton-coupled divalent | | | | |
| | metal ion transporters), | | | | |
| | member 1 | | | | |
| 217473_x_at | solute carrier family 11 | 4.9535835 | 3.43E−005 | 0.0023969 | 2.320530767 |
| | (proton-coupled divalent | | | | |
| | metal ion transporters), | | | | |
| | member 1 | | | | |
| 210422_x_at | solute carrier family 11 | 4.9323812 | 3.63E−005 | 0.0024831 | 2.245625928 |
| | (proton-coupled divalent | | | | |
| | metal ion transporters), | | | | |
| | member 1 | | | | |
| 210423_s_at | solute carrier family 11 | 4.6657676 | 7.44E−005 | 0.004175 | 1.515295173 |
| | (proton-coupled divalent | | | | |
| | metal ion transporters), | | | | |
| | member 1 | | | | |
| 220281_at | solute carrier family 12 | −3.427437 | 0.0019636 | 0.0358275 | −1.59942765 |
| | (sodium/potassium/chloride | | | | |
| | transporters), member 1 | | | | |
| 206599_at | solute carrier family 16, | 3.4590496 | 0.0018108 | 0.0339653 | −1.499928251 |
| | member 5 (monocarboxylic | | | | |
| | acid transporter 6) | | | | |
| 211576_s_at | solute carrier family 19 | 3.4761341 | 0.0017331 | 0.0332077 | −1.50089401 |
| | (folate transporter), member 1 | | | | |
| 202800_at | solute carrier family 1 (glial | 4.971459 | 3.27E−005 | 0.0023134 | 2.343838903 |
| | high affinity glutamate | | | | |
| | transporter), member 3 | | | | |
| 205896_at | solute carrier family 22 | 3.6470645 | 0.0011143 | 0.0250298 | −1.053619868 |
| | (organic cation transporter), | | | | |
| | member 4 | | | | |
| 218653_at | solute carrier family 25 | −3.235428 | 0.0031964 | 0.0481577 | −2.067745763 |
| | (mitochondrial carrier; | | | | |
| | ornithine transporter) | | | | |
| | member 15 | | | | |
| 202499_s_at | solute carrier family 2 | 8.9748804 | 1.34E−009 | 2.06E−006 | 11.92184489 |
| | (facilitated glucose | | | | |
| | transporter), member 3 | | | | |
| 202497_x_at | solute carrier family 2 | 5.0164432 | 2.90E−005 | 0.0021512 | 2.473164956 |
| | (facilitated glucose | | | | |
| | transporter), member 3 | | | | |
| 222088_s_at | solute carrier family 2 | 3.4188404 | 0.0020072 | 0.0362752 | −1.609263061 |
| | (facilitated glucose | | | | |
| | transporter), member 3 | | | | |
| 218494_s_at | SLC2A4 regulator | −3.832694 | 0.0006859 | 0.0181313 | −0.635309642 |
| 203306_s_at | solute carrier family 35 | 3.8991801 | 0.0005758 | 0.0163307 | −0.43629199 |
| | (CMP-sialic acid | | | | |
| | transporter), member A1 | | | | |
| 213119_at | solute carrier family 36 | 4.2862534 | 0.000206 | 0.0083605 | 0.565098669 |
| | (proton/amino acid | | | | |
| | symporter), member 1 | | | | |
| 213113_s_at | solute carrier family 43, | 3.7618019 | 0.0008261 | 0.0204299 | −0.775589198 |
| | member 3 | | | | |
| 218682_s_at | solute carrier family 4 | 3.8893751 | 0.0005909 | 0.0165114 | −0.462284949 |
| | (anion exchanger), member | | | | |
| | 1, adaptor protein | | | | |
| 210286_s_at | solute carrier family 4, | −5.006119 | 2.98E−005 | 0.0021909 | 2.38112589 |
| | sodium bicarbonate | | | | |
| | cotransporter, member 7 | | | | |
| 212295_s_at | solute carrier family 7 | −3.313905 | 0.0026218 | 0.0426754 | −1.878844931 |
| | (cationic amino acid | | | | |
| | transporter, y+ system), | | | | |
| | member 1 | | | | |
| 216603_at | solute carrier family 7 | 3.6168825 | 0.0012051 | 0.0261479 | −1.089682314 |
| | (cationic amino acid | | | | |
| | transporter, y+ system), | | | | |
| | member 8 | | | | |
| 201349_at | solute carrier family 9 | −3.530699 | 0.0015061 | 0.0302551 | −1.354915643 |
| | (sodium/hydrogen | | | | |
| | exchanger), member 3 | | | | |
| | regulator 1 | | | | |
| 206565_x_at | SMA3 | 3.5038829 | 0.0016138 | 0.0315997 | −1.428792702 |
| 215043_s_at | SMA3 /// SMA5 | 3.7113906 | 0.0009424 | 0.0221982 | −0.904576228 |
| 204099_at | SWI/SNF related, matrix | 3.3101737 | 0.0026467 | 0.0428616 | −1.843889944 |
| | associated, actin dependent | | | | |
| | regulator of chromatin, | | | | |
| | subfamily d, member 3 | | | | |
| 219695_at | sphingomyelin | −5.447888 | 9.10E−006 | 0.0009894 | 3.54577275 |
| | phosphodiesterase 3, neutral | | | | |
| | membrane (neutral | | | | |
| | sphingomyelinase II) | | | | |
| 220358_at | Jun dimerization protein | −3.566844 | 0.0013719 | 0.0283313 | −1.23314089 |
| | p21SNFT | | | | |
| 200826_at | small nuclear | −4.306171 | 0.0001953 | 0.0081193 | 0.586694209 |
| | ribonucleoprotein D2 | | | | |
| | polypeptide 16.5 kDa | | | | |
| 202567_at | small nuclear | −3.346852 | 0.0024115 | 0.0407024 | −1.774021463 |
| | ribonucleoprotein D3 | | | | |
| | polypeptide 18 kDa | | | | |
| 203832_at | small nuclear | −4.640416 | 7.97E−005 | 0.0043626 | 1.441496179 |
| | ribonucleoprotein | | | | |
| | polypeptide F | | | | |
| 201522_x_at | small nuclear | −4.049405 | 0.0003871 | 0.0127021 | −0.053295008 |
| | ribonucleoprotein | | | | |
| | polypeptide N /// SNRPN | | | | |
| | upstream reading frame | | | | |
| 206042_x_at | small nuclear | −4.022786 | 0.0004154 | 0.0132558 | −0.135235744 |
| | ribonucleoprotein | | | | |
| | polypeptide N /// SNRPN | | | | |
| | upstream reading frame | | | | |
| 200067_x_at | sorting nexin 3 | 3.3174733 | 0.0025982 | 0.0424462 | −1.842097372 |
| 203372_s_at | suppressor of cytokine | −4.740673 | 6.09E−005 | 0.0036255 | 1.695663438 |
| | signaling 2 | | | | |
| 203373_at | suppressor of cytokine | −3.944244 | 0.0005113 | 0.01509 | −0.311689967 |
| | signaling 2 | | | | |
| 206359_at | suppressor of cytokine | 4.9229353 | 3.73E−005 | 0.0025162 | 2.247238767 |
| | signaling 3 | | | | |
| 200642_at | superoxide dismutase 1, | −4.912801 | 3.83E−005 | 0.0025624 | 2.156758545 |
| | soluble (amyotrophic lateral | | | | |
| | sclerosis 1 (adult)) | | | | |
| 203509_at | sortilin-related receptor, | 3.7001214 | 0.0009705 | 0.0226994 | −0.945497816 |
| | L(DLR class) A repeats- | | | | |
| | containing | | | | |
| 210985_s_at | SP100 nuclear antigen | 4.5269315 | 0.0001081 | 0.0054492 | 1.132116103 |
| 220299_at | spermatogenesis associated 6 | 5.762379 | 3.93E−006 | 0.0005314 | 4.348169794 |
| 201997_s_at | spen homolog, | 3.8170987 | 0.0007146 | 0.018646 | −0.655826888 |
| | transcriptional regulator | | | | |
| | (Drosophila) | | | | |
| 202444_s_at | SPFH domain family, | 4.64707 | 7.83E−005 | 0.0043151 | 1.506652756 |
| | member 1 | | | | |
| 202441_at | SPFH domain family, | 3.3661947 | 0.0022958 | 0.0395338 | −1.724854079 |
| | member 1 | | | | |
| 216981_x_at | sialophorin (leukosialin, | −5.578129 | 6.43E−006 | 0.0007574 | 3.865874533 |
| | CD43) | | | | |
| 206057_x_at | sialophorin (leukosialin, | −4.828079 | 4.81E−005 | 0.0030535 | 1.921625535 |
| | CD43) | | | | |
| 202523_s_at | sparc/osteonectin, cwcv and | −5.770771 | 3.85E−006 | 0.0005261 | 4.365488779 |
| | kazal-like domains | | | | |
| | proteoglycan (testican) 2 | | | | |
| 202524_s_at | sparc/osteonectin, cwcv and | −3.816023 | 0.0007166 | 0.0186551 | −0.635340832 |
| | kazal-like domains | | | | |
| | proteoglycan (testican) 2 | | | | |
| 203127_s_at | serine palmitoyltransferase, | 4.4110372 | 0.0001475 | 0.0066804 | 0.858510802 |
| | long chain base subunit 2 | | | | |
| 213329_at | SLIT-ROBO Rho GTPase | 4.3965269 | 0.0001533 | 0.0068205 | 0.870848753 |
| | activating protein 2 | | | | |
| 218140_x_at | signal recognition particle | −3.35975 | 0.0023337 | 0.0398444 | −1.76519771 |
| | receptor, B subunit | | | | |
| 219204_s_at | serine racemase | −3.991728 | 0.000451 | 0.0139028 | −0.202762777 |
| 207040_s_at | suppression of | −3.59272 | 0.001283 | 0.0271506 | −1.220819315 |
| | tumorigenicity 13 (colon | | | | |
| | carcinoma) (Hsp70 | | | | |
| | interacting protein) | | | | |
| 216905_s_at | suppression of | 5.4450151 | 9.17E−006 | 0.0009922 | 3.555270009 |
| | tumorigenicity 14 (colon | | | | |
| | carcinoma) | | | | |
| 202005_at | suppression of | 4.5802168 | 9.37E−005 | 0.0049464 | 1.30725577 |
| | tumorigenicity 14 (colon | | | | |
| | carcinoma) | | | | |
| 204542_at | ST6 (alpha-N-acetyl- | 4.3773085 | 0.0001614 | 0.0071099 | 0.772266698 |
| | neuraminyl-2,3-beta- | | | | |
| | galactosyl-1,3)-N- | | | | |
| | acetylgalactosaminide | | | | |
| | alpha-2,6-sialyltransferase 2 | | | | |
| 38487_at | stabilin 1 | 9.0955932 | 1.02E−009 | 1.75E−006 | 12.20225685 |
| 204150_at | stabilin 1 | 6.2310727 | 1.14E−006 | 0.000238 | 5.543221025 |
| 209023_s_at | stromal antigen 2 | 4.1219927 | 0.0003192 | 0.0111654 | 0.096595212 |
| AFFX- | signal transducer and | −7.243677 | 8.51E−008 | 4.12E−005 | 8.040419882 |
| HUMISGF3 | activator of transcription 1, | | | | |
| A/M97935_MA_at | 91 kDa | | | | |
| AFFX- | signal transducer and | −7.033501 | 1.45E−007 | 5.64E−005 | 7.530750606 |
| HUMISGF3 | activator of transcription 1, | | | | |
| A/M97935_5_at | 91 kDa | | | | |
| 209969_s_at | signal transducer and | −5.581926 | 6.36E−006 | 0.0007574 | 3.875635569 |
| | activator of transcription 1, | | | | |
| | 91 kDa | | | | |
| AFFX- | signal transducer and | −4.787202 | 5.37E−005 | 0.0033141 | 1.813319891 |
| HUMISGF3 | activator of transcription 1, | | | | |
| A/M97935_MB_at | 91 kDa | | | | |
| 200887_s_at | signal transducer and | −4.673361 | 7.29E−005 | 0.0041477 | 1.513266429 |
| | activator of transcription 1, | | | | |
| | 91 kDa | | | | |
| AFFX- | signal transducer and | −4.529907 | 0.0001072 | 0.0054247 | 1.148390006 |
| HUMISGF3 | activator of transcription 1, | | | | |
| A/M97935_3_at | 91 kDa | | | | |
| 206118_at | signal transducer and | −6.450878 | 6.44E−007 | 0.0001605 | 6.093085989 |
| | activator of transcription 4 | | | | |
| 218424_s_at | STEAP family member 3 | 5.6295576 | 5.60E−006 | 0.0007014 | 4.035422827 |
| 202694_at | serine/threonine kinase 17a | −3.813817 | 0.0007208 | 0.0187415 | −0.676053224 |
| | (apoptosis-inducing) | | | | |
| 202695_s_at | serine/threonine kinase 17a | −3.430679 | 0.0019474 | 0.0356839 | −1.60893435 |
| | (apoptosis-inducing) | | | | |
| 208854_s_at | serine/threonine kinase 24 | −3.249854 | 0.0030824 | 0.0473038 | −2.047899674 |
| | (STE20 homolog, yeast) | | | | |
| 200783_s_at | stathmin 1/oncoprotein 18 | −3.37846 | 0.0022252 | 0.0387169 | −1.751618512 |
| 217714_x_at | stathmin 1/oncoprotein 18 | −3.271482 | 0.0029188 | 0.0456867 | −1.986663218 |
| 203767_s_at | steroid sulfatase | 4.3973474 | 0.000153 | 0.0068192 | 0.901273824 |
| | (microsomal), arylsulfatase | | | | |
| | C, isozyme S | | | | |
| 209238_at | syntaxin 3 | 3.4430674 | 0.0018866 | 0.0349155 | −1.559737295 |
| 210580_x_at | sulfotransferase family, | 4.1558468 | 0.0002917 | 0.0105514 | 0.236859619 |
| | cytosolic, 1A, phenol- | | | | |
| | preferring, member 3 /// | | | | |
| | sulfotransferase family, | | | | |
| | cytosolic, 1A, phenol- | | | | |
| | preferring, member 4 | | | | |
| 207601_at | sulfotransferase family, | 3.8640797 | 0.0006316 | 0.0171291 | −0.525205863 |
| | cytosolic, 1B, member 1 | | | | |
| 208739_x_at | SMT3 suppressor of mif | −3.281078 | 0.0028489 | 0.0449592 | −1.972956534 |
| | two 3 homolog 2 (S. cerevisiae) | | | | |
| 206506_s_at | suppressor of Ty 3 homolog | −3.491789 | 0.0016648 | 0.0323425 | −1.481624359 |
| | (S. cerevisiae) | | | | |
| 205224_at | surfeit 2 | −3.317998 | 0.0025948 | 0.0424462 | −1.843483405 |
| 207540_s_at | spleen tyrosine kinase | 6.8718487 | 2.18E−007 | 7.06E−005 | 7.135740333 |
| 212990_at | synaptojanin 1 | 3.3132433 | 0.0026262 | 0.0427158 | −1.89078544 |
| 220613_s_at | synaptotagmin-like 2 | −3.774238 | 0.0007996 | 0.0199959 | −0.76223741 |
| 221616_s_at | TAF9B RNA polymerase II, | 3.6660346 | 0.0010606 | 0.024078 | −1.015849145 |
| | TATA box binding protein | | | | |
| | (TBP)-associated factor, | | | | |
| | 31 kDa | | | | |
| 202307_s_at | transporter 1, ATP-binding | −3.601775 | 0.0012533 | 0.0267978 | −1.208287212 |
| | cassette, sub-family B | | | | |
| | (MDR/TAP) | | | | |
| 204769_s_at | transporter 2, ATP-binding | −8.325113 | 6.09E−009 | 5.66E−006 | 10.55890931 |
| | cassette, sub-family B | | | | |
| | (MDR/TAP) | | | | |
| 208829_at | TAP binding protein | −3.242838 | 0.0031373 | 0.0476847 | −2.056860461 |
| | (tapasin) | | | | |
| 219443_at | taspase, threonine aspartase, 1 | −4.217241 | 0.0002477 | 0.0094014 | 0.383060286 |
| 206916_x_at | tyrosine aminotransferase | −3.673253 | 0.0010409 | 0.0238135 | −1.031831811 |
| 209154_at | Tax1 (human T-cell | 6.3171051 | 9.13E−007 | 0.0002065 | 5.761271761 |
| | leukemia virus type I) | | | | |
| | binding protein 3 | | | | |
| 221858_at | TBC1 domain family, | 5.8959506 | 2.76E−006 | 0.0004129 | 4.689934159 |
| | member 12 | | | | |
| 222173_s_at | TBC1 domain family, | 5.9207656 | 2.59E−006 | 0.0003896 | 4.761967547 |
| | member 2 | | | | |
| 201813_s_at | TBC1 domain family, | 3.738163 | 0.0008788 | 0.021299 | −0.861861132 |
| | member 5 | | | | |
| 202495_at | tubulin-specific chaperone c | −4.096025 | 0.000342 | 0.0116702 | 0.0477012 |
| 212685_s_at | transducin (beta)-like 2 | 3.2619037 | 0.0029902 | 0.0464971 | −1.92502939 |
| 220684_at | T-box 21 | −4.287217 | 0.0002055 | 0.0083543 | 0.517434305 |
| 336_at | thromboxane A2 receptor | 3.8262027 | 0.0006977 | 0.0183344 | −0.636813205 |
| 204045_at | transcription elongation | −4.288456 | 0.0002048 | 0.0083418 | 0.582506611 |
| | factor A (SII)-like 1 | | | | |
| 202819_s_at | transcription elongation | 3.8102716 | 0.0007275 | 0.0188048 | −0.625046444 |
| | factor B (SIII), polypeptide | | | | |
| | 3 (110 kDa, elongin A) | | | | |
| 213730_x_at | transcription factor 3 (E2A | −3.39113 | 0.0021544 | 0.0378505 | −1.694396157 |
| | immunoglobulin enhancer | | | | |
| | binding factors E12/E47) | | | | |
| 204158_s_at | T-cell, immune regulator 1, | 3.3641796 | 0.0023076 | 0.0395937 | −1.77014464 |
| | ATPase, H+ transporting, | | | | |
| | lysosomal V0 subunit A3 | | | | |
| 204043_at | transcobalamin II; | 8.4547789 | 4.49E−009 | 4.54E−006 | 10.77422771 |
| | macrocytic anemia | | | | |
| 219715_s_at | tyrosyl-DNA | −4.812385 | 5.02E−005 | 0.0031672 | 1.88271269 |
| 203449_s_at | phosphodiesterase 1 | −4.026205 | 0.0004116 | 0.013197 | −0.094304254 |
| | telomeric repeat binding | | | | |
| | factor (NIMA-interacting) 1 | | | | |
| 202719_s_at | testis derived transcript (3 | −5.043891 | 2.69E−005 | 0.002062 | 2.513087156 |
| | LIM domains) | | | | |
| 202720_at | testis derived transcript (3 | −3.22533 | 0.0032786 | 0.0491966 | −2.076235048 |
| | LIM domains) | | | | |
| 218099_at | testis expressed sequence 2 | 4.7093484 | 6.62E−005 | 0.0038597 | 1.6201073 |
| 218605_at | transcription factor B2, | −4.088729 | 0.0003487 | 0.0118082 | 0.062719767 |
| | mitochondrial | | | | |
| 212457_at | transcription factor binding | 4.6050896 | 8.76E−005 | 0.0046709 | 1.352977834 |
| | to IGHM enhancer 3 | | | | |
| 216262_s_at | TGFB-induced factor 2 | −3.434437 | 0.0019287 | 0.0354894 | −1.602477257 |
| | (TALE family homeobox) | | | | |
| 212040_at | trans-golgi network protein 2 | 3.6330386 | 0.0011556 | 0.0254961 | −1.127589619 |
| 218492_s_at | THAP domain containing 7 | −3.490257 | 0.0016714 | 0.0324418 | −1.465161566 |
| 203887_s_at | thrombomodulin | 7.2217472 | 8.99E−008 | 4.20E−005 | 7.98099287 |
| 203888_at | thrombomodulin | 5.3756025 | 1.10E−005 | 0.0011342 | 3.366455464 |
| 201110_s_at | thrombospondin 1 | 3.8880711 | 0.0005929 | 0.0165364 | −0.456473608 |
| 201109_s_at | thrombospondin 1 | 3.6443605 | 0.0011221 | 0.0251089 | −1.076980645 |
| 212208_at | thyroid hormone receptor | 3.8776807 | 0.0006094 | 0.0167851 | −0.499403529 |
| | associated protein 2 | | | | |
| 203167_at | TIMP metallopeptidase | 3.3762907 | 0.0022375 | 0.0388607 | 1.678384544 |
| | inhibitor 2 | | | | |
| 204924_at | toll-like receptor 2 | 5.8914326 | 2.79E−006 | 0.0004151 | 4.67410031 |
| 206271_at | toll-like receptor 3 | −3.362162 | 0.0023195 | 0.0396727 | −1.788070455 |
| 214501_s_at | toll-like receptor 4 /// H2A | 4.635748 | 8.07E−005 | 0.0043967 | 1.43280053 |
| | histone family, member Y | | | | |
| 210166_at | toll-like receptor 5 | 4.104991 | 0.0003339 | 0.0115189 | 0.056663989 |
| 220832_at | toll-like receptor 8 | 3.7958707 | 0.0007555 | 0.0193307 | −0.692038473 |
| 219892_at | transmembrane 6 | 3.8952499 | 0.0005818 | 0.0164114 | −0.412464962 |
| | superfamily member 1 | | | | |
| 208184_s_at | transmembrane protein 1 | −3.529962 | 0.0015089 | 0.0302645 | −1.344894068 |
| 218930_s_at | transmembrane protein | −3.30508 | 0.0026811 | 0.0432556 | −1.927577145 |
| | 106B | | | | |
| 201361_at | transmembrane protein 109 | −4.012189 | 0.0004272 | 0.0134258 | −0.171802991 |
| 201934_at | Transmembrane protein 113 | −3.514453 | 0.0015705 | 0.0309805 | −1.398727123 |
| 218477_at | transmembrane protein 14A | −4.759601 | 5.78E−005 | 0.0035017 | 1.754649888 |
| 212989_at | transmembrane protein 23 | 5.2872176 | 1.40E−005 | 0.0013052 | 3.153543724 |
| 218615_s_at | transmembrane protein 39A | 6.0037925 | 2.08E−006 | 0.0003453 | 4.979344825 |
| 220990_s_at | transmembrane protein 49 | 6.0647884 | 1.77E−006 | 0.0003031 | 5.13407909 |
| | /// microRNA 21 | | | | |
| 219600_s_at | transmembrane protein 50B | −4.542525 | 0.0001037 | 0.0052977 | 1.220843469 |
| 212204_at | transmembrane protein 87A | −3.600801 | 0.0012564 | 0.0268042 | −1.197661812 |
| 212281_s_at | transmembrane protein 97 | −4.659473 | 7.57E−005 | 0.0042231 | 1.507037246 |
| 212279_at | transmembrane protein 97 | −4.015123 | 0.0004239 | 0.0133407 | −0.137448782 |
| 212282_at | transmembrane protein 97 | −3.549258 | 0.0014356 | 0.0292954 | −1.314433665 |
| 201645_at | tenascin C (hexabrachion) | 3.3249374 | 0.0025495 | 0.0420212 | −1.848438808 |
| 206025_s_at | tumor necrosis factor, alpha- | 6.8901834 | 2.08E−007 | 7.06E−005 | 7.090403074 |
| | induced protein 6 | | | | |
| 206026_s_at | tumor necrosis factor, alpha- | 4.8686295 | 4.31E−005 | 0.0028028 | 2.113663493 |
| | induced protein 6 | | | | |
| 211163_s_at | tumor necrosis factor | 3.2864659 | 0.0028104 | 0.0446357 | −1.890963078 |
| | receptor superfamily, | | | | |
| 209354_at | member 10c, decoy without | −3.483609 | 0.0017002 | 0.032801 | −1.480781959 |
| | an intracellular domain | | | | |
| | tumor necrosis factor | | | | |
| | receptor superfamily, | | | | |
| | member 14 (herpesvirus | | | | |
| | entry mediator) | | | | |
| 207643_s_at | tumor necrosis factor | 3.4506284 | 0.0018504 | 0.0343685 | −1.495664907 |
| | receptor superfamily, | | | | |
| | member 1A | | | | |
| 210314_x_at | tumor necrosis factor | 4.6582433 | 7.60E−005 | 0.0042231 | 1.50299243 |
| | (ligand) superfamily, | | | | |
| | member 13 /// tumor | | | | |
| | necrosis factor (ligand) | | | | |
| | superfamily, member 12- | | | | |
| | member 13 | | | | |
| 209500_x_at | tumor necrosis factor | 3.3587991 | 0.0023394 | 0.0398777 | −1.755497828 |
| | (ligand) superfamily, | | | | |
| | member 13 /// tumor | | | | |
| | necrosis factor (ligand) | | | | |
| | superfamily, member 12- | | | | |
| | member 13 | | | | |
| 218467_at | tumor necrosis factor | −3.779794 | 0.0007881 | 0.0199198 | −0.710816557 |
| | superfamily, member 5- | | | | |
| | induced protein 1 | | | | |
| 213107_at | TRAF2 and NCK | −4.289367 | 0.0002043 | 0.0083368 | 0.526899388 |
| | interacting kinase | | | | |
| 217853_at | transportin 3 | 3.6748085 | 0.0010367 | 0.0237807 | −0.976929327 |
| | tensin 3 | 4.9664964 | 3.31E−005 | 0.0023297 | 2.290198091 |
| 201812_s_at | translocase of outer | −3.513856 | 0.0015729 | 0.0309892 | −1.418772076 |
| | mitochondrial membrane 7 | | | | |
| | homolog (yeast) /// | | | | |
| | hypothetical protein | | | | |
| | LOC201725 | | | | |
| 201519_at | translocase of outer | −4.4208 | 0.0001437 | 0.0065345 | 0.893230892 |
| | mitochondrial membrane 70 | | | | |
| | homolog A (S. cerevisiae) | | | | |
| 204529_s_at | thymus high mobility group | −3.767864 | 0.0008131 | 0.0202657 | −0.762876334 |
| | box protein TOX | | | | |
| 201746_at | tumor protein p53 (Li- | −5.590339 | 6.22E−006 | 0.0007534 | 3.913976383 |
| | Fraumeni syndrome) | | | | |
| 211300_s_at | tumor protein p53 (Li- | −3.759786 | 0.0008304 | 0.0205152 | −0.801869935 |
| | Fraumeni syndrome) | | | | |
| 210886_x_at | TP53 activated protein 1 | −3.857224 | 0.0006431 | 0.0173489 | −0.55131381 |
| 210609_s_at | tumor protein p53 inducible | 4.5208633 | 0.0001099 | 0.0055225 | 1.163240291 |
| | protein 3 | | | | |
| 201688_s_at | tumor protein D52 | −3.206253 | 0.0034394 | 0.0506887 | −2.043214283 |
| 214195_at | tripeptidyl peptidase I | 4.3567851 | 0.0001706 | 0.0073659 | 0.759708629 |
| 200743_s_at | tripeptidyl peptidase I | 3.9060532 | 0.0005655 | 0.0161776 | −0.440759924 |
| | tyrosylprotein | | | | |
| 204140_at | sulfotransferase 1 | 9.5912477 | 3.38E−010 | 7.52E−007 | 12.97733866 |
| 211902_x_at | T cell receptor alpha locus | −5.936042 | 2.48E−006 | 0.0003843 | 4.793610525 |
| 215540_at | T cell receptor alpha locus | −5.423983 | 9.70E−006 | 0.0010296 | 3.470652626 |
| 216133_at | T cell receptor alpha locus | −5.368402 | 1.13E−005 | 0.0011458 | 3.320535206 |
| 217394_at | T cell receptor alpha locus | −3.979443 | 0.0004659 | 0.014279 | −0.265634691 |
| 209671_x_at | T cell receptor alpha locus | −6.33373 | 8.74E−007 | 0.0002028 | 5.800489831 |
| | /// T cell receptor alpha | | | | |
| | constant | | | | |
| 210972_x_at | T cell receptor alpha locus | −7.348774 | 6.54E−008 | 3.56E−005 | 8.283645607 |
| | /// T cell receptor delta | | | | |
| | variable 2 /// T cell receptor | | | | |
| | alpha variable 20 /// T cell | | | | |
| | receptor alpha constant | | | | |
| 215524_x_at | T cell receptor alpha locus | −5.771987 | 3.84E−006 | 0.0005261 | 4.361822067 |
| | /// YME1-like 1 (S. cerevisiae) | | | | |
| | /// T cell receptor | | | | |
| | delta variable 2 /// T cell | | | | |
| | receptor alpha variable 20 /// | | | | |
| | T cell receptor alpha | | | | |
| | constant | | | | |
| 209670_at | T cell receptor alpha | −5.614548 | 5.83E−006 | 0.000718 | 3.984879368 |
| | constant | | | | |
| 205641_s_at | TNFRSF1 A-associated via | −3.864291 | 0.0006313 | 0.0171291 | −0.533163843 |
| | death domain | | | | |
| 221571_at | TNF receptor-associated | −3.474931 | 0.0017385 | 0.0332359 | −1.490489904 |
| | factor 3 | | | | |
| 208315_x_at | TNF receptor-associated | −3.336893 | 0.0024733 | 0.0412217 | −1.83471622 |
| | factor 3 | | | | |
| 205558_at | TNF receptor-associated | −5.118072 | 2.20E−005 | 0.0017933 | 2.685989529 |
| | factor 6 | | | | |
| 202369_s_at | translocation associated | 3.399223 | 0.0021104 | 0.0374709 | −1.693080639 |
| | membrane protein 2 | | | | |
| 217958_at | trafficking protein particle | −5.294302 | 1.37E−005 | 0.0012969 | 3.149080792 |
| | complex 4 | | | | |
| 217959_s_at | trafficking protein particle | −3.618858 | 0.001199 | 0.02607 | −1.161971812 |
| | complex 4 | | | | |
| 204985_s_at | trafficking protein particle | −4.42802 | 0.0001409 | 0.0064487 | 0.913874605 |
| | complex 6A | | | | |
| 217147_s_at | T cell receptor associated | −5.1554 | 1.99E−005 | 0.0016706 | 2.803623231 |
| | transmembrane adaptor 1 | | | | |
| 213193_x_at | T cell receptor beta variable | −6.464127 | 6.22E−007 | 0.0001576 | 6.127595576 |
| | 19 /// T cell receptor beta | | | | |
| | constant 1 | | | | |
| 210915_x_at | T cell receptor beta variable | −5.995374 | 2.12E−006 | 0.0003463 | 4.958787768 |
| | 19 /// T cell receptor beta | | | | |
| | constant 1 | | | | |
| 211796_s_at | T cell receptor beta variable | −6.27007 | 1.03E−006 | 0.0002232 | 5.647887941 |
| | 21-1 /// T cell receptor beta | | | | |
| | variable 19 /// T cell | | | | |
| | receptor beta variable 5-4 /// | | | | |
| | T cell receptor beta variable | | | | |
| | 3-1 /// T cell receptor beta | | | | |
| | constant 1 | | | | |
| 217381_s_at | T cell receptor gamma | | | | |
| | variable 5 /// hypothetical | −3.28808 | 0.0027989 | 0.0445174 | −1.952613068 |
| | protein LOC648852 | | | | |
| 202241_at | tribbles homolog 1 | 7.5132583 | 4.35E−008 | 2.62E−005 | 8.679827422 |
| | (Drosophila) | | | | |
| 202479_s_at | tribbles homolog 2 | −4.330821 | 0.0001828 | 0.0077777 | 0.645636246 |
| | (Drosophila) | | | | |
| 203846_at | tripartite motif-containing | −4.901312 | 3.95E−005 | 0.0026038 | 2.113629506 |
| | 32 | | | | |
| 219405_at | tripartite motif-containing | −5.108003 | 2.27E−005 | 0.0018222 | 2.645489367 |
| | 68 | | | | |
| 212656_at | Ts translation elongation | −3.850017 | 0.0006554 | 0.0175534 | −0.57523954 |
| | factor, mitochondrial | | | | |
| 200973_s_at | tetraspanin 3 | −4.043354 | 0.0003933 | 0.0128508 | −0.044157262 |
| 209264_s_at | tetraspanin 4 | −3.264792 | 0.0029685 | 0.0462889 | −1.993384865 |
| 205652_s_at | tubulin tyrosine ligase-like | −4.18953 | 0.0002667 | 0.0099065 | 0.332588841 |
| | family, member 1 | | | | |
| 211714_x_at | tubulin, beta | −4.034927 | 0.0004022 | 0.0130266 | −0.104911491 |
| 209026_x_at | tubulin, beta | −3.291809 | 0.0027727 | 0.0441712 | −1.959242697 |
| 208864_s_at | thioredoxin | 3.3255919 | 0.0025453 | 0.0420125 | −1.862627759 |
| 208959_s_at | thioredoxin domain | 3.4060129 | 0.0020741 | 0.0370631 | −1.664100407 |
| | containing 4 (endoplasmic | | | | |
| | reticulum) | | | | |
| 209340_at | UDP-N-acteylglucosamine | −4.03047 | 0.000407 | 0.0131052 | −0.098477318 |
| | pyrophosphorylase 1 | | | | |
| 214755_at | UDP-N-acteylglucosamine | 3.8955918 | 0.0005813 | 0.0164114 | −0.440015337 |
| | pyrophosphorylase 1-like 1 | | | | |
| 221700_s_at | ubiquitin A-52 residue | −5.019221 | 2.88E−005 | 0.0021433 | 2.447535269 |
| | ribosomal protein fusion | | | | |
| | product 1 | | | | |
| 217823_s_at | ubiquitin-conjugating | 7.4560627 | 5.01E−008 | 2.94E−005 | 8.546204618 |
| | enzyme E2, J1 (UBC6 | | | | |
| | homolog, yeast) | | | | |
| 217826_s_at | ubiquitin-conjugating | 5.2557813 | 1.52E−005 | 0.0013909 | 3.041318423 |
| | enzyme E2, J1 (UBC6 | | | | |
| | homolog, yeast) | | | | |
| 217825_s_at | ubiquitin-conjugating | 4.7504707 | 5.93E−005 | 0.0035791 | 1.7317692 |
| | enzyme E2, J1 (UBC6 | | | | |
| | homolog, yeast) | | | | |
| 217824_at | ubiquitin-conjugating | 4.4448746 | 0.0001347 | 0.0062793 | 0.949423013 |
| | enzyme E2, J1 (UBC6 | | | | |
| | homolog, yeast) | | | | |
| 217978_s_at | ubiquitin-conjugating | −4.228821 | 0.0002401 | 0.0093056 | 0.412979469 |
| | enzyme E2Q (putative) 1 | | | | |
| 219172_at | ubiquitin domain containing 1 | 3.4313618 | 0.001944 | 0.0356815 | −1.582019284 |
| 202330_s_at | uracil-DNA glycosylase | −3.214258 | 0.003371 | 0.050078 | −2.070496452 |
| 203234_at | uridine phosphorylase 1 | 4.4662231 | 0.0001272 | 0.0060731 | 1.057050974 |
| 210681_s_at | ubiquitin specific peptidase | 3.2950986 | 0.0027497 | 0.0439378 | −1.948321615 |
| | 15 | | | | |
| 207211_at | ubiquitin specific peptidase 2 | 3.2165756 | 0.0033515 | 0.0499874 | −2.093423662 |
| 203965_at | ubiquitin specific peptidase | −3.683953 | 0.0010123 | 0.023375 | −0.970196762 |
| | 20 | | | | |
| 206405_x_at | ubiquitin specific peptidase | 3.4566809 | 0.0018219 | 0.0340576 | −1.555816652 |
| | 6 (Tre-2 oncogene) | | | | |
| 204255_s_at | vitamin D (1,25- | 3.3295297 | 0.00252 | 0.0417806 | −1.865806845 |
| | dihydroxyvitamin D3) | | | | |
| | receptor | | | | |
| 211527_x_at | vascular endothelial growth | 4.3473077 | 0.000175 | 0.0075115 | 0.710749277 |
| | factor | | | | |
| 210512_s_at | vascular endothelial growth | 4.3250235 | 0.0001857 | 0.0078519 | 0.634031227 |
| | factor | | | | |
| 212171_x_at | vascular endothelial growth | 3.3953161 | 0.0021315 | 0.0376896 | −1.674466924 |
| | factor | | | | |
| 208622_s_at | villin 2 (ezrin) | −3.587221 | 0.0013014 | 0.0274098 | −1.204190074 |
| 203459_s_at | vacuolar protein sorting 16 | 3.9525631 | 0.0005002 | 0.0149201 | −0.323834974 |
| | (yeast) | | | | |
| 217837_s_at | vacuolar protein sorting 24 | 3.6530442 | 0.0010971 | 0.0247936 | −1.051987141 |
| | homolog (S. cerevisiae) | | | | |
| 204590_x_at | vacuolar protein sorting 33 | −3.601451 | 0.0012543 | 0.0267978 | −1.207101626 |
| | homolog A (S. cerevisiae) | | | | |
| 204787_at | V-set and immunoglobulin | 9.4475204 | 4.64E−010 | 8.61E−007 | 12.85523733 |
| | domain containing 4 | | | | |
| 200629_at | tryptophanyl-tRNA | −5.272927 | 1.45E−005 | 0.0013482 | 3.116657464 |
| | synthetase | | | | |
| 200628_s_at | tryptophanyl-tRNA | −4.221181 | 0.0002451 | 0.009383 | 0.353722972 |
| | synthetase | | | | |
| 212606_at | WD repeat and FYVE | 6.1896123 | 1.27E−006 | 0.000249 | 5.439218127 |
| | domain containing 3 | | | | |
| 212602_at | WD repeat and FYVE | 4.1210426 | 0.00032 | 0.0111761 | 0.176181465 |
| | domain containing 3 | | | | |
| 212598_at | WD repeat and FYVE | 3.8915864 | 0.0005875 | 0.0164871 | −0.435503889 |
| | domain containing 3 | | | | |
| 209461_x_at | WD repeat domain 18 | −3.514309 | 0.0015711 | 0.0309805 | −1.391071876 |
| 218851_s_at | WD repeat domain 33 | −3.792828 | 0.0007616 | 0.0194616 | −0.726323818 |
| 215905_s_at | WD repeat domain 57 (U5 | −4.514933 | 0.0001116 | 0.0055344 | 1.150267568 |
| | snRNP specific) | | | | |
| 221532_s_at | WD repeat domain 61 | −3.407047 | 0.0020687 | 0.0369951 | −1.593954553 |
| 215156_at | WD repeat domain 61 | 3.3016317 | 0.0027046 | 0.0434514 | −1.893174705 |
| 214061_at | WD repeat domain 67 | −3.575841 | 0.0013403 | 0.0279853 | −1.15398179 |
| 219193_at | WD repeat domain 70 | −3.363782 | 0.0023099 | 0.0395937 | −1.744365645 |
| 219478_at | WAP four-disulfide core | 3.4387424 | 0.0019076 | 0.03521 | −1.536993233 |
| | domain 1 | | | | |
| 210561_s_at | WD repeat and SOCS box- | 4.0085842 | 0.0004313 | 0.0135115 | −0.178043218 |
| | containing 1 | | | | |
| 201296_s_at | WD repeat and SOCS box- | 3.7312421 | 0.0008948 | 0.0215787 | −0.883481455 |
| | containing 1 | | | | |
| 206366_x_at | chemokine (C motif) ligand 2 | −3.267934 | 0.0029451 | 0.0460201 | −1.994411017 |
| 202932_at | v-yes-1 Yamaguchi sarcoma | −6.020191 | 1.99E−006 | 0.0003357 | 5.003651416 |
| | viral oncogene homolog 1 | | | | |
| 213996_at | yippee-like 1 (Drosophila) | −3.882592 | 0.0006016 | 0.0166932 | −0.407780108 |
| 214631_at | zinc finger and BTB domain | −3.281852 | 0.0028434 | 0.044935 | −1.953826399 |
| | containing 33 | | | | |
| 204181_s_at | zinc finger and BTB domain | 4.4329552 | 0.0001391 | 0.0064034 | 0.942572026 |
| | containing 43 | | | | |
| 220104_at | zinc finger CCCH-type, | −5.025738 | 2.83E−005 | 0.0021203 | 2.463004001 |
| | antiviral 1 | | | | |
| 213853_at | zinc finger, CSL-type | −4.051569 | 0.0003848 | 0.0126796 | −0.053886714 |
| | containing 3 | | | | |
| 212982_at | zinc finger, DHHC-type | 3.6939685 | 0.0009862 | 0.0229583 | −0.937959088 |
| | containing 17 | | | | |
| 218077_s_at | zinc finger, DHHC-type | 3.3148115 | 0.0026158 | 0.0426086 | −1.89439339 |
| | containing 3 | | | | |
| 203603_s_at | zinc finger homeobox 1b | 3.4667365 | 0.0017755 | 0.033635 | −1.52157029 |
| 217781_s_at | zinc finger protein 106 | 4.5749938 | 9.50E−005 | 0.0050044 | 1.262299233 |
| | homolog (mouse) | | | | |
| 209724_s_at | zinc finger protein 161 | −4.093231 | 0.0003445 | 0.0117213 | 0.06143106 |
| | homolog (mouse) | | | | |
| 207090_x_at | zinc finger protein 30 | −3.718246 | 0.0009257 | 0.0219904 | −0.912694737 |
| | homolog (mouse) | | | | |
| 201531_at | zinc finger protein 36, C3H | 5.3015247 | 1.35E−005 | 0.0012774 | 3.160231961 |
| | type, homolog (mouse) | | | | |
| 210282_at | zinc finger, MYM-type 2 | 3.3388242 | 0.0024612 | 0.0411468 | −1.792009743 |
| 213698_at | zinc finger, MYM-type 6 | −3.426359 | 0.001969 | 0.0358275 | −1.61184175 |
| 216350_s_at | zinc finger protein 10 | −4.247211 | 0.0002286 | 0.0089711 | 0.431689143 |
| 207605_x_at | zinc finger protein 117 | −3.369845 | 0.0022745 | 0.0393506 | −1.749220533 |
| 216960_s_at | zinc finger protein 133 | −3.714698 | 0.0009343 | 0.022054 | −0.887168408 |
| 219854_at | zinc finger protein 14 | −3.764607 | 0.00082 | 0.0203709 | −0.718788206 |
| 204523_at | zinc finger protein 140 | −3.24699 | 0.0031047 | 0.0474828 | −2.029248631 |
| 213452_at | zinc finger protein 184 | −4.035509 | 0.0004016 | 0.0130255 | −0.110930248 |
| 204327_s_at | zinc finger protein 202 | −3.527591 | 0.0015182 | 0.0303953 | −1.368262557 |
| 203985_at | zinc finger protein 212 | −3.928858 | 0.0005325 | 0.0155102 | −0.383188113 |
| 218005_at | zinc finger protein 22 (KOX | −4.62558 | 8.29E−005 | 0.0044741 | 1.421087611 |
| | 15) | | | | |
| 217403_s_at | zinc finger protein 227 | −3.412141 | 0.0020419 | 0.0367257 | −1.645642234 |
| 206900_x_at | zinc finger protein 253 | −5.475987 | 8.44E−006 | 0.0009268 | 3.601028141 |
| 213778_x_at | zinc finger protein 276 | −5.659234 | 5.18E−006 | 0.0006707 | 4.084696095 |
| 220055_at | zinc finger protein 287 | −4.102977 | 0.0003357 | 0.0115515 | 0.047648379 |
| 215429_s_at | zinc finger protein 428 | −3.238631 | 0.0031707 | 0.0479008 | −2.041757374 |
| 205928_at | zinc finger protein 443 | −4.409745 | 0.000148 | 0.00669 | 0.856775472 |
| 206053_at | zinc finger protein 510 | −3.289375 | 0.0027898 | 0.0444036 | −1.960045895 |
| 206648_at | zinc finger protein 571 | −3.557317 | 0.0014061 | 0.0287974 | −1.316878093 |
| 217547_x_at | zinc finger protein 675 | −4.178928 | 0.0002743 | 0.0101026 | 0.270088801 |
| 213658_at | Zinc finger protein 710 | −4.51761 | 0.0001108 | 0.0055225 | 1.151761708 |
| 206180_x_at | zinc finger protein 747 | −4.822356 | 4.88E−005 | 0.0030921 | 1.948607569 |
| 222120_at | zinc finger protein 764 | −3.382796 | 0.0022007 | 0.0384616 | −1.731593303 |
| 212544_at | zinc finger, HIT type 3 | −3.774469 | 0.0007991 | 0.0199959 | −0.767304552 |
| 211950_at | zinc finger, UBR1 type 1 | 4.9957989 | 3.06E−005 | 0.0022088 | 2.360604795 |
| 218639_s_at | ZXD family zinc finger C | 3.7250128 | 0.0009095 | 0.0217998 | −0.907201906 |

\hat{ }Probe set ID number is the Affymetrix ID number on the HU133A array.
*Moderated t-statistic. This has the same interpretation as an ordinary t-statistic except that the standard errors have been moderated across genes, i.e., shrunk towards a common value, using a simple Bayesian model. Positive t-statistic indicates that the gene is upregulated following hemorrhagic stroke. Negative t-statistic indicates that the gene is downregulated following hemorrhagic stroke.
^{$}P-value uncorrected p value
^{#}Adjusted p-value is the corrected value after correction for multitple comparisons using the FDR method.
^{@}The B-statistic (lods or B) is the log-odds that the gene is differentially expressed.

TABLE 4
stroke related-genes using Holm correction and comparison
to non-stroke subjects.

| Probe Set | | t- | | Adjusted | |
|---|---|---|---|---|---|
| ID\hat{ } | Gene Name | statistic* | P Value^{$} | P Value^{#} | B^{@} |
| 211372_s_at | interleukin 1 receptor, type II | 26.109122 | 1.01E−020 | 2.24E−016 | 30.71443211 |
| 205257_s_at | amphiphysin (Stiff-Man | 24.606614 | 4.70E−020 | 1.05E−015 | 30.88004619 |
| | syndrome with breast cancer | | | | |
| | 128 kDa autoantigen) | | | | |
| 205403_at | interleukin 1 receptor, type II | 19.405844 | 2.07E−017 | 4.62E−013 | 26.80773514 |
| 216233_at | CD163 molecule | 18.497494 | 6.95E−017 | 1.55E−012 | 25.43080936 |
| 200919_at | polyhomeotic-like 2 | 13.491127 | 1.57E−013 | 3.50E−009 | 19.73316172 |
| | (Drosophila) | | | | |
| 214535_s_at | ADAM metallopeptidase with | 13.353671 | 2.00E−013 | 4.46E−009 | 19.55417663 |
| | thrombospondin type 1 motif, 2 | | | | |
| 219157_at | kelch-like 2, Mayven | 10.76177 | 2.80E−011 | 6.24E−007 | 15.40078527 |
| | (Drosophila) | | | | |
| 206028_s_at | c-mer proto-oncogene tyrosine | 10.469242 | 5.13E−011 | 1.14E−006 | 14.90589976 |
| | kinase | | | | |
| 215049_x_at | CD163 molecule | 9.7258472 | 2.51E−010 | 5.60E−006 | 13.52665406 |
| 204140_at | tyrosylprotein sulfotransferase 1 | 9.5912477 | 3.38E−010 | 7.52E−006 | 12.97733866 |
| 203645_s_at | CD163 molecule | 9.529413 | 3.87E−010 | 8.62E−006 | 13.11769962 |
| 204787_at | V-set and immunoglobulin | 9.4475204 | 4.64E−010 | 1.03E−005 | 12.85523733 |
| | domain containing 4 | | | | |
| 38487_at | stabilin 1 | 9.0955932 | 1.02E−009 | 2.28E−005 | 12.20225685 |
| 202499_s_at | solute carrier family 2 | 8.9748804 | 1.34E−009 | 2.99E−005 | 11.92184489 |
| | (facilitated glucose transporter), | | | | |
| | member 3 | | | | |
| 206522_at | maltase-glucoamylase (alpha- | 8.9425055 | 1.45E−009 | 3.22E−005 | 11.67816349 |
| | glucosidase) /// similar to | | | | |
| | Maltase-glucoamylase, intestinal | | | | |
| 206674_at | fms-related tyrosine kinase 3 | 8.9158813 | 1.54E−009 | 3.43E−005 | 11.78456326 |
| 46665_at | sema domain, immunoglobulin | −8.88408 | 1.65E−009 | 3.68E−005 | 11.76724808 |
| | domain (Ig), transmembrane | | | | |
| | domain (TM) and short | | | | |
| | cytoplasmic domain, | | | | |
| | (semaphorin) 4C | | | | |
| 219358_s_at | centaurin, alpha 2 | 8.8817081 | 1.66E−009 | 3.70E−005 | 11.79433186 |
| 209286_at | CDC42 effector protein (Rho | 8.7039359 | 2.51E−009 | 5.58E−005 | 11.36556799 |
| | GTPase binding) 3 | | | | |
| 217502_at | interferon-induced protein with | −8.647679 | 2.86E−009 | 6.36E−005 | 11.27869101 |
| | tetratricopeptide repeats 2 | | | | |
| 201601_x_at | interferon induced | −8.572445 | 3.40E−009 | 7.58E−005 | 11.07737096 |
| | transmembrane protein 1 (9-27) | | | | |
| 204043_at | transcobalamin II; macrocytic | 8.4547789 | 4.49E−009 | 9.99E−005 | 10.77422771 |
| | anemia | | | | |
| 220001_at | peptidyl arginine deiminase, | 8.3835748 | 5.30E−009 | 0.0001181 | 10.63185834 |
| | type IV | | | | |
| 204769_s_at | transporter 2, ATP-binding | −8.325113 | 6.09E−009 | 0.0001356 | 10.55890931 |
| | cassette, sub-family B | | | | |
| | (MDR/TAP) | | | | |
| 216667_at | ribonuclease, RNase A family, 2 | 7.9404174 | 1.53E−008 | 0.0003405 | 9.485566694 |
| | (liver, eosinophil-derived | | | | |
| | neurotoxin) | | | | |
| 204713_s_at | coagulation factor V | 7.8705559 | 1.81E−008 | 0.0004033 | 9.39995824 |
| | (proaccelerin, labile factor) | | | | |
| 213006_at | CCAAT/enhancer binding | 7.8208216 | 2.04E−008 | 0.0004551 | 9.380113378 |
| | protein (C/EBP), delta | | | | |
| 207460_at | granzyme M (lymphocyte met- | −7.750349 | 2.43E−008 | 0.0005404 | 9.235332652 |
| | ase 1) | | | | |
| 207275_s_at | acyl-CoA synthetase long-chain | 7.7487799 | 2.44E−008 | 0.0005424 | 9.200907246 |
| | family member 1 | | | | |
| 214696_at | hypothetical protein MGC14376 | 7.7417859 | 2.48E−008 | 0.0005518 | 9.178335493 |
| 203428_s_at | ASF1 anti-silencing function 1 | −7.698443 | 2.76E−008 | 0.0006135 | 9.105468688 |
| | homolog A (S. cerevisiae) | | | | |
| 208826_x_at | histidine triad nucleotide binding | −7.684964 | 2.85E−008 | 0.0006341 | 9.064674732 |
| | protein 1 | | | | |
| 211936_at | heat shock 70 kDa protein 5 | 7.6812023 | 2.88E−008 | 0.0006399 | 9.061677362 |
| | (glucose-regulated protein, | | | | |
| | 78 kDa) | | | | |
| 202531_at | interferon regulatory factor 1 | −7.657986 | 3.04E−008 | 0.0006774 | 9.022177601 |
| 212658_at | lipoma HMGIC fusion partner- | 7.5928187 | 3.57E−008 | 0.0007951 | 8.837614782 |
| | like 2 | | | | |
| 201785_at | ribonuclease, RNase A family, 1 | 7.5450772 | 4.02E−008 | 0.0008944 | 8.69840649 |
| | (pancreatic) | | | | |
| 202241_at | tribbles homolog 1 (Drosophila) | 7.5132583 | 4.35E−008 | 0.0009675 | 8.679827422 |
| 217823_s_at | ubiquitin-conjugating enzyme | 7.4560627 | 5.01E−008 | 0.0011148 | 8.546204618 |
| | E2, J1 (UBC6 homolog, yeast) | | | | |
| 214877_at | CDK5 regulatory subunit | −7.380157 | 6.05E−008 | 0.0013462 | 8.358822206 |
| | associated protein 1-like 1 | | | | |
| 202145_at | lymphocyte antigen 6 complex, | −7.366572 | 6.26E−008 | 0.0013924 | 8.33334838 |
| | locus E | | | | |
| 210972_x_at | T cell receptor alpha locus /// T | −7.348774 | 6.54E−008 | 0.0014555 | 8.283645607 |
| | cell receptor delta variable 2 /// | | | | |
| | T cell receptor alpha variable 20 | | | | |
| | /// T cell receptor alpha constant | | | | |
| 221688_s_at | IMP3, U3 small nucleolar | −7.319613 | 7.04E−008 | 0.0015654 | 8.216503503 |
| | ribonucleoprotein, homolog | | | | |
| | (yeast) | | | | |
| 204070_at | retinoic acid receptor responder | −7.315638 | 7.11E−008 | 0.0015809 | 8.198820117 |
| | (tazarotene induced) 3 | | | | |
| 201963_at | acyl-CoA synthetase long-chain | 7.2748901 | 7.87E−008 | 0.0017504 | 8.113119287 |
| | family member 1 | | | | |
| 202730_s_at | programmed cell death 4 | −7.258288 | 8.20E−008 | 0.0018246 | 8.071670715 |
| | (neoplastic transformation | | | | |
| | inhibitor) | | | | |
| AFFX- | signal transducer and activator | −7.243677 | 8.51E−008 | 0.0018926 | 8.040419882 |
| HUMISGF3A/ | of transcription 1, 91 kDa | | | | |
| M97935_MA_at | | | | | |
| 203887_s_at | thrombomodulin | 7.2217472 | 8.99E−008 | 0.0019995 | 7.98099287 |
| 203674_at | helicase with zinc finger | 7.2191971 | 9.05E−008 | 0.0020123 | 7.981565104 |
| 202861_at | period homolog 1 (Drosophila) | 7.2015321 | 9.46E−008 | 0.0021035 | 7.937670727 |
| 202436_s_at | cytochrome P450, family 1, | 7.1543503 | 1.07E−007 | 0.0023683 | 7.822723722 |
| | subfamily B, polypeptide 1 | | | | |
| 211571_s_at | chondroitin sulfate proteoglycan | 7.0973548 | 1.23E−007 | 0.0027342 | 7.681872527 |
| | 2 (versican) | | | | |
| 221011_s_at | hypothetical protein | −7.094931 | 1.24E−007 | 0.0027509 | 7.670411186 |
| | DKFZp566J091 /// hypothetical | | | | |
| | protein DKFZp566J091 /// | | | | |
| | similar to hypothetical protein | | | | |
| | DKFZp566J091 | | | | |
| 200644_at | MARCKS-like 1 | −7.088418 | 1.26E−007 | 0.0027964 | 7.662378032 |
| 209163_at | cytochrome b-561 | −7.083343 | 1.27E−007 | 0.0028323 | 7.652027367 |
| 221081_s_at | DENN/MADD domain | −7.06017 | 1.35E−007 | 0.003003 | 7.573105401 |
| | containing 2D | | | | |
| AFFX- | signal transducer and activator | −7.033501 | 1.45E−007 | 0.0032126 | 7.530750606 |
| HUMISGF3A/ | of transcription 1, 91 kDa | | | | |
| M97935_5_at | | | | | |
| 221039_s_at | development and differentiation | 7.0328086 | 1.45E−007 | 0.0032181 | 7.52906031 |
| | enhancing factor 1 | | | | |
| 208189_s_at | myosin VIIA | 7.0276054 | 1.47E−007 | 0.0032606 | 7.51220835 |
| 217722_s_at | neugrin, neurite outgrowth | −7.00942 | 1.54E−007 | 0.0034142 | 7.472039365 |
| | associated | | | | |
| 64064_at | GTPase, IMAP family member 5 | −6.980471 | 1.65E−007 | 0.0036742 | 7.401416652 |
| 218660_at | dysferlin, limb girdle muscular | 6.9457601 | 1.81E−007 | 0.0040127 | 7.308255352 |
| | dystrophy 2B (autosomal | | | | |
| | recessive) | | | | |
| 204714_s_at | coagulation factor V | 6.9047733 | 2.00E−007 | 0.0044537 | 7.202864473 |
| | (proaccelerin, labile factor) | | | | |
| 215001_s_at | glutamate-ammonia ligase | 6.9032827 | 2.01E−007 | 0.0044704 | 7.211931747 |
| | (glutamine synthetase) | | | | |
| 206025_s_at | tumor necrosis factor, alpha- | 6.8901834 | 2.08E−007 | 0.0046219 | 7.090403074 |
| | induced protein 6 | | | | |
| 209508_x_at | CASP8 and FADD-like | 6.8867892 | 2.10E−007 | 0.0046619 | 7.171950728 |
| | apoptosis regulator | | | | |
| 205931_s_at | cAMP responsive element | 6.8831696 | 2.12E−007 | 0.0047049 | 7.144372934 |
| | binding protein 5 | | | | |
| 204446_s_at | arachidonate 5-lipoxygenase | 6.8751354 | 2.16E−007 | 0.0048021 | 7.143490943 |
| 207540_s_at | spleen tyrosine kinase | 6.8718487 | 2.18E−007 | 0.0048423 | 7.135740333 |
| 207485_x_at | butyrophilin, subfamily 3, | −6.870456 | 2.19E−007 | 0.0048593 | 7.132260801 |
| | member A1 | | | | |
| 202435_s_at | cytochrome P450, family 1, | 6.8642415 | 2.22E−007 | 0.0049367 | 7.116902873 |
| | subfamily B, polypeptide 1 | | | | |
| 210039_s_at | protein kinase C, theta | −6.853394 | 2.28E−007 | 0.0050751 | 7.089585199 |
| 219209_at | interferon induced with helicase | −6.843417 | 2.34E−007 | 0.0052058 | 7.064971115 |
| | C domain 1 | | | | |
| 218689_at | Fanconi anemia, | −6.820166 | 2.49E−007 | 0.0055243 | 7.008995717 |
| | complementation group F | | | | |
| 37577_at | Rho GTPase activating protein | 6.7634786 | 2.88E−007 | 0.0063868 | 6.869000176 |
| | 19 | | | | |
| 214643_x_at | bridging integrator 1 | −6.752528 | 2.96E−007 | 0.0065683 | 6.842410212 |
| 218805_at | GTPase, IMAP family member 5 | −6.740512 | 3.05E−007 | 0.0067736 | 6.81182532 |
| 218025_s_at | peroxisomal D3, D2-enoyl-CoA | −6.668995 | 3.67E−007 | 0.0081398 | 6.635968187 |
| | isomerase | | | | |
| 218092_s_at | HIV-1 Rev binding protein /// | 6.6361304 | 3.99E−007 | 0.0088584 | 6.555967735 |
| | region containing hypothetical | | | | |
| | protein LOC285086; HIV-1 Rev | | | | |
| | binding protein | | | | |
| 203535_at | S100 calcium binding protein | 6.627653 | 4.08E−007 | 0.0090537 | 6.534375705 |
| | A9 (calgranulin B) | | | | |
| 213537_at | major histocompatibility | −6.587999 | 4.52E−007 | 0.0100286 | 6.436948993 |
| | complex, class II, DP alpha 1 | | | | |
| 205020_s_at | ADP-ribosylation factor-like 4A | 6.5781587 | 4.63E−007 | 0.0102863 | 6.412365317 |
| 217552_x_at | complement component (3b/4b) | 6.5779604 | 4.64E−007 | 0.0102911 | 6.412072722 |
| | receptor 1 (Knops blood group) | | | | |
| 202964_s_at | regulatory factor X, 5 | −6.568302 | 4.75E−007 | 0.0105506 | 6.387230752 |
| | (influences HLA class II | | | | |
| | expression) | | | | |
| 215754_at | scavenger receptor class B, | 6.5663251 | 4.78E−007 | 0.0106042 | 6.383543044 |
| | member 2 | | | | |
| 213688_at | calmodulin 1 (phosphorylase | −6.510788 | 5.51E−007 | 0.0122425 | 6.240465245 |
| | kinase, delta) | | | | |
| 210640_s_at | G protein-coupled receptor 30 | 6.4845034 | 5.90E−007 | 0.0131049 | 6.173410568 |
| 205786_s_at | integrin, alpha M (complement | 6.4795743 | 5.98E−007 | 0.0132729 | 6.164969498 |
| | component 3 receptor 3 subunit) | | | | |
| 213193_x_at | T cell receptor beta variable 19 | −6.464127 | 6.22E−007 | 0.013815 | 6.127595576 |
| | /// T cell receptor beta constant 1 | | | | |
| 206118_at | signal transducer and activator | −6.450878 | 6.44E−007 | 0.0142978 | 6.093085989 |
| | of transcription 4 | | | | |
| 203140_at | B-cell CLL/lymphoma 6 (zinc | 6.4484079 | 6.48E−007 | 0.0143891 | 6.082317265 |
| | finger protein 51) | | | | |
| 211829_s_at | G protein-coupled receptor 30 | 6.4243249 | 6.90E−007 | 0.0153175 | 5.992630343 |
| 209471_s_at | farnesyltransferase, CAAX box, | −6.417811 | 7.02E−007 | 0.0155784 | 6.013878869 |
| | alpha | | | | |
| 201280_s_at | disabled homolog 2, mitogen- | 6.3730121 | 7.89E−007 | 0.0175037 | 5.892530411 |
| | responsive phosphoprotein | | | | |
| | (Drosophila) | | | | |
| 218561_s_at | chromosome 6 open reading | −6.367632 | 8.00E−007 | 0.0177499 | 5.890852776 |
| | frame 149 | | | | |
| 215646_s_at | chondroitin sulfate proteoglycan | 6.3339611 | 8.73E−007 | 0.0193773 | 5.797392458 |
| | 2 (versican) | | | | |
| 209671_x_at | T cell receptor alpha locus /// T | −6.33373 | 8.74E−007 | 0.0193881 | 5.800489831 |
| | cell receptor alpha constant | | | | |
| 206335_at | galactosamine (N-acetyl)-6- | 6.3239096 | 8.96E−007 | 0.0198903 | 5.779394683 |
| | sulfate sulfatase (Morquio | | | | |
| | syndrome, | | | | |
| | mucopolysaccharidosis type | | | | |
| | IVA) | | | | |
| 209154_at | Tax1 (human T-cell leukemia | 6.3171051 | 9.13E−007 | 0.0202457 | 5.761271761 |
| | virus type I) binding protein 3 | | | | |
| 213236_at | SAM and SH3 domain | 6.3133264 | 9.22E−007 | 0.0204455 | 5.755701955 |
| | containing 1 | | | | |
| 211991_s_at | major histocompatibility | −6.311143 | 9.27E−007 | 0.0205614 | 5.749261512 |
| | complex, class II, DP alpha 1 | | | | |
| 212888_at | Dicer1, Dcr-1 homolog | 6.2936145 | 9.70E−007 | 0.0215234 | 5.693791253 |
| | (Drosophila) | | | | |
| 202917_s_at | S100 calcium binding protein | 6.291558 | 9.75E−007 | 0.0216384 | 5.69316671 |
| | A8 (calgranulin A) | | | | |
| 211796_s_at | T cell receptor beta variable 21-1 | −6.27007 | 1.03E−006 | 0.0228873 | 5.647887941 |
| | /// T cell receptor beta variable | | | | |
| | 19 /// T cell receptor beta | | | | |
| | variable 5-4 /// T cell receptor | | | | |
| | beta variable 3-1 /// T cell | | | | |
| | receptor beta constant 1 | | | | |
| 219889_at | frequently rearranged in | 6.25963 | 1.06E−006 | 0.0235196 | 5.619733039 |
| | advanced T-cell lymphomas | | | | |
| 201185_at | HtrA serine peptidase 1 | 6.2530319 | 1.08E−006 | 0.0239279 | 5.605563874 |
| 200953_s_at | cyclin D2 | −6.234765 | 1.13E−006 | 0.0250983 | 5.558570668 |
| 204150_at | stabilin 1 | 6.2310727 | 1.14E−006 | 0.025341 | 5.543221025 |
| 205425_at | huntingtin interacting protein 1 | 6.2228987 | 1.17E−006 | 0.025888 | 5.53011363 |
| 203298_s_at | Jumonji, AT rich interactive | 6.2222872 | 1.17E−006 | 0.0259284 | 5.518414859 |
| | domain 2 | | | | |
| 219574_at | membrane-associated ring finger | 6.2140115 | 1.19E−006 | 0.0264953 | 5.496195004 |
| | (C3HC4) 1 | | | | |
| 217119_s_at | chemokine (C—X—C motif) | −6.212669 | 1.20E−006 | 0.0265874 | 5.495676727 |
| | receptor 3 | | | | |
| 203936_s_at | matrix metallopeptidase 9 | 6.2084895 | 1.21E−006 | 0.026879 | 5.494417575 |
| | (gelatinase B, 92 kDa gelatinase, | | | | |
| | 92 kDa type IV collagenase) | | | | |
| 206111_at | ribonuclease, RNase A family, 2 | 6.1911281 | 1.27E−006 | 0.028129 | 5.45081304 |
| | (liver, eosinophil-derived | | | | |
| | neurotoxin) | | | | |
| 212606_at | WD repeat and FYVE domain | 6.1896123 | 1.27E−006 | 0.0282398 | 5.439218127 |
| | containing 3 | | | | |
| 209906_at | complement component 3a | 6.1809183 | 1.30E−006 | 0.0288897 | 5.413796702 |
| | receptor 1 | | | | |
| 218091_at | HIV-1 Rev binding protein /// | 6.1478546 | 1.42E−006 | 0.0315068 | 5.340512512 |
| | region containing hypothetical | | | | |
| | protein LOC285086; HIV-1 Rev | | | | |
| | binding protein | | | | |
| 202208_s_at | ADP-ribosylation factor-like 4C | −6.147552 | 1.42E−006 | 0.0315304 | 5.341709786 |
| 209135_at | aspartate beta-hydroxylase | 6.1302992 | 1.49E−006 | 0.0329905 | 5.298766404 |
| 214438_at | H2.0-like homeobox 1 | 6.1182254 | 1.54E−006 | 0.0340524 | 5.26739525 |
| | (Drosophila) | | | | |
| 216969_s_at | kinesin family member 22 | −6.117807 | 1.54E−006 | 0.0340884 | 5.245713904 |
| 200952_s_at | cyclin D2 | −6.106075 | 1.59E−006 | 0.0351544 | 5.218632002 |
| 204619_s_at | chondroitin sulfate proteoglycan | 6.0890716 | 1.66E−006 | 0.0367606 | 5.177048193 |
| | 2 (versican) | | | | |
| 211997_x_at | H3 histone, family 3B (H3.3B) | 6.0804473 | 1.70E−006 | 0.0376026 | 5.168767149 |
| 218559_s_at | v-maf musculoaponeurotic | 6.0797981 | 1.70E−006 | 0.0376652 | 5.148454877 |
| | fibrosarcoma oncogene homolog | | | | |
| | B (avian) | | | | |
| 207721_x_at | histidine triad nucleotide binding | −6.078022 | 1.71E−006 | 0.03784 | 5.159287966 |
| | protein 1 | | | | |
| 202820_at | aryl hydrocarbon receptor | 6.0756321 | 1.72E−006 | 0.038077 | 5.158254442 |
| 203828_s_at | interleukin 32 | −6.070581 | 1.74E−006 | 0.0385849 | 5.138343653 |
| 200814_at | proteasome (prosome, | −6.067133 | 1.76E−006 | 0.038935 | 5.126788139 |
| | macropain) activator subunit 1 | | | | |
| | (PA28 alpha) | | | | |
| 204620_s_at | chondroitin sulfate proteoglycan | 6.0654142 | 1.77E−006 | 0.0391098 | 5.120383055 |
| | 2 (versican) | | | | |
| 220990_s_at | transmembrane protein 49 /// | 6.0647884 | 1.77E−006 | 0.0391726 | 5.13407909 |
| | microRNA 21 | | | | |
| 214022_s_at | interferon induced | −6.053528 | 1.82E−006 | 0.0403497 | 5.105034551 |
| | transmembrane protein 1 (9-27) | | | | |
| 202932_at | v-yes-1 Yamaguchi sarcoma | −6.020191 | 1.99E−006 | 0.0440529 | 5.003651416 |
| | viral oncogene homolog 1 | | | | |
| 31826_at | KIAA0674 | 6.0122705 | 2.03E−006 | 0.0449807 | 4.995084148 |
| 218615_s_at | transmembrane protein 39A | 6.0037925 | 2.08E−006 | 0.0459958 | 4.979344825 |
| 202192_s_at | growth arrest-specific 7 | 5.9982522 | 2.11E−006 | 0.046671 | 4.960463643 |
| 210915_x_at | T cell receptor beta variable 19 | −5.995374 | 2.12E−006 | 0.0470247 | 4.958787768 |
| | /// T cell receptor beta constant 1 | | | | |
| 204116_at | interleukin 2 receptor, gamma | −5.991858 | 2.14E−006 | 0.0474609 | 4.934872705 |
| | (severe combined | | | | |
| | immunodeficiency) | | | | |
| 213275_x_at | cathepsin B | 5.9916022 | 2.14E−006 | 0.0474908 | 4.931225528 |
| 205590_at | RAS guanyl releasing protein 1 | −5.976124 | 2.23E−006 | 0.0494691 | 4.913421176 |
| | (calcium and DAG-regulated) | | | | |

\hat{ }Probe set ID number is the Affymetrix ID number on the HU133A array.
*Moderated t-statistic. This has the same interpretation as an ordinary t-statistic except that the standard errors have been moderated across genes, i.e., shrunk towards a common value, using a simple Bayesian model. Positive t-statistic indicates that the gene is upregulated following hemorrhagic stroke. Negative t-statistic indicates that the gene is downregulated following hemorrhagic stroke.
^{$}P-value uncorrected p value
^{#}Adjusted p-value is the corrected value after correction for multitple comparisons using the Holm method.
^{@}The B-statistic (lods or B) is the log-odds that the gene is differentially expressed.

The PAM list of 30 genes (37 gene probes; Table 5) was generated from the shrunken centroid approach in the index cohort and used to classify stroke in the first test cohort. The ranking was obtained from the statistical evaluation of the individual genes.

TABLE 5
ICH PAM Gene List with Putated Pathophysiological Classes

| | | |
|---|---|---|
| PAM | | |
| No. | Probe set ID\hat{ } | Gene Name |

Acute Inflammatory Response

| | | |
|---|---|---|
| 6 | NM_004244 | CD163 molecule |
| 16 | NM_001736 | Complement component 5a receptor 1 |
| 19 | NM_004668 | Maltase-glucoamylase (alpha-glucosidase) /// similar to |
| | | Maltase-glucoamylase, intestinal |
| 29 | NM_004566 | 6-phosphofructo-2-kinase/fructose-2,6-biphosphatase 3 |

Cell Adhesion

| | | |
|---|---|---|
| 2 | NM_021122 | Acyl-CoA synthetase long-chain family member 1 |
| 21 | NM_021599 | ADAM metallopeptidase with thrombospondin type 1 motif, 2 |

Suppression of Immune Response

| | | |
|---|---|---|
| 1 | NM_004633 | Interleukin 1 receptor, type II |
| 3 | NM_007268 | V-set and immunoglobulin domain containing 4 |
| 4* | NM_001706 | B-cell CLL/lymphoma 6 (zinc finger protein 51) |
| 7* | M15565 | T cell receptor alpha locus /// T cell receptor delta variable 2 |
| | | /// T cell receptor alpha variable 20 /// T cell receptor alpha |
| | | constant |
| 10* | NM_004585 | Retinoic acid receptor responder (tazarotene induced) 3 |
| 15* | NM_018384 | GTPase, IMAP family member 5 |
| 18* | NM_005317 | Granzyme M (lymphocyte met-ase 1) |
| 23* | NM_014034 | ASF1 anti-silencing function 1 homolog A (S. cerevisiae) |
| 25 | NM_003749 | Insulin receptor substrate 2 |
| 28* | NM_002001 | Fc fragment of IgE, high affinity I, receptor for; alpha polypeptide |

Hypoxia

| | | |
|---|---|---|
| 20 | NM_006931 | Solute carrier family 2 (facilitated glucose transporter), |
| | | member 3 |

Hematoma/Vascular Repair Response

| | | |
|---|---|---|
| 9 | NM_004054 | Complement component 3a receptor 1 |
| 11 | NM_016021 | Ubiquitin-conjugating enzyme E2, J1 (UBC6 homolog, |
| | | yeast) |
| 12 | NM_005461 | v-maf musculoaponeurotic fibrosarcoma oncogene homolog |
| | | B (avian) |
| 14 | NM_004504 | HIV-1 Rev binding protein /// region containing |
| | | hypothetical protein LOC285086; HIV-1 Rev binding |
| | | protein |
| 17 | NM_006343 | c-mer proto-oncogene tyrosine kinase |
| 27 | NM_000130 | Coagulation factor V (proaccelerin, labile factor) |
| 30 | NM_020995 | Haptoglobin /// haptoglobin-related protein |

Response to the Altered Cerebral Microenvironment

| | | |
|---|---|---|
| 13 | NM_001635 | Amphiphysin (Stiff-Man syndrome with breast cancer |
| | | 128 kDa autoantigen) |

Signal Transduction/Uncertain

| | | |
|---|---|---|
| 5 | NM_025195 | Tribbles homolog 1 (Drosophila) |
| 8 | NM_018404 | Centaurin, alpha 2 |
| 22 | NM_000104 | Cytochrome P450, family 1, subfamily B, polypeptide 1 |
| 24* | NM_030915 | Hypothetical protein DKFZp566J091 /// similar to |
| | | hypothetical protein DKFZp566J091 /// similar to |
| | | hypothetical protein |
| 26 | NM_015136 | Stabilin 1 |

\hat{ }probe set ID number is the Affymetrix ID number on the HU133A affay.
*Genes down-regulated in ICH relative to the referent group; the remaining genes were up-regulated in ICH.
NB: not all gene functions are as yet fully understood.

Tables 6-8 show the results of the hemorrhage versus ischemic stroke (HI lists) using the false discovery rate (FDR) (Table 6), Holm (Table 7), or PAM correction (Table 8). There were 483 (FDR), 27 (Holm), or 380 (PAM) gene probes that were significantly different between hemorrhage and control, representing 446, 28, and 316 genes, respectively. The differential expression of these genes indicates the presence of mechanisms to inactivate and to slow down white cell activation and differentiation.

After multiple comparison correction (MCC) using FDR correction, 483 gene probes, corresponding to 446 genes were found to be significantly different (Table 6). As shown in Table 6, several genes were upregulated (positive T-statistic, such as a value that is at least 3.6) or downregulated (negative t-statistic, such as a value that is less than −3.6) following a hemorrhagic stroke.

TABLE 6
Hemorrhagic stroke related-genes using FDR correction and
comparison to IS subjects.

| Probe Set | | | | Adjusted | |
|---|---|---|---|---|---|
| ID\hat{ } | Gene Name | t-statistic* | P Value^{$} | P Value^{#} | B^{@} |
| 205257_s_at | amphiphysin (Stiff-Man | 14.975963 | 6.99E−15 | 1.56E−010 | 20.7629274 |
| | syndrome with breast cancer | | | | |
| | 128 kDa autoantigen) | | | | |
| 211372_s_at | interleukin 1 receptor, type II | 10.712554 | 2.10E−011 | 2.34E−007 | 14.82446351 |
| 216233_at | CD163 molecule | 9.737206 | 1.75E−010 | 1.30E−006 | 12.88835863 |
| 214535_s_at | ADAM metallopeptidase | 8.4530582 | 3.46E−009 | 1.54E−005 | 10.50746674 |
| | with thrombospondin type 1 | | | | |
| | motif, 2 | | | | |
| 221011_s_at | hypothetical protein | −8.515744 | 2.98E−009 | 1.54E−005 | 10.70166657 |
| | DKFZp566J091 /// | | | | |
| | hypothetical protein | | | | |
| | DKFZp566J091 /// similar to | | | | |
| | hypothetical protein | | | | |
| | DKFZp566J091 /// similar to | | | | |
| | hypothetical protein | | | | |
| | DKFZp566J091 | | | | |
| 206028_s_at | c-mer proto-oncogene | 8.2851019 | 5.20E−009 | 1.93E−005 | 10.25765534 |
| | tyrosine kinase | | | | |
| 205403_at | interleukin 1 receptor, type II | 7.6873216 | 2.27E−008 | 7.24E−005 | 9.05172448 |
| 218494_s_at | SLC2A4 regulator | −7.333288 | 5.57E−008 | 0.0001551 | 8.265256103 |
| 205396_at | SMAD, mothers against DPP | −7.227747 | 7.29E−008 | 0.0001806 | 8.021975608 |
| | homolog 3 (Drosophila) | | | | |
| 205484_at | signaling threshold regulating | −7.018339 | 1.25E−007 | 0.000255 | 7.53111354 |
| | transmembrane adaptor 1 | | | | |
| 204116_at | interleukin 2 receptor, | −7.015854 | 1.26E−007 | 0.000255 | 7.521746309 |
| | gamma (severe combined | | | | |
| | immunodeficiency) | | | | |
| 218813_s_at | SH3-domain GRB2-like | −6.94035 | 1.53E−007 | 0.0002624 | 7.368885163 |
| | endophilin B2 | | | | |
| 218615_s_at | transmembrane protein 39A | 6.9645637 | 1.44E−007 | 0.0002624 | 7.302004604 |
| 209671_x_at | T cell receptor alpha locus /// | −6.753733 | 2.49E−007 | 0.0003963 | 6.8625403 |
| | T cell receptor alpha locus /// | | | | |
| | T cell receptor alpha constant | | | | |
| | /// T cell receptor alpha | | | | |
| | constant | | | | |
| 213805_at | abhydrolase domain | 6.71886 | 2.73E−007 | 0.0004053 | 6.807097433 |
| | containing 5 | | | | |
| 208611_s_at | spectrin, alpha, non- | −6.67564 | 3.06E−007 | 0.0004256 | 6.685920872 |
| | erythrocytic 1 (alpha-fodrin) | | | | |
| 208602_x_at | CD6 molecule | −6.604865 | 3.68E−007 | 0.0004806 | 6.552492682 |
| 221688_s_at | IMP3, U3 small nucleolar | −6.584643 | 3.88E−007 | 0.0004806 | 6.468878576 |
| | ribonucleoprotein, homolog | | | | |
| | (yeast) | | | | |
| 213275_x_at | cathepsin B | 6.4800111 | 5.12E−007 | 0.0005701 | 6.240830095 |
| 202499_s_at | solute carrier family 2 | 6.4812483 | 5.10E−007 | 0.0005701 | 6.188901235 |
| | (facilitated glucose | | | | |
| | transporter), member 3 | | | | |
| 218866_s_at | polymerase (RNA) III (DNA | −6.410061 | 6.16E−007 | 0.0006535 | 5.994655791 |
| | directed) polypeptide K, 12.3 kDa | | | | |
| 215049_x_at | CD163 molecule | 6.3394666 | 7.43E−007 | 0.0007197 | 5.872838463 |
| 211734_s_at | Fc fragment of IgE, high | −6.35615 | 7.11E−007 | 0.0007197 | 5.759237554 |
| | affinity I, receptor for; alpha | | | | |
| | polypeptide /// Fc fragment | | | | |
| | of IgE, high affinity I, | | | | |
| | receptor for; alpha | | | | |
| | polypeptide | | | | |
| 218805_at | GTPase, IMAP family | −6.236583 | 9.77E−007 | 0.0008709 | 5.638421367 |
| | member 5 /// GTPase, IMAP | | | | |
| | family member 5 | | | | |
| 211893_x_at | CD6 molecule | −6.245476 | 9.54E−007 | 0.0008709 | 5.68152223 |
| 203392_s_at | C-terminal binding protein 1 | −6.037307 | 1.67E−006 | 0.0014275 | 5.141550008 |
| 202191_s_at | growth arrest-specific 7 | 6.0036117 | 1.82E−006 | 0.0015049 | 5.046148265 |
| 217119_s_at | chemokine (C—X—C motif) | −5.922251 | 2.27E−006 | 0.0018063 | 4.858914423 |
| | receptor 3 | | | | |
| 207485_x_at | butyrophilin, subfamily 3, | −5.852698 | 2.74E−006 | 0.0021037 | 4.692097613 |
| | member A1 | | | | |
| 206025_s_at | tumor necrosis factor, alpha- | 5.8296612 | 2.91E−006 | 0.002164 | 4.546600129 |
| | induced protein 6 | | | | |
| 209163_at | cytochrome b-561 | −5.806405 | 3.10E−006 | 0.00223 | 4.575944433 |
| 215235_at | Spectrin, alpha, non- | −5.790736 | 3.24E−006 | 0.0022538 | 4.518417015 |
| | erythrocytic 1 (alpha-fodrin) | | | | |
| 37652_at | calcineurin binding protein 1 | −5.770015 | 3.42E−006 | 0.0022576 | 4.491015302 |
| 211628_x_at | ferritin, heavy polypeptide | 5.7676869 | 3.44E−006 | 0.0022576 | 4.47546201 |
| | pseudogene 1 /// ferritin, | | | | |
| | heavy polypeptide | | | | |
| | pseudogene 1 | | | | |
| 206100_at | carboxypeptidase M | 5.7319817 | 3.79E−006 | 0.002263 | 4.367654765 |
| 202964_s_at | regulatory factor X, 5 | −5.709916 | 4.03E−006 | 0.002263 | 4.330720664 |
| | (influences HLA class II | | | | |
| | expression) | | | | |
| 218689_at | Fanconi anemia, | −5.706747 | 4.06E−006 | 0.002263 | 4.322616624 |
| | complementation group F | | | | |
| 217991_x_at | single stranded DNA binding | −5.755493 | 3.56E−006 | 0.002263 | 4.439714989 |
| | protein 3 | | | | |
| 216442_x_at | fibronectin 1 | 5.7225944 | 3.89E−006 | 0.002263 | 4.333587406 |
| 200644_at | MARCKS-like 1 | −5.714933 | 3.97E−006 | 0.002263 | 4.332208376 |
| 204446_s_at | arachidonate 5-lipoxygenase | 5.6741338 | 4.44E−006 | 0.0024117 | 4.243000963 |
| 212114_at | hypothetical LOC552889 | −5.651779 | 4.71E−006 | 0.0025014 | 4.184857541 |
| 218342_s_at | KIAA1815 | −5.599329 | 5.44E−006 | 0.0025052 | 4.053204843 |
| 205456_at | CD3e molecule, epsilon | −5.586956 | 5.62E−006 | 0.0025052 | 4.025205549 |
| | (CD3-TCR complex) | | | | |
| 206674_at | fms-related tyrosine kinase 3 | 5.6074524 | 5.32E−006 | 0.0025052 | 4.064813729 |
| 213958_at | CD6 molecule /// CD6 | −5.622575 | 5.10E−006 | 0.0025052 | 4.104754729 |
| | molecule | | | | |
| 217763_s_at | RAB31, member RAS | 5.6052473 | 5.35E−006 | 0.0025052 | 4.049252269 |
| | oncogene family | | | | |
| 212259_s_at | pre-B-cell leukemia | −5.593187 | 5.53E−006 | 0.0025052 | 4.044003635 |
| | transcription factor | | | | |
| | interacting protein 1 | | | | |
| 204699_s_at | chromosome 1 open reading | −5.588481 | 5.60E−006 | 0.0025052 | 4.023513761 |
| | frame 107 | | | | |
| 214049_x_at | CD7 molecule | −5.589243 | 5.59E−006 | 0.0025052 | 4.033206881 |
| 202523_s_at | sparc/osteonectin, cwcv and | −5.563828 | 5.99E−006 | 0.002565 | 3.968512677 |
| | kazal-like domains | | | | |
| | proteoglycan (testican) 2 | | | | |
| 205888_s_at | janus kinase and microtubule | −5.569139 | 5.90E−006 | 0.002565 | 3.967342998 |
| | interacting protein 2 /// | | | | |
| | myelin transcription factor 1- | | | | |
| | like | | | | |
| 221937_at | CDNA FLJ34482 fis, clone | −5.552571 | 6.17E−006 | 0.0025947 | 3.916108112 |
| | HLUNG2004067 | | | | |
| 212888_at | Dicer1, Dcr-1 homolog | 5.5438219 | 6.32E−006 | 0.0026079 | 3.915198554 |
| | (Drosophila) | | | | |
| 204362_at | src family associated | 5.5222821 | 6.70E−006 | 0.0026196 | 3.861457206 |
| | phosphoprotein 2 | | | | |
| 220088_at | complement component 5a | 5.5323249 | 6.52E−006 | 0.0026196 | 3.884326856 |
| | receptor 1 | | | | |
| 212658_at | lipoma HMGIC fusion | 5.5231286 | 6.69E−006 | 0.0026196 | 3.82684045 |
| | partner-like 2 | | | | |
| 218402_s_at | Hermansky-Pudlak syndrome 4 | −5.50108 | 7.10E−006 | 0.0027272 | 3.792709125 |
| 208644_at | poly (ADP-ribose) | −5.467293 | 7.78E−006 | 0.0028909 | 3.710285612 |
| | polymerase family, member 1 | | | | |
| 201991_s_at | kinesin family member 5B /// | 5.4571556 | 8.00E−006 | 0.0028909 | 3.696501649 |
| | immediate early response 2 | | | | |
| 201785_at | ribonuclease, RNase A | 5.4266367 | 8.69E−006 | 0.0028909 | 3.591472965 |
| | family, 1 (pancreatic) | | | | |
| 213274_s_at | cathepsin B | 5.4299799 | 8.61E−006 | 0.0028909 | 3.620367389 |
| 210915_x_at | T cell receptor beta variable | −5.445797 | 8.25E−006 | 0.0028909 | 3.643846693 |
| | 19 /// T cell receptor beta | | | | |
| | constant 1 | | | | |
| 218865_at | MOCO sulphurase C- | 5.4290539 | 8.64E−006 | 0.0028909 | 3.599777398 |
| | terminal domain containing 1 | | | | |
| 38487_at | stabilin 1 | 5.4530577 | 8.09E−006 | 0.0028909 | 3.675376285 |
| 201109_s_at | thrombospondin 1 | 5.4287715 | 8.64E−006 | 0.0028909 | 3.619786242 |
| 218600_at | LIM domain containing 2 | −5.431841 | 8.57E−006 | 0.0028909 | 3.632836189 |
| 207460_at | granzyme M (lymphocyte | −5.384832 | 9.74E−006 | 0.0031918 | 3.506924838 |
| | met-ase 1) | | | | |
| 202436_s_at | cytochrome P450, family 1, | 5.3669918 | 1.02E−005 | 0.0033022 | 3.465418187 |
| | subfamily B, polypeptide 1 | | | | |
| 209154_at | Tax1 (human T-cell leukemia | 5.3376298 | 1.11E−005 | 0.0035262 | 3.393044034 |
| | virus type I) binding protein 3 | | | | |
| 210968_s_at | reticulon 4 | 5.3206717 | 1.16E−005 | 0.003641 | 3.346020743 |
| 202880_s_at | pleckstrin homology, Sec7 | −5.305509 | 1.21E−005 | 0.003742 | 3.303783051 |
| | and coiled-coil domains | | | | |
| | 1(cytohesin 1) | | | | |
| 200919_at | polyhomeotic-like 2 | 5.2969148 | 1.24E−005 | 0.0037782 | 3.273511465 |
| | (Drosophila) | | | | |
| 207433_at | interleukin 10 | 5.2675667 | 1.34E−005 | 0.0040378 | 3.198157953 |
| 218092_s_at | HIV-1 Rev binding protein /// | 5.2534916 | 1.39E−005 | 0.0040854 | 3.157401509 |
| | region containing | | | | |
| | hypothetical protein | | | | |
| | LOC285086; HIV-1 Rev | | | | |
| | binding protein | | | | |
| 215127_s_at | RNA binding motif, single | 5.2549129 | 1.39E−005 | 0.0040854 | 3.183246082 |
| | stranded interacting protein 1 | | | | |
| | /// chromosome 2 open | | | | |
| | reading frame 12 /// region | | | | |
| | containing chromosome 2 | | | | |
| | open reading frame 12; RNA | | | | |
| | binding motif, single | | | | |
| | stranded interacting protein 1 | | | | |
| 213622_at | collagen, type IX, alpha 2 | −5.234239 | 1.47E−005 | 0.0042498 | 3.130282812 |
| 64064_at | GTPase, IMAP family | −5.204148 | 1.59E−005 | 0.0044968 | 3.054172752 |
| | member 5 | | | | |
| 202479_s_at | tribbles homolog 2 | −5.206314 | 1.58E−005 | 0.0044968 | 3.05899879 |
| | (Drosophila) | | | | |
| 218328_at | coenzyme Q4 homolog (S. cerevisiae) | −5.196191 | 1.63E−005 | 0.0045381 | 3.033875386 |
| 218871_x_at | chondroitin sulfate | 5.1847882 | 1.68E−005 | 0.0046237 | 3.004991701 |
| | GalNAcT-2 | | | | |
| 203828_s_at | interleukin 32 /// interleukin | −5.163169 | 1.78E−005 | 0.004845 | 2.946319633 |
| | 32 | | | | |
| 210985_s_at | SP100 nuclear antigen | 5.0964818 | 2.14E−005 | 0.0057428 | 2.779851237 |
| 204768_s_at | flap structure-specific | −5.078538 | 2.25E−005 | 0.0059595 | 2.734696971 |
| | endonuclease 1 | | | | |
| 206181_at | signaling lymphocytic | −5.068667 | 2.31E−005 | 0.00598 | 2.709663481 |
| | activation molecule family | | | | |
| | member 1 | | | | |
| 202931_x_at | bridging integrator 1 | −5.071661 | 2.29E−005 | 0.00598 | 2.71321811 |
| 204861_s_at | baculoviral IAP repeat- | 5.0456785 | 2.46E−005 | 0.0060846 | 2.651407007 |
| | containing 1 /// similar to | | | | |
| | Baculoviral IAP repeat- | | | | |
| | containing protein 1 | | | | |
| | (Neuronal apoptosis | | | | |
| | inhibitory protein) | | | | |
| 213193_x_at | T cell receptor beta variable | −5.046633 | 2.45E−005 | 0.0060846 | 2.647005328 |
| | 19 /// T cell receptor beta | | | | |
| | variable 19 /// T cell receptor | | | | |
| | beta constant 1 /// T cell | | | | |
| | receptor beta constant 1 | | | | |
| 204140_at | tyrosylprotein | 5.0502371 | 2.43E−005 | 0.0060846 | 2.650095058 |
| | sulfotransferase 1 | | | | |
| 208304_at | chemokine (C-C motif) | −5.049961 | 2.43E−005 | 0.0060846 | 2.661145479 |
| | receptor 3 | | | | |
| 221602_s_at | Fas apoptotic inhibitory | −5.025333 | 2.60E−005 | 0.0062926 | 2.598353681 |
| | molecule 3 /// Fas apoptotic | | | | |
| | inhibitory molecule 3 | | | | |
| 211902_x_at | T cell receptor alpha locus | −5.026903 | 2.59E−005 | 0.0062926 | 2.603796518 |
| 206335_at | galactosamine (N-acetyl)-6- | 5.0177249 | 2.65E−005 | 0.0063557 | 2.577144281 |
| | sulfate sulfatase (Morquio | | | | |
| | syndrome, | | | | |
| | mucopolysaccharidosis type | | | | |
| | IVA) | | | | |
| 204971_at | cystatin A (stefin A) | 4.9962548 | 2.81E−005 | 0.006668 | 2.525788373 |
| 213107_at | TRAF2 and NCK interacting | −4.987247 | 2.88E−005 | 0.0067622 | 2.500803335 |
| | kinase | | | | |
| 216133_at | T cell receptor alpha locus | −4.967461 | 3.04E−005 | 0.0069907 | 2.453159884 |
| 208709_s_at | nardilysin (N-arginine | 4.969361 | 3.03E−005 | 0.0069907 | 2.457708459 |
| | dibasic convertase) | | | | |
| 209570_s_at | DNA segment on | −4.960273 | 3.10E−005 | 0.0070566 | 2.433586736 |
| | chromosome 4 (unique) 234 | | | | |
| | expressed sequence | | | | |
| 202730_s_at | programmed cell death 4 | −4.955701 | 3.14E−005 | 0.0070731 | 2.422354409 |
| | (neoplastic transformation | | | | |
| | inhibitor) | | | | |
| 217957_at | chromosome 16 open reading | −4.945286 | 3.23E−005 | 0.0071332 | 2.396869242 |
| | frame 80 | | | | |
| 218685_s_at | single-strand-selective | 4.9459068 | 3.23E−005 | 0.0071332 | 2.398570464 |
| | monofunctional uracil-DNA | | | | |
| | glycosylase 1 | | | | |
| 200953_s_at | cyclin D2 | −4.928398 | 3.39E−005 | 0.0071898 | 2.35417029 |
| 204787_at | V-set and immunoglobulin | 4.9247091 | 3.42E−005 | 0.0071898 | 2.343885219 |
| | domain containing 4 | | | | |
| 219809_at | WD repeat domain 55 | −4.932335 | 3.35E−005 | 0.0071898 | 2.362263324 |
| 204460_s_at | RAD1 homolog (S. pombe) | −4.934145 | 3.33E−005 | 0.0071898 | 2.368885367 |
| 208686_s_at | bromodomain containing 2 | −4.928024 | 3.39E−005 | 0.0071898 | 2.349607531 |
| 213078_x_at | 1-acylglycerol-3-phosphate | −4.904068 | 3.62E−005 | 0.0074661 | 2.292881888 |
| | O-acyltransferase 7 | | | | |
| | (lysophosphatidic acid | | | | |
| | acyltransferase, eta) | | | | |
| 221811_at | per1-like domain containing 1 | −4.905078 | 3.61E−005 | 0.0074661 | 2.2948526 |
| 213444_at | hypothetical protein | −4.890559 | 3.75E−005 | 0.0076758 | 2.258785295 |
| | LOC643641 | | | | |
| 200998_s_at | cytoskeleton-associated | 4.8819424 | 3.84E−005 | 0.0077872 | 2.237004762 |
| | protein 4 | | | | |
| 202694_at | serine/threonine kinase 17a | −4.863093 | 4.05E−005 | 0.0081249 | 2.186146729 |
| | (apoptosis-inducing) | | | | |
| 201283_s_at | trafficking protein, kinesin | 4.8530582 | 4.16E−005 | 0.0082761 | 2.163769161 |
| | binding 1 | | | | |
| 203751_x_at | jun D proto-oncogene | −4.848441 | 4.21E−005 | 0.008307 | 2.149596198 |
| 206099_at | protein kinase C, eta | −4.838764 | 4.33E−005 | 0.0083812 | 2.127958815 |
| 212464_s_at | fibronectin 1 | 4.8392298 | 4.32E−005 | 0.0083812 | 2.128955463 |
| 214177_s_at | pre-B-cell leukemia | −4.835538 | 4.36E−005 | 0.0083825 | 2.119523059 |
| | transcription factor | | | | |
| | interacting protein 1 | | | | |
| 201557_at | vesicle-associated membrane | −4.831628 | 4.41E−005 | 0.0084001 | 2.107009666 |
| | protein 2 (synaptobrevin 2) | | | | |
| 221893_s_at | aarF domain containing | −4.801951 | 4.78E−005 | 0.0090322 | 2.031162299 |
| | kinase 2 | | | | |
| 213539_at | CD3d molecule, delta (CD3- | −4.787434 | 4.98E−005 | 0.0092876 | 1.998617882 |
| | TCR complex) | | | | |
| 206170_at | adrenergic, beta-2-, receptor, | −4.785587 | 5.00E−005 | 0.0092876 | 1.987559985 |
| | surface | | | | |
| 221249_s_at | family with sequence | −4.777379 | 5.12E−005 | 0.0094197 | 1.971226037 |
| | similarity 117, member A /// | | | | |
| | family with sequence | | | | |
| | similarity 117, member A | | | | |
| 201935_s_at | eukaryotic translation | 4.7711051 | 5.20E−005 | 0.0095039 | 1.953370101 |
| | initiation factor 4 gamma, 3 | | | | |
| 204852_s_at | protein tyrosine phosphatase, | −4.767225 | 5.26E−005 | 0.009527 | 1.944747197 |
| | non-receptor type 7 | | | | |
| 203887_s_at | thrombomodulin | 4.7398428 | 5.67E−005 | 0.0099677 | 1.876175854 |
| 208361_s_at | polymerase (RNA) III (DNA | −4.738944 | 5.68E−005 | 0.0099677 | 1.875057617 |
| | directed) polypeptide D, | | | | |
| | 44 kDa | | | | |
| 219207_at | enhancer of mRNA | −4.739895 | 5.67E−005 | 0.0099677 | 1.876596065 |
| | decapping 3 homolog (S. cerevisiae) | | | | |
| 204730_at | regulating synaptic | −4.739609 | 5.67E−005 | 0.0099677 | 1.875078253 |
| | membrane exocytosis 3 | | | | |
| 203611_at | telomeric repeat binding | −4.734063 | 5.76E−005 | 0.0100225 | 1.857637447 |
| | factor 2 | | | | |
| 208591_s_at | phosphodiesterase 3B, | −4.710967 | 6.13E−005 | 0.010592 | 1.800739865 |
| | cGMP-inhibited | | | | |
| 210202_s_at | bridging integrator 1 | −4.667609 | 6.90E−005 | 0.0110257 | 1.695480391 |
| 215646_s_at | chondroitin sulfate | 4.6817028 | 6.64E−005 | 0.0110257 | 1.730262924 |
| | proteoglycan 2 (versican) /// | | | | |
| | chondroitin sulfate | | | | |
| | proteoglycan 2 (versican) | | | | |
| 209663_s_at | integrin, alpha 7 | 4.6731631 | 6.80E−005 | 0.0110257 | 1.711874895 |
| 201110_s_at | thrombospondin 1 | 4.6732479 | 6.80E−005 | 0.0110257 | 1.706741634 |
| 203547_at | CD4 molecule /// CD4 | −4.685996 | 6.56E−005 | 0.0110257 | 1.743145893 |
| | molecule | | | | |
| 211711_s_at | phosphatase and tensin | 4.6662725 | 6.93E−005 | 0.0110257 | 1.690191217 |
| | homolog (mutated in | | | | |
| | multiple advanced cancers 1) | | | | |
| | /// phosphatase and tensin | | | | |
| | homolog (mutated in | | | | |
| | multiple advanced cancers 1) | | | | |
| 209827_s_at | interleukin 16 (lymphocyte | −4.669241 | 6.87E−005 | 0.0110257 | 1.702124982 |
| | chemoattractant factor) | | | | |
| 213812_s_at | calcium/calmodulin- | 4.6701242 | 6.85E−005 | 0.0110257 | 1.6983144 |
| | dependent protein kinase | | | | |
| | kinase 2, beta | | | | |
| 217891_at | chromosome 16 open reading | −4.683736 | 6.60E−005 | 0.0110257 | 1.729480573 |
| | frame 58 | | | | |
| 218559_s_at | v-maf musculoaponeurotic | 4.6797903 | 6.68E−005 | 0.0110257 | 1.720156399 |
| | fibrosarcoma oncogene | | | | |
| | homolog B (avian) | | | | |
| 213079_at | TSR2, 20S rRNA | −4.680972 | 6.65E−005 | 0.0110257 | 1.729047731 |
| | accumulation, homolog (S. cerevisiae) | | | | |
| 215761_at | Dmx-like 2 | 4.6626878 | 7.00E−005 | 0.0110551 | 1.681331225 |
| 201294_s_at | WD repeat and SOCS box- | 4.6523386 | 7.20E−005 | 0.0112916 | 1.659128684 |
| | containing 1 | | | | |
| 218367_x_at | ubiquitin specific peptidase | −4.648163 | 7.28E−005 | 0.0113411 | 1.647062445 |
| | 21 | | | | |
| 210972_x_at | T cell receptor alpha locus /// | −4.630054 | 7.65E−005 | 0.0118325 | 1.595348487 |
| | T cell receptor delta variable | | | | |
| | 2 /// T cell receptor alpha | | | | |
| | variable 20 /// T cell receptor | | | | |
| | alpha constant | | | | |
| 200952_s_at | cyclin D2 | −4.621284 | 7.83E−005 | 0.0120353 | 1.571234703 |
| 201361_at | transmembrane protein 109 | −4.618119 | 7.90E−005 | 0.0120373 | 1.569927468 |
| 207347_at | excision repair cross- | −4.6162 | 7.94E−005 | 0.0120373 | 1.561743434 |
| | complementing rodent repair | | | | |
| | deficiency, complementation | | | | |
| | group 6 | | | | |
| 201709_s_at | nipsnap homolog 1 (C. elegans) | −4.60624 | 8.16E−005 | 0.0122851 | 1.535280231 |
| 218466_at | TBC1 domain family, | −4.597874 | 8.35E−005 | 0.0124841 | 1.510604461 |
| | member 17 | | | | |
| 213689_x_at | Ribosomal protein L5 | −4.592234 | 8.48E−005 | 0.012593 | 1.501959271 |
| 205681_at | BCL2-related protein A1 | 4.589087 | 8.55E−005 | 0.0126173 | 1.500196962 |
| 213572_s_at | serpin peptidase inhibitor, | 4.585417 | 8.64E−005 | 0.0126603 | 1.490007576 |
| | clade B (ovalbumin), | | | | |
| | member 1 | | | | |
| 219715_s_at | tyrosyl-DNA | −4.577109 | 8.83E−005 | 0.0127906 | 1.458594073 |
| | phosphodiesterase 1 | | | | |
| 211941_s_at | phosphatidylethanolamine | −4.576864 | 8.84E−005 | 0.0127906 | 1.471399883 |
| | binding protein 1 | | | | |
| 212747_at | ankyrin repeat and sterile | 4.5738191 | 8.91E−005 | 0.0128139 | 1.455791519 |
| | alpha motif domain | | | | |
| | containing 1A | | | | |
| 210243_s_at | UDP-Gal:betaGlcNAc beta | −4.556171 | 9.35E−005 | 0.01316 | 1.408640624 |
| | 1,4-galactosyltransferase, | | | | |
| | polypeptide 3 | | | | |
| 211936_at | heat shock 70 kDa protein 5 | 4.5532167 | 9.43E−005 | 0.01316 | 1.40095313 |
| | (glucose-regulated protein, | | | | |
| | 78 kDa) | | | | |
| 207838_x_at | pre-B-cell leukemia | −4.556 | 9.36E−005 | 0.01316 | 1.403361394 |
| | transcription factor | | | | |
| | interacting protein 1 | | | | |
| 203645_s_at | CD163 molecule | 4.5508475 | 9.49E−005 | 0.01316 | 1.395886119 |
| 205598_at | TRAF interacting protein | 4.5500975 | 9.51E−005 | 0.01316 | 1.396685547 |
| 203428_s_at | ASF1 anti-silencing function | −4.559797 | 9.26E−005 | 0.01316 | 1.421413806 |
| | 1 homolog A (S. cerevisiae) | | | | |
| 209546_s_at | apolipoprotein L, 1 | −4.538782 | 9.81E−005 | 0.0134881 | 1.357788032 |
| 201189_s_at | inositol 1,4,5-triphosphate | −4.530502 | 0.0001003 | 0.0137111 | 1.354606409 |
| | receptor, type 3 | | | | |
| 206271_at | toll-like receptor 3 | −4.521199 | 0.0001029 | 0.0139771 | 1.316875162 |
| 209409_at | growth factor receptor-bound | 4.5115037 | 0.0001056 | 0.014182 | 1.308934426 |
| | protein 10 | | | | |
| 203747_at | aquaporin 3 (Gill blood | −4.5114 | 0.0001057 | 0.014182 | 1.2884238 |
| | group) | | | | |
| 214022_s_at | interferon induced | −4.503533 | 0.0001079 | 0.0144021 | 1.287129364 |
| | transmembrane protein 1 (9- | | | | |
| | 27) | | | | |
| 213817_at | CDNA FLJ13601 fis, clone | 4.5011241 | 0.0001086 | 0.0144106 | 1.266737499 |
| | PLACE1010069 | | | | |
| 218133_s_at | NIF3 NGG1 interacting | −4.493705 | 0.0001109 | 0.0146175 | 1.260831487 |
| | factor 3-like 1 (S. pombe) | | | | |
| 209508_x_at | CASP8 and FADD-like | 4.4843314 | 0.0001137 | 0.0149069 | 1.221866586 |
| | apoptosis regulator /// | | | | |
| | CASP8 and FADD-like | | | | |
| | apoptosis regulator | | | | |
| 204070_at | retinoic acid receptor | −4.478979 | 0.0001154 | 0.0150371 | 1.210867441 |
| | responder (tazarotene | | | | |
| | induced) 3 | | | | |
| 218620_s_at | HemK methyltransferase | −4.472243 | 0.0001175 | 0.0152261 | 1.20381234 |
| | family member 1 | | | | |
| 202743_at | phosphoinositide-3-kinase, | −4.466616 | 0.0001193 | 0.0153716 | 1.181228916 |
| | regulatory subunit 3 (p55, | | | | |
| | gamma) | | | | |
| 210495_x_at | fibronectin 1 | 4.464238 | 0.0001201 | 0.0153824 | 1.176619284 |
| 220299_at | spermatogenesis associated 6 | 4.459989 | 0.0001215 | 0.0154723 | 1.158131959 |
| 212017_at | hypothetical protein | −4.455862 | 0.0001229 | 0.015558 | 1.15769254 |
| | LOC130074 | | | | |
| 205775_at | family with sequence | −4.444741 | 0.0001267 | 0.0157699 | 1.134986188 |
| | similarity 50, member B | | | | |
| 211900_x_at | CD6 molecule | −4.444671 | 0.0001267 | 0.0157699 | 1.116737561 |
| 214877_at | CDK5 regulatory subunit | −4.448146 | 0.0001255 | 0.0157699 | 1.132845939 |
| | associated protein 1-like 1 | | | | |
| 205603_s_at | diaphanous homolog 2 | 4.4350343 | 0.00013 | 0.0160088 | 1.096361835 |
| | (Drosophila) | | | | |
| 215001_s_at | glutamate-ammonia ligase | 4.433024 | 0.0001308 | 0.0160088 | 1.09074053 |
| | (glutamine synthetase) | | | | |
| 212400_at | family with sequence | −4.434852 | 0.0001301 | 0.0160088 | 1.100438265 |
| | similarity 102, member A | | | | |
| 202747_s_at | integral membrane protein | −4.427383 | 0.0001328 | 0.0160794 | 1.083439154 |
| | 2A | | | | |
| 213587_s_at | ATPase, H+ transporting V0 | −4.429166 | 0.0001321 | 0.0160794 | 1.09280248 |
| | subunit E2-like (rat) | | | | |
| 201185_at | HtrA serine peptidase 1 | 4.4223939 | 0.0001346 | 0.0161236 | 1.087068097 |
| 203674_at | helicase with zinc finger | 4.4226665 | 0.0001345 | 0.0161236 | 1.063318956 |
| 212144_at | unc-84 homolog B (C. elegans) | −4.420401 | 0.0001353 | 0.0161245 | 1.063721092 |
| 204890_s_at | lymphocyte-specific protein | −4.413172 | 0.000138 | 0.0163568 | 1.058240482 |
| | tyrosine kinase | | | | |
| 203265_s_at | mitogen-activated protein | −4.407301 | 0.0001402 | 0.0165319 | 1.033787005 |
| | kinase kinase 4 | | | | |
| 221804_s_at | family with sequence | 4.399421 | 0.0001433 | 0.0168006 | 1.013176243 |
| | similarity 45, member B /// | | | | |
| | family with sequence | | | | |
| | similarity 45, member A | | | | |
| 211571_s_at | chondroitin sulfate | 4.3908505 | 0.0001466 | 0.0171062 | 1.004946838 |
| | proteoglycan 2 (versican) | | | | |
| 219988_s_at | chromosome 1 open reading | −4.388658 | 0.0001475 | 0.0171187 | 0.978540081 |
| | frame 164 | | | | |
| 201717_at | mitochondrial ribosomal | −4.372909 | 0.0001539 | 0.0172903 | 0.941661842 |
| | protein L49 | | | | |
| 208829_at | TAP binding protein | −4.378401 | 0.0001517 | 0.0172903 | 0.954879686 |
| | (tapasin) | | | | |
| 211796_s_at | T cell receptor beta variable | −4.380038 | 0.000151 | 0.0172903 | 0.977041216 |
| | 21-1 /// T cell receptor beta | | | | |
| | variable 19 /// T cell receptor | | | | |
| | beta variable 5-4 /// T cell | | | | |
| | receptor beta variable 3-1 /// | | | | |
| | T cell receptor beta constant 1 | | | | |
| 221325_at | potassium channel, subfamily | 4.3699453 | 0.0001552 | 0.0172903 | 0.942547754 |
| | K, member 13 | | | | |
| 41644_at | SAM and SH3 domain | 4.3744041 | 0.0001533 | 0.0172903 | 0.964958746 |
| | containing 1 | | | | |
| 210038_at | protein kinase C, theta | −4.374494 | 0.0001533 | 0.0172903 | 0.969885936 |
| 204925_at | cystinosis, nephropathic | 4.3810979 | 0.0001506 | 0.0172903 | 0.98280358 |
| 210166_at | toll-like receptor 5 | 4.370998 | 0.0001547 | 0.0172903 | 0.949179511 |
| 202917_s_at | S100 calcium binding protein | 4.3637294 | 0.0001578 | 0.0174969 | 0.929743182 |
| | A8 (calgranulin A) | | | | |
| 204908_s_at | B-cell CLL/lymphoma 3 | 4.3586482 | 0.00016 | 0.017652 | 0.903624485 |
| 206207_at | Charcot-Leyden crystal | −4.346141 | 0.0001655 | 0.0176728 | 0.888625097 |
| | protein /// Charcot-Leyden | | | | |
| | crystal protein | | | | |
| 200599_s_at | heat shock protein 90 kDa | 4.3405109 | 0.0001681 | 0.0176728 | 0.854446089 |
| | beta (Grp94), member 1 | | | | |
| 213778_x_at | zinc finger protein 276 | −4.340402 | 0.0001681 | 0.0176728 | 0.861261704 |
| 210517_s_at | A kinase (PRKA) anchor | −4.34106 | 0.0001678 | 0.0176728 | 0.859500941 |
| | protein (gravin) 12 | | | | |
| 218454_at | hypothetical protein | 4.3550338 | 0.0001616 | 0.0176728 | 0.904304182 |
| | FLJ22662 | | | | |
| 203140_at | B-cell CLL/lymphoma 6 | 4.3450185 | 0.000166 | 0.0176728 | 0.871019511 |
| | (zinc finger protein 51) /// B- | | | | |
| | cell CLL/lymphoma 6 (zinc | | | | |
| | finger protein 51) | | | | |
| 200057_s_at | non-POU domain containing, | −4.346887 | 0.0001652 | 0.0176728 | 0.88320629 |
| | octamer-binding /// non-POU | | | | |
| | domain containing, octamer- | | | | |
| | binding | | | | |
| 213229_at | Dicer1, Dcr-1 homolog | 4.3474835 | 0.0001649 | 0.0176728 | 0.877171318 |
| | (Drosophila) | | | | |
| 207428_x_at | cell division cycle 2-like 1 | −4.350843 | 0.0001634 | 0.0176728 | 0.882919048 |
| | (PITSLRE proteins) | | | | |
| 213988_s_at | spermidine/spermine N1- | 4.3507687 | 0.0001635 | 0.0176728 | 0.886572778 |
| | acetyltransferase 1 | | | | |
| 204150_at | stabilin 1 | 4.334514 | 0.0001708 | 0.0177698 | 0.856325129 |
| 200675_at | CD81 molecule | −4.333828 | 0.0001712 | 0.0177698 | 0.851775218 |
| 221519_at | F-box and WD-40 domain | −4.333201 | 0.0001715 | 0.0177698 | 0.860433931 |
| | protein 4 | | | | |
| 221610_s_at | signal-transducing adaptor | 4.3293676 | 0.0001732 | 0.0178723 | 0.83885944 |
| | protein-2 | | | | |
| 205745_x_at | ADAM metallopeptidase | 4.32695 | 0.0001744 | 0.0179069 | 0.823378731 |
| | domain 17 (tumor necrosis | | | | |
| | factor, alpha, converting | | | | |
| | enzyme) | | | | |
| 220386_s_at | echinoderm microtubule | −4.316855 | 0.0001792 | 0.0180706 | 0.803413006 |
| | associated protein like 4 | | | | |
| 202621_at | interferon regulatory factor 3 | −4.31958 | 0.0001779 | 0.0180706 | 0.806149427 |
| 203556_at | zinc fingers and homeoboxes 2 | −4.32148 | 0.000177 | 0.0180706 | 0.833182794 |
| 201561_s_at | calsyntenin 1 | −4.317668 | 0.0001788 | 0.0180706 | 0.81157157 |
| 217552_x_at | complement component | 4.3146165 | 0.0001803 | 0.0180987 | 0.809684654 |
| | (3b/4b) receptor 1 (Knops | | | | |
| | blood group) | | | | |
| 206662_at | glutaredoxin | 4.2962876 | 0.0001895 | 0.0189285 | 0.752114887 |
| | (thioltransferase) | | | | |
| 203956_at | MORC family CW-type zinc | −4.291476 | 0.000192 | 0.0189285 | 0.742158497 |
| | finger 2 | | | | |
| 218043_s_at | 5-azacytidine induced 2 | 4.2924838 | 0.0001915 | 0.0189285 | 0.737577647 |
| 202464_s_at | 6-phosphofructo-2- | 4.2917202 | 0.0001919 | 0.0189285 | 0.729065572 |
| | kinase/fructose-2,6- | | | | |
| | biphosphatase 3 | | | | |
| 205119_s_at | formyl peptide receptor 1 /// | 4.2883385 | 0.0001936 | 0.0189575 | 0.747259094 |
| | formyl peptide receptor 1 | | | | |
| 220684_at | T-box 21 | −4.287658 | 0.000194 | 0.0189575 | 0.72011433 |
| 218323_at | ras homolog gene family, | 4.2837373 | 0.000196 | 0.0190762 | 0.723842895 |
| | member T1 | | | | |
| 206026_s_at | tumor necrosis factor, alpha- | 4.2782985 | 0.000199 | 0.019275 | 0.734046121 |
| | induced protein 6 | | | | |
| 217774_s_at | hypothetical protein | −4.276277 | 0.0002 | 0.0192969 | 0.69862666 |
| | HSPC152 | | | | |
| 209504_s_at | pleckstrin homology domain | −4.271353 | 0.0002027 | 0.0194715 | 0.68575459 |
| | containing, family B | | | | |
| | (evectins) member 1 | | | | |
| 221658_s_at | interleukin 21 receptor | −4.26704 | 0.0002051 | 0.0195702 | 0.684477646 |
| 205027_s_at | mitogen-activated protein | 4.2663136 | 0.0002055 | 0.0195702 | 0.667758676 |
| | kinase kinase kinase 8 | | | | |
| 208141_s_at | deoxyhypusine | −4.246833 | 0.0002166 | 0.0205415 | 0.624881393 |
| | hydroxylase/monooxygenase | | | | |
| 216705_s_at | adenosine deaminase | −4.240822 | 0.0002202 | 0.0207897 | 0.627297384 |
| 207556_s_at | diacylglycerol kinase, zeta | −4.234852 | 0.0002238 | 0.0209504 | 0.593023687 |
| | 104 kDa | | | | |
| 210039_s_at | protein kinase C, theta | −4.235346 | 0.0002235 | 0.0209504 | 0.601633662 |
| 204031_s_at | poly(rC) binding protein 2 | −4.227464 | 0.0002283 | 0.0210842 | 0.598729627 |
| 219622_at | RAB20, member RAS | 4.2263317 | 0.000229 | 0.0210842 | 0.578985349 |
| | oncogene family | | | | |
| 212171_x_at | vascular endothelial growth | 4.2272519 | 0.0002284 | 0.0210842 | 0.58151039 |
| | factor | | | | |
| 214219_x_at | mitogen-activated protein | −4.228186 | 0.0002278 | 0.0210842 | 0.591854364 |
| | kinase kinase kinase kinase 1 | | | | |
| 213261_at | lupus brain antigen 1 | −4.219214 | 0.0002334 | 0.0214053 | 0.555151583 |
| 202459_s_at | lipin 2 | −4.211581 | 0.0002383 | 0.0217618 | 0.527731801 |
| 202872_at | ATPase, H+ transporting, | 4.201101 | 0.0002451 | 0.0222953 | 0.533194426 |
| | lysosomal 42 kDa, V1 subunit | | | | |
| | C1 | | | | |
| 218372_at | mediator of RNA polymerase | −4.197807 | 0.0002473 | 0.0224031 | 0.504980508 |
| | II transcription, subunit 9 | | | | |
| | homolog (S. cerevisiae) | | | | |
| 218250_s_at | CCR4-NOT transcription | −4.193976 | 0.0002499 | 0.0225443 | 0.506291173 |
| | complex, subunit 7 | | | | |
| 208959_s_at | thioredoxin domain | 4.1909732 | 0.0002519 | 0.0226134 | 0.49087302 |
| | containing 4 (endoplasmic | | | | |
| | reticulum) | | | | |
| 204045_at | transcription elongation | −4.189856 | 0.0002527 | 0.0226134 | 0.507340016 |
| | factor A (SII)-like 1 | | | | |
| 203846_at | tripartite motif-containing 32 | −4.183559 | 0.000257 | 0.0229091 | 0.463736175 |
| 210201_x_at | bridging integrator 1 | −4.177284 | 0.0002614 | 0.0231362 | 0.460702626 |
| 205425_at | huntingtin interacting protein 1 | 4.1769515 | 0.0002616 | 0.0231362 | 0.469009904 |
| 201555_at | MCM3 minichromosome | −4.168572 | 0.0002676 | 0.0232804 | 0.432832347 |
| | maintenance deficient 3 (S. cerevisiae) | | | | |
| 206061_s_at | Dicer1, Dcr-1 homolog | 4.1719164 | 0.0002652 | 0.0232804 | 0.438363066 |
| | (Drosophila) | | | | |
| 204683_at | intercellular adhesion | −4.164289 | 0.0002707 | 0.0232804 | 0.423015046 |
| | molecule 2 | | | | |
| 213218_at | zinc finger protein 187 | −4.170657 | 0.0002661 | 0.0232804 | 0.426234481 |
| 200707_at | protein kinase C substrate | −4.163854 | 0.0002711 | 0.0232804 | 0.411710809 |
| | 80K-H | | | | |
| 200941_at | heat shock factor binding | 4.1616413 | 0.0002727 | 0.0232804 | 0.407850451 |
| | protein 1 | | | | |
| 206053_at | zinc finger protein 510 | −4.167219 | 0.0002686 | 0.0232804 | 0.417971932 |
| 214971_s_at | ST6 beta-galactosamide | −4.162897 | 0.0002718 | 0.0232804 | 0.423445105 |
| | alpha-2,6-sialyltranferase 1 | | | | |
| 205349_at | guanine nucleotide binding | 4.1688761 | 0.0002674 | 0.0232804 | 0.461078912 |
| | protein (G protein), alpha 15 | | | | |
| | (Gq class) | | | | |
| 204646_at | dihydropyrimidine | 4.1588683 | 0.0002747 | 0.0233656 | 0.40894219 |
| | dehydrogenase | | | | |
| 219133_at | 3-oxoacyl-ACP synthase, | −4.156085 | 0.0002768 | 0.0233706 | 0.418569087 |
| | mitochondrial | | | | |
| 202626_s_at | v-yes-1 Yamaguchi sarcoma | 4.1559692 | 0.0002769 | 0.0233706 | 0.399893823 |
| | viral related oncogene | | | | |
| | homolog /// v-yes-1 | | | | |
| | Yamaguchi sarcoma viral | | | | |
| | related oncogene homolog | | | | |
| 204900_x_at | Sin3A-associated protein, | 4.1464433 | 0.0002841 | 0.0238881 | 0.376280988 |
| | 30 kDa | | | | |
| 202537_s_at | chromatin modifying protein | 4.1361638 | 0.0002921 | 0.024182 | 0.341960991 |
| | 2B | | | | |
| 217815_at | suppressor of Ty 16 homolog | −4.138862 | 0.00029 | 0.024182 | 0.363534725 |
| | (S. cerevisiae) | | | | |
| 203548_s_at | lipoprotein lipase | 4.1349694 | 0.000293 | 0.024182 | 0.36999773 |
| 221818_at | integrator complex subunit 5 | −4.136004 | 0.0002922 | 0.024182 | 0.346903905 |
| 210681_s_at | ubiquitin specific peptidase | 4.1381723 | 0.0002905 | 0.024182 | 0.356142615 |
| | 15 | | | | |
| 209710_at | GATA binding protein 2 | −4.128679 | 0.000298 | 0.0245046 | 0.32613342 |
| 212355_at | KIAA0323 | −4.12548 | 0.0003006 | 0.0245356 | 0.324273542 |
| 212722_s_at | phosphatidylserine receptor | 4.1264637 | 0.0002998 | 0.0245356 | 0.332085323 |
| 214958_s_at | transmembrane channel-like 6 | −4.12397 | 0.0003018 | 0.0245456 | 0.330470069 |
| 209603_at | GATA binding protein 3 | −4.12156 | 0.0003038 | 0.0246157 | 0.304194358 |
| 209969_s_at | signal transducer and | −4.119215 | 0.0003057 | 0.0246819 | 0.306207408 |
| | activator of transcription 1, | | | | |
| | 91 kDa | | | | |
| 218754_at | nucleolar protein 9 | −4.10818 | 0.0003149 | 0.0252432 | 0.273787689 |
| 214756_x_at | postmeiotic segregation | −4.108296 | 0.0003148 | 0.0252432 | 0.276643584 |
| | increased 2-like 1 | | | | |
| 201344_at | ubiquitin-conjugating | 4.1024994 | 0.0003198 | 0.025449 | 0.258365265 |
| | enzyme E2D 2 (UBC4/5 | | | | |
| | homolog, yeast) | | | | |
| 202731_at | programmed cell death 4 | −4.103329 | 0.0003191 | 0.025449 | 0.284601931 |
| | (neoplastic transformation | | | | |
| | inhibitor) | | | | |
| 211648_at | Immunoglobulin heavy | 4.0944945 | 0.0003267 | 0.0258982 | 0.248883337 |
| | constant gamma 1 (G1m | | | | |
| | marker) /// Immunoglobulin | | | | |
| | heavy constant gamma 1 | | | | |
| | (G1m marker) | | | | |
| 215346_at | CD40 molecule, TNF | −4.092313 | 0.0003287 | 0.0258982 | 0.240544537 |
| | receptor superfamily member 5 | | | | |
| 203066_at | B cell RAG associated | 4.0920359 | 0.0003289 | 0.0258982 | 0.25311067 |
| | protein | | | | |
| 209537_at | exostoses (multiple)-like 2 | −4.077792 | 0.0003418 | 0.0266273 | 0.203023498 |
| 219159_s_at | SLAM family member 7 | −4.080316 | 0.0003394 | 0.0266273 | 0.222709614 |
| 216969_s_at | kinesin family member 22 | −4.07853 | 0.0003411 | 0.0266273 | 0.193535217 |
| 220387_s_at | HERV-H LTR-associating 3 | −4.073605 | 0.0003456 | 0.0268348 | 0.20425335 |
| 201779_s_at | ring finger protein 13 | 4.0679281 | 0.0003509 | 0.0269746 | 0.175231198 |
| 200093_s_at | histidine triad nucleotide | −4.070374 | 0.0003486 | 0.0269746 | 0.19881709 |
| | binding protein 1 /// histidine | | | | |
| | triad nucleotide binding | | | | |
| | protein 1 | | | | |
| 207489_at | hypothetical protein | 4.0678029 | 0.0003511 | 0.0269746 | 0.176979585 |
| | FLJ12331 | | | | |
| 218927_s_at | carbohydrate (chondroitin 4) | −4.064101 | 0.0003546 | 0.0270063 | 0.165293021 |
| | sulfotransferase 12 | | | | |
| 209426_s_at | alpha-methylacyl-CoA | −4.063537 | 0.0003551 | 0.0270063 | 0.171629407 |
| | racemase | | | | |
| 209755_at | nicotinamide nucleotide | 4.0653812 | 0.0003534 | 0.0270063 | 0.185557441 |
| | adenylyltransferase 2 | | | | |
| 204112_s_at | histamine N- | 4.0608161 | 0.0003577 | 0.0271119 | 0.171906051 |
| | methyltransferase | | | | |
| 207515_s_at | polymerase (RNA) I | −4.056624 | 0.0003618 | 0.027309 | 0.156342731 |
| | polypeptide C, 30 kDa | | | | |
| 213688_at | calmodulin 1 (phosphorylase | −4.055596 | 0.0003628 | 0.027309 | 0.142794242 |
| | kinase, delta) | | | | |
| 52285_f_at | centrosomal protein 76 kDa | −4.052297 | 0.000366 | 0.0274593 | 0.140854405 |
| 212813_at | junctional adhesion molecule 3 | 4.0470772 | 0.0003712 | 0.027471 | 0.138852612 |
| 209311_at | BCL2-like 2 | 4.0474557 | 0.0003708 | 0.027471 | 0.122347222 |
| 207525_s_at | GIPC PDZ domain | −4.049586 | 0.0003687 | 0.027471 | 0.126101887 |
| | containing family, member 1 | | | | |
| 212914_at | chromobox homolog 7 | −4.046636 | 0.0003716 | 0.027471 | 0.133686568 |
| 204327_s_at | zinc finger protein 202 | −4.04592 | 0.0003723 | 0.027471 | 0.14997588 |
| 208842_s_at | golgi reassembly stacking | −4.03977 | 0.0003785 | 0.0278362 | 0.111199985 |
| | protein 2, 55 kDa | | | | |
| 212064_x_at | MYC-associated zinc finger | −4.034684 | 0.0003837 | 0.0280338 | 0.09579129 |
| | protein (purine-binding | | | | |
| | transcription factor) | | | | |
| 212126_at | CDNA clone | −4.034963 | 0.0003834 | 0.0280338 | 0.108591742 |
| | IMAGE: 4842353 | | | | |
| 204676_at | chromosome 16 open reading | −4.027684 | 0.000391 | 0.0284269 | 0.086194587 |
| | frame 51 | | | | |
| 220631_at | O-sialoglycoprotein | −4.027059 | 0.0003916 | 0.0284269 | 0.090819836 |
| | endopeptidase-like 1 | | | | |
| 211033_s_at | peroxisomal biogenesis | −4.023656 | 0.0003952 | 0.028428 | 0.066578881 |
| | factor 7 /// peroxisomal | | | | |
| | biogenesis factor 7 | | | | |
| 215500_at | similar to RIKEN cDNA | 4.0244786 | 0.0003944 | 0.028428 | 0.083250954 |
| | 4933437K13 | | | | |
| 220418_at | intraflagellar transport 52 | −4.023419 | 0.0003955 | 0.028428 | 0.077567049 |
| | homolog (Chlamydomonas) | | | | |
| | /// ubiquitin associated and | | | | |
| | SH3 domain containing, A | | | | |
| 205928_at | zinc finger protein 443 | −4.01962 | 0.0003995 | 0.0286268 | 0.064655088 |
| 205831_at | CD2 molecule /// CD2 | −4.01401 | 0.0004056 | 0.0288751 | 0.05806134 |
| | molecule | | | | |
| 213315_x_at | chromosome X open reading | −4.014765 | 0.0004048 | 0.0288751 | 0.052859156 |
| | frame 40A | | | | |
| 209497_s_at | RNA binding motif protein | −4.012089 | 0.0004077 | 0.0289317 | 0.044594049 |
| | 4B | | | | |
| 216873_s_at | ATPase, Class I, type 8B, | −4.008088 | 0.0004121 | 0.0291509 | 0.043302344 |
| | member 2 | | | | |
| 205539_at | advillin | 4.0029224 | 0.0004178 | 0.0294638 | 0.031523988 |
| 216212_s_at | dyskeratosis congenita 1, | −3.994598 | 0.0004273 | 0.0297517 | 0.010360566 |
| | dyskerin | | | | |
| 219358_s_at | centaurin, alpha 2 | 3.9973106 | 0.0004242 | 0.0297517 | −0.008807446 |
| 218109_s_at | major facilitator superfamily | 3.9951674 | 0.0004266 | 0.0297517 | 0.001722182 |
| | domain containing 1 | | | | |
| 206296_x_at | mitogen-activated protein | −3.994793 | 0.000427 | 0.0297517 | −0.001302101 |
| | kinase kinase kinase kinase 1 | | | | |
| 217762_s_at | RAB31, member RAS | 3.9933458 | 0.0004287 | 0.0297587 | −0.004464026 |
| | oncogene family | | | | |
| 202846_s_at | phosphatidylinositol glycan | −3.990974 | 0.0004314 | 0.0298554 | −0.014964062 |
| | anchor biosynthesis, class C | | | | |
| 201002_s_at | ubiquitin-conjugating | −3.983794 | 0.0004398 | 0.0303374 | −0.016321939 |
| | enzyme E2 variant 1 /// | | | | |
| | ubiquitin-conjugating | | | | |
| | enzyme E2 variant 1 | | | | |
| 222115_x_at | cytokine-like nuclear factor | −3.982681 | 0.0004411 | 0.0303374 | −0.027366086 |
| | n-pac | | | | |
| 217391_x_at | — | 3.980629 | 0.0004435 | 0.0304107 | −0.037631483 |
| 41220_at | septin 9 | −3.979069 | 0.0004454 | 0.0304443 | −0.021180025 |
| 208970_s_at | SMAD, mothers against DPP | 3.9729852 | 0.0004527 | 0.0308495 | −0.054689218 |
| | homolog 3 (Drosophila) /// | | | | |
| | uroporphyrinogen | | | | |
| | decarboxylase | | | | |
| 214828_s_at | similar to CGI-96 | −3.967178 | 0.0004598 | 0.0312371 | −0.081021288 |
| 218091_at | HIV-1 Rev binding protein /// | 3.9632708 | 0.0004646 | 0.0312793 | −0.061456168 |
| | region containing | | | | |
| | hypothetical protein | | | | |
| | LOC285086; HIV-1 Rev | | | | |
| | binding protein | | | | |
| 210754_s_at | v-yes-1 Yamaguchi sarcoma | 3.963821 | 0.000464 | 0.0312793 | −0.084845468 |
| | viral related oncogene | | | | |
| | homolog | | | | |
| 219541_at | Lck interacting | −3.964165 | 0.0004635 | 0.0312793 | −0.080576597 |
| | transmembrane adaptor 1 | | | | |
| 210216_x_at | RAD1 homolog (S. pombe) | −3.959257 | 0.0004696 | 0.0313329 | −0.09463497 |
| 204393_s_at | acid phosphatase, prostate | 3.9605081 | 0.0004681 | 0.0313329 | −0.095402516 |
| 204442_x_at | latent transforming growth | −3.959723 | 0.0004691 | 0.0313329 | −0.099902777 |
| | factor beta binding protein 4 | | | | |
| 207583_at | ATP-binding cassette, sub- | −3.951658 | 0.0004793 | 0.0317859 | −0.114546044 |
| | family D (ALD), member 2 | | | | |
| 222126_at | HIV-1 Rev binding protein- | −3.952247 | 0.0004785 | 0.0317859 | −0.116910646 |
| | like | | | | |
| 218319_at | pellino homolog 1 | 3.9454397 | 0.0004873 | 0.0318448 | −0.136966432 |
| | (Drosophila) | | | | |
| 212589_at | Sterol carrier protein 2 | −3.948873 | 0.0004829 | 0.0318448 | −0.115344056 |
| 205707_at | interleukin 17 receptor A | 3.9465168 | 0.0004859 | 0.0318448 | −0.108165828 |
| 208185_x_at | — | 3.9484589 | 0.0004834 | 0.0318448 | −0.121055823 |
| 211950_at | zinc finger, UBR1 type 1 | 3.945484 | 0.0004873 | 0.0318448 | −0.11985623 |
| 210825_s_at | phosphatidylethanolamine | −3.94408 | 0.0004891 | 0.0318589 | −0.095532326 |
| | binding protein 1 | | | | |
| 201115_at | polymerase (DNA directed), | −3.941516 | 0.0004925 | 0.0318589 | −0.131001694 |
| | delta 2, regulatory subunit | | | | |
| | 50 kDa | | | | |
| 204960_at | protein tyrosine phosphatase, | −3.942208 | 0.0004916 | 0.0318589 | −0.113534623 |
| | receptor type, C-associated | | | | |
| | protein | | | | |
| 214326_x_at | jun D proto-oncogene | −3.940911 | 0.0004933 | 0.0318589 | −0.145668928 |
| 213042_s_at | ATPase, Ca++ transporting, | −3.936716 | 0.0004988 | 0.0320323 | −0.157152656 |
| | ubiquitous | | | | |
| 213006_at | CCAAT/enhancer binding | 3.9369089 | 0.0004986 | 0.0320323 | −0.130899193 |
| | protein (C/EBP), delta | | | | |
| 47530_at | chromosome 9 open reading | −3.934343 | 0.000502 | 0.0320813 | −0.158976205 |
| | frame 156 | | | | |
| 210512_s_at | vascular endothelial growth | 3.9339929 | 0.0005025 | 0.0320813 | −0.149581669 |
| | factor | | | | |
| 210422_x_at | solute carrier family 11 | 3.9294917 | 0.0005085 | 0.0323766 | −0.14310151 |
| | (proton-coupled divalent | | | | |
| | metal ion transporters), | | | | |
| | member 1 | | | | |
| 209286_at | CDC42 effector protein (Rho | 3.9265457 | 0.0005126 | 0.0325394 | −0.163682739 |
| | GTPase binding) 3 | | | | |
| 219068_x_at | ATPase family, AAA domain | −3.924785 | 0.000515 | 0.0325998 | −0.162373165 |
| | containing 3A | | | | |
| 200845_s_at | peroxiredoxin 6 | 3.9159249 | 0.0005273 | 0.0332856 | −0.20431199 |
| 203988_s_at | fucosyltransferase 8 (alpha | −3.912827 | 0.0005317 | 0.0334671 | −0.199059775 |
| | (1,6) fucosyltransferase) | | | | |
| 203683_s_at | vascular endothelial growth | −3.909987 | 0.0005357 | 0.0336267 | −0.195981447 |
| | factor B | | | | |
| 209354_at | tumor necrosis factor | −3.907402 | 0.0005394 | 0.0337643 | −0.21662648 |
| | receptor superfamily, | | | | |
| | member 14 (herpesvirus | | | | |
| | entry mediator) | | | | |
| 217635_s_at | polymerase (DNA directed), | −3.904003 | 0.0005443 | 0.0339764 | −0.232121769 |
| | gamma | | | | |
| 211339_s_at | IL2-inducible T-cell kinase | −3.901629 | 0.0005478 | 0.0340782 | −0.206828361 |
| 203482_at | chromosome 10 open reading | −3.900787 | 0.000549 | 0.0340782 | −0.242686951 |
| | frame 6 | | | | |
| 209156_s_at | collagen, type VI, alpha 2 | −3.897256 | 0.0005542 | 0.034305 | −0.251559356 |
| 213285_at | transmembrane protein 30B | −3.894317 | 0.0005586 | 0.0343839 | −0.238535141 |
| 217791_s_at | aldehyde dehydrogenase 18 | −3.894507 | 0.0005583 | 0.0343839 | −0.255323498 |
| | family, member A1 | | | | |
| 215754_at | scavenger receptor class B, | 3.8860504 | 0.000571 | 0.0350532 | −0.275987004 |
| | member 2 | | | | |
| 203047_at | serine/threonine kinase 10 | −3.883114 | 0.0005755 | 0.0352314 | −0.279759302 |
| 210116_at | SH2 domain protein 1A, | −3.880086 | 0.0005802 | 0.0354195 | −0.255134188 |
| | Duncan's disease | | | | |
| | (lymphoproliferative | | | | |
| | syndrome) | | | | |
| 214125_s_at | Neuron derived neurotrophic | −3.876791 | 0.0005853 | 0.0354462 | −0.289727884 |
| | factor | | | | |
| 212023_s_at | antigen identified by | −3.876825 | 0.0005852 | 0.0354462 | −0.286680948 |
| | monoclonal antibody Ki-67 | | | | |
| 209616_s_at | carboxylesterase 1 | 3.8767312 | 0.0005854 | 0.0354462 | −0.292084222 |
| | (monocyte/macrophage | | | | |
| | serine esterase 1) | | | | |
| 202073_at | optineurin | −3.875272 | 0.0005877 | 0.0354878 | −0.285403549 |
| 221731_x_at | chondroitin sulfate | 3.8609447 | 0.0006105 | 0.0365704 | −0.330808767 |
| | proteoglycan 2 (versican) | | | | |
| 212613_at | butyrophilin, subfamily 3, | −3.862766 | 0.0006076 | 0.0365704 | −0.330570695 |
| | member A2 | | | | |
| 214339_s_at | mitogen-activated protein | −3.861586 | 0.0006095 | 0.0365704 | −0.320110777 |
| | kinase kinase kinase kinase 1 | | | | |
| 202135_s_at | ARP1 actin-related protein 1 | −3.852512 | 0.0006244 | 0.0372003 | −0.359346618 |
| | homolog B, centractin beta | | | | |
| | (yeast) | | | | |
| 211152_s_at | HtrA serine peptidase 2 | −3.852533 | 0.0006243 | 0.0372003 | −0.35602825 |
| 207351_s_at | SH2 domain protein 2A | −3.851459 | 0.0006261 | 0.0372052 | −0.366540306 |
| AFFX- | signal transducer and | −3.850283 | 0.0006281 | 0.0372224 | −0.365134087 |
| HUMISGF3A/ | activator of transcription 1, | | | | |
| M97935_MA_at | 91 kDa | | | | |
| 209434_s_at | phosphoribosyl | −3.848344 | 0.0006313 | 0.0372721 | −0.369082504 |
| | pyrophosphate | | | | |
| | amidotransferase | | | | |
| 204173_at | myosin, light polypeptide 6B, | 3.8463196 | 0.0006347 | 0.0372721 | −0.363345319 |
| | alkali, smooth muscle and | | | | |
| | non-muscle | | | | |
| 201998_at | ST6 beta-galactosamide | −3.847461 | 0.0006328 | 0.0372721 | −0.337176917 |
| | alpha-2,6-sialyltranferase 1 | | | | |
| 216843_x_at | postmeiotic segregation | −3.845803 | 0.0006356 | 0.0372721 | −0.367585906 |
| | increased 2-like 1 | | | | |
| 202625_at | v-yes-1 Yamaguchi sarcoma | 3.8445294 | 0.0006378 | 0.0372809 | −0.379578446 |
| | viral related oncogene | | | | |
| | homolog /// v-yes-1 | | | | |
| | Yamaguchi sarcoma viral | | | | |
| | related oncogene homolog | | | | |
| 202497_x_at | solute carrier family 2 | 3.8427574 | 0.0006408 | 0.0372809 | −0.33433002 |
| | (facilitated glucose | | | | |
| | transporter), member 3 | | | | |
| 206714_at | arachidonate 15- | 3.8430539 | 0.0006403 | 0.0372809 | −0.362033812 |
| | lipoxygenase, type B | | | | |
| 204128_s_at | replication factor C (activator | −3.841715 | 0.0006426 | 0.037287 | −0.372724167 |
| | 1) 3, 38 kDa | | | | |
| 215796_at | T-cell receptor active alpha- | −3.838028 | 0.0006489 | 0.0374114 | −0.400204002 |
| | chain V-region (V-J-C) | | | | |
| | mRNA, partial cds, clone | | | | |
| | AG212 | | | | |
| 204572_s_at | protein (peptidylprolyl | −3.838347 | 0.0006483 | 0.0374114 | −0.401933215 |
| | cis/trans isomerase) NIMA- | | | | |
| | interacting, 4 (parvulin) | | | | |
| 214908_s_at | transformation/transcription | −3.836564 | 0.0006514 | 0.0374114 | −0.391154844 |
| | domain-associated protein | | | | |
| 217821_s_at | WW domain binding protein | −3.837195 | 0.0006503 | 0.0374114 | −0.404526892 |
| | 11 | | | | |
| 211794_at | FYN binding protein (FYB- | 3.8340346 | 0.0006558 | 0.0375384 | −0.399834274 |
| | 120/130) | | | | |
| 214439_x_at | bridging integrator 1 | −3.833354 | 0.000657 | 0.0375384 | −0.397155838 |
| 213353_at | ATP-binding cassette, sub- | −3.829942 | 0.000663 | 0.0377835 | −0.356092576 |
| | family A (ABC1), member 5 | | | | |
| 214530_x_at | erythrocyte membrane | −3.828885 | 0.0006649 | 0.0377931 | −0.41604328 |
| | protein band 4.1 | | | | |
| | (elliptocytosis 1, RH-linked) | | | | |
| 204985_s_at | trafficking protein particle | −3.826231 | 0.0006696 | 0.0379636 | −0.385827346 |
| | complex 6A | | | | |
| 201951_at | activated leukocyte cell | 3.8224735 | 0.0006763 | 0.0382009 | −0.435681891 |
| | adhesion molecule | | | | |
| 212263_at | quaking homolog, KH | 3.8210222 | 0.0006789 | 0.0382009 | −0.441662788 |
| | domain RNA binding | | | | |
| | (mouse) | | | | |
| 217969_at | chromosome 11 open reading | −3.82165 | 0.0006778 | 0.0382009 | −0.424792327 |
| | frame2 | | | | |
| 208808_s_at | high-mobility group box 2 | 3.8192885 | 0.000682 | 0.0382805 | −0.443258871 |
| 201411_s_at | pleckstrin homology domain | 3.8121453 | 0.0006951 | 0.0387025 | −0.444049065 |
| | containing, family B | | | | |
| | (evectins) member 2 | | | | |
| 211914_x_at | neurofibromin 1 | 3.8114314 | 0.0006964 | 0.0387025 | −0.461956408 |
| | (neurofibromatosis, von | | | | |
| | Recklinghausen disease, | | | | |
| | Watson disease) /// | | | | |
| | neurofibromin 1 | | | | |
| | (neurofibromatosis, von | | | | |
| | Recklinghausen disease, | | | | |
| | Watson disease) | | | | |
| 220001_at | peptidyl arginine deiminase, | 3.8106552 | 0.0006978 | 0.0387025 | −0.443505201 |
| | type IV | | | | |
| 220155_s_at | bromodomain containing 9 | −3.809596 | 0.0006998 | 0.0387025 | −0.456574514 |
| 202624_s_at | calcineurin binding protein 1 | −3.81021 | 0.0006987 | 0.0387025 | −0.469611909 |
| 202150_s_at | neural precursor cell | −3.809509 | 0.0007 | 0.0387025 | −0.44189728 |
| | expressed, developmentally | | | | |
| | down-regulated 9 | | | | |
| 217381_s_at | T cell receptor gamma | −3.80848 | 0.0007019 | 0.0387122 | −0.46847961 |
| | variable 5 /// hypothetical | | | | |
| | protein LOC648852 | | | | |
| 212723_at | phosphatidylserine receptor | 3.8062119 | 0.0007061 | 0.0388498 | −0.479127975 |
| 219383_at | hypothetical protein | −3.796882 | 0.0007238 | 0.0389768 | −0.500819992 |
| | FLJ14213 | | | | |
| 201369_s_at | zinc finger protein 36, C3H | −3.796696 | 0.0007242 | 0.0389768 | −0.490238808 |
| | type-like 2 | | | | |
| 217985_s_at | bromodomain adjacent to | 3.8035661 | 0.0007111 | 0.0389768 | −0.487528654 |
| | zinc finger domain, 1A | | | | |
| 201661_s_at | acyl-CoA synthetase long- | 3.8021203 | 0.0007138 | 0.0389768 | −0.491456065 |
| | chain family member 3 | | | | |
| 201601_x_at | interferon induced | −3.796901 | 0.0007238 | 0.0389768 | −0.484034883 |
| | transmembrane protein 1 (9-27) | | | | |
| 220034_at | interleukin-1 receptor- | 3.7972108 | 0.0007232 | 0.0389768 | −0.498328947 |
| | associated kinase 3 | | | | |
| 202739_s_at | phosphorylase kinase, beta | 3.7982853 | 0.0007211 | 0.0389768 | −0.490460156 |
| 212094_at | paternally expressed 10 | −3.80208 | 0.0007139 | 0.0389768 | −0.478413746 |
| 218217_at | serine carboxypeptidase 1 | 3.8009907 | 0.000716 | 0.0389768 | −0.477198569 |
| 214390_s_at | branched chain | 3.7890009 | 0.0007391 | 0.0396844 | −0.484386105 |
| | aminotransferase 1, cytosolic | | | | |
| 201188_s_at | inositol 1,4,5-triphosphate | −3.785887 | 0.0007452 | 0.0399171 | −0.51545757 |
| | receptor, type 3 | | | | |
| 208831_x_at | suppressor of Ty 6 homolog | −3.780085 | 0.0007568 | 0.0404383 | −0.502670551 |
| | (S. cerevisiae) | | | | |
| 202208_s_at | ADP-ribosylation factor-like | −3.778858 | 0.0007592 | 0.040473 | −0.504582473 |
| | 4C | | | | |
| 222148_s_at | ras homolog gene family, | 3.7750684 | 0.0007669 | 0.0407838 | −0.538642793 |
| | member T1 | | | | |
| 204891_s_at | lymphocyte-specific protein | −3.771732 | 0.0007737 | 0.0410478 | −0.526449904 |
| | tyrosine kinase | | | | |
| 217947_at | CKLF-like MARVEL | 3.7649198 | 0.0007878 | 0.0416956 | −0.565292502 |
| | transmembrane domain | | | | |
| | containing 6 | | | | |
| 209565_at | ring finger protein 113A | −3.760506 | 0.000797 | 0.041887 | −0.584342956 |
| 201921_at | guanine nucleotide binding | 3.7608345 | 0.0007963 | 0.041887 | −0.575062313 |
| | protein (G protein), gamma | | | | |
| | 10 /// hypothetical protein | | | | |
| | LOC552891 /// GNG10 | | | | |
| | pseudogene | | | | |
| 211856_x_at | CD28 molecule | −3.761964 | 0.000794 | 0.041887 | −0.589027345 |
| 206039_at | RAB33A, member RAS | −3.759074 | 0.0008001 | 0.0419472 | −0.585075087 |
| | oncogene family | | | | |
| 202192_s_at | growth arrest-specific 7 | 3.7575204 | 0.0008033 | 0.0420211 | −0.568609359 |
| 217421_at | piwi-like 2 (Drosophila) | 3.7536741 | 0.0008116 | 0.042056 | −0.599493208 |
| 217473_x_at | solute carrier family 11 | 3.7551697 | 0.0008084 | 0.042056 | −0.561255747 |
| | (proton-coupled divalent | | | | |
| | metal ion transporters), | | | | |
| | member 1 | | | | |
| 219125_s_at | recombination activating | −3.755061 | 0.0008086 | 0.042056 | −0.606773906 |
| | gene 1 activating protein 1 | | | | |
| 202435_s_at | cytochrome P450, family 1, | 3.7544251 | 0.00081 | 0.042056 | −0.598408605 |
| | subfamily B, polypeptide 1 | | | | |
| 217328_at | Protease, serine, 1 (trypsin 1) | −3.752362 | 0.0008144 | 0.0421044 | −0.586497887 |
| 211067_s_at | growth arrest-specific 7 /// | 3.7500978 | 0.0008193 | 0.0421616 | −0.592937929 |
| | growth arrest-specific 7 | | | | |
| 212772_s_at | ATP-binding cassette, sub- | −3.75094 | 0.0008175 | 0.0421616 | −0.597470148 |
| | family A (ABC1), member 2 | | | | |
| 201971_s_at | ATPase, H+ transporting, | 3.7484271 | 0.0008229 | 0.0422507 | −0.582559368 |
| | lysosomal 70 kDa, V1 subunit A | | | | |
| 203297_s_at | Jumonji, AT rich interactive | 3.7466006 | 0.0008269 | 0.0423577 | −0.618887474 |
| | domain 2 | | | | |
| 212082_s_at | myosin, light polypeptide 6, | 3.7413518 | 0.0008384 | 0.0426702 | −0.638012205 |
| | alkali, smooth muscle and | | | | |
| | non-muscle | | | | |
| 212316_at | nucleoporin 210 kDa | −3.742284 | 0.0008364 | 0.0426702 | −0.625770215 |
| 202039_at | TGFB1-induced anti- | −3.741221 | 0.0008387 | 0.0426702 | −0.620447968 |
| | apoptotic factor 1 /// myosin | | | | |
| | XVIIIA | | | | |
| 213198_at | activin A receptor, type IB | 3.7369379 | 0.0008483 | 0.042773 | −0.62607927 |
| 211316_x_at | CASP8 and FADD-like | 3.7379973 | 0.0008459 | 0.042773 | −0.646108656 |
| | apoptosis regulator | | | | |
| 215313_x_at | major histocompatibility | −3.736735 | 0.0008487 | 0.042773 | −0.636234404 |
| | complex, class I, A | | | | |
| 209446_s_at | chromosome 7 open reading | 3.7360149 | 0.0008504 | 0.042773 | −0.631120386 |
| | frame 44 | | | | |
| 218150_at | ADP-ribosylation factor-like | 3.7364786 | 0.0008493 | 0.042773 | −0.638696549 |
| | 5A | | | | |
| 204860_s_at | baculoviral IAP repeat- | 3.7317847 | 0.0008599 | 0.0431564 | −0.646735279 |
| | containing 1 /// similar to | | | | |
| | Baculoviral IAP repeat- | | | | |
| | containing protein 1 | | | | |
| | (Neuronal apoptosis | | | | |
| | inhibitory protein) /// similar | | | | |
| | to Baculoviral IAP repeat- | | | | |
| | containing protein 1 | | | | |
| | (Neuronal apoptosis | | | | |
| | inhibitory protein) | | | | |
| 204619_s_at | chondroitin sulfate | 3.72927 | 0.0008656 | 0.0433464 | −0.656075159 |
| | proteoglycan 2 (versican) | | | | |
| 201677_at | Chromosome 3 open reading | −3.728121 | 0.0008683 | 0.0433806 | −0.673304821 |
| | frame 37 | | | | |
| 202932_at | v-yes-1 Yamaguchi sarcoma | −3.726647 | 0.0008717 | 0.0433811 | −0.664383959 |
| | viral oncogene homolog 1 | | | | |
| 211841_s_at | tumor necrosis factor | −3.726423 | 0.0008722 | 0.0433811 | −0.675139954 |
| | receptor superfamily, | | | | |
| | member 25 | | | | |
| 209173_at | anterior gradient 2 homolog | 3.725149 | 0.0008751 | 0.0434304 | −0.65418982 |
| | (Xenopus laevis) | | | | |
| 202570_s_at | discs, large (Drosophila) | 3.7225989 | 0.000881 | 0.0436266 | −0.662592397 |
| | homolog-associated protein 4 | | | | |
| 211594_s_at | mitochondrial ribosomal | −3.71579 | 0.000897 | 0.0443194 | −0.689958857 |
| | protein L9 /// mitochondrial | | | | |
| | ribosomal protein L9 | | | | |
| 218084_x_at | FXYD domain containing | −3.711432 | 0.0009074 | 0.0444954 | −0.691534168 |
| | ion transport regulator 5 | | | | |
| 207590_s_at | centromere protein I | −3.710838 | 0.0009088 | 0.0444954 | −0.686579571 |
| 203293_s_at | lectin, mannose-binding, 1 | −3.71011 | 0.0009106 | 0.0444954 | −0.678123352 |
| 209026_x_at | tubulin, beta | −3.711747 | 0.0009066 | 0.0444954 | −0.695581501 |
| 205541_s_at | G1 to S phase transition 2 /// | −3.711401 | 0.0009075 | 0.0444954 | −0.687044345 |
| | G1 to S phase transition 2 | | | | |
| 205652_s_at | tubulin tyrosine ligase-like | −3.701274 | 0.000932 | 0.0449283 | −0.69485195 |
| | family, member 1 | | | | |
| 208199_s_at | zinc finger protein 161 | −3.701156 | 0.0009323 | 0.0449283 | −0.739570191 |
| | homolog (mouse) | | | | |
| 214452_at | branched chain | 3.7038226 | 0.0009258 | 0.0449283 | −0.662431636 |
| | aminotransferase 1, cytosolic | | | | |
| 205050_s_at | mitogen-activated protein | −3.705111 | 0.0009226 | 0.0449283 | −0.726134729 |
| | kinase 8 interacting protein 2 | | | | |
| 217270_s_at | dual-specificity tyrosine-(Y)- | −3.702839 | 0.0009282 | 0.0449283 | −0.713090932 |
| | phosphorylation regulated | | | | |
| | kinase 1B | | | | |
| 201093_x_at | succinate dehydrogenase | −3.700663 | 0.0009335 | 0.0449283 | −0.728740051 |
| | complex, subunit A, | | | | |
| | flavoprotein (Fp) | | | | |
| 212575_at | chromosome 19 open reading | −3.702288 | 0.0009295 | 0.0449283 | −0.721469223 |
| | frame 6 | | | | |
| 217322_x_at | — | −3.692308 | 0.0009543 | 0.0458297 | −0.752867335 |
| 212934_at | hypothetical protein | 3.6900358 | 0.00096 | 0.0460058 | −0.739440735 |
| | LOC137886 | | | | |
| 210095_s_at | insulin-like growth factor | −3.684731 | 0.0009736 | 0.0461856 | −0.76631001 |
| | binding protein 3 | | | | |
| 203489_at | CD27-binding (Siva) protein | −3.684495 | 0.0009742 | 0.0461856 | −0.771305084 |
| 202727_s_at | interferon gamma receptor 1 | 3.6870365 | 0.0009677 | 0.0461856 | −0.757780699 |
| 202556_s_at | microspherule protein 1 | −3.68587 | 0.0009706 | 0.0461856 | −0.768725021 |
| 202725_at | polymerase (RNA) II (DNA | −3.684549 | 0.000974 | 0.0461856 | −0.763280163 |
| | directed) polypeptide A, | | | | |
| | 220 kDa | | | | |
| 216885_s_at | WD repeat domain 42A | −3.681171 | 0.0009827 | 0.0464929 | −0.778461472 |
| 205726_at | diaphanous homolog 2 | 3.6750662 | 0.0009987 | 0.047146 | −0.781925661 |
| | (Drosophila) | | | | |
| 201220_x_at | C-terminal binding protein 2 | 3.6729085 | 0.0010043 | 0.0473143 | −0.784485981 |
| 200839_s_at | cathepsin B | 3.6698829 | 0.0010124 | 0.0475919 | −0.767004526 |
| 209575_at | interleukin 10 receptor, beta | 3.6685673 | 0.0010159 | 0.0476564 | −0.809494026 |
| 218908_at | alveolar soft part sarcoma | −3.660231 | 0.0010384 | 0.0486107 | −0.836123189 |
| | chromosome region, | | | | |
| | candidate 1 | | | | |
| 222173_s_at | TBC1 domain family, | 3.6557402 | 0.0010507 | 0.0490849 | −0.808895577 |
| | member 2 | | | | |
| 219843_at | intracisternal A particle- | −3.65418 | 0.0010551 | 0.0491835 | −0.84256988 |
| | promoted polypeptide | | | | |
| 222043_at | clusterin | 3.6521802 | 0.0010606 | 0.0492944 | −0.85080773 |
| 216525_x_at | postmeiotic segregation | −3.650357 | 0.0010657 | 0.0492944 | −0.857002971 |
| | increased 2-like 3 | | | | |
| 202800_at | solute carrier family 1 (glial | 3.6501531 | 0.0010663 | 0.0492944 | −0.826807271 |
| | high affinity glutamate | | | | |
| | transporter), member 3 | | | | |
| 211913_s_at | c-mer proto-oncogene | 3.6508587 | 0.0010643 | 0.0492944 | −0.833924169 |
| | tyrosine kinase /// c-mer | | | | |
| | proto-oncogene tyrosine | | | | |
| | kinase | | | | |
| 49306_at | Ras association | 3.6444617 | 0.0010823 | 0.0499334 | −0.86192015 |
| | (RalGDS/AF-6) domain | | | | |
| | family 4 | | | | |

\hat{ }Probe set ID number is the Affymetrix ID number on the HU133A array.
* Moderated t-statistic. This has the same interpretation as an ordinary t-statistic except that the standard errors have been moderated across genes, i.e., shrunk towards a common value, using a simple Bayesian model. Positive t-statistic indicates that the gene is upregulated following hemorrhagic stroke. Negative t-statistic indicates that the gene is downregulated following hemorrhagic stroke.
^{$}P-value uncorrected p value
^{#}Adjusted p-value is the corrected value after correction for multitple comparisons using the FDR method.
^{@}The B-statistic (lods or B) is the log-odds that the gene is differentially expressed.

After multiple comparison correction (MCC) using Holm correction, 27 gene probes, corresponding to 25 genes were found to be significantly different (Table 7). As shown in Table 7, several genes were upregulated (positive T-statistic, such as a value that is at least 6) or downregulated (negative t-statistic, such as a value that is less than −6) following a hemorrhagic stroke.

TABLE 7
Hemorrhagic stroke related-genes using Holm correction and
comparison to IS subjects.

| Probe Set | | t- | | Adjusted | |
|---|---|---|---|---|---|
| ID\hat{ } | Gene Name | statistic* | P Value^{$} | P Value^{#} | B^{@} |
| 205257_s_at | amphiphysin (Stiff-Man | 14.975963 | 6.99E−015 | 1.56E−010 | 20.7629274 |
| | syndrome with breast cancer | | | | |
| | 128 kDa autoantigen) | | | | |
| 211372_s_at | interleukin 1 receptor, type | 10.712554 | 2.10E−011 | 4.68E−007 | 14.82446351 |
| | II | | | | |
| 216233_at | CD163 molecule | 9.737206 | 1.75E−010 | 3.91E−006 | 12.88835863 |
| 221011_s_at | hypothetical protein | −8.515744 | 2.98E−009 | 6.64E−005 | 10.70166657 |
| | DKFZp566J091 /// | | | | |
| | hypothetical protein | | | | |
| | DKFZp566J091 /// similar | | | | |
| | to hypothetical protein | | | | |
| | DKFZp566J091 /// similar | | | | |
| | to hypothetical protein | | | | |
| | DKFZp566J091 | | | | |
| 214535_s_at | ADAM metallopeptidase | 8.4530582 | 3.46E−009 | 7.72E−005 | 10.50746674 |
| | with thrombospondin type 1 | | | | |
| | motif, 2 | | | | |
| 206028_s_at | c-mer proto-oncogene | 8.2851019 | 5.20E−009 | 0.0001159 | 10.25765534 |
| | tyrosine kinase | | | | |
| 205403_at | interleukin 1 receptor, type | 7.6873216 | 2.27E−008 | 0.0005067 | 9.05172448 |
| | II | | | | |
| 218494_s_at | SLC2A4 regulator | −7.333288 | 5.57E−008 | 0.0012403 | 8.265256103 |
| 205396_at | SMAD, mothers against | −7.227747 | 7.29E−008 | 0.0016244 | 8.021975608 |
| | DPP homolog 3 | | | | |
| | (Drosophila) | | | | |
| 205484_at | signaling threshold | −7.018339 | 1.25E−007 | 0.0027856 | 7.53111354 |
| | regulating transmembrane | | | | |
| | adaptor 1 | | | | |
| 204116_at | interleukin 2 receptor, | −7.015854 | 1.26E−007 | 0.0028034 | 7.521746309 |
| | gamma (severe combined | | | | |
| | immunodeficiency) | | | | |
| 218615_s_at | transmembrane protein 39A | 6.9645637 | 1.44E−007 | 0.0032017 | 7.302004604 |
| 218813_s_at | SH3-domain GRB2-like | −6.94035 | 1.53E−007 | 0.0034093 | 7.368885163 |
| | endophilin B2 | | | | |
| 209671_x_at | T cell receptor alpha locus | −6.753733 | 2.49E−007 | 0.0055453 | 6.8625403 |
| | /// T cell receptor alpha | | | | |
| | locus /// T cell receptor | | | | |
| | alpha constant /// T cell | | | | |
| | receptor alpha constant | | | | |
| 213805_at | abhydrolase domain | 6.71886 | 2.73E−007 | 0.0060754 | 6.807097433 |
| | containing 5 | | | | |
| 208611_s_at | spectrin, alpha, non- | −6.67564 | 3.06E−007 | 0.0068044 | 6.685920872 |
| | erythrocytic 1 (alpha-fodrin) | | | | |
| 208602_x_at | CD6 molecule | −6.604865 | 3.68E−007 | 0.0081957 | 6.552492682 |
| 221688_s_at | IMP3, U3 small nucleolar | −6.584643 | 3.88E−007 | 0.0086437 | 6.468878576 |
| | ribonucleoprotein, homolog | | | | |
| | (yeast) | | | | |
| 202499_s_at | solute carrier family 2 | 6.4812483 | 5.10E−007 | 0.0113561 | 6.188901235 |
| | (facilitated glucose | | | | |
| | transporter), member 3 | | | | |
| 213275_x_at | cathepsin B | 6.4800111 | 5.12E−007 | 0.0113928 | 6.240830095 |
| 218866_s_at | polymerase (RNA) III | −6.410061 | 6.16E−007 | 0.0137114 | 5.994655791 |
| | (DNA directed) polypeptide | | | | |
| | K, 12.3 kDa | | | | |
| 211734_s_at | Fc fragment of IgE, high | −6.35615 | 7.11E−007 | 0.0158204 | 5.759237554 |
| | affinity I, receptor for; alpha | | | | |
| | polypeptide | | | | |
| 215049_x_at | CD163 molecule | 6.3394666 | 7.43E−007 | 0.0165371 | 5.872838463 |
| 211893_x_at | CD6 molecule | −6.245476 | 9.54E−007 | 0.0212397 | 5.68152223 |
| 218805_at | GTPase, IMAP family | −6.236583 | 9.77E−007 | 0.0217486 | 5.638421367 |
| | member 5\ | | | | |
| 203392_s_at | C-terminal binding protein 1 | −6.037307 | 1.67E−006 | 0.037074 | 5.141550008 |
| 202191_s_at | growth arrest-specific 7 | 6.0036117 | 1.82E−006 | 0.0405844 | 5.046148265 |

\hat{ }Probe set ID number is the Affymetrix ID number on the HU133A array.
*Moderated t-statistic. This has the same interpretation as an ordinary t-statistic except that the standard errors have been moderated across genes, i.e., shrunk towards a common value, using a simple Bayesian model. Positive t-statistic indicates that the gene is upregulated following hemorrhagic stroke. Negative t-statistic indicates that the gene is downregulated following hemorrhagic stroke.
^{$}P-value uncorrected p value
^{#}Adjusted p-value is the corrected value after correction for multitple comparisons using the Holm method.
^{@}The B-statistic (lods or B) is the log-odds that the gene is differentially expressed.

After multiple comparison correction (MCC) using PAM correction (shrunken centroid algorithm), 380 gene probes, corresponding to 316 genes were found to be significantly different (Table 8). The two numeric values for each gene shown in Table 8 were generated from the shrunken centroid algorithm technique, and provide an indication of the strength of each gene for the classification of hemorrhagic stroke/ischemic stroke in the dataset, and therefore identifies genes (or proteins) which distinguish best between the disease and control conditions. As shown in Table 8, several genes provide a significant ability to differentiate control from hemorrhagic stroke subjects. The data shown in Table 8 was obtained using the subjects described in Example 1, as well as an additional subject who had an ICH as the result of trauma, not stroke.

TABLE 8
Hemorrhagic stroke related-genes using PAM correction and comparison to
IS subjects.

| Probe Set ID\hat{ } | Gene Name | 1-score | 2-score |
|---|---|---|---|
| 205403_at | interleukin 1 receptor, type II | −0.3392 | 0.7161 |
| 211372_s_at | interleukin 1 receptor, type II | −0.3105 | 0.6554 |
| 211893_x_at | CD6 antigen | 0.2733 | −0.577 |
| 206025_s_at | tumor necrosis factor, alpha-induced protein 6 | −0.2433 | 0.5137 |
| 205456_at | CD3E antigen, epsilon polypeptide (TiT3 complex) | 0.213 | −0.4496 |
| 211734_s_at | Fc fragment of IgE, high affinity I, receptor for; alpha | 0.2116 | −0.4468 |
| | polypeptide | | |
| 204116_at | interleukin 2 receptor, gamma (severe combined | 0.2051 | −0.4329 |
| | immunodeficiency) | | |
| 221011_s_at | likely ortholog of mouse limb-bud and heart gene | 0.205 | −0.4327 |
| 218494_s_at | SLC2A4 regulator | 0.2049 | −0.4325 |
| 218813_s_at | SH3-domain GRB2-like endophilin B2 | 0.2017 | −0.4257 |
| 212259_s_at | pre-B-cell leukemia transcription factor interacting | 0.1991 | −0.4202 |
| | protein 1 | | |
| 214551_s_at | CD7 antigen (p41) | 0.1935 | −0.4085 |
| 205257_s_at | amphiphysin (Stiff-Man syndrome with breast cancer | −0.1879 | 0.3968 |
| | 128 kDa autoantigen) | | |
| 202464_s_at | 6-phosphofructo-2-kinase/fructose-2,6-biphosphatase 3 | −0.1786 | 0.3771 |
| 208602_x_at | CD6 antigen | 0.1742 | −0.3678 |
| 206026_s_at | tumor necrosis factor, alpha-induced protein 6 | −0.1686 | 0.3559 |
| 218805_at | GTPase, IMAP family member 5 | 0.1653 | −0.3489 |
| 214049_x_at | CD7 antigen (p41) | 0.1649 | −0.348 |
| 202499_s_at | solute carrier family 2 (facilitated glucose | −0.1638 | 0.3459 |
| | transporter), member 3 | | |
| 205027_s_at | mitogen-activated protein kinase kinase kinase 8 | −0.16 | 0.3379 |
| 202478_at | tribbles homolog 2 (Drosophila) | 0.158 | −0.3335 |
| 210972_x_at | T cell receptor alpha locus | 0.1575 | −0.3325 |
| 64064_at | GTPase, IMAP family member 5 | 0.1517 | −0.3202 |
| 221602_s_at | Fas apoptotic inhibitory molecule 3 | 0.1492 | −0.315 |
| 213817_at | MRNA; cDNA DKLFZp586B0220 (from clone | −0.146 | 0.3082 |
| | DKFZp586B0220) | | |
| 206028_s_at | c-mer proto-oncogene tyrosine kinase | −0.1443 | 0.3047 |
| 218871_x_at | chondroitin sulfate GalNAcT-2 | −0.1421 | 0.3001 |
| 201110_s_at | thrombospondin 1 | −0.1391 | 0.2937 |
| 209671_x_at | T cell receptor alpha locus | 0.1349 | −0.2848 |
| 201963_at | acyl-CoA synthetase long-chain family member 1 | −0.1311 | 0.2767 |
| 206100_at | carboxypeptidase M | −0.126 | 0.2659 |
| 211902_x_at | T cell receptor alpha locus | 0.1252 | −0.2643 |
| 213275_x_at | cathepsin B | −0.1234 | 0.2604 |
| 215049_x_at | CD163 antigen | −0.123 | 0.2596 |
| 208611_s_at | spectrin, alpha, non-erythrocytic 1 (alpha-fodrin) | 0.1209 | −0.2552 |
| 200953_s_at | cyclin D2 | 0.1209 | −0.2551 |
| 201109_s_at | thrombospondin 1 | −0.1208 | 0.255 |
| 211900_x_at | CD6 antigen | 0.1199 | −0.2532 |
| 206674_at | fms-related tyrosine kinase 3 | −0.1199 | 0.2531 |
| 202437_s_at | cytochrome P450, family 1, subfamily B, polypeptide 1 | −0.1181 | 0.2492 |
| 204861_s_at | baculoviral IAP repeat-containing 1 | −0.118 | 0.2492 |
| 214535_s_at | ADAM metallopeptidase with thrombospondin type 1 | −0.1177 | 0.2485 |
| | motif, 2 | | |
| 207838_x_at | pre-B-cell leukemia transcription factor interacting | 0.1151 | −0.243 |
| | protein 1 | | |
| 220684_at | T-box 21 | 0.1145 | −0.2418 |
| 37652_at | calcineurin binding protein 1 | 0.1123 | −0.2371 |
| 206207_at | Charcot-Leyden crystal protein | 0.1121 | −0.2368 |
| 204787_at | V-set and immunoglobulin domain containing 4 | −0.1117 | 0.2358 |
| 205484_at | signaling threshold regulating transmembrane adaptor 1 | 0.1111 | −0.2346 |
| 220034_at | interleukin-1 receptor-associated kinase 3 | −0.1111 | 0.2345 |
| 204446_s_at | arachidonate 5-lipoxygenase | −0.1101 | 0.2324 |
| 210146_x_at | leukocyte immunoglobulin-like receptor, subfamily B | −0.1092 | 0.2305 |
| | (with TM and ITIM domains), member 2 | | |
| 206170_at | adrenergic, beta-2-, receptor, surface | 0.1076 | −0.2272 |
| 208304_at | chemokine (C-C motif) receptor 3 | 0.107 | −0.226 |
| 201921_at | guanine nucleotide binding protein (G protein), | −0.1043 | 0.2202 |
| | gamma 10 | | |
| 202436_s_at | cytochrome P450, family 1, subfamily B, polypeptide 1 | −0.1034 | 0.2184 |
| 220088_at | complement component 5 receptor 1 (C5a ligand) | −0.103 | 0.2174 |
| 212400_at | chromosome 9 open reading frame 132 | 0.1029 | −0.2171 |
| 202523_s_at | sparc/osteonectin, cwcv and kazal-like domains | 0.1021 | −0.2156 |
| | proteoglycan (testican) 2 | | |
| 218092_s_at | HIV-1 Rev binding protein | −0.1015 | 0.2143 |
| 203140_at | B-cell CLL/lymphoma 6 (zinc finger protein 51) | −0.101 | 0.2132 |
| 213193_x_at | T cell receptor beta variable 19 | 0.1005 | −0.2122 |
| 218600_at | hypothetical protein MGC10986 | 0.1 | −0.2112 |
| 209409_at | growth factor receptor-bound protein 10 | −0.0989 | 0.2088 |
| 217739_s_at | pre-B-cell colony enhancing factor 1 | −0.0989 | 0.2087 |
| 210915_x_at | T cell receptor beta variable 19 | 0.0984 | −0.2077 |
| 210202_s_at | bridging integrator 1 | 0.0982 | −0.2072 |
| 206980_s_at | fms-related tyrosine kinase 3 ligand | 0.0975 | −0.2059 |
| 204900_x_at | sin3-associated polypeptide, 30 kDa | −0.097 | 0.2047 |
| 217738_at | pre-B-cell colony enhancing factor 1 | −0.0966 | 0.204 |
| 202524_s_at | sparc/osteonectin, cwcv and kazal-like domains | 0.0935 | −0.1974 |
| | proteoglycan (testican) 2 | | |
| 213539_at | CD3D antigen, delta polypeptide (TiT3 complex) | 0.0926 | −0.1955 |
| 213805_at | abhydrolase domain containing 5 | −0.0923 | 0.1948 |
| 213229_at | Dicer1, Dcr-1 homolog (Drosophila) | −0.0919 | 0.1939 |
| 203645_s_at | CD163 antigen | −0.0914 | 0.1929 |
| 202479_s_at | tribbles homolog 2 (Drosophila) | 0.091 | −0.1922 |
| 200998_s_at | cytoskeleton-associated protein 4 | −0.0909 | 0.1919 |
| 201189_s_at | inositol 1,4,5-triphosphate receptor, type 3 | 0.0904 | −0.1909 |
| 217985_s_at | bromodomain adjacent to zinc finger domain, 1A | −0.0903 | 0.1906 |
| 204070_at | retinoic acid receptor responder (tazarotene induced) 3 | 0.0897 | −0.1894 |
| 217762_s_at | RAB31, member RAS oncogene family | −0.0892 | 0.1883 |
| 201785_at | ribonuclease, RNase A family, 1 (pancreatic) | −0.0892 | 0.1882 |
| 201661_s_at | acyl-CoA synthetase long-chain family member 3 | −0.089 | 0.188 |
| 218689_at | Fanconi anemia, complementation group F | 0.0885 | −0.1868 |
| 207521_s_at | ATPase, Ca++ transporting, ubiquitous | 0.0878 | −0.1854 |
| 219157_at | kelch-like 2, Mayven (Drosophila) | −0.0878 | 0.1853 |
| 211796_s_at | T cell receptor beta variable 21-1 | 0.0878 | −0.1853 |
| 208829_at | TAP binding protein (tapasin) | 0.0873 | −0.1844 |
| 204362_at | src family associated phosphoprotein 2 | −0.0871 | 0.1839 |
| 202912_at | adrenomedullin | −0.0866 | 0.1828 |
| 205624_at | carboxypeptidase A3 (mast cell) | 0.0862 | −0.1819 |
| 206697_s_at | haptoglobin | −0.0856 | 0.1807 |
| 204891_s_at | lymphocyte-specific protein tyrosine kinase | 0.0854 | −0.1802 |
| 205254_x_at | transcription factor 7 (T-cell specific, HMG-box) | 0.0852 | −0.1799 |
| 219315_s_at | chromosome 16 open reading frame 30 | 0.0852 | −0.1799 |
| 209710_at | GATA binding protein 2 | 0.0851 | −0.1796 |
| 213261_at | lupus brain antigen 1 | 0.0843 | −0.178 |
| 214326_x_at | jun Dproto-oncogene | 0.082 | −0.1732 |
| 209570_s_at | DNA segment on chromosome 4 (unique) 234 | 0.0813 | −0.1717 |
| | expressed sequence | | |
| 202459_s_at | lipin 2 | 0.08 | −0.169 |
| 217838_s_at | Enah/Vasp-like | 0.0798 | −0.1685 |
| 216233_at | CD163 antigen | −0.0788 | 0.1664 |
| 219607_s_at | membrane-spanning 4-domains, subfamily A, | −0.0782 | 0.1652 |
| | member 4 | | |
| 209163_at | cytochrome b-561 | 0.078 | −0.1647 |
| 210279_at | G protein-coupled receptor 18 | 0.0779 | −0.1644 |
| 205119_s_at | formyl peptide receptor 1 | −0.0762 | 0.161 |
| 207460_at | granzyme M (lymphocyte met-ase 1) | 0.0758 | −0.16 |
| 209504_s_at | pleckstrin homology domain containing, family B | 0.0752 | −0.1588 |
| | (evectins) member 1 | | |
| 221601_s_at | Fas apoptotic inhibitory molecule 3 | 0.0733 | −0.1548 |
| 201367_s_at | zinc finger protein 36, C3H type-like 2 | 0.0702 | −0.1482 |
| 203828_s_at | interleukin 32 | 0.0698 | −0.1474 |
| 209782_s_at | D site of albumin promoter (albumin D-box) binding | 0.0696 | −0.1469 |
| | protein | | |
| 202435_s_at | cytochrome P450, family 1, subfamily B, polypeptide 1 | −0.0686 | 0.1447 |
| 205844_at | vanin 1 | −0.0683 | 0.1442 |
| 205681_at | BCL2-related protein A1 | −0.0681 | 0.1438 |
| 213689_x_at | Ribosomal protein L5 | 0.0681 | −0.1437 |
| 210201_x_at | bridging integrator 1 | 0.068 | −0.1436 |
| 201925_s_at | decay accelerating factor for complement (CD55, | −0.0676 | 0.1427 |
| | Cromer blood group system) | | |
| 220330_s_at | SAM domain, SH3 domain and nuclear localisation | −0.0675 | 0.1425 |
| | signals, 1 | | |
| 204890_s_at | lymphocyte-specific protein tyrosine kinase | 0.0669 | −0.1413 |
| 206061_s_at | Dicer1, Dcr-1 homolog (Drosophila) | −0.0665 | 0.1404 |
| 204960_at | protein tyrosine phosphatase, receptor type, C- | 0.0662 | −0.1398 |
| | associated protein | | |
| 210116_at | SH2 domain protein 1A, Duncan's disease | 0.0656 | −0.1385 |
| | (lymphoproliferative syndrome) | | |
| 200644_at | MARCKS-like 1 | 0.0655 | −0.1383 |
| 214439_x_at | bridging integrator 1 | 0.0649 | −0.1371 |
| 202191_s_at | growth arrest-specific 7 | −0.0649 | 0.137 |
| 219812_at | hypothetical protein MGC2463 | 0.0649 | −0.137 |
| 200965_s_at | actin binding LIM protein 1 | 0.0649 | −0.137 |
| 213397_x_at | ribonuclease, RNase A family, 4 | −0.0638 | 0.1347 |
| 206181_at | signaling lymphocytic activation molecule family | 0.0636 | −0.1342 |
| | member 1 | | |
| 220485_s_at | signal-regulatory protein beta 2 | 0.063 | −0.133 |
| 207339_s_at | lymphotoxin beta (TNF superfamily, member 3) | 0.0626 | −0.1322 |
| 210512_s_at | vascular endothelial growth factor | −0.0624 | 0.1316 |
| 202931_x_at | bridging integrator 1 | 0.061 | −0.1288 |
| 215001_s_at | glutamate-ammonia ligase (glutamine synthetase) | −0.0605 | 0.1276 |
| 208686_s_at | bromodomain containing 2 | 0.0597 | −0.126 |
| 211339_s_at | IL2-inducible T-cell kinase | 0.0586 | −0.1236 |
| 214958_s_at | epidermodysplasia verruciformis 1 | 0.0582 | −0.1229 |
| 217552_x_at | complement component (3b/4b) receptor 1, including | −0.0578 | 0.1221 |
| | Knops blood group system | | |
| 205141_at | angiogenin, ribonuclease, RNase A family, 5 | −0.0578 | 0.1219 |
| 217763_s_at | RAB31, member RAS oncogene family | −0.0577 | 0.1217 |
| 209616_s_at | carboxylesterase 1 (monocyte/macrophage serine | −0.0558 | 0.1179 |
| | esterase 1) | | |
| 209670_at | T cell receptor alpha constant | 0.0558 | −0.1178 |
| 221249_s_at | C/EBP-induced protein | 0.0557 | −0.1175 |
| 206118_at | signal transducer and activator of transcription 4 | 0.0555 | −0.1173 |
| 211275_s_at | glycogenin | −0.0548 | 0.1157 |
| 204619_s_at | chondroitin sulfate proteoglycan 2 (versican) | −0.0544 | 0.1149 |
| 220570_at | resistin | −0.0544 | 0.1148 |
| 201926_s_at | decay accelerating factor for complement (CD55, | −0.0537 | 0.1133 |
| | Cromer blood group system) | | |
| 210517_s_at | A kinase (PRKA) anchor protein (gravin) 12 | 0.0535 | −0.113 |
| 213958_at | CD6 antigen | 0.0532 | −0.1123 |
| 203765_at | grancalcin, EF-hand calcium binding protein | −0.0517 | 0.1091 |
| 204908_s_at | B-cell CLL/lymphoma 3 | −0.0515 | 0.1087 |
| 211005_at | linker for activation of T cells | 0.0514 | −0.1084 |
| 211711_s_at | phosphatase and tensin homolog (mutated in multiple | −0.0511 | 0.1079 |
| | advanced cancers 1) | | |
| 218559_s_at | v-maf musculoaponeurotic fibrosarcoma oncogene | −0.0509 | 0.1075 |
| | homolog B (avian) | | |
| 222043_at | clusterin (complement lysis inhibitor, SP-40,40, | −0.0509 | 0.1074 |
| | sulfated glycoprotein 2, testosterone-repressed | | |
| | prostate message 2, apolipoprotein J) | | |
| 219423_x_at | tumor necrosis factor receptor superfamily, member | 0.0509 | −0.1074 |
| | 25 | | |
| 218319_at | pellino homolog 1 (Drosophila) | −0.0508 | 0.1073 |
| 211596_s_at | leucine-rich repeats and immunoglobulin-like | 0.0499 | −0.1053 |
| | domains 1 | | |
| 222235_s_at | chondroitin sulfate GalNAcT-2 | −0.0497 | 0.105 |
| 210426_x_at | RAR-related orphan receptor A | 0.0497 | −0.1049 |
| 203751_x_at | jun D proto-oncogene | 0.0488 | −0.103 |
| 203887_s_at | thrombomodulin | −0.0485 | 0.1024 |
| 204860_s_at | baculoviral IAP repeat-containing 1 | −0.0484 | 0.1022 |
| 207275_s_at | acyl-CoA synthetase long-chain family member 1 | −0.0482 | 0.1018 |
| 202861_at | period homolog 1 (Drosophila) | −0.0482 | 0.1017 |
| 205831_at | CD2 antigen (p50), sheep red blood cell receptor | 0.0477 | −0.1006 |
| 220418_at | ubiquitin associated and SH3 domain containing, A | 0.0469 | −0.0989 |
| 212641_at | human immunodeficiency virus type I enhancer | 0.0466 | −0.0984 |
| | binding protein 2 | | |
| 217969_at | chromosome 11 open reading frame2 | 0.0466 | −0.0983 |
| 212575_at | chromosome 19 open reading frame 6 | 0.0459 | −0.097 |
| 202381_at | ADAM metallopeptidase domain 9 (meltrin gamma) | −0.0455 | 0.0961 |
| 211936_at | heat shock 70 kDa protein 5 (glucose-regulated | −0.0455 | 0.0961 |
| | protein, 78 kDa) | | |
| 217986_s_at | bromodomain adjacent to zinc finger domain, 1A | −0.0454 | 0.0958 |
| 221210_s_at | N-acetylneuraminate pyruvate lyase | −0.0453 | 0.0955 |
| | (dihydrodipicolinate synthase) | | |
| 202747_s_at | integral membrane protein 2A | 0.0447 | −0.0943 |
| 212914_at | chromobox homolog 7 | 0.0444 | −0.0937 |
| 213274_s_at | cathepsin B | −0.0442 | 0.0933 |
| 212658_at | lipoma HMGIC fusion partner-like 2 | −0.0434 | 0.0917 |
| 203413_at | NEL-like 2 (chicken) | 0.0431 | −0.0909 |
| 205425_at | huntingtin interacting protein 1 | −0.043 | 0.0908 |
| 204112_s_at | histamine N-methyltransferase | −0.0429 | 0.0906 |
| 209154_at | Tax1 (human T-cell leukemia virus type I) binding | −0.0428 | 0.0904 |
| | protein 3 | | |
| 202208_s_at | ADP-ribosylation factor-like 7 | 0.0424 | −0.0896 |
| 200707_at | protein kinase C substrate 80K-H | 0.0422 | −0.0892 |
| 209960_at | hepatocyte growth factor (hepapoietin A; scatter | −0.0422 | 0.0891 |
| | factor) | | |
| 211764_s_at | ubiquitin-conjugating enzyme E2D 1 (UBC4/5 | −0.0419 | 0.0885 |
| | homolog, yeast) | | |
| 215761_at | Dmx-like 2 | −0.0419 | 0.0884 |
| 207067_s_at | histidine decarboxylase | 0.0411 | −0.0867 |
| 200675_at | CD81 antigen (target of antiproliferative antibody 1) | 0.0405 | −0.0855 |
| 203385_at | diacylglycerol kinase, alpha 80 kDa | 0.04 | −0.0845 |
| 204614_at | serpin peptidase inhibitor, clade B (ovalbumin), | −0.0399 | 0.0842 |
| | member 2 | | |
| 204198_s_at | runt-related transcription factor 3 | 0.0398 | −0.0841 |
| 212574_x_at | chromosome 19 open reading frame 6 | 0.0398 | −0.0839 |
| 218328_at | coenzyme Q4 homolog (yeast) | 0.0387 | −0.0817 |
| 206111_at | ribonuclease, RNase A family, 2 (liver, eosinophil- | −0.0377 | 0.0796 |
| | derived neurotoxin) | | |
| 201853_s_at | cell division cycle 25B | 0.0376 | −0.0793 |
| 200663_at | CD63 antigen (melanoma 1 antigen) | −0.0371 | 0.0783 |
| 211282_x_at | tumor necrosis factor receptor superfamily, member | 0.037 | −0.0781 |
| | 25 | | |
| 219541_at | Lck interacting transmembrane adaptor 1 | 0.0367 | −0.0775 |
| 215127_s_at | RNA binding motif, single stranded interacting | −0.0366 | 0.0773 |
| | protein 1 | | |
| 215796_at | T cell receptor alpha variable 20 | 0.0365 | −0.0771 |
| 204140_at | tyrosylprotein sulfotransferase 1 | −0.0365 | 0.077 |
| 208808_s_at | high-mobility group box 2 | −0.0363 | 0.0767 |
| 203965_at | ubiquitin specific peptidase 20 | 0.0361 | −0.0761 |
| 210031_at | CD3Z antigen, zeta polypeptide (TiT3 complex) | 0.0358 | −0.0756 |
| 205603_s_at | diaphanous homolog 2 (Drosophila) | −0.0356 | 0.0751 |
| 218927_s_at | carbohydrate (chondroitin 4) sulfotransferase 12 | 0.0354 | −0.0748 |
| 209156_s_at | collagen, type VI, alpha 2 | 0.0353 | −0.0745 |
| 204393_s_at | acid phosphatase, prostate | −0.0348 | 0.0736 |
| 203548_s_at | lipoprotein lipase | −0.0347 | 0.0732 |
| 205745_x_at | ADAM metallopeptidase domain 17 (tumor necrosis | −0.0344 | 0.0726 |
| | factor, alpha, converting enzyme) | | |
| 218454_at | hypothetical protein FLJ22662 | −0.0341 | 0.072 |
| 210166_at | toll-like receptor 5 | −0.0336 | 0.071 |
| 205568_at | aquaporin 9 | −0.0331 | 0.0699 |
| 204985_s_at | trafficking protein particle complex 6A | 0.0331 | −0.0698 |
| 202739_s_at | phosphorylase kinase, beta | −0.0324 | 0.0684 |
| 209185_s_at | insulin receptor substrate 2 | −0.0322 | 0.0681 |
| 213198_at | activin A receptor, type IB | −0.0322 | 0.068 |
| 212989_at | transmembrane protein 23 | −0.032 | 0.0675 |
| 210640_s_at | G protein-coupled receptor 30 | −0.032 | 0.0675 |
| 203827_at | WD40 repeat protein Interacting with | −0.0318 | 0.067 |
| | phosphoInositides of 49 kDa | | |
| 203574_at | nuclear factor, interleukin 3 regulated | −0.0317 | 0.0668 |
| 204971_at | cystatin A (stefin A) | −0.0316 | 0.0666 |
| 204269_at | pim-2 oncogene | 0.0316 | −0.0666 |
| 31874_at | growth arrest-specific 2 like 1 | −0.0314 | 0.0662 |
| 207734_at | lymphocyte transmembrane adaptor 1 | 0.0311 | −0.0657 |
| 203392_s_at | C-terminal binding protein 1 | 0.0307 | −0.0647 |
| 212263_at | quaking homolog, KH domain RNA binding (mouse) | −0.0292 | 0.0616 |
| 206522_at | maltase-glucoamylase (alpha-glucosidase) | −0.029 | 0.0612 |
| 212665_at | TCDD-inducible poly(ADP-ribose) polymerase | −0.0286 | 0.0603 |
| 210095_s_at | insulin-like growth factor binding protein 3 | 0.0285 | −0.0601 |
| 218217_at | serine carboxypeptidase 1 | −0.0284 | 0.0599 |
| 214447_at | v-ets erythroblastosis virus E26 oncogene homolog 1 | 0.0283 | −0.0597 |
| | (avian) | | |
| 210825_s_at | prostatic binding protein | 0.0282 | −0.0596 |
| 58780_s_at | hypothetical protein FLJ10357 | −0.0282 | 0.0596 |
| 217119_s_at | chemokine (C—X—C motif) receptor 3 | 0.0282 | −0.0594 |
| 213926_s_at | HIV-1 Rev binding protein | −0.028 | 0.0592 |
| 218618_s_at | fibronectin type III domain containing 3B | −0.0277 | 0.0585 |
| 221658_s_at | interleukin 21 receptor | 0.0272 | −0.0574 |
| 210039_s_at | protein kinase C, theta | 0.0271 | −0.0572 |
| 208644_at | poly (ADP-ribose) polymerase family, member 1 | 0.027 | −0.057 |
| 38487_at | stabilin 1 | −0.0269 | 0.0568 |
| 212589_at | Sterol carrier protein 2 | 0.0262 | −0.0552 |
| 210948_s_at | lymphoid enhancer-binding factor 1 | 0.0259 | −0.0547 |
| 205863_at | S100 calcium binding protein A12 (calgranulin C) | −0.0257 | 0.0542 |
| 218728_s_at | cornichon homolog 4 (Drosophila) | −0.0255 | 0.0539 |
| 207351_s_at | SH2 domain protein 2A | 0.0254 | −0.0537 |
| 205798_at | interleukin 7 receptor | 0.025 | −0.0527 |
| 221731_x_at | chondroitin sulfate proteoglycan 2 (versican) | −0.0248 | 0.0524 |
| 209184_s_at | insulin receptor substrate 2 | −0.0247 | 0.0521 |
| 209619_at | CD74 antigen (invariant polypeptide of major | 0.0247 | −0.0521 |
| | histocompatibility complex, class II antigen- | | |
| | associated) | | |
| 209906_at | complement component 3a receptor 1 | −0.0233 | 0.0493 |
| 206296_x_at | mitogen-activated protein kinase kinase kinase kinase 1 | 0.0225 | −0.0475 |
| 211856_x_at | CD28 antigen (Tp44) | 0.0218 | −0.046 |
| 204951_at | ras homolog gene family, member H | 0.0215 | −0.0454 |
| 202624_s_at | calcineurin binding protein 1 | 0.0215 | −0.0453 |
| 201677_at | Chromosome 3 open reading frame 37 | 0.0214 | −0.0452 |
| 201555_at | MCM3 minichromosome maintenance deficient 3 (S. cerevisiae) | 0.0214 | −0.0451 |
| 210873_x_at | apolipoprotein B mRNA editing enzyme, catalytic | −0.0214 | 0.0451 |
| | polypeptide-like 3A | | |
| 216667_at | ribonuclease, RNase A family, 2 (liver, eosinophil- | −0.0213 | 0.0449 |
| | derived neurotoxin) | | |
| 216133_at | T cell receptor V alpha gene segment V-alpha-w23, | 0.0212 | −0.0448 |
| | clone IGRa01 | | |
| 200765_x_at | catenin (cadherin-associated protein), alpha 1, 102 kDa | −0.0212 | 0.0448 |
| 205590_at | RAS guanyl releasing protein 1 (calcium and DAG- | 0.0204 | −0.0431 |
| | regulated) | | |
| 206666_at | granzyme K (granzyme 3; tryptase II) | 0.0204 | −0.043 |
| 217147_s_at | T cell receptor associated transmembrane adaptor 1 | 0.0202 | −0.0426 |
| 209379_s_at | KIAA1128 | 0.0201 | −0.0424 |
| 201951_at | activated leukocyte cell adhesion molecule | −0.0199 | 0.042 |
| 203547_at | CD4 antigen (p55) | 0.0195 | −0.0412 |
| 219922_s_at | latent transforming growth factor beta binding protein 3 | 0.0195 | −0.0412 |
| 208470_s_at | haptoglobin | −0.0186 | 0.0393 |
| 212144_at | unc-84 homolog B (C. elegans) | 0.0183 | −0.0386 |
| 214219_x_at | mitogen-activated protein kinase kinase kinase kinase 1 | 0.0181 | −0.0382 |
| 206714_at | arachidonate 15-lipoxygenase, second type | −0.0178 | 0.0376 |
| 219622_at | RAB20, member RAS oncogene family | −0.0178 | 0.0375 |
| 214696_at | hypothetical protein MGC14376 | −0.0172 | 0.0362 |
| 215923_s_at | pleckstrin and Sec7 domain containing 4 | 0.0171 | −0.036 |
| 215967_s_at | lymphocyte antigen 9 | 0.017 | −0.0358 |
| 210038_at | protein kinase C, theta | 0.017 | −0.0358 |
| 211794_at | FYN binding protein (FYB-120/130) | −0.0165 | 0.0349 |
| 204103_at | chemokine (C-C motif) ligand 4 | 0.0161 | −0.0339 |
| 212464_s_at | fibronectin 1 | −0.0159 | 0.0336 |
| 218091_at | HIV-1 Rev binding protein | −0.0159 | 0.0335 |
| 202074_s_at | optineurin | 0.0155 | −0.0328 |
| 209135_at | aspartate beta-hydroxylase | −0.0154 | 0.0325 |
| 213986_s_at | chromosome 19 open reading frame 6 | 0.0148 | −0.0313 |
| 210607_at | fms-related tyrosine kinase 3 ligand | 0.0145 | −0.0307 |
| 207824_s_at | MYC-associated zinc finger protein (purine-binding | 0.0139 | −0.0293 |
| | transcription factor) | | |
| 213572_s_at | serpin peptidase inhibitor, clade B (ovalbumin), | −0.0137 | 0.0288 |
| | member 1 | | |
| 201952_at | activated leukocyte cell adhesion molecule | −0.0136 | 0.0287 |
| 219358_s_at | centaurin, alpha 2 | −0.0136 | 0.0286 |
| 214771_x_at | myosin phosphatase-Rho interacting protein | 0.0132 | −0.0278 |
| 216969_s_at | kinesin family member 22 | 0.013 | −0.0275 |
| 201557_at | vesicle-associated membrane protein 2 (synaptobrevin | 0.0126 | −0.0266 |
| | 2) | | |
| 206150_at | tumor necrosis factor receptor superfamily, member 7 | 0.0126 | −0.0265 |
| 205819_at | macrophage receptor with collagenous structure | −0.0125 | 0.0263 |
| 212449_s_at | lysophospholipase I | −0.0123 | 0.026 |
| 213587_s_at | ATPase, H+ transporting V0 subunit E isoform 2-like | 0.0123 | −0.0259 |
| | (rat) | | |
| 221851_at | hypothetical protein BC002926 | 0.0122 | −0.0257 |
| 203556_at | zinc fingers and homeoboxes 2 | 0.0121 | −0.0254 |
| 39582_at | Cylindromatosis (turban tumor syndrome) | 0.012 | −0.0253 |
| 217729_s_at | amino-terminal enhancer of split | 0.0119 | −0.025 |
| 214877_at | Proteasome (prosome, macropain) 26S subunit, non- | 0.0116 | −0.0244 |
| | ATPase, 12 | | |
| 212316_at | nucleoporin 210 kDa | 0.0115 | −0.0242 |
| 201313_at | enolase 2 (gamma, neuronal) | 0.0113 | −0.0238 |
| 210844_x_at | catenin (cadherin-associated protein), alpha 1, 102 kDa | −0.0111 | 0.0234 |
| 214022_s_at | interferon induced transmembrane protein 1 (9-27) | 0.0107 | −0.0227 |
| 212642_s_at | human immunodeficiency virus type I enhancer | 0.0107 | −0.0225 |
| | binding protein 2 | | |
| 211272_s_at | diacylglycerol kinase, alpha 80 kDa | 0.0107 | −0.0225 |
| 209308_s_at | BCL2/adenovirus E1B 19 kDa interacting protein 2 | −0.0106 | 0.0224 |
| 212990_at | synaptojanin 1 | −0.0102 | 0.0216 |
| 209286_at | CDC42 effector protein (Rho GTPase binding) 3 | −0.0098 | 0.0206 |
| 211841_s_at | tumor necrosis factor receptor superfamily, member | 0.0096 | −0.0202 |
| | 25 | | |
| 205349_at | guanine nucleotide binding protein (G protein), alpha | −0.0096 | 0.0202 |
| | 15 (Gq class) | | |
| 219859_at | C-type lectin domain family 4, member E | −0.0095 | 0.02 |
| 200952_s_at | cyclin D2 | 0.0093 | −0.0196 |
| 201561_s_at | calsyntenin 1 | 0.009 | −0.019 |
| 212606_at | WD repeat and FYVE domain containing 3 | −0.0085 | 0.0179 |
| 201188_s_at | inositol 1,4,5-triphosphate receptor, type 3 | 0.0084 | −0.0177 |
| 201601_x_at | interferon induced transmembrane protein 1 (9-27) | 0.0083 | −0.0176 |
| 210986_s_at | tropomyosin 1 (alpha) | −0.0083 | 0.0176 |
| 218865_at | MOCO sulphurase C-terminal domain containing 1 | −0.0082 | 0.0174 |
| 201369_s_at | zinc finger protein 36, C3H type-like 2 | 0.0079 | −0.0166 |
| 208636_at | Actinin, alpha 1 | −0.0077 | 0.0163 |
| 200671_s_at | spectrin, beta, non-erythrocytic 1 | 0.0077 | −0.0162 |
| 219988_s_at | chromosome 1 open reading frame 164 | 0.0075 | −0.0159 |
| 202928_s_at | PHD finger protein 1 | 0.0075 | −0.0158 |
| 212414_s_at | septin 6 | 0.0072 | −0.0152 |
| 220001_at | peptidyl arginine deiminase, type IV | −0.0071 | 0.0151 |
| 33197_at | myosin VIIA | −0.0069 | 0.0145 |
| 208723_at | ubiquitin specific peptidase 11 | 0.0068 | −0.0144 |
| 204442_x_at | latent transforming growth factor beta binding protein 4 | 0.0067 | −0.0141 |
| 208807_s_at | chromodomain helicase DNA binding protein 3 | 0.0066 | −0.0139 |
| 205191_at | retinitis pigmentosa 2 (X-linked recessive) | −0.0065 | 0.0136 |
| 203608_at | aldehyde dehydrogenase 5 family, member A1 | 0.0062 | −0.0131 |
| | (succinate-semialdehyde dehydrogenase) | | |
| 204646_at | dihydropyrimidine dehydrogenase | −0.0059 | 0.0125 |
| 203159_at | glutaminase | 0.0054 | −0.0114 |
| 205471_s_at | dachshund homolog 1 (Drosophila) | −0.0051 | 0.0107 |
| 213295_at | Cylindromatosis (turban tumor syndrome) | 0.0049 | −0.0103 |
| 207485_x_at | butyrophilin, subfamily 3, member A1 | 0.0048 | −0.0102 |
| 218043_s_at | 5-azacytidine induced 2 | −0.0048 | 0.0102 |
| 201554_x_at | glycogenin | −0.0048 | 0.0102 |
| 218854_at | squamous cell carcinoma antigen recognized by T | −0.0048 | 0.0101 |
| | cells 2 | | |
| 209555_s_at | CD36 antigen (collagen type I receptor, | −0.0047 | 0.0099 |
| | thrombospondin receptor) | | |
| 218668_s_at | RAP2C, member of RAS oncogene family | −0.0046 | 0.0096 |
| 200864_s_at | RAB11A, member RAS oncogene family | −0.0046 | 0.0096 |
| 213241_at | plexin C1 | −0.0045 | 0.0094 |
| 37145_at | granulysin | 0.0043 | −0.009 |
| 205718_at | integrin, beta 7 | 0.0042 | −0.0088 |
| 209604_s_at | GATA binding protein 3 | 0.0041 | −0.0086 |
| 205963_s_at | DnaJ (Hsp40) homolog, subfamily A, member 3 | 0.0039 | −0.0083 |
| 209603_at | GATA binding protein 3 | 0.0039 | −0.0082 |
| 201185_at | HtrA serine peptidase 1 | −0.0038 | 0.008 |
| 202039_at | TGFB1-induced anti-apoptotic factor 1 | 0.0036 | −0.0076 |
| 214975_s_at | myotubularin related protein 1 | 0.0035 | −0.0074 |
| 202146_at | interferon-related developmental regulator 1 | −0.0034 | 0.0072 |
| 205488_at | granzyme A (granzyme 1, cytotoxic T-lymphocyte- | 0.003 | −0.0064 |
| | associated serine esterase 3) | | |
| 221519_at | F-box and WD-40 domain protein 4 | 0.0029 | −0.0061 |
| 214452_at | branched chain aminotransferase 1, cytosolic | −0.0028 | 0.0058 |
| 204777_s_at | mal, T-cell differentiation protein | 0.0027 | −0.0057 |
| 216920_s_at | T cell receptor gamma constant 2 | 0.0023 | −0.0049 |
| 217507_at | Solute carrier family 11 (proton-coupled divalent | −0.0023 | 0.0049 |
| | metal ion transporters), member 1 | | |
| 215646_s_at | chondroitin sulfate proteoglycan 2 (versican) | −0.0022 | 0.0046 |
| 210538_s_at | baculoviral IAP repeat-containing 3 | 0.0022 | −0.0046 |
| 213622_at | collagen, type IX, alpha 2 | 0.0017 | −0.0035 |
| 210980_s_at | N-acylsphingosine amidohydrolase (acid ceramidase) 1 | −0.0016 | 0.0033 |
| 212888_at | Dicer1, Dcr-1 homolog (Drosophila) | −0.0015 | 0.0032 |
| 200941_at | heat shock factor binding protein 1 | −0.0015 | 0.0031 |
| 205931_s_at | cAMP responsive element binding protein 5 | −0.0015 | 0.0031 |
| 207674_at | Fc fragment of IgA, receptor for | −0.0014 | 0.0029 |
| 208857_s_at | protein-L-isoaspartate (D-aspartate) O- | −0.0013 | 0.0028 |
| | methyltransferase | | |
| 218323_at | ras homolog gene family, member T1 | −0.0012 | 0.0026 |
| 220054_at | interleukin 23, alpha subunit p19 | 0.0012 | −0.0024 |
| 201361_at | hypothetical protein MGC5508 | 9.00E−04 | −0.0019 |
| 216442_x_at | fibronectin 1 | −7.00E−04 | 0.0014 |
| 209600_s_at | acyl-Coenzyme A oxidase 1, palmitoyl | −4.00E−04 | 9.00E−04 |
| 215806_x_at | T cell receptor gamma constant 2 | 4.00E−04 | −9.00E−04 |
| 221012_s_at | tripartite motif-containing 8 | −2.00E−04 | 5.00E−04 |
| 201560_at | chloride intracellular channel 4 | −2.00E−04 | 4.00E−04 |
| 209815_at | patched homolog (Drosophila) | 1.00E−04 | −2.00E−04 |

\hat{ }Probe set ID number is the Affymetrix ID number on the HU133A array

The ability of the 380 probes in Table 8 to accurately classify subjects as having not had a hemorrhagic stroke or having had a hemorrhagic stroke was determined. The ability of those probes to accurately classify an IS subject as not having had a hemorrhagic stroke was 18/19, and to accurately classify a subject as having had a hemorrhagic stroke was 7/9. This indicates that the disclosed methods can determine whether a subject has had a hemorrhagic stroke (such as an ICH) with a sensitivity of at least 78% and a specificity (or accuracy) of at least 90% (such as at least 94%).

Therefore, as shown in the tables above, several genes not previously associated with hemorrhagic stroke, such as IL1R2, haptoglobin, amphiphysin, TAP2, CD163, granzyme M, and Sema4C were identified. As opposed to ischemic stroke (IS), where around 90% of the genes were up-regulated (see PCT/US2005/018744), in hemorrhagic stroke about 50-60% of genes were up-regulated; a prominent down-regulation of genes related to immune function was found. ICH and IS were both associated with elevated CD163 expression, a marker of conversion of blood-borne monocytes to tissue macrophages. Other genes common to both types of stroke, such as GAS7 and glutamine ligase, indicate a response to the altered cerebral microenvironment. Another gene up-regulated in both IS and ICH is factor V. Up-regulated factor V expression may represent a risk factor for both IS and ICH, or be reflective of the body's effort to maintain a balance between bleeding and clotting.

### Example 5

### Reverse Transcription and Real-Time Polymerase Chain Reactions

This example describes the use of quantitative real-time polymerase chain reaction

(PCR) to confirm results obtained using the microarrays described in Example 4.

RNA (2 μg) from 6 ICH subjects and 7 “normal” subjects was retro-transcribed to complementary deoxyribonucleic acid in a final volume of 21 μL with the SuperScript First-Strand Synthesis System (Invitrogen, Catalogue # 108080-051) following manufacturer's instructions. Genes were selected for analysis on the basis of their significantly increased (5 genes) or decreased (3 genes) expression in ICH subjects compared to control (non-stroke) subjects. Primers were obtained from the published literature and ordered from Invitrogen (Carlsbad, Calif.) as listed in Table 9.

TABLE 9
Primers for real time-PCR

| | | |
|---|---|---|
| | Representative | |
| Gene Symbol | Public ID | Primer Sequence (SEQ ID NO:) |

| | | | |
|---|---|---|---|
| Up-regulated in | | | |
| ICH on array | | | |
| IL1R2* | NM_004633 | F-CTACGCACCACAGTCAAGGAAG | (1) |
| | | R-TGCATCCATATTCCCCCCA | (2) |
| IL1R2** | NM_004633 | F-GGCCAGCAATACAACATCAC | (3) |
| | | R-CCCAGAAACACCTTACACG | (4) |
| AMPH | NM_001635 | F-TAGCAGCAATCAAAGGCATGC | (5) |
| | | R-TAGCAGCAATCAAAGGCATGC | (6) |
| CD163 | NM_004244 | F-ACAGGTCGCTCATCCCGTC | (7) |
| | | R-CCCAAGGATCCCGACTGC | (8) |
| F5 | NM_000130 | F-AAATCCCATGAGTTTCACGCC | (9) |
| | | R-CAGACCCCTAACTGGTGCTGTT | (10) |
| S100A9 | NM_002965 | F-CGGCTTTGAGACAGAGTGCAA | (11) |
| | | R-CGCACCAGCTCTTTGAATTCC | (12) |
| Down-regulated | | | |
| in ICH on array | | | |
| SEMA4C | NM_017789 | F-TGTGGATGGTGAGCTGTACTCG | (13) |
| | | R-GTTGAGCCAAAAGGCCAGGTA | (14) |
| IRF1 | NM_002198 | F-TGCCAGATATCGAGGAGGTGAA | (15) |
| | | R-TGACTTCCTCTTGGCCTTGCT | (16) |
| CD6 | NM_006725 | F-TGACCACCTTCTACAATTCCC | (17) |
| | | R-AACTCTTCAAGTCCTTCCTCC | (18) |
| Not significantly | | | |
| altered on array | | | |
| CASC3 | NM_007359 | F-TTCCCCACCCAGGTTTACATC | (19) |
| | | R-AAAGTTCATGACGCCTGGAGC | (20) |
| NUCB1 | NM_006184 | F-GAATGTGGACACCAACCAGGA | (21) |
| | | R-TTCAAAGCGCCTCAGCTCTTC | (22) |
| FDFT1 | NM_004462 | F-CGCAACGCAGTGTGCATATT | (23) |
| | | R-ACCGCCAGTCTGGTTGGTAAA | (24) |

F-forward, R-reverse, *, **separate gene probes/primers used for real time PCR

The quantitative real-time PCR reaction was run in an Opticon cycler (MJ Research) with the Sybr Green PCR master mix (Applied Biosystems) following manufacturer's instructions. Thermocycling was performed in a final volume of 15 μL consisting of 3 μL cDNA (diluted 1:100) and 400 nmol/L primers (Table 9). Glyceraldehyde-3-phosphate dehydrogenase (GAPDH) was used as the normalizing housekeeping gene in all samples.

For every sample, both the housekeeping and target genes were amplified in triplicate in the same run, using the following cycle scheme: after initial denaturation of the samples at 95° C. for 5 minutes, 47 cycles of 95° C. for 30 seconds, 60° C. for 30 seconds, and 72° C. for 30 seconds. Fluorescence was measured in every cycle, and a melting curve was run after the PCR by increasing the temperature from 60° C. to 90° C. (1.0° C. increments). A defined single peak was obtained for all amplicons, confirming the specificity of the amplification. PCR results between patients and referents were compared through the use of non-parametric statistics (Mann-Whitney U tests). If the melting curve showed more than one peak or the peak did not fall with those of the other samples the sample was excluded. All real-time PCR data were normalized before comparison with the GAPDH sample level. The results of the real time PCR experiments are reported as ratios.

Three of the ICH genes of interest were also tested in two additional non ICH referent patients who had other forms of brain pathology (one patient with a traumatic intracerebral hemorrhage and one patient with an ischemic stroke and a microbleed).

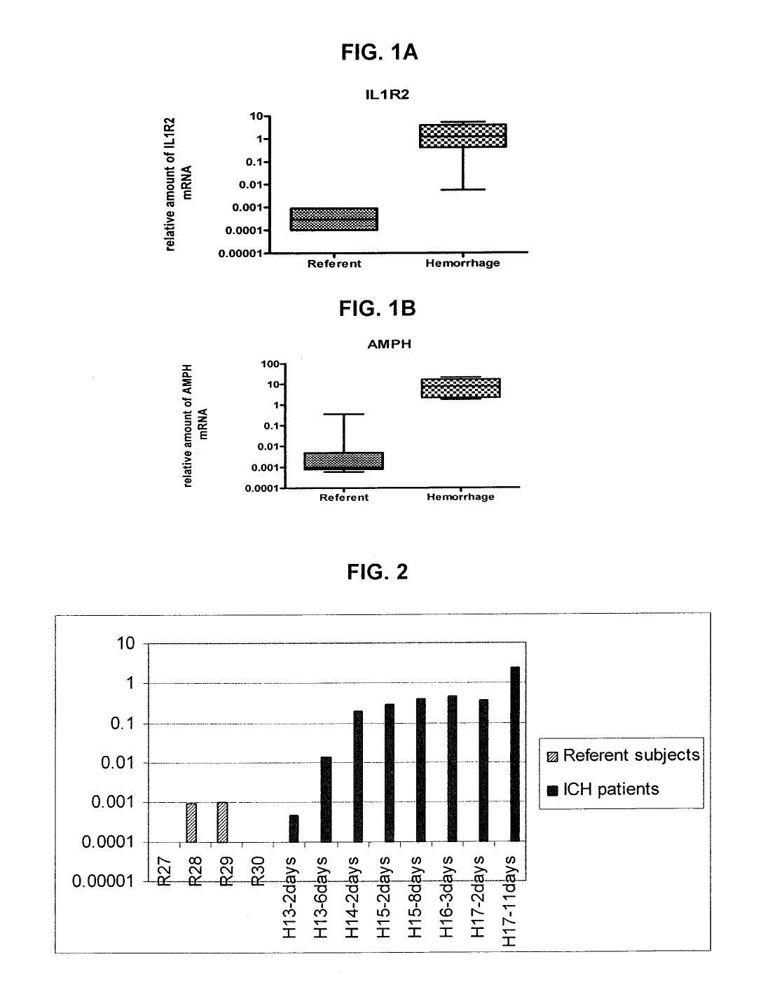

As shown in Table 10, real-time PCR confirmed altered mRNA expression in 8/8 genes (10/10 gene probes) differentially up- or down-regulated in the ICH group compared to the referent group. IL1R2 and amphiphysin expression were elevated several hundred fold in the ICH patients relative to the referents (FIGS. 1A and 1B). These genes appear to be minimally expressed under physiological conditions in PBMCs, if at all. Up-regulated IL1R2 expression was found in two non ICH patients with brain pathology (a patient with a traumatic ICH and a patient with an ischemic stroke and a microbleed), which was intermediate between the levels for ICH and the values of four referent subjects in the index cohort. Using two further genes (SEMAC4C and IRF1) real time PCR was also able to differentiate these two cases showing up-regulated gene expression that was again intermediate between the referent and the ICH levels. Therefore, the disclosed hemorrhagic stroke-associated molecules can be used for diagnosis of a hemorrhage, whether due to stroke or trauma.

TABLE 10
Correlation of expression data with real time-PCR values#

| | Affymetrix | ICH | | Control | | |
|---|---|---|---|---|---|---|
| Gene | probe ID | Relative gene mRNA | | Relative gene mRNA | | p |
| Up-regulated in | | | | | | |
| ICH on array | | | | | | |
| IL1R2* | NM_004633 | 2.74 | (1.3, 8.6) | 0.0005 | (0.0004, 0.02) | 0.0218 |
| IL1R2** | NM_004633 | 1.22 | (0.41, 3.91) | 0.0003 | (0.0001, 0.0009) | 0.0231 |
| AMPH | NM_001635 | 7.831 | (2.19, 17.22) | 0.001 | (0.0008, 0.0049) | 0.0128 |
| CD163 | NM_004244 | 1.5 | (0.71, 2.17) | 0.49 | (0.32, 0.82) | 0.0085 |
| F5 | NM_000130 | 0.98 | (0.28, 1.55) | 0.48 | (0.22, 0.98) | 0.0618 |
| S100A9 | NM_002965 | 46.31 | (16.87, 87.94) | 5.15 | (3.15, 5.56) | 0.0076 |
| Down-regulated | | | | | | |
| in ICH on array | | | | | | |
| SEMA4C | NM_017789 | 0.028 | (0.016, 0.048) | 0.16 | (0.13, 0.23) | 0.0009 |
| IRF1 | NM_002198 | 1.55 | (1.19, 2.09) | 5.58 | (4.01, 8.15) | <0.0001 |
| CD6 | NM_006725 | 0.54 | (0.24, 1.01) | 2.29 | (1.74, 3.66) | 0.0021 |
| Not | | | | | | |
| significantly | | | | | | |
| altered on array | | | | | | |
| CASC3 | NM_007359 | 1.93 | (0.32, 6.18) | 2.5 | (1.39, 5.09) | 0.7262 |
| NUCB1 | NM_006184 | 1.37 | (0.73, 3.05) | 1.82 | (1.1, 3.25) | 0.4961 |
| FDFT1 | NM_004462 | 1.07 | (0.40, 2.15) | 0.71 | (0.58, 1.42) | 0.6128 |

#Results are presented as medians (inter-quartile range)
*, ** separate gene probes/primers used for real time PCR
Genes were altered on the FDR list or the Holm list

### Example 6

### Independent Validation Data Sets

This example describes methods used to independently validate the results described herein. Further validation was performed in two independent test cohorts (7 ICH patients and 10 referent subjects) by (1) determining the accuracy of the PAM list for the classification of ICH in a first and independent test cohort and (2) performing real time PCR in a second test cohort.

In the first validation, the accuracy of the PAM listing generated from the ICH versus “normal” control comparison (Table 5) was used to classify the prospectively obtained samples from 4 ICH patients and 6 referent subjects. Inclusion and exclusion criteria were the same for both ICH patients and referent control subjects as described in Examples 1 and 3-4 for the index cohort. When applied to the first cohort (4 ICH cases and 6 referent subjects) the ICH PAM list of 30 genes (37 gene probes) showed a sensitivity of 75% and a specificity of 100%: all 6 referent subjects were correctly classified with the correct classification of 3 out of 4 prospectively analyzed ICH patients. This indicates that the disclosed methods can determine whether a subject has had a hemorrhagic stroke (such as an ICH) with a specificity of at least 90% (such as at least 95% or 100%) and a sensitivity of at least 75% (such as at least 75%, at least 80%, or even at least 90%).

In the second validation, a cohort of 5 ICH patients (2 of these were also in the first cohort used for PAM classification) studied at 8 time-points post ICH, and 4 normal subjects were used in real time PCR studies to examine genes elevated in the index cohort. In the second test cohort (5 ICH cases [8 time points] and 4 referent subjects) real time PCR confirmed increased amphiphysin expression in 7/8 ICH samples and none of the referent subjects (FIG. 2). The median value for the referent group was 0.0005 (range 9.54×10⁻⁵-0.00101) and for the ICH group was 0.35 (range 0.000456-2.413, p=0.017, Mann Whitney U test). The 8 time-points ranged from 2 days until 11 days. In one subject the amphiphysin level was not increased in the earliest sample (at 48 hours) but had risen on the second sample (4 days later). Therefore, amphiphysin expression was validated with >95% accuracy using real time PCR.

These results demonstrated and validated a significantly altered gene expression in PBMCs during ICH.

### Example 7

### Classes of Gene Expression Altered Following Hemorrhagic Stroke

As shown in Examples 4 and 5 above, a distinct genomic profile of intracerebral hemorrhagic stroke in PBMCs was identified. This example describes seven classes of hemorrhagic stroke-related genes were identified that are upregulated or down-regulated following hemorrhagic stroke: acute inflammatory response, cell adhesion, immune suppression, response to hypoxia, hematoma/vascular repair response, response to the altered cerebral microenvironment and transcription factor/unknown (Table 5). Two of the most significantly up-regulated genes were interleukin receptor 1, type II (IL1R2, p=2.24×10⁻¹⁶) and amphiphysin (p=1.05×10⁻¹⁵). CD163 was also prominently up-regulated. Other genes of interest were acyl-CoA synthetase, which was markedly up-regulated and the ABC protein TAP2, which was markedly down-regulated.

The first are genes involved in the acute inflammatory response, such as CD163. Such genes can initiate or promote an acute inflammatory response (such as promoting or enhancing the exudation of plasma proteins and leukocytes into the surrounding tissue. In a specific example, expression of one or more of such genes is altered (such as upregulated or downregulated) in response to injury to a blood vessel, for example in response to an ICH.

The second are genes involved in cell adhesion, such as acyl-CoA synthetase long-chain family member 1. Such genes can promote or enhance cell adhesion, such as the binding of one cell to another cell, or the binding of a cell or to a surface or matrix. In a specific example, expression of one or more of such genes is altered (such as upregulated or downregulated) in response to injury to a blood vessel, for example in response to an ICH.

The third are genes involved in suppression of the immune response, such as IL1R2. Such genes may reduce available IL1, thereby reducing the activation of cells of the immune system. For example, such genes may reduce or inhibit white blood cell proliferation. In a specific example, expression of one or more of such genes is altered (such as upregulated or downregulated) in response to injury to a blood vessel, for example in response to an ICH.

The fourth are genes involved in response to hypoxia, such as solute carrier family 2, member 3. Expression of such genes is altered (such as upregulated or down-regulated) in response to decreased available oxygen in the blood and tissues. In a specific example, expression of one or more of such genes is altered (such as upregulated or down-regulated) in response to injury to a blood vessel, for example in response to an ICH.

The fifth are genes involved in hematoma/vascular repair response, such as haptoglobin, factor 5, and two genes related to induction of megakaryocyte formation, v-maf musculoaopneurotic fibrosarcoma oncogene homolog B and HIV-1 Rev binding protein. Such genes can promote healing of damaged blood vessels, such as those that have hemorrhaged. In a specific example, expression of one or more of such genes is altered (such as upregulated or downregulated) in response to injury to a blood vessel, for example in response to an ICH.

The sixth are genes involved in response to the altered cerebral microenvironment, such as amphiphysin. Such genes can be involved in enhanced synaptic vesicle recycling in the brain, or as in the case of GAS7 be associated with neuronal recovery and repair. In a specific example, expression of one or more of such genes is altered (such as upregulated or downregulated) in response to injury to a blood vessel, for example in response to an ICH. Amphiphysin is a novel target for ICH as this gene was up-regulated several hundred-fold and was not expressed to any degree in the PBMCs of the referent control subjects.

The seventh are genes involved in signal transduction, such as centaurin alpha 2 and cytochrome P450. Such genes can converse one signal into another type of signal, for example to increase signal transmission between cells or with a cell. In a specific example, expression of one or more of such genes is altered (such as upregulated or down-regulated) in response to injury to a blood vessel, for example in response to an ICH.

In summary, the gene classes demonstrate both specific and non-specific gene expression in PBMCs during hemorrhagic stroke, such as intracerebral hemorrhagic stroke. ICH was associated with up-regulation of genes associated with inactivation of interleukin-1 and suppression of inflammatory responses (e.g. IL1R2) and enhancement of synaptic vesicle endocytosis and recycling in the brain (e.g. amphiphysin). These results indicate that ICH is associated with a profound immune suppression response on the one hand, while, on the other hand, associated with the induction of genes related to acute inflammation and to macrophage functions such as cell adhesion, (e.g., CD163 and acyl-CoA synthetase long-chain family member 1, involved in membrane synthesis). The prominent immune suppression response (e.g., up-regulation of anti-inflammatory genes such as IL1R2 and insulin receptor substrate 2 and down-regulation of other immune response genes) may reflect the body's effort to conserve other blood functions and to focus on digestion of the hematoma.

### Example 8

### Correlational Graph Analyses

Eighty-four gene networks, derived from the Holm corrected differentially expressed gene list between the ICH and the referent groups (Table 4), with significant correlation coefficients after false discovery multiple comparison correction were identified (Table 11). Network 3 was indicative of a direct response to vessel injury in PBMCs. Other networks were indicative of a co-ordinated and synchronized DNA replication response (network 4) as well as with activation of white blood cells (networks 7 and 8), cellular motility (network 6), with white blood cell differentiation (network 10) and with cellular responses (networks 9 and 16, Appendix 5b). Network analyses revealed networks in PBMCs indicative of a direct response to vessel injury and a co-ordinated and synchronized DNA replication response.

TABLE 11
Networks identified from Holm-corrected ICH versus control.

| | |
|---|---|
| Network | Function |

(Growth regulation genes)

| | |
|---|---|
| SEMA4C | Growth-cone guidance growing tissue |
| HLA-DPA1 | Antigen presentation |
| DAB2 | Growth of tissue embryonic development |
| TAXIBP3 | (TIP1) cell motility T cell leukemia virus binding protein |
| TAP2 | Transporter 2 ABC/B MDR/TAP antigen presentation |
| IMP3 | U3 snoRNA |

2. (antigen presentation)

| | |
|---|---|
| HLA-DPA1 | Antigen presentation |
| STAB1 | Stabilin1 scavenger receptor PM<-> EE traffic |

3. (direct response to vessel injury)

| | |
|---|---|
| ARHGAP19 | Rho GTPase activating protein |
| HLA-DPA1 | Antigen presentation |
| ITGAM | IntegrinaM macrophage receptor C3B complement related CD11B |
| | recruitment of leukocytes to site of vessel-injury |
| CALM1 | Calmodulin1 growth cells cycle signal |
| PDCD4 | Programmed cell death 4 nucleus proliferating cells NKT |
| KIF22 | Kinesin22 cell division motor |
| MARCH1 | Membrane associated ring finger (CSHCU)1 down-regulation of |
| (219574_at) | MHC1 by ubiquitin ligase |

4. (DNA repair cell replication)

| | |
|---|---|
| IMAP | U3 snoRNA |
| SEMA4C | Growth-cone guidance growing tissue |
| KIF22 | Kinesin22 cell division motor |
| FANCF | Fanconi's anemia complementation F adaptor DNA binding repair |
| ASFA1 | Histone chaperone DNA replication repair senescence |

5. (Cell
cycle?)

| | |
|---|---|
| DENND2D | DENN/MADD domain containing 2D |
| SASH1 | SAM and SH3 daomain containing reduced in cancer cell cycle |
| ARL4A | Meiosis |

6. (Motility)

| | |
|---|---|
| DDEF1 | Development differentiation enhancing factor-1 GAP activity motility |
| TAXIBP3 | (TIP1) cell motility T cell leukemia virus binding protein |
| DAB2 | Growth of tissue embryonic development |
| MERTK | Thrombotic response platelet activation |
| SLC2A3 | Facilitated glucose transport induced in hypoxia |
| DICER1 | RNA helicase (RNAi) |
| CDC42EP3 | Rho GTPAse negative regulator induce pseudopodia |

7.
(Activation?)

| | |
|---|---|
| TMEM49 | VMP1 vacuole formation |
| YES1 | Oncogene TK |

8. (activation of White blood cells)

| | |
|---|---|
| PADI4 | Peptidyl arginine deaminase granulocyte, macrophage development |
| | inflammation |
| BTN3A1 | Lipid metabolism butyrophylin |
| MMP9 | Matrix metalloproteinase arthritis IL8 mobolization of hematopoetic |
| | progenitors |

9. (cellular response)

| | |
|---|---|
| MARCH1 | Membrane associated ring finger (CSHCU)1 down-regulation of |
| (219574_at) | MHC1 by ubiquitin ligase |
| ARHGAP19 | Rho GTPase activating protein |
| KIF22 | Kinesin22 cell division motor |
| TAP2 | Transporter 2 ABC/B MDR/TAP antigen presentation |
| PDCD4 | Programmed cell death 4 nucleus proliferating cells NKT |
| CYP1B1 | Steroid metabolism signaling eye |
| CTSB | Cathepsin B cysteine proteinase |
| LYGE | Lymphocyte antigen 6 hematopoetic signaling |

10. (activation of response through differentiation)

| | |
|---|---|
| CENTA2 | Binds PIP2 signal |
| MAFB | Regulate megakaryocite differentiation |
| BIN1 | Bridging integrator adaptor nucleus cytoplasm phosphoinositides |
| | (AMPH related) |
| CFLAR | Caspase 8 and FADD like apoptosis regulator |
| IL2RG | IL2 receptor (scid) |

11. (antiviral defence)

| | |
|---|---|
| CALM1 | Calmodulin1 growth cells cycle signal |
| IFIH1 | Interferon induced with helicase C domain activates antiviral (RNA |
| | virus) response induce terminal differentiation |
| TAXIBP3 | (TIP1) cell motility T cell leukemia virus binding protein |
| BTN3A1 | Lipid metabolism butyrophylin |
| PDCD4 | Programmed cell death 4 nucleus proliferating cells NKT |
| NGRN | Neugrin neurite outgrowth differentiation |
| H3F3B | Histon 3B |
| IRF1 | Interferon regulatory factor transcription factor antiviral defence |

12

| | |
|---|---|
| KLHL2 | Kelch like 2 redistribute cytoskeleton punctation neuron depolarization |
| | differentiation/Macrophage response |
| H3F3B | Histon 3B |

13.
(Transcription)

| | |
|---|---|
| FANCF | Fanconi's anemia complementation F adaptor DNA binding repair |
| IMP3 | U3 snoRNA |
| PER1 | Period homolog circadian expression |

14. (immune response)

| | |
|---|---|
| DYSF | Dysferlin limb-gridle muscular dystrophy 2B calcium mediated |
| | membrane fusion autoimmune disease |
| HLA-DPA1 | Antigen presentation |

15. (?)

| | |
|---|---|
| MGC14376 | ? |
| TMEM39A | Transmembrane |

16. (cellular esponse)
C6orf149

| | |
|---|---|
| LYRM4 | LYR motif containing mitochondria? |
| CXCR3 | chimokine receptor 3 migration recruitment |
| RFX5 | Regulatory factor 5 HLA II expression nuclear protein activates MHC |
| | promoters collagen |
| HSP5A | HSP70 glucose regulation BIP |

17

| | |
|---|---|
| MAFB | Regulate megakaryocite differentiation |
| CALM1 | Calmodulin1 growth cells cycle signal |
| TAXIBP3 | (TIP1) cell motility T cell leukemia virus binding protein |
| CENTA2 | Binds PIP2 signal |

18

| | |
|---|---|
| PECI | Peroxisomal enoyl CoA isomerase b Oxidation FA |
| PSME1 | Proteasome activation subunite 1 makes immuno proteasome |

19. (motility)

| | |
|---|---|
| UBE2J1 | Ubiquitin conjugating enzyme ER degradation |
| CDC42EP3 | Rho GTPAse negative regulator induce pseudopodia |

20.
(differentiation)

| | |
|---|---|
| NGRN | Neugrin neurite outgrowth differentiation |
| HLA-DPA1 | Antigen presentation |
| CXCR3 | chimokine receptor 3 migration recruitment |
| KIF22 | Kinesin22 cell division motor |
| IFIH1 | Interferon induced with helicase C domain activates antiviral (RNA |
| | virus) response induce terminal differentiation |
| PDCD4 | Programmed cell death 4 nucleus proliferating cells NKT |
| GALNS | Galactosamine 6 sulfate sulfatase MPSIVA |
| FNTA | Fernisyl transferase CAAX box connects fernisyl to protein cysteins |
| MARCKSL1 | MARCKS like brain organization |

21

| | |
|---|---|
| CR1 | Complement receptor red/white blood cells membrane malaria receptor |
| HTRA1 | Serine peptidase 1 reguate IGF1 response cell growth |

22

| | |
|---|---|
| IFIT2 | Interferon induced tetratricopeptide |
| CDKAL1 | CDK5 regulatory subunit like iron binding |
| AHR | Aryl hydrocarbon receptor transcription factor aromatic activates CYP |
| | cell adheasion migration |
| HLA-DPA1 | Antigen presentation |
| IRF1 | Interferon regulatory factor transcription factor antiviral defence |
| TAP2 | Transporter 2 ABC/B MDR/TAP antigen presentation |
| LYGE | Lymphocyte antigen 6 hematopoetic signaling |
| PSME1 | Proteasome activation subunite 1 makes immuno proteasome |

23

| | |
|---|---|
| CXCR3 | chimokine receptor 3 migration recruitment |
| CALM1 | Calmodulin1 growth cells cycle signal |
| TAXIBP3 | (TIP1) cell motility T cell leukemia virus binding protein |
| YES1 | Oncogene TK |
| H3F3B | Histon 3B |
| NGRN | Neugrin neurite outgrowth differentiation |
| HLA-DPA1 | Antigen presentation |
| C6orf149 | |
| LYRM4 | LYR motif containing mitochondria? |
| RFX5 | Regulatory factor 5 HLA II expression nuclear protein activates MHC |
| | promoters collagen |
| SCARB2 | Scavenger receptor B2 lysosome endosome LIMP2 |
| RNASE1 | Pancreatic RNAase |
| CYB561 | Cytochrome B senescence iron |

24

| | |
|---|---|
| KIF22 | Kinesin22 cell division motor |
| ARHGAP19 | Rho GTPase activating protein |
| MARCH1 | Membrane associated ring finger (CSHCU)1 down-regulation of |
| (219574_at) | MHC1 by ubiquitin ligase |
| HSP5A | HSP70 glucose regulation BIP |
| TAP2 | Transporter 2 ABC/B MDR/TAP antigen presentation |
| NGRN | Neugrin neurite outgrowth differentiation |
| RFX5 | Regulatory factor 5 HLA II expression nuclear protein activates MHC |
| | promoters collagen |
| IMP3 | U3 snoRNA |
| PER1 | Period homolog circadian expression |
| HELZ | Helicase zink finger |

25

| | |
|---|---|
| SCARB2 | Scavenger receptor B2 lysosome endosome LIMP2 |
| LYGE | Lymphocyte antigen 6 hematopoetic signaling |
| HELZ | Helicase zink finger |
| H3F3B | Histon 3B |
| CXCR3 | chimokine receptor 3 migration recruitment |
| PDCD4 | Programmed cell death 4 nucleus proliferating cells NKT |
| ASFA1 | Histone chaperone DNA replication repair senescence |
| SASH1 | SAM and SH3 daomain containing reduced in cancer cell cycle |
| CDKAL1 | CDK5 regulatory subunit like iron binding |
| C3AR1 | Complement component 3a receptor 1 protein receptor to anaphylaxsis |
| | C3a activate macrophages |
| F5 | Coagulation factor V (proaccelerin labile factor) thrombosis |
| GLUL | Glutamate amonia ligase (glutamine synthase regulate body pH |
| | removing amonia |
| JARID2 | Jumonji Nuclear prevents cell replication |

26

| | |
|---|---|
| GLUL | Glutamate amonia ligase (glutamine synthase regulate body pH |
| | removing amonia |
| CDKAL1 | CDK5 regulatory subunit like iron binding |
| SCARB2 | Scavenger receptor B2 lysosome endosome LIMP2 |
| RFX5 | Regulatory factor 5 HLA II expression nuclear protein activates MHC |
| | promoters collagen |
| IMP3 | U3 snoRNA |

27

| | |
|---|---|
| CDKAL1 | CDK5 regulatory subunit like iron binding |
| GLUL | Glutamate amonia ligase (glutamine synthase regulate body pH |
| | removing amonia |
| SCARB2 | Scavenger receptor B2 lysosome endosome LIMP2 |
| JARID2 | Jumonji Nuclear prevents cell replication |
| C3AR1 | Complement component 3a receptor 1 protein receptor to anaphylaxsis |
| | C3a activate macrophages |
| HSP5A | HSP70 glucose regulation BIP |
| RNASE1 | Pancreatic RNAase |
| IFIT2 | Interferon induced tetratricopeptide |
| TAXIBP3 | (TIP1) cell motility T cell leukemia virus binding protein |
| ARL4A | Meiosis |
| ITGAM | IntegrinaM macrophage receptor C3B complement related CD11B |
| | recruitment of leukocytes to site of vessel-injury |
| RNASE2 | RNAase (liver eosinophil derived neurotoxin) immune response |
| THBD | Thrombomodulin activates degradation of factors Va and VIIIa reduces |
| | thrombin |

28

| | |
|---|---|
| MGC14376 | ? |
| H3F3B | Histon 3B |
| LYGE | Lymphocyte antigen 6 hematopoetic signaling |
| TMEM39A | Transmembrane |
| TRIB1 | Tribbles homolog 1 Signal transduction regulation |

29

| | |
|---|---|
| BIN1 | Bridging integrator adaptor nucleus cytoplasm phosphoinositides |
| | (AMPH related) |
| H3F3B | Histon 3B |
| TAXIBP3 | (TIP1) cell motility T cell leukemia virus binding protein |
| BTN3A1 | Lipid metabolism butyrophylin |
| PDCD4 | Programmed cell death 4 nucleus proliferating cells NKT |
| CENTA2 | Binds PIP2 signal |

30

| | |
|---|---|
| HLX1 | H2.0 like Homeobox hematopoetic cells differentiation immune |
| | activation |
| HLA-DPA1 | Antigen presentation |

31

| | |
|---|---|
| CALM1 | Calmodulin1 growth cells cycle signal |
| ARHGAP19 | Rho GTPase activating protein |
| TAP2 | Transporter 2 ABC/B MDR/TAP antigen presentation |
| CXCR3 | chimokine receptor 3 migration recruitment |
| IFIH1 | Interferon induced with helicase C domain activates antiviral (RNA |
| | virus) response induce terminal differentiation |
| MAFB | Regulate megakaryocite differentiation |
| DAB2 | Growth of tissue embryonic development |

32

| | |
|---|---|
| HLA-DPA1 | Antigen presentation |
| ARHGAP19 | Rho GTPase activating protein |
| CXCR3 | chimokine receptor 3 migration recruitment |
| HLX1 | H2.0 like Homeobox hematopoetic cells differentiation immune |
| | activation |
| THBD | Thrombomodulin activates degradation of factors Va and VIIIa reduces |
| | thrombin |
| HELZ | Helicase zink finger |
| NGRN | Neugrin neurite outgrowth differentiation |
| CFLAR | Caspase 8 and FADD like apoptosis regulator |
| MERTK | Thrombotic response platelet activation |
| SEMA4C | Growth-cone guidance growing tissue |
| IFIT2 | Interferon induced tetratricopeptide |
| STAB1 | Stabilin1 scavenger receptor PM<-> EE traffic |
| DYSF | Dysferlin limb-gridle muscular dystrophy 2B calcium mediated |
| | membrane fusion autoimmune disease |
| CEBPD | CCAAT/enhancer binding protein (C/EBP), delta transcription |
| | activation differentiation macrophages |

33

| | |
|---|---|
| CTSB | Cathepsin B cysteine proteinase |
| HELZ | Helicase zink finger |
| JARID2 | Jumonji Nuclear prevents cell replication |
| CFLAR | Caspase 8 and FADD like apoptosis regulator |
| MARCH1 | Membrane associated ring finger (CSHCU)1 down-regulation of |
| (219574_at) | MHC1 by ubiquitin ligase |
| BCL6 | Zink finger prot 51 modulate the transcription of START-dependent IL- |
| | 4 responses of B cells |

34

| | |
|---|---|
| SASH1 | SAM and SH3 daomain containing reduced in cancer cell cycle |
| ARL4A | Meiosis |
| MARCKSL1 | MARCKS like brain organization |
| CYB561 | Cytochrome B senescence iron |
| HSP5A | HSP70 glucose regulation BIP |
| MMP9 | Matrix metalloproteinase arthritis IL8 mobolization of hematopoetic |
| | progenitors |
| DENND2D | DENN/MADD domain containing 2D |
| H3F3B | Histon 3B |
| SCARB2 | Scavenger receptor B2 lysosome endosome LIMP2 |
| GPR30 | G protein receptor estrogen response |

35

| | |
|---|---|
| CEBPD | CCAAT/enhancer binding protein (C/EBP), delta transcription |
| | activation differentiation macrophages |
| HLA-DPA1 | Antigen presentation |
| ITGAM | IntegrinaM macrophage receptor C3B complement related CD11B |
| | recruitment of leukocytes to site of vessel-injury |

36

| | |
|---|---|
| DICER1 | RNA helicase (RNAi) |
| DDEF1 | Development differentiation enhancing factor-1 GAP activity motility |
| IL2RG | IL2 receptor scid |
| JARID2 | Jumonji Nuclear prevents cell replication |

37

| | |
|---|---|
| H3F3B | Histon 3B |
| SCARB2 | Scavenger receptor B2 lysosome endosome LIMP2 |
| GPR30 | G protein receptor estrogen response |
| SASH1 | SAM and SH3 daomain containing reduced in cancer cell cycle |
| CXCR3 | chimokine receptor 3 migration recruitment |
| IFIH1 | Interferon induced with helicase C domain activates antiviral (RNA |
| | virus) response induce terminal differentiation |
| THBD | Thrombomodulin activates degradation of factors Va and VIIIa reduces |
| | thrombin |
| BIN1 | Bridging integrator adaptor nucleus cytoplasm phosphoinositides |
| | (AMPH related) |
| MGC14376 | ? |
| PER1 | Period homolog circadian expression |
| KLHL2 | Kelch like 2 redistribute cytoskeleton punctation neuron depolarization |
| | differentiation? Macrophage response |

38

| | |
|---|---|
| HSP5A | HSP70 glucose regulation BIP |
| MMP9 | Matrix metalloproteinase arthritis IL8 mobolization of hematopoetic |
| | progenitors |
| TAP2 | Transporter 2 ABC/B MDR/TAP antigen presentation |
| SASH1 | SAM and SH3 daomain containing reduced in cancer cell cycle |
| TAXIBP3 | (TIP1) cell motility T cell leukemia virus binding protein |
| CDKAL1 | CDK5 regulatory subunit like iron binding |
| KIF22 | Kinesin22 cell division motor |
| C6orf149 | |
| LYRM4 | LYR motif containing mitochondria? |

39

| | |
|---|---|
| GPR30 | G protein receptor estrogen response |
| SASH1 | SAM and SH3 daomain containing reduced in cancer cell cycle |
| H3F3B | Histon 3B |

40

| | |
|---|---|
| C3AR1 | Complement component 3a receptor 1 protein receptor to anaphylaxsis |
| | C3a activate macrophages |
| TAP2 | Transporter 2 ABC/B MDR/TAP antigen presentation |
| SCARB2 | Scavenger receptor B2 lysosome endosome LIMP2 |
| CDKAL1 | CDK5 regulatory subunit like iron binding |

41

| | |
|---|---|
| CFLAR | Caspase 8 and FADD like apoptosis regulator |
| TAXIBP3 | (TIP1) cell motility T cell leukemia virus binding protein |
| CYB561 | Cytochrome B senescence iron |
| CTSB | Cathepsin B cysteine proteinase |
| HELZ | Helicase zink finger |
| HLA-DPA1 | Antigen presentation |
| LYGE | Lymphocyte antigen 6 hematopoetic signaling |
| CENTA2 | Binds PIP2 signal |
| IRF1 | Interferon regulatory factor transcription factor antiviral defence |
| CDC42EP3 | Rho GTPAse negative regulator induce pseudopodia |
| GALNS | Galactosamine 6 sulfate sulfatase MPSIVA |

42

| | |
|---|---|
| FNTA | Fernisyl transferase CAAX box connects fernisyl to protein cysteins |
| NGRN | Neugrin neurite outgrowth differentiation |
| DAB2 | Growth of tissue embryonic development |

43

| | |
|---|---|
| CDC42EP3 | Rho GTPAse negative regulator induce pseudopodia |
| HELZ | Helicase zink finger |
| CFLAR | Caspase 8 and FADD like apoptosis regulator |
| DAB2 | Growth of tissue embryonic development |
| MMP9 | Matrix metalloproteinase arthritis IL8 mobolization of hematopoetic |
| | progenitors |
| DDEF1 | Development differentiation enhancing factor-1 GAP activity motility |
| UBE2J1 | Ubiquitin conjugating enzyme ER degradation |

44

| | |
|---|---|
| CYB561 | Cytochrome B senescence iron |
| CFLAR | Caspase 8 and FADD like apoptosis regulator |
| IRF1 | Interferon regulatory factor transcription factor antiviral defence |
| SASH1 | SAM and SH3 daomain containing reduced in cancer cell cycle |
| CXCR3 | chimokine receptor 3 migration recruitment |
| RFX5 | Regulatory factor 5 HLA II expression nuclear protein activates MHC |
| | promoters collagen |
| PSME1 | Proteasome activation subunite 1 makes immuno proteasome |
| SYK | Spleen tyrosine kinase |

45

| | |
|---|---|
| TAXIBP3 | (TIP1) cell motility T cell leukemia virus binding protein |
| DDEF1 | Development differentiation enhancing factor-1 GAP activity motility |
| IL2RG | IL2 receptor scid |
| DAB2 | Growth of tissue embryonic development |
| CFLAR | Caspase 8 and FADD like apoptosis regulator |
| CXCR3 | chimokine receptor 3 migration recruitment |
| CDKAL1 | CDK5 regulatory subunit like iron binding |
| HSP5A | HSP70 glucose regulation BIP |
| BIN1 | Bridging integrator adaptor nucleus cytoplasm phosphoinositides |
| | (AMPH related) |
| IFIH1 | Interferon induced with helicase C domain activates antiviral (RNA |
| | virus) response induce terminal differentiation |
| SEMA4C | Growth-cone guidance growing tissue |
| MAFB | Regulate megakaryocite differentiation |
| IL32 | Induce by T cell NK cell activation activates TNF in macrophages |

46

| | |
|---|---|
| SYK | Spleen tyrosine kinase |
| CYB561 | Cytochrome B senescence iron |

47

| | |
|---|---|
| BTN3A1 | Lipid metabolism butyrophylin |
| PADI4 | Peptidyl arginine deaminase granulocyte, macrophage development |
| | inflammation |
| IFIH1 | Interferon induced with helicase C domain activates antiviral (RNA virus) |
| | response induce terminal differentiation |
| DAB2 | Growth of tissue embryonic development |
| HTRA1 | Serine peptidase 1 reguate IGF1 response cell growth |
| SLC2A3 | Facilitated glucose transport induced in hypoxia |
| PDCD4 | Programmed cell death 4 nucleus proliferating cells NKT |
| BIN1 | Bridging integrator adaptor nucleus cytoplasm phosphoinositides |
| | (AMPH related) |

48

| | |
|---|---|
| FLT3 | fms-related tyrosine kinase 3 receptor regulates hematopoiesis |
| VSIG4 | V-set and immunoglobulin domain containing specific expression on |
| | resting macrophages suggests important for the maintenance of T cell |
| | unresponsiveness in healthy tissues |

49

| | |
|---|---|
| MGAM | maltase-glucoamylase, brush border membrane granulocytes |
| AHR | Aryl hydrocarbon receptor transcription factor aromatic activates CYP |
| | cell adheasion migration |

50

| | |
|---|---|
| GALNS | Galactosamine 6 sulfate sulfatase MPSIVA |
| NGRN | Neugrin neurite outgrowth differentiation |
| CFLAR | Caspase 8 and FADD like apoptosis regulator |

51

| | |
|---|---|
| RNASE2 | RNAase (liver eosinophil derived neurotoxin) immune response |
| CDKAL1 | CDK5 regulatory subunit like iron binding |

52

| | |
|---|---|
| MERTK | Thrombotic response platelet activation |
| DDEF1 | Development differentiation enhancing factor-1 GAP activity motility |
| HLA-DPA1 | Antigen presentation |

53

| | |
|---|---|
| CREB5 | cAMP responsive element binding protein 5 |
| HIP1 | membrane-associated protein colocalizes with huntingtin hematopoietic |
| | malignancies |

54

| | |
|---|---|
| ITGAM | IntegrinaM macrophage receptor C3B complement related CD11B |
| | recruitment of leukocytes to site of vessel-injury |
| ARHGAP19 | Rho GTPase activating protein |
| CDKAL1 | CDK5 regulatory subunit like iron binding |
| CEBPD | CCAAT/enhancer binding protein (C/EBP), delta transcription |
| | activation differentiation macrophages |
| TAP2 | Transporter 2 ABC/B MDR/TAP antigen presentation |

55

| | |
|---|---|
| HIP1 | membrane-associated protein colocalizes with huntingtin hematopoietic |
| | malignancies |
| CREB5 | cAMP responsive element binding protein 5 |

56

| | |
|---|---|
| ARL4A | Meiosis |
| CDKAL1 | CDK5 regulatory subunit like iron binding |
| SASH1 | SAM and SH3 daomain containing reduced in cancer cell cycle |
| DENND2D | DENN/MADD domain containing 2D |

57

| | |
|---|---|
| VSIG4 | V-set and immunoglobulin domain containing specific expression on |
| | resting macrophages suggests important for the maintenance of T cell |
| | unresponsiveness in healthy tissues |
| FLT3 | fms-related tyrosine kinase 3 receptor regulates hematopoiesis |

58

| | |
|---|---|
| TAP2 | Transporter 2 ABC/B MDR/TAP antigen presentation |
| ITGAM | IntegrinaM macrophage receptor C3B complement related CD11B |
| | recruitment of leukocytes to site of vessel-injury |
| HSP5A | HSP70 glucose regulation BIP |
| LYGE | Lymphocyte antigen 6 hematopoetic signaling |
| C3AR1 | Complement component 3a receptor 1 protein receptor to anaphylaxsis |
| | C3a activate macrophages |
| SEMA4C | Growth-cone guidance growing tissue |
| KIF22 | Kinesin22 cell division motor |
| JARID2 | Jumonji Nuclear prevents cell replication |
| MARCH1 | Membrane associated ring finger (CSHCU)1 down-regulation of |
| (219574_at) | MHC1 by ubiquitin ligase |
| IFIT2 | Interferon induced tetratricopeptide |
| CALM1 | Calmodulin1 growth cells cycle signal |
| YES1 | Oncogene TK |
| ALOX5 | arachidonate 5-lipoxygenase Prcursor for leukotrien immune rsponse |
| | vascular hypoxia |
| S100A8 | Inflammation activation by macrophages and granulocytes leukocyte |
| | trafficking and arachidonic acid metabolism |

59

| | |
|---|---|
| F5 | Coagulation factor V (proaccelerin labile factor) thrombosis |
| SCARB2 | Scavenger receptor B2 lysosome endosome LIMP2 |

60

| | |
|---|---|
| ALOX5 | arachidonate 5-lipoxygenase Prcursor for leukotrien immune rsponse |
| | vascular hypoxia |
| TAP2 | Transporter 2 ABC/B MDR/TAP antigen presentation |

61

| | |
|---|---|
| IL2RG | IL2 receptor scid |
| CENTA2 | Binds PIP2 signal |
| TAXIBP3 | (TIP1) cell motility T cell leukemia virus binding protein |
| IL32 | Induce by T cell NK cell activation activates TNF in macrophages |
| DICER1 | RNA helicase (RNAi) |

62

| | |
|---|---|
| MMP9 | Matrix metalloproteinase arthritis IL8 mobolization of hematopoetic |
| | progenitors |
| HSP5A | HSP70 glucose regulation BIP |
| SASH1 | SAM and SH3 daomain containing reduced in cancer cell cycle |
| AHR | Aryl hydrocarbon receptor transcription factor aromatic activates CYP |
| | cell adheasion migration |
| HTRA1 | Serine peptidase 1 reguate IGF1 response cell growth |
| PADI4 | Peptidyl arginine deaminase granulocyte, macrophage development |
| | inflammation |
| CDC42EP3 | Rho GTPAse negative regulator induce pseudopodia |

63

| | |
|---|---|
| THBD | Thrombomodulin activates degradation of factors Va and VIIIa reduces |
| | thrombin |
| CDKAL1 | CDK5 regulatory subunit like iron binding |
| HLA-DPA1 | Antigen presentation |
| H3F3B | Histon 3B |

64

| | |
|---|---|
| IL32 | Induce by T cell NK cell activation activates TNF in macrophages |
| IL2RG | IL2 receptor scid |
| TAXIBP3 | (TIP1) cell motility T cell leukemia virus binding protein |
| JARID2 | Jumonji Nuclear prevents cell replication |

65

| | |
|---|---|
| HELZ | Helicase zink finger |
| KIF22 | Kinesin22 cell division motor |
| JARID2 | Jumonji Nuclear prevents cell replication |
| HLA-DPA1 | Antigen presentation |
| SCARB2 | Scavenger receptor B2 lysosome endosome LIMP2 |
| CDC42EP3 | Rho GTPAse negative regulator induce pseudopodia |
| YES1 | Oncogene TK |
| CFLAR | Caspase 8 and FADD like apoptosis regulator |
| CTSB | Cathepsin B cysteine proteinase |

66

| | |
|---|---|
| ASFA1 | Histone chaperone DNA replication repair senescence |
| SCARB2 | Scavenger receptor B2 lysosome endosome LIMP2 |
| IMP3 | U3 snoRNA |

67

| | |
|---|---|
| JARID2 | Jumonji Nuclear prevents cell replication |
| YES1 | Oncogene TK |
| HELZ | Helicase zink finger |
| SCARB2 | Scavenger receptor B2 lysosome endosome LIMP2 |
| CDKAL1 | CDK5 regulatory subunit like iron binding |
| CTSB | Cathepsin B cysteine proteinase |
| IL32 | Induce by T cell NK cell activation activates TNF in macrophages |
| AHR | Aryl hydrocarbon receptor transcription factor aromatic activates CYP |
| | cell adheasion migration |
| DICER1 | RNA helicase (RNAi) |
| TAP2 | Transporter 2 ABC/B MDR/TAP antigen presentation |
| DAB2 | Growth of tissue embryonic development |
| TRIB1 | Tribbles homolog 1 Signal transduction regulation |

68

| | |
|---|---|
| BCL6 | Zink finger prot 51 modulate the transcription of START-dependent IL- |
| | 4 responses of B cells |
| CTSB | Cathepsin B cysteine proteinase |

69

| | |
|---|---|
| RFX5 | Regulatory factor 5 HLA II expression nuclear protein activates MHC |
| | promoters collagen |
| CXCR3 | chimokine receptor 3 migration recruitment |
| KIF22 | Kinesin22 cell division motor |
| AHR | Aryl hydrocarbon receptor transcription factor aromatic activates CYP |
| | cell adheasion migration |
| CYB561 | Cytochrome B senescence iron |
| C6orf149 | |
| LYRM4 | LYR motif containing mitochondria? |
| GLUL | Glutamate amonia ligase (glutamine synthase regulate body pH |
| | removing amonia |

70

| | |
|---|---|
| YES1 | Oncogene TK |
| JARID2 | Jumonji Nuclear prevents cell replication |
| HELZ | Helicase zink finger |
| TAP2 | Transporter 2 ABC/B MDR/TAP antigen presentation |
| CXCR3 | chimokine receptor 3 migration recruitment |
| TMEM49 | VMP1 vacuole formation |

71

| | |
|---|---|
| S100A8 | Inflammation activation by macrophages and granulocytes leukocyte |
| | trafficking and arachidonic acid metabolism |
| TAP2 | Transporter 2 ABC/B MDR/TAP antigen presentation |

72

| | |
|---|---|
| PER1 | Period homolog circadian expression |
| KIF22 | Kinesin22 cell division motor |
| H3F3B | Histon 3B |
| FANCF | Fanconi's anemia complementation F adaptor DNA binding repair |

73

| | |
|---|---|
| AHR | Aryl hydrocarbon receptor transcription factor aromatic activates CYP |
| | cell adheasion migration |
| JARID2 | Jumonji Nuclear prevents cell replication |
| RFX5 | Regulatory factor 5 HLA II expression nuclear protein activates MHC |
| | promoters collagen |
| MMP9 | Matrix metalloproteinase arthritis IL8 mobolization of hematopoetic |
| | progenitors |
| IFIT2 | Interferon induced tetratricopeptide |
| MGAM | maltase-glucoamylase, brush border membrane granulocytes |
| SLC2A3 | Facilitated glucose transport induced in hypoxia |

74

| | |
|---|---|
| PDCD4 | Programmed cell death 4 nucleus proliferating cells NKT |
| SCARB2 | Scavenger receptor B2 lysosome endosome LIMP2 |
| ARHGAP19 | Rho GTPase activating protein |
| NGRN | Neugrin neurite outgrowth differentiation |
| BIN1 | Bridging integrator adaptor nucleus cytoplasm phosphoinositides |
| | (AMPH related) |
| IFIH1 | Interferon induced with helicase C domain activates antiviral (RNA virus) |
| | response induce terminal differentiation |
| MARCH1 | Membrane associated ring finger (CSHCU) 1 down-regulation of |
| (219574_at) | MHC1 by ubiquitin ligase |
| BTN3A1 | Lipid metabolism butyrophylin |

75

| | |
|---|---|
| IRF1 | Interferon regulatory factor transcription factor antiviral defence |
| CYB561 | Cytochrome B senescence iron |
| CFLAR | Caspase 8 and FADD like apoptosis regulator |
| PSME1 | Proteasome activation subunite 1 makes immuno proteasome |
| IFIT2 | Interferon induced tetratricopeptide |
| IFIH1 | Interferon induced with helicase C domain activates antiviral (RNA virus) |
| | response induce terminal differentiation |
| LYGE | Lymphocyte antigen 6 hematopoetic signaling |

76

| | |
|---|---|
| SLC2A3 | Facilitated glucose transport induced in hypoxia |
| AHR | Aryl hydrocarbon receptor transcription factor aromatic activates CYP |
| | cell adheasion migration |
| BTN3A1 | Lipid metabolism butyrophylin |
| DDEF1 | Development differentiation enhancing factor-1 GAP activity motility |

77

| | |
|---|---|
| CYP1B1 | Steroid metabolism signaling eye |
| MARCH1 | Membrane associated ring finger (CSHCU)1 down-regulation of |
| (219574_at) | MHC1 by ubiquitin ligase |

78

| | |
|---|---|
| TRIB1 | Tribbles homolog 1 Signal transduction regulation |
| JARID2 | Jumonji Nuclear prevents cell replication |
| MGC14376 | ? |

79

| | |
|---|---|
| LYGE | Lymphocyte antigen 6 hematopoetic signaling |
| MGC14376 | ? |
| CFLAR | Caspase 8 and FADD like apoptosis regulator |
| MARCH1 | Membrane associated ring finger (CSHCU)1 down-regulation of |
| (219574_at) | MHC1 by ubiquitin ligase |
| IRF1 | Interferon regulatory factor transcription factor antiviral defence |
| IFIT2 | Interferon induced tetratricopeptide |
| SCARB2 | Scavenger receptor B2 lysosome endosome LIMP2 |
| TAP2 | Transporter 2 ABC/B MDR/TAP antigen presentation |

80

| | |
|---|---|
| CXCR3 | chimokine receptor 3 migration recruitment |
| CDKAL1 | CDK5 regulatory subunit like iron binding |
| RNASE1 | Pancreatic RNAase |

81

| | |
|---|---|
| DAB2 | Growth of tissue embryonic development |
| BTN3A1 | Lipid metabolism butyrophylin |
| JARID2 | Jumonji Nuclear prevents cell replication |
| DDEF1 | Development differentiation enhancing factor-1 GAP activity motility |
| CDC42EP3 | Rho GTPAse negative regulator induce pseudopodia |
| TAXIBP3 | (TIP1) cell motility T cell leukemia virus binding protein |
| CALM1 | Calmodulin1 growth cells cycle signal |
| SEMA4C | Growth-cone guidance growing tissue |
| FNTA | Fernisyl transferase CAAX box connects fernisyl to protein cysteins |

82

| | |
|---|---|
| HTRA1 | Serine peptidase 1 reguate IGF1 response cell growth |
| BTN3A1 | Lipid metabolism butyrophylin |
| MMP9 | Matrix metalloproteinase arthritis IL8 mobolization of hematopoetic |
| | progenitors |
| CR1 | Complement receptor red/white blood cells membrane malaria receptor |

83

| | |
|---|---|
| PSME1 | Proteasome activation subunite 1 makes immuno proteasome |
| IRF1 | Interferon regulatory factor transcription factor antiviral defence |
| CYB561 | Cytochrome B senescence iron |
| IFIT2 | Interferon induced tetratricopeptide |
| PECI | Peroxisomal enoyl CoA isomerase b Oxidation FA |

84

| | |
|---|---|
| MARCKSL1 | MARCKS like brain organization |
| SASH1 | SAM and SH3 daomain containing reduced in cancer cell cycle |
| NGRN | Neugrin neurite outgrowth differentiation |

### Example 9

### Differential Expression Associated with Hemorrhagic Stroke

This example describes particular changes in expression, such as gene or protein expression, that are associated with hemorrhagic stroke, such as intracerebral hemorrhagic stroke. Although particular hemorrhagic stroke-related molecules are listed in this example, one skilled in the art will appreciated that other molecules can be used based on the teachings in this disclosure.

In particular examples, detecting differential expression includes detecting differences in expression (such as an increase, decrease, or both). The method can further include determining the magnitude of the difference in expression, wherein the magnitude of the change is associated with hemorrhagic stroke. Particular examples of hemorrhagic stroke-related molecules that are differentially expressed in association with the diagnosis of a hemorrhagic stroke, such as an ICH stroke, and their direction of change (upregulated or downregulated), and the magnitude of the change (as expressed as a percent, t-statistic, and fold change) are provided in Table 12.

TABLE 12
Exemplary patterns of expression associated with hemorrhagic stroke

| Hemorrhagic Stroke | Change in | |
|---|---|---|
| Molecule | Expression | Magnitude of the change |
| CD163 | upregulated | t-statistic of at least 5 (such as |
| | | at least 8) |
| | | at least 50% |
| | | at least 4-fold |
| IL1R2 | upregulated | t-statistic of at least 10 (such |
| | | as at least 19) |
| | | at least 50% |
| | | at least 4-fold |
| Acyl-CoA synthease long | upregulated | t-statistic of at least 6 (such as |
| chain family member 1 | | at least 7) |
| | | at least 50% |
| | | at least 4-fold |
| Amphiphysin | upregulated | t-statistic of at least 20 (such |
| | | as at least 24) |
| | | at least 50% |
| | | at least 4-fold |
| haptoglobin | upregulated | t-statistic of at least 4 (such as |
| | | at least 5) |
| | | at least 50% |
| | | at least 4-fold |
| TAP2 | downregulated | t-statistic of no more than −5 |
| | | (such as no more than −8 |
| | | at least 50% |
| | | at least 4-fold |
| semaphorin 4C | downregulated | t-statistic of no more than −5 |
| (Sema4C) | | (such as no more than −8.5) |
| | | at least 50% |
| | | at least 4-fold |
| Granzyme M | downregulated | t-statistic of no more than − |
| | | (such as no more than −7.5) |
| | | at least 50% |
| | | at least 4-fold |

Therefore, IL1R2, Acyl-CoA synthease long chain family member 1, amphiphysin, and CD163 are upregulated by a magnitude of at least 50%, at least 4-fold or have a t-statistic of at least 5. That is, IL1R2, Acyl-CoA synthease long chain family member 1, amphiphysin, and CD163 are upregulated by an amount associated with hemorrhagic stroke, for example at least 50% or at least 4-fold (or have a t-statistic of at least 5). In addition, TAP2 and Sema4C are downregulated by a magnitude of at least 50%, at least 4-fold or have a t-statistic of no more than −5. That is, TAP2 and Sema4C are downregulated by an amount associated with hemorrhagic stroke, for example at least 50% or at least 4-fold (or have a t-statistic of no more than −5).

One example of a pattern of expression of proteins that have been found to be associated with hemorrhagic stroke, such as upregulation of IL1R2, Acyl-CoA synthease long chain family member 1, and amphiphysin wherein the magnitude of change is at least 4-fold for each of IL1R2, Acyl-CoA synthease long chain family member 1, and amphiphysin. Another example of a pattern of expression of proteins that have been found to be associated with hemorrhagic stroke is as downregulation of TAP2 and Sema4C for example wherein the magnitude of change is at least 4-fold for each of these proteins.

### Example 10

### Adjustment for Race, Gender, Age, and Time of Blood Draw

This example describes methods used to adjust the stroke gene profile for race, age, gender, and time of blood draw.

The data obtained in Example 3 (CEL files of 8 patients with confirmed ICH, 19 ischemic stroke subjects and 18 referent control subjects) was analyzed as follows. Sample outlier analysis was performed using covariance-based Principal Component Analysis (PCA) and Pearson Correlation Analysis. PCA was used to identify those samples causing cross-sample compression by component biplot; Pearson Correlation Analysis was used to identify any sample having a cross-sample correlation value less than 0.70 70% of the time. Samples identified by either method were classified as outliers and removed from further analysis. LOWESS (LOcally WEighted Scatter plot Smoothing) was used for noise analysis. Sample data was divided into groups based on disease class, where the data within each group was used to calculate the coefficient of variation (C.V.) and the median RMA (Robust Multi-array Analysis) expression value for each gene probe. LOWESS was then used to model C.V. by median RMA expression within each group; rendering class-specific noise curves. The resulting noise curves were then interrogated to find the greatest median RMA expression value at which C.V. decreases as median RMA expression decreases. This value was used to define system noise. RMA expression values less than system noise were reset to equal the value of system noise. The mean RMA expression value within each disease class for each gene probe was calculated and used to remove those gene probes from further analysis that do not have at least one class with a mean RMA expression value greater than system noise.

To determine the effect of gender and race on gene expression, Analysis of Variance (ANOVA) was used. RMA expression values for all samples were paired with the corresponding gender or race of the person the sample was collected from. ANOVA was performed on a gene fragment by gene fragment basis using gender or race as a factor. Resulting significance values were captured post ANOVA and interrogated using a false-discovery rate (FDR) multiple comparison correction (MCC) procedure. Gene fragments having a significance value less than 0.05 under FDR MCC condition were classified as significantly associated with gender or race (Table 13). Such genes are ideally not used to determine if a subject has suffered a stroke, or to classify a stroke as hemorrhagic or ischemic, as expression of these genes is associated with gender or race.

To determine the effect of age on gene expression, Spearman Correlation Analysis was used. RMA expression values for all samples were paired with the corresponding age of the person the sample was collected from. Spearman Correlation Analysis was performed on a gene fragment by gene fragment basis. Resulting significance values were captured post analysis and interrogated using a false-discovery rate (FDR) multiple comparison correction (MCC) procedure. Gene fragments having a significance value less than 0.05 under FDR MCC condition were classified as significantly associated with age. As shown in Table 13, no gene expression was significantly associated with age.

To determine the effect of draw time on gene expression, Pearson Correlation Analysis was used. RMA expression values for all samples were paired with the corresponding draw time that the sample was collected. Pearson Correlation Analysis was performed on a gene fragment by gene fragment basis. Resulting significance values were captured post analysis and interrogated using a false-discovery rate (FDR) multiple comparison correction (MCC) procedure. Gene fragments having a significance value less than 0.05 under FDR MCC condition were classified as significantly associated with draw time (Table 13). The genes listed in Table 13 with p-values significant for draw time may reflect changes in expression that occur over time following a stroke. Therefore, such markers can be used to determine if a subject has suffered a stroke or classify the stroke as ischemic or hemorrhagic. Therefore, in some examples, the methods provided herein do the genes listed in Table 13 with p-values significant for draw time, and in some examples, the arrays provided herein include one or more of the markers listed in Table 13 with p-values significant for draw time.

As shown in Table 13, 24 gene probes had p-values significant for gender (noted to be genes on the X or Y chromosome), 6 gene probes had p-values significant for race, no gene probes had p-values significant for age, and 137 gene probes had p-values significant for time of blood draw. Therefore, the genes listed in Table 13 with p-values significant for gender or race are not ideal candidates for identification of subjects who have suffered a stroke or classification of whether the subject had an ischemic or hemorrhagic stroke, as expression of these genes was correlated with non-stroke factors (gender, race). Therefore, in some examples, the methods provided herein do not use any of the genes listed in Table 13 with p-values significant for gender or race, and in some examples, the arrays provided herein do not include the markers listed in Table 13 with p-values significant for gender or race.

TABLE 13
Genes with significant p-values for gender, race, age, or draw time.

| Probe Set ID\hat{ } | Gender | Race | Age | Draw Time | Gene Name |
|---|---|---|---|---|---|
| 201909_at | Yes | No | No | No | ribosomal protein S4, Y-linked 1 |
| 221728_x_at | Yes | No | No | No | X (inactive)-specific transcript |
| 214218_s_at | Yes | No | No | No | X (inactive)-specific transcript |
| 206700_s_at | Yes | No | No | No | jumonji, AT rich interactive |
| | | | | | domain 1D |
| 205000_at | Yes | No | No | No | DEAD (Asp-Glu-Ala-Asp) box |
| | | | | | polypeptide 3, Y-linked |
| 205001_s_at | Yes | No | No | No | DEAD (Asp-Glu-Ala-Asp) box |
| | | | | | polypeptide 3, Y-linked |
| 210322_x_at | Yes | No | No | No | ubiquitously transcribed |
| | | | | | tetratricopeptide repeat gene, Y- |
| | | | | | linked |
| 204409_s_at | Yes | No | No | No | eukaryotic translation initiation |
| | | | | | factor 1A, Y-linked |
| 204410_at | Yes | No | No | No | eukaryotic translation initiation |
| | | | | | factor 1A, Y-linked |
| 203992_s_at | Yes | No | No | No | ubiquitously transcribed |
| | | | | | tetratricopeptide repeat, X |
| | | | | | chromosome |
| 201019_s_at | Yes | No | No | No | eukaryotic translation initiation |
| | | | | | factor 1A, X-linked /// eukaryotic |
| | | | | | translation initiation factor 1A |
| | | | | | pseudogene 1 |
| 208067_x_at | Yes | No | No | No | ubiquitously transcribed |
| | | | | | tetratricopeptide repeat gene, Y- |
| | | | | | linked |
| 204061_at | Yes | No | No | No | protein kinase, X-linked |
| 203974_at | Yes | No | No | No | haloacid dehalogenase-like |
| | | | | | hydrolase domain containing 1A |
| 206279_at | Yes | No | No | No | protein kinase, Y-linked |
| 201016_at | Yes | No | No | No | eukaryotic translation initiation |
| | | | | | factor 1A, X-linked |
| 206769_at | Yes | No | No | No | thymosin, beta 4, Y-linked |
| 216342_x_at | Yes | No | No | No | similar to 40S ribosomal protein |
| | | | | | S4, X isoform |
| 200933_x_at | Yes | No | No | No | ribosomal protein S4, X-linked |
| 201018_at | Yes | No | No | No | eukaryotic translation initiation |
| | | | | | factor 1A, X-linked |
| 208174_x_at | Yes | No | No | No | zinc finger (CCCH type), RNA- |
| | | | | | binding motif and serine/arginine |
| | | | | | rich 2 |
| 213876_x_at | Yes | No | No | No | zinc finger (CCCH type), RNA- |
| | | | | | binding motif and serine/arginine |
| | | | | | rich 2 |
| 206624_at | Yes | No | No | No | ubiquitin specific peptidase 9, Y- |
| | | | | | linked (fat facets-like, |
| | | | | | Drosophila) |
| 203990_s_at | Yes | No | No | No | ubiquitously transcribed |
| | | | | | tetratricopeptide repeat, X |
| | | | | | chromosome |
| 205048_s_at | No | Yes | No | No | phosphoserine phosphatase |
| 219038_at | No | Yes | No | No | MORC family CW-type zinc |
| | | | | | finger 4 |
| 214912_at | No | Yes | No | No | — |
| 205085_at | No | Yes | No | No | origin recognition complex, |
| | | | | | subunit 1-like (yeast) |
| 212911_at | No | Yes | No | No | DnaJ (Hsp40) homolog, |
| | | | | | subfamily C, member 16 |
| 208919_s_at | No | Yes | No | No | NAD kinase |
| 209446_s_at | No | No | No | Yes | chromosome 7 open reading |
| | | | | | frame 44 |
| 217523_at | No | No | No | Yes | CD44 molecule (Indian blood |
| | | | | | group) |
| 215221_at | No | No | No | Yes | Forkhead box P1 |
| 215404_x_at | No | No | No | Yes | fibroblast growth factor receptor |
| | | | | | 1 (fms-related tyrosine kinase 2, |
| | | | | | Pfeiffer syndrome) |
| 222152_at | No | No | No | Yes | Programmed cell death 6 |
| 222186_at | No | No | No | Yes | Zinc finger, AN1-type domain 6 |
| 215577_at | No | No | No | Yes | Ubiquitin-conjugating enzyme |
| | | | | | E2E 1 (UBC4/5 homolog, yeast) |
| 215375_x_at | No | No | No | Yes | Leucine rich repeat (in FLII) |
| | | | | | interacting protein 1 |
| 222133_s_at | No | No | No | Yes | PHD finger protein 20-like 1 |
| 222357_at | No | No | No | Yes | zinc finger and BTB domain |
| | | | | | containing 20 |
| 217653_x_at | No | No | No | Yes | — |
| 210210_at | No | No | No | Yes | myelin protein zero-like 1 |
| 221616_s_at | No | No | No | Yes | TAF9B RNA polymerase II, |
| | | | | | TATA box binding protein |
| | | | | | (TBP)-associated factor, 31 kDa |
| 222214_at | No | No | No | Yes | CDNA: FLJ21335 fis, clone |
| | | | | | COL02546 |
| 220113_x_at | No | No | No | Yes | polymerase (RNA) I polypeptide |
| | | | | | B, 128 kDa |
| 215888_at | No | No | No | Yes | Androgen-induced proliferation |
| | | | | | inhibitor |
| 216211_at | No | No | No | Yes | Chromosome 10 open reading |
| | | | | | frame 18 |
| 204055_s_at | No | No | No | Yes | CTAGE family, member 5 |
| 205317_s_at | No | No | No | Yes | solute carrier family 15 |
| | | | | | (H+/peptide transporter), |
| | | | | | member 2 |
| 206088_at | No | No | No | Yes | leucine rich repeat containing 37, |
| | | | | | member A2 |
| 206965_at | No | No | No | Yes | Kruppel-like factor 12 |
| 210282_at | No | No | No | Yes | zinc finger, MYM-type 2 |
| 210528_at | No | No | No | Yes | major histocompatibility |
| | | | | | complex, class I-related |
| 210742_at | No | No | No | Yes | CDC14 cell division cycle 14 |
| | | | | | homolog A (S. cerevisiae) |
| 214163_at | No | No | No | Yes | Chromosome 1 open reading |
| | | | | | frame 41 |
| 214405_at | No | No | No | Yes | CUG triplet repeat, RNA binding |
| | | | | | protein 2 |
| 215105_at | No | No | No | Yes | hypothetical gene CG030 |
| 215151_at | No | No | No | Yes | dedicator of cytokinesis 10 |
| 215175_at | No | No | No | Yes | pecanex homolog (Drosophila) |
| 215190_at | No | No | No | Yes | PCI domain containing 1 |
| | | | | | (herpesvirus entry mediator) |
| 215191_at | No | No | No | Yes | CDNA FLJ14085 fis, clone |
| | | | | | HEMBB1002534 |
| 215204_at | No | No | No | Yes | SUMO1/sentrin specific |
| | | | | | peptidase 6 |
| 215385_at | No | No | No | Yes | Fatso |
| 215392_at | No | No | No | Yes | Ubiquitin specific peptidase 3 |
| 215599_at | No | No | No | Yes | SMA4 /// similar to SMA4 |
| 215750_at | No | No | No | Yes | KIAA1659 protein |
| 215786_at | No | No | No | Yes | Remodeling and spacing factor 1 |
| 216000_at | No | No | No | Yes | KIAA0484 protein |
| 216006_at | No | No | No | Yes | WAS/WASL interacting protein |
| | | | | | family, member 2 |
| 216012_at | No | No | No | Yes | Unidentified mRNA, partial |
| | | | | | sequence |
| 216109_at | No | No | No | Yes | Thyroid hormone receptor |
| | | | | | associated protein 2 |
| 216170_at | No | No | No | Yes | Eukaryotic translation elongation |
| | | | | | factor 1 gamma |
| 216197_at | No | No | No | Yes | activating transcription factor 7 |
| | | | | | interacting protein |
| 216527_at | No | No | No | Yes | HLA complex group 18 |
| 216614_at | No | No | No | Yes | Inositol 1,4,5-triphosphate |
| | | | | | receptor, type 2 |
| 216765_at | No | No | No | Yes | Mitogen-activated protein kinase |
| | | | | | kinase 5 |
| 219871_at | No | No | No | Yes | hypothetical protein FLJ13197 /// |
| | | | | | hypothetical protein LOC727852 |
| | | | | | /// hypothetical protein |
| | | | | | LOC731366 |
| 220085_at | No | No | No | Yes | helicase, lymphoid-specific |
| 220221_at | No | No | No | Yes | vacuolar protein sorting 13 |
| | | | | | homolog D (S. cerevisiae) |
| 220609_at | No | No | No | Yes | hypothetical protein LOC202181 |
| 220694_at | No | No | No | Yes | DDEF1 intronic transcript 1 |
| 220704_at | No | No | No | Yes | IKAROS family zinc finger 1 |
| | | | | | (Ikaros) |
| 221617_at | No | No | No | Yes | TAF9B RNA polymerase II, |
| | | | | | TATA box binding protein |
| | | | | | (TBP)-associated factor, 31 kDa |
| 222286_at | No | No | No | Yes | small nuclear RNA activating |
| | | | | | complex, polypeptide 3, 50 kDa |
| 222310_at | No | No | No | Yes | splicing factor, arginine/serine- |
| | | | | | rich 15 |
| 222313_at | No | No | No | Yes | CCR4-NOT transcription |
| | | | | | complex, subunit 2 |
| 222358_x_at | No | No | No | Yes | Asparagine-linked glycosylation |
| | | | | | 13 homolog (S. cerevisiae) |
| 217671_at | No | No | No | Yes | Regulatory factor X, 3 |
| | | | | | (influences HLA class II |
| | | | | | expression) |
| 222371_at | No | No | No | Yes | Protein inhibitor of activated |
| | | | | | STAT, 1 |
| 217482_at | No | No | No | Yes | CDNA FLJ11925 fis, clone |
| | | | | | HEMBB1000354 |
| 215383_x_at | No | No | No | Yes | spastic paraplegia 21, maspardin |
| | | | | | (autosomal recessive, Mast |
| | | | | | syndrome) |
| 216682_s_at | No | No | No | Yes | family with sequence similarity |
| | | | | | 48, member A |
| 212991_at | No | No | No | Yes | F-box protein 9 |
| 221184_at | No | No | No | Yes | — |
| 220814_at | No | No | No | Yes | — |
| 205603_s_at | No | No | No | Yes | diaphanous homolog 2 |
| | | | | | (Drosophila) |
| 207324_s_at | No | No | No | Yes | desmocollin 1 |
| 219906_at | No | No | No | Yes | hypothetical protein FLJ10213 |
| 220969_s_at | No | No | No | Yes | — |
| 211435_at | No | No | No | Yes | — |
| 203742_s_at | No | No | No | Yes | thymine-DNA glycosylase /// |
| | | | | | similar to G/T mismatch-specific |
| | | | | | thymine DNA glycosylase /// |
| | | | | | similar to G/T mismatch-specific |
| | | | | | thymine DNA glycosylase |
| 222306_at | No | No | No | Yes | Hypothetical protein MGC61571 |
| 207365_x_at | No | No | No | Yes | ubiquitin specific peptidase 34 |
| 208854_s_at | No | No | No | Yes | serine/threonine kinase 24 |
| | | | | | (STE20 homolog, yeast) |
| 217164_at | No | No | No | Yes | — |
| 217715_x_at | No | No | No | Yes | Zinc finger protein 354A |
| 214153_at | No | No | No | Yes | ELOVL family member 5, |
| | | | | | elongation of long chain fatty |
| | | | | | acids (FEN1/Elo2, SUR4/Elo3- |
| | | | | | like, yeast) |
| 206061_s_at | No | No | No | Yes | Dicer1, Dcr-1 homolog |
| | | | | | (Drosophila) |
| 217985_s_at | No | No | No | Yes | bromodomain adjacent to zinc |
| | | | | | finger domain, 1A |
| 215169_at | No | No | No | Yes | solute carrier family 35, member |
| | | | | | E2 |
| 222282_at | No | No | No | Yes | PAP associated domain |
| | | | | | containing 4 |
| 222061_at | No | No | No | Yes | CD58 molecule |
| 202518_at | No | No | No | Yes | B-cell CLL/lymphoma 7B |
| 202682_s_at | No | No | No | Yes | ubiquitin specific peptidase 4 |
| | | | | | (proto-oncogene) |
| 205740_s_at | No | No | No | Yes | hypothetical protein MGC10433 |
| 222266_at | No | No | No | Yes | Chromosome 19 open reading |
| | | | | | frame 2 |
| 208241_at | No | No | No | Yes | neuregulin 1 |
| 219957_at | No | No | No | Yes | RUN and FYVE domain |
| | | | | | containing 2 |
| 215322_at | No | No | No | Yes | LON peptidase N-terminal |
| | | | | | domain and ring finger 1 |
| 213229_at | No | No | No | Yes | Dicer1, Dcr-1 homolog |
| | | | | | (Drosophila) |
| 203273_s_at | No | No | No | Yes | tumor suppressor candidate 2 |
| 220777_at | No | No | No | Yes | kinesin family member 13A |
| 201727_s_at | No | No | No | Yes | ELAV (embryonic lethal, |
| | | | | | abnormal vision, Drosophila)- |
| | | | | | like 1 (Hu antigen R) |
| 211034_s_at | No | No | No | Yes | AF-1 specific protein |
| | | | | | phosphatase |
| 207436_x_at | No | No | No | Yes | KIAA0894 protein |
| 221192_x_at | No | No | No | Yes | hypothetical protein ET |
| 214594_x_at | No | No | No | Yes | ATPase, Class I, type 8B, |
| | | | | | member 1 |
| 213158_at | No | No | No | Yes | Homo sapiens, clone |
| | | | | | IMAGE: 4214654, mRNA |
| 215374_at | No | No | No | Yes | Poly(A) polymerase alpha |
| 212542_s_at | No | No | No | Yes | pleckstrin homology domain |
| | | | | | interacting protein |
| 221915_s_at | No | No | No | Yes | RAN binding protein 1 |
| 206848_at | No | No | No | Yes | homeobox A7 |
| 216524_x_at | No | No | No | Yes | Roundabout, axon guidance |
| | | | | | receptor, homolog 2 (Drosophila) |
| 215761_at | No | No | No | Yes | Dmx-like 2 |
| 215083_at | No | No | No | Yes | Paraspeckle component 1 |
| 221718_s_at | No | No | No | Yes | A kinase (PRKA) anchor protein |
| | | | | | 13 |
| 215179_x_at | No | No | No | Yes | Placental growth factor, vascular |
| | | | | | endothelial growth factor-related |
| | | | | | protein |
| 215528_at | No | No | No | Yes | Mannosyl (alpha-1,6-)- |
| | | | | | glycoprotein beta-1,6-N-acetyl- |
| | | | | | glucosaminyltransferase |
| 206169_x_at | No | No | No | Yes | zinc finger CCCH-type |
| | | | | | containing 7B |
| 212847_at | No | No | No | Yes | Far upstream element (FUSE) |
| | | | | | binding protein 1 |
| 201628_s_at | No | No | No | Yes | Ras-related GTP binding A |
| 215754_at | No | No | No | Yes | scavenger receptor class B, |
| | | | | | member 2 |
| 213956_at | No | No | No | Yes | centrosomal protein 350 kDa |
| 215545_at | No | No | No | Yes | — |
| 215188_at | No | No | No | Yes | serine/threonine kinase 24 |
| | | | | | (STE20 homolog, yeast) |
| 222366_at | No | No | No | Yes | Activity-dependent |
| | | | | | neuroprotector |
| 208498_s_at | No | No | No | Yes | amylase, alpha 1A; salivary /// |
| | | | | | amylase, alpha 1B; salivary /// |
| | | | | | amylase, alpha 1C; salivary /// |
| | | | | | amylase, alpha 2A; pancreatic /// |
| | | | | | amylase, alpha 2B (pancreatic) /// |
| | | | | | similar to Pancreatic alpha- |
| | | | | | amylase precursor (PA) (1,4- |
| | | | | | alpha-D-glucan |
| | | | | | glucanohydrolase) |
| 207525_s_at | No | No | No | Yes | GIPC PDZ domain containing |
| | | | | | family, member 1 |
| 203255_at | No | No | No | Yes | F-box protein 11 |
| 209385_s_at | No | No | No | Yes | proline synthetase co-transcribed |
| | | | | | homolog (bacterial) |
| 213089_at | No | No | No | Yes | glucuronidase, beta pseudogene 1 |
| 204373_s_at | No | No | No | Yes | centrosomal protein 350 kDa |
| 213705_at | No | No | No | Yes | CDNA FLJ30007 fis, clone |
| | | | | | 3NB692000012 |
| 216187_x_at | No | No | No | Yes | Kinesin 2 |
| 208602_x_at | No | No | No | Yes | CD6 molecule |
| 214902_x_at | No | No | No | Yes | LIM domain containing preferred |
| | | | | | translocation partner in lipoma |
| 221855_at | No | No | No | Yes | hypothetical protein LOC644096 |
| 202781_s_at | No | No | No | Yes | skeletal muscle and kidney |
| | | | | | enriched inositol phosphatase |
| 215287_at | No | No | No | Yes | ELISC-1 |
| 215588_x_at | No | No | No | Yes | RIO kinase 3 (yeast) |
| 216870_x_at | No | No | No | Yes | deleted in lymphocytic leukemia, |
| | | | | | 2 /// deleted in lymphocytic |
| | | | | | leukemia 2-like |
| 213531_s_at | No | No | No | Yes | RAB3 GTPase activating protein |
| | | | | | subunit 1 (catalytic) |
| 214441_at | No | No | No | Yes | syntaxin 6 |
| 214289_at | No | No | No | Yes | Proteasome (prosome, |
| | | | | | macropain) subunit, beta type, 1 |
| 220078_at | No | No | No | Yes | ubiquitin specific peptidase 48 |
| 212745_s_at | No | No | No | Yes | Bardet-Biedl syndrome 4 |
| 201602_s_at | No | No | No | Yes | protein phosphatase 1, regulatory |
| | | | | | (inhibitor) subunit 12A |
| 214722_at | No | No | No | Yes | Notch homolog 2 (Drosophila) |
| | | | | | N-terminal like |

\hat{ }Probe set ID number is the Affymetrix ID number on the HU133A array.

### Example 11

### Genes Associated with Stroke

This example describes methods used to identify genes whose expression differed significantly between normal subjects and those who have had a stroke (either IS or ICH). Such genes can be used as an initial diagnostic for stroke. For example, if a positive result is obtained, the hemorrhagic stroke-associated molecules provided herein (see for example Tables 2-8 and 15-16) can be used to determine if the subject suffered a hemorrhagic stroke. The ischemic stroke-associated molecules disclosed in PCT/US2005/018744 (and in Table 18 herein) and herein (Table 17) can be used to determine if the subject suffered an ischemic stroke.

The data obtained in Example 3 (CEL files of 8 patients with confirmed ICH, 19 ischemic stroke subjects and 18 referent control subjects) was analyzed as follows. The two-group Welch-modified t-test was used under sample-drop-and-replace condition. Sample data corresponding to samples negative for stroke were grouped into one group; while sample data corresponding to samples positive for ischemic or hemorrhagic stroke were grouped into a second group. The Welch-modified t-test was performed between the groups on a gene fragment by gene fragment basis under sample-drop-and-replace condition. With each test performed, the fold-change between group means was taken. Gene fragments that maintained a significance value less than 0.05 under False Discovery Rate Multiple Comparison Correction procedure and a fold-change magnitude>=1.25 100% of the time were noted as those Affymetrix gene fragments (and thus stroke-associated genes and proteins) that can serve as diagnostic markers for a stroke event (whether ischemic or hemmorhagic).

As shown in Table 14, genes (15 genes, 18 gene probes) common to both stroke types (ICS and IS) were identified. Expression of these genes was significantly upregulated in subjects who suffered a stroke, relative to normal subjects.

TABLE 14
Genes with significant differences in expression between normal and stroke

| Probe Set | | | |
|---|---|---|---|
| ID\hat{ } | FC* | PV^{#} | Gene Title |
| 201963_at | 1.992420623 | 3.02E−05 | acyl-CoA synthetase long-chain family |
| | | | member 1 |
| 207275_s_at | 1.953482897 | 1.21E−05 | acyl-CoA synthetase long-chain family |
| | | | member 1 |
| 203140_at | 1.554408709 | 3.34E−05 | B-cell CLL/lymphoma 6 (zinc finger protein |
| | | | 51) |
| 213006_at | 1.647911789 | 4.30E−05 | CCAAT/enhancer binding protein (C/EBP), |
| | | | delta |
| 204714_s_at | 1.729309374 | 1.65E−05 | coagulation factor V (proaccelerin, labile |
| | | | factor) |
| 203184_at | 1.549372636 | 4.29E−05 | fibrillin 2 (congenital contractural |
| | | | arachnodactyly) |
| 218035_s_at | 1.578732465 | 3.42E−05 | RNA-binding protein |
| 209189_at | 2.160661253 | 4.13E−05 | v-fos FBJ murine osteosarcoma viral |
| | | | oncogene homolog |
| 203674_at | 1.251042323 | 6.29E−05 | helicase with zinc finger |
| 210128_s_at | 1.351754436 | 4.32E−05 | leukotriene B4 receptor |
| 205147_x_at | 1.435218781 | 3.45E−06 | neutrophil cytosolic factor 4, 40 kDa |
| 207677_s_at | 1.491292788 | 3.92E−06 | neutrophil cytosolic factor 4, 40 kDa |
| 216913_s_at | 1.329522337 | 4.67E−05 | ribosomal RNA processing 12 homolog (S. cerevisiae) |
| 204924_at | 1.694642786 | 5.34E−06 | toll-like receptor 2 |
| 202241_at | 1.803331924 | 1.48E−06 | tribbles homolog 1 (Drosophila) |
| 217823_s_at | 1.52051708 | 7.30E−08 | ubiquitin-conjugating enzyme E2, J1 (UBC6 |
| | | | homolog, yeast) |
| 217825_s_at | 1.377971306 | 1.52E−05 | ubiquitin-conjugating enzyme E2, J1 (UBC6 |
| | | | homolog, yeast) |
| 201531_at | 1.339829955 | 4.45E−07 | zinc finger protein 36, C3H type, homolog |
| | | | (mouse) |

\hat{ }Probe set ID number is the Affymetrix ID number on the HU133A array.
*FC is the fold change between normal and stroke samples.
^{#}PV is the p-value.

### Example 12

### Genes Associated with Ischemic and Hemorrhagic Stroke

This example describes methods used to identify genes whose expression differed significantly between normal subjects and those who have had an ischemic stroke or those who have had a hemorrhagic stroke. Such genes can be used as an initial diagnostic for ischemic stroke or a hemorrhagic stroke, or can be used following an initial stroke diagnosis (see Example 11).

The data obtained in Example 3 (CEL files of 8 patients with confirmed ICH, 19 ischemic stroke subjects and 18 referent control subjects) was analyzed as follows. Sample data corresponding to samples positive for hemorrhagic stroke were grouped into one group; while sample data corresponding to samples positive for ischemic stroke were grouped into a second group. The Welch-modified t-test was performed between the groups on a gene fragment by gene fragment basis under sample-drop-and-replace condition. With each test performed, the fold-change between group means was taken. Gene fragments that maintained a significance value less than 0.05 under False Discovery Rate Multiple Comparison Correction procedure and a fold-change magnitude>=1.25 100% of the time were flagged as those Affymetrix gene fragments (and thus stroke-associated genes and proteins) that can serve as markers to classify a stroke event (e.g to determine whether a stroke is ischemic or hemmorhagic in nature).

Table 15 provides five genes that can differentiate between ischemic and hemorrhagic stroke. Such genes are upregulated in ICH subjects relative to IS subjects. Therefore, increased expression of such genes relative to an IS control sample indicates that the subject has suffered a hemorrhagic stroke.

TABLE 15
Genes upregulated in hemorrhagic relative to IS

| Probe ID\hat{ } | FC* | PV# | Probe Set ID |
|---|---|---|---|
| 202523_s_at | 1.508538715 | 2.34E−07 | sparc/osteonectin, cwcv |
| | | | and kazal-like domains |
| | | | proteoglycan (testican) 2 |
| 207485_x_at | 1.405292538 | 2.19E−05 | butyrophilin, subfamily 3, |
| | | | member A1 |
| 211893_x_at | 2.071937302 | 1.13E−05 | CD6 molecule |
| 218813_s_at | 1.595131944 | 1.25E−05 | SH3-domain GRB2-like |
| | | | endophilin B2 |
| 37652_at | 1.347975554 | 1.88E−06 | calcineurin binding protein 1 |

\hat{ }Probe set ID number is the Affymetrix ID number on the HU133A array.
*FC is the fold change between hemorrhagic and ischemic stroke samples.
#PV is the p-value.

The data obtained in Example 3 (CEL files of 8 patients with confirmed ICH, 19 ischemic stroke subjects and 18 referent control subjects) was analyzed as follows to identify genes differentially regulated in response to hemorrhagic stroke. Sample data corresponding to samples negative for stroke were grouped into one group; while sample data corresponding to samples positive for hemorrhagic stroke were grouped into a second group. The Welch-modified t-test was performed between the groups on a gene fragment by gene fragment basis under sample-drop-and-replace condition. With each test performed, the fold-change between group means was taken. Gene fragments that maintained a significance value less than 0.05 under False Discovery Rate Multiple Comparison Correction procedure and a fold-change magnitude>=1.25 100% of the time were flagged as those Affymetrix gene fragments (and thus stroke-associated genes and proteins) that are differentially regulated in response to hemorrhagic-type stroke and thus can serve as markers to classify a stroke event as hemmorhagic in nature.

Table 16 provides genes that can be used to diagnose hemorrhagic stroke. For example, genes with a positive FC value are upregulated in hemmorhagic subjects relative to normal subjects, while genes with a negative FC value are downregulated in hemmorhagic subjects relative to normal subjects.

TABLE 16
Genes differentially expressed in normal versus hemorrhagic samples

| Probe ID\hat{ } | FC* | PV# | Probe Set ID |
|---|---|---|---|
| 200952_s_at | −1.515215545 | 1.69E−05 | cyclin D2 |
| 201991_s_at | 1.306520882 | 2.67E−06 | kinesin family member 5B |
| 202523_s_at | −1.435487585 | 2.65E−05 | sparc/osteonectin, cwcv and kazal-like |
| | | | domains proteoglycan (testican) 2 |
| 203674_at | 1.475070526 | 2.92E−06 | helicase with zinc finger |
| 211316_x_at | 1.387176009 | 3.22E−06 | CASP8 and FADD-like apoptosis regulator |
| 211856_x_at | −1.370753455 | 5.47E−05 | CD28 molecule |
| 212259_s_at | −1.509605727 | 7.03E−05 | pre-B-cell leukemia transcription factor |
| | | | interacting protein 1 |
| 212263_at | 1.44075598 | 6.56E−06 | quaking homolog, KH domain RNA binding |
| | | | (mouse) |
| 212361_s_at | 1.493099895 | 6.60E−05 | ATPase, Ca++ transporting, cardiac muscle, |
| | | | slow twitch 2 |
| 212888_at | 1.386901014 | 1.22E−05 | Dicer1, Dcr-1 homolog (Drosophila) |
| 214447_at | −1.841768304 | 6.97E−05 | v-ets erythroblastosis virus E26 oncogene |
| | | | homolog 1 (avian) |
| 215127_s_at | 1.321162413 | 3.79E−05 | RNA binding motif, single stranded interacting |
| | | | protein 1 |
| 216969_s_at | −1.481516754 | 1.20E−05 | kinesin family member 22 /// similar to |
| | | | Kinesin-like protein KIF22 (Kinesin-like |
| | | | DNA-binding protein) (Kinesin-like protein 4) |
| 217119_s_at | −1.364953776 | 5.46E−05 | chemokine (C—X—C motif) receptor 3 |
| 218559_s_at | 2.103933153 | 1.88E−05 | v-maf musculoaponeurotic fibrosarcoma |
| | | | oncogene homolog B (avian) |
| 219358_s_at | 1.68969976 | 5.70E−06 | centaurin, alpha 2 |
| 219999_at | 1.317017278 | 3.55E−05 | mannosidase, alpha, class 2A, member 2 |
| 221039_s_at | 1.357768211 | 1.19E−05 | development and differentiation enhancing |
| | | | factor 1 |

\hat{ }Probe set ID number is the Affymetrix ID number on the HU133A array.
*FC is the fold change between normal and hemorrhagic stroke samples.
#PV is the p-value.

The data obtained in Example 3 (CEL files of 8 patients with confirmed ICH, 19 ischemic stroke subjects and 18 referent control subjects) was analyzed as follows to identify genes differentially regulated in response to ischemic stroke. Sample data corresponding to samples negative for stroke were grouped into one group; while sample data corresponding to samples positive for ischemic stroke were grouped into a second group. The Welch-modified t-test was performed between the groups on a gene fragment by gene fragment basis under sample-drop-and-replace condition. With each test performed, the fold-change between group means was taken. Gene fragments that maintained a significance value less than 0.05 under False Discovery Rate Multiple Comparison Correction procedure and a fold-change magnitude>=1.25 100% of the time were flagged as those Affymetrix gene fragments (and thus stroke-associated genes and proteins) that are differentially regulated in response to ischemic-type stroke and thus can serve as markers to classify a stroke event as ischemic in nature.

Table 17 provides a gene that can be used to diagnose ischemic stroke. For example, this gene is upregulated in IS subjects relative to normal subjects. This gene can be used in combination with other ischemic-stroke related molecules (such as those listed in Table 18) for diagnosis of ischemic stroke identified.

TABLE 17
Normal versus ischemic stroke

| | | | Probe Set | |
|---|---|---|---|---|
| Probe ID\hat{ } | FC* | PV# | ID | Gene Title |
| 217823_s_at | 1.394233157 | 1.99E−06 | 217823_s_at | ubiquitin- |
| | | | | conjugating |
| | | | | enzyme |
| | | | | E2, J1 |
| | | | | (UBC6 |
| | | | | homolog, |
| | | | | yeast) |

\hat{ }Probe set ID number is the Affymetrix ID number on the HU133A array.
*FC is the fold change between normal and ischemic stroke samples.
#PV is the p-value.

### Example 13

### Diagnosis and Classification of Stroke

This example describes methods that can be used to diagnose a subject as having had a stroke, such as an ischemic (IS) or hemorrhagic (such as an ICH) stroke.

Evaluation of the subject can be performed as early as one day (or within 24 hours) after the stroke is suspected, 2-11 or 7-14 days after the stroke is suspected, or at least 90 days after the stroke is suspected. The disclosed methods can be performed following the onset of signs and symptoms associated with a stroke, such as IS or ICH. Particular examples of signs and symptoms associated with a stroke include but are not limited to: headache, sensory loss (such as numbness, particularly confined to one side of the body or face), paralysis (such as hemiparesis), pupillary changes, blindness (including bilateral blindness), ataxia, memory impairment, dysarthria, somnolence, and other effects on the central nervous system recognized by those of skill in the art.

A sample can be obtained from the subject (such as a PBMC sample) and analyzed using the disclosed methods, for example, within 1 hour, within 6 hours, within 12 hours, within 24 hours, or within 48 hours of having signs or symptoms associated with stroke. In another example, a sample is obtained at least 7 days later following the onset of signs and symptoms associated with stroke, such as within 2-11 or 7-14 days of having signs or symptoms associated with stroke, or within 90 days. In particular examples, the assay can be performed after a sufficient period of time for the differential regulation of the genes (or proteins) to occur, for example at least 24 hours or at least 48 hours after onset of the symptom or constellation of symptoms that have indicated a potential stroke (such as a cerebral hemorrhagic or ischemic event). In other examples it occurs prior to performing any imaging tests are performed to find anatomic evidence of stroke. The assays described herein in particular examples can detect the stroke even before definitive brain imaging evidence of the stroke is known.

For example, PBMCs can be isolated from the subject (such as a human subject) following stroke, for example at least 24 hours, at least 48 hours, or at least 72 hours after the stroke. In particular examples, PBMCs are obtained from the subject at day 1 (within 24 hours of onset of symptoms), at day 7-14 and at day 90 post stroke. In particular examples, the subject is suspected of having suffered an ICH. In other examples, the subject is suspected of having suffered an IS.

### Determining if the Subject has Suffered a Stroke

In particular examples, the method includes detecting expression of at least four of the stroke-related molecules listed in Table 14, such as at least 5, 6, 7, 8, 9, 10, 11, 12, 13, 14, or all 15 of those listed in Table 14. The molecules listed in Table 14 are upregulated in subjects who have suffered a stroke, relative to a subject who has not suffered a stroke. For example, nucleic acid molecules or proteins isolated from the PBMCs can be contacted with an array that includes probes that can detect at least four of the stroke-related molecules listed in Table 14, such as an array that includes probes that can detect all of the genes (or proteins) listed in Table 14. Expression of the stroke-related genes (or proteins) can be determined using the methods described in the above examples.

Detection of significant upregulation of at least four stroke-related molecules listed in Table 14, such as upregulation of v-fos FBJ murine osteosarcoma viral oncogene homolog, acyl-CoA synthetase long-chain family member 1, coagulation factor V (proaccelerin, labile factor), and tribbles homolog 1 (Drosophila), indicates that the subject has suffered a stroke. For example, detection of significant upregulation of all of the stroke-related molecules listed in Table 14 indicates that the subject has suffered a stroke. In contrast, detection of significant upregulation in less than four stroke-related molecules listed in Table 14 (such as 3, 2, 1 or none) indicates that the subject has not suffered a stroke. In particular examples, the differential expression is determined by calculating a fold-change in expression, by calculating a ratio of expression detected in the subject relative to a reference expression value (such as an expression value or range expected from a normal (e.g. non-stroke) sample). For example, detection of at least a 1.2 fold increase in expression (such as at least 1.4, at least 1.5, or at least 2 fold increase) in the test subject's sample, relative to a normal reference value, indicates that expression is increased in the test subject's sample. In particular examples, the increased expression is determined by calculating a t-statistic value, wherein a t-statistic value of at least 3, at least 5, at least 6, or at least 15 indicates that expression is increased.

If the assay indicates that the subject has suffered a stroke, further analysis can be performed to determine what type of stroke the patient had (such as an IS or ICH). In some examples, this first step (determining if the subject has had a stroke) is omitted, and an assay is only performed to determine whether the patient has had an IS or hemorrhagic stroke. In some examples, this first step (determining if the subject has had a stroke) is performed at essentially the same time as an assay performed to determine whether the patient has had an IS or hemorrhagic stroke (e.g. a single array is used to perform multiple analyses).

### Determining if the Subject has Suffered an Ischemic or Hemorrhagic Stroke

In particular examples, the method includes determining whether the subject has suffered a hemorrhagic stroke, such as an ICH, or an ischemic stroke. For example, the five stroke-related molecules listed in Table 15 can be used to determine if the subject has had an ICH or an IS. In particular example, the method includes detecting expression of at least four of the stroke-related molecules listed in Table 15, such as all five of the molecules listed in Table 15. The genes listed in Table 15 are upregulated in subjects who have suffered a hemorrhagic stroke, relative to a subject who has suffered an IS. For example, nucleic acid molecules or proteins isolated from the PBMCs can be contacted with an array that includes probes that can detect at least four of the stroke-related molecules listed in Table 15, such as an array that includes probes that can detect all of the genes (or proteins) listed in Table 15. Expression of the stroke-related genes (or proteins) can be determined using the methods described in the above examples.

Detection of significant upregulation of at least four stroke-related molecules listed in Table 15, such as upregulation of sparc/osteonectin, cwcv and kazal-like domains proteoglycan (testican) 2, butyrophilin, subfamily 3, member A1, CD6 molecule, and SH3-domain GRB2-like endophilin B2), indicates that the subject has suffered a hemorrhagic stroke (not an IS). For example, detection of significant upregulation of all of the stroke-related molecules listed in Table 15 indicates that the subject has suffered a hemorrhagic stroke (not an IS). In contrast, detection of no significant upregulation in the stroke-related molecules listed in Table 15 indicates that the subject has not suffered an ICH, but may have suffered an IS. In particular examples, the differential expression is determined by calculating a fold-change in expression, by calculating a ratio of expression detected in the subject relative to a reference expression value (such as an expression value or range expected from a IS sample). For example, detection of at least a 1.2 fold increase in expression (such as at least 1.4, at least 1.5, or at least 2 fold increase) in the test subject's sample, relative to an IS reference value, indicates that expression is increased in the test subject's sample, and thus the subject has suffered a hemorrhagic stroke (and not an IS). In contrast, detection of less than a 1 fold increase in expression (less than a 0.5 fold increase) in the test subject's sample, relative to an IS reference value, indicates that expression is not significantly altered in the test subject's sample, and thus the subject may have suffered an IS (and not a hemorrhagic stroke). In particular examples, the differential expression is determined by calculating a t-statistic value, wherein a t-statistic value of at least 3, at least 5, at least 6, or at least 15 indicates that expression is increased, while a t-statistic value of no more than −3, no more than −5, or no more than −6 indicates that expression is decreased. For example, detection of at least a t-value of at least 3 for all of the genes listed in Table 15 indicates that expression is increased in the test subject's sample, and thus the subject has suffered a hemorrhagic stroke (and not an IS).

### Determining if the Subject has Suffered a Hemorrhagic Stroke

In particular examples, the method includes determining whether the subject has suffered a hemorrhagic stroke, such as an ICH. For example, the 18 hemorrhagic stroke-related molecules listed in Table 16 can be used to determine if the subject has had an ICH. In particular example, the method includes detecting expression of at least four of the hemorrhagic stroke-related molecules listed in Table 16, such as all of the molecules listed in Table 16. The genes listed in Table 16 are upregulated (positive FC value) or downregulated (negative FC value) in subjects who have suffered a hemorrhagic stroke, relative to a normal subject (e.g. a subject who has not suffered a stroke). For example, nucleic acid molecules or proteins isolated from the PBMCs can be contacted with an array that includes probes that can detect at least four of the stroke-related molecules listed in Table 16, such as an array that includes probes that can detect all of the genes (or proteins) listed in Table 16. Expression of the stroke-related genes (or proteins) can be determined using the methods described in the above examples. Detection of significant upregulation or down regulation of at least four hemorrhagic stroke-related molecules listed in Table 16, such as upregulation of v-maf musculoaponeurotic fibrosarcoma oncogene homolog B, and centaurin, alpha 2 and downregulation of v-ets erythroblastosis virus E26 oncogene homolog 1 and, sparc/osteonectin, cwcv and kazal-like domains proteoglycan (testican) 2 indicates that the subject has suffered a hemorrhagic stroke. For example, detection of significant altered expression of all of the stroke-related molecules listed in Table 16 indicates that the subject has suffered a hemorrhagic stroke. In contrast, detection of no significant altered expression in the hemorrhagic stroke-related molecules listed in Table 16 indicates that the subject has not suffered an ICH. In particular examples, the differential expression is determined by calculating a fold-change in expression, by calculating a ratio of expression detected in the subject relative to a reference expression value (such as an expression value or range expected from a normal sample). For example, detection of at least a 1.2 fold increase in expression (such as at least 1.4, at least 1.5, or at least 2 fold increase) in the test subject's sample, relative to a normal reference value, indicates that expression is increased in the test subject's sample. Detection of at least a −1.2 fold decrease in expression (such as at least −1.4, at least −1.5, or at least −2 fold decrease) in the test subject's sample, relative to a normal reference value, indicates that expression is decreased in the test subject's sample. In particular examples, the increased expression is determined by calculating a t-statistic value, wherein a t-statistic value of at least 3, at least 5, at least 6, or at least 15 indicates that expression is increased, and a t-statistic value of less than −3, less than −5, less than −6, or less than −15 indicates that expression is decreased.

In particular examples, the method determining whether the subject has suffered a hemorrhagic stroke, such as an ICH, includes detecting differential expression in at least four hemorrhagic stroke-related molecules, such detecting differential expression of IL1R2, haptoglobin, amphiphysin, CD163, and TAP2. In one example, the method includes detecting differential expression in at least the 30 genes (or corresponding proteins) listed in Table 5. For example, nucleic acid molecules or proteins isolated from the PBMCs can be contacted with a hemorrhagic stroke detection array, such as an array that includes probes that can detect at least four of the hemorrhagic stroke-related molecules listed in Tables 2-8 and 15-16, such as an array that includes probes that can detect all of the genes (or proteins) listed in Table 5, 8 or 16. Expression of the hemorrhagic stroke-related genes (or proteins) can be determined using the methods described in the above examples.

Detection of significant differential expression (such as upregulation or downregulation) of at least four hemorrhagic stroke-related molecules, such as IL1R2, haptoglobin, amphiphysin, CD163, and TAP2, or at least the 30 genes (or corresponding proteins) listed in Table 5, indicates that the subject has suffered a hemorrhagic stroke. In particular examples, the differential expression is determined by calculating a t-statistic value, wherein a t-statistic value of at least 3, at least 5, at least 6, or at least 15 indicates that expression is increased, while a t-statistic value of no more than −3, no more than −5, or no more than −6 indicates that expression is decreased.

The observed differential expression of the hemorrhagic stroke-related genes (or proteins) can be compared to a reference value, such as values that represent expression levels expected if no stroke occurred, or if an ischemic stroke occurred. For example if the subject shows expression levels similar to that expected if the stroke was ischemic, then it is predicted that the subject did not suffer a hemorrhagic stroke, but instead suffered an IS. If the subject shows expression levels similar to that expected if no stroke occurred, then it is predicted that the subject did not suffer a hemorrhagic stroke.

### Determining if the Subject has Suffered an Ischemic Stroke

In particular examples, if it is determined that the subject has suffered a stroke, the method further includes determining if the stroke was ischemic. For example, the ischemic stroke-related molecule listed in Table 17 can be used to determine if the subject has had an IS. In particular examples, the method includes detecting expression of ubiquitin-conjugating enzyme E2, J1 (Table 17) and at least four of the IS-related molecules listed in Table 18 such as all of the molecules listed in Table 18. Ubiquitin-conjugating enzyme E2, J1 (Table 17) is upregulated (positive FC value) in subjects who have suffered an IS, relative to a normal subject (e.g. a subject who has not suffered a stroke). For example, nucleic acid molecules or proteins isolated from the PBMCs can be contacted with an array that includes probes that can detect ubiquitin-conjugating enzyme E2, J1 and at least four of the stroke-related molecules listed in Table 18, such as an array that includes probes that can detect ubiquitin-conjugating enzyme E2, J1 and all of the genes (or proteins) listed in Table 18. Expression of the IS-related genes (or proteins) can be determined using the methods described in the above examples. Detection of significant upregulation of ubiquitin-conjugating enzyme E2, J1 and at least four IS stroke-related molecules listed in Table 18, such as upregulation of ubiquitin-conjugating enzyme E2, J1 and the molecules listed in Table 18, indicates that the subject has suffered an IS. In contrast, detection of no significant altered expression in ubiquitin-conjugating enzyme E2, J1 and the IS-related molecules listed in Table 18, indicates that the subject has not suffered an IS.

TABLE 18
Ischemic stroke related-genes using PAM correction (from
PCT/US2005/018744).

| Affymetrix | |
|---|---|
| Probe ID | Name and Function |
| | White Blood Cell Activation and Differentiation |
| 215049_x_at | CD163 |
| 218454_at | Hypothetical protein FLJ22662 Laminin A motif |
| 211404_s_at | Amyloid beta (A4) precursor-like protein 2 |
| 221210_s_at | N-acetylneuraminate pyruvate lysase |
| 209189_at | v-fos FBJ murine osteosarcoma viral oncogene homolog |
| 204924_at | Toll-like receptor 2 |
| 211571_s_at | Chondroitin sulfate proteoglycan 2 (versican) |
| 211612_s_at | Interleukin 13 receptor, alpha 1 |
| 201743_at | CD14 antigen |
| 205715_at | Bone marrow stromal cell antigen 1/CD157 |
| 202878_s_at | Complement component 1, q subcomponent, receptor 1 |
| 219788_at | Paired immunoglobin-like type 2 receptor alpha |
| 214511_x_at | Fc fragment of IgG, high affinity Ia, receptor for (CD64) |
| | Vascular Repair |
| 203888_at | Thrombomodulin |
| 207691_x_at | Ectonucleoside triphosphate diphosphohydrolase 1 |
| 206488_s_at | CD36 antigen (collagen type I receptor, thrombospondin receptor) |
| | Response to Hypoxia |
| 202912_at | Adrenomedullin |
| 201041_s_at | Dual specificity phosphatase 1 |
| 203922_s_at | Cytochrome b-245, beta polypeptide (chronic granulomatous disease) |
| 208771_s_at | Leukotriene A4 hydrolase |
| 201328_at | Erythroblastosis virus E26 oncogene homolog 2 (avian) |
| 209949_at | Neutrophil cytosolic factor 2 (65 kDa, chronic granulomatous disease, |
| | autosomal 2) |
| | Response to Altered Cerebral Microenvironment |
| 208818_s_at | Catechol-O-methyltransferase |
| 200648_s_at | Glutamate-ammonia ligase (glutamine ligase) |
| 202917_s_at | S100 calcium binding protein A8 (calgranulin A) |
| 204860_s_at | Neuronal apoptosis inhibitory protein: Homo sapiens transcribed |
| | sequence with strong similarity to protein sp: Q13075 (H. sapiens) |
| | BIR1_HUMAN Baculoviral IAP repeat-containing protein 1 |
| 212807_s_at | Sortilin |
| 202446_s_at | Phospholipid scramblase 1 |
| 211067_s_at | Growth-arrest-specific 7 |
| 204222_s_at | GLI pathogenesis-related 1 (glioma) |

In particular examples, the differential expression is determined by calculating a fold-change in expression, by calculating a ratio of expression detected in the subject relative to a reference expression value (such as an expression value or range expected from a normal sample). For example, detection of at least a 1.2 fold increase in expression (such as at least 1.4, at least 1.5, or at least 2 fold increase) in the test subject's sample, relative to a normal reference value, indicates that expression is increased in the test subject's sample. In some examples, the differential expression is determined by calculating a t-statistic value, wherein a t-statistic value of at least 3, at least 5, at least 6, or at least 15 indicates that expression is increased.

The observed differential expression of the IS-stroke-related genes (or proteins) can be compared to a reference value, such as values that represent expression levels expected if no stroke occurred, or if a hemorrhagic stroke occurred. For example if the subject shows expression levels similar to that expected if the stroke was hemorrhagic, then it is predicted that the subject did not suffer an ischemic stroke, but instead suffered a hemorrhagic stroke. If the subject shows expression levels similar to that expected if the no stroke occurred, then it is predicted that the subject did not suffer an ischemic stroke.

### Example 14

### Predicting Severity and Neurological Recovery of Hemorrhagic Stroke

This example describes methods that can be used to determine the severity and likely neurological recovery of a subject who has had an intracerebral hemorrhagic stroke, for example by determining the expression levels of at least four of the hemorrhagic stroke-related molecules listed in Tables 2-8 and 15-16. Although particular timepoints and hemorrhagic stroke-associated genes are described, one skilled in the art will appreciate that other timepoints and genes (or proteins) can be used.

Stratification or assessing the likely neurological recovery of the subject can be performed as early as one day (or within 24 hours) after the hemorrhagic stroke, 2-11 or 7-14 days after the hemorrhagic stroke, or at least 90 days after the hemorrhagic stroke. The disclosed methods can be performed following the onset of signs and symptoms associated with ICH. Particular examples of signs and symptoms associated with an ICH stroke include but are not limited to: headache, sensory loss (such as numbness, particularly confined to one side of the body or face), paralysis (such as hemiparesis), pupillary changes, blindness (including bilateral blindness), ataxia, memory impairment, dysarthria, somnolence, and other effects on the central nervous system recognized by those of skill in the art.

A sample can be obtained from the subject (such as a PBMC sample) and analyzed using the disclosed methods, for example, within 1 hour, within 6 hours, within 12 hours, within 24 hours, or within 48 hours of having signs or symptoms associated with ICH stroke. In another example, a sample is obtained at least 7 days later following the onset of signs and symptoms associated with ICH stroke, such as within 2-11 or 7-14 days of having signs or symptoms associated with ICH stroke, or within 90 days. In particular examples, the assay can be performed after a sufficient period of time for the differential regulation of the genes (or proteins) to occur, for example at least 24 hours after onset of the symptom or constellation of symptoms that have indicated a potential cerebral hemorrhagic event. In other examples it occurs prior to performing any imaging tests are performed to find anatomic evidence of hemorrhagic stroke. The assay described herein in particular examples is able to detect the hemorrhagic stroke even before definitive brain imaging evidence of the stroke is known.

For example, PBMCs can be isolated from the subject (such as a human subject) following hemorrhagic stroke, for example at least 24 hours, at least 48 hours, or at least 72 hours after the stroke. In particular examples, PBMCs are obtained from the subject at day 1 (within 24 hours of onset of symptoms), at day 7-14 and at day 90 post stroke.

In particular examples, the method includes detecting differential expression in at least four hemorrhagic stroke-related molecules, such detecting differential expression of IL1R2, haptoglobin, amphiphysin, CD163, and TAP2. In one example, the method includes detecting differential expression in at least the 30 genes (or corresponding proteins) listed in Table 5. For example, nucleic acid molecules or proteins isolated from the PBMCs can be contacted with a hemorrhagic stroke detection array, such as an array that includes probes that can detect at least four of the hemorrhagic stroke-related molecules listed in Tables 2-8 and 15-16, such as an array that includes probes that can detect all of the genes (or proteins) listed in Table 5, 8, 15, 16, or combinations thereof. Expression of the hemorrhagic stroke-related genes (or proteins) can be determined using the methods described in the above examples.

Detection of significant differential expression (such as upregulation or downregulation) of at least four hemorrhagic stroke-related molecules, such as IL1R2, haptoglobin, amphiphysin, CD163 (and in some examples TAP2), or at least the 25 genes (or corresponding proteins) listed in Table 5, indicates that the stroke was severe and the subject has a lower probability of neurological recovery (for example as compared to an amount of expected neurological recovery in a subject who did not have differential expression of IL1R2, haptoglobin, amphiphysin, CD163 (and in some examples TAP2), or the 30 genes/proteins listed in Table 5). In particular examples, the differential expression is determined by calculating a t-statistic value, wherein a t-statistic value of at least 3, at least 5, at least 6, or at least 15 indicates that expression is increased, while a t-statistic value of no more than −3, no more than −5, or no more than −6 indicates that expression is decreased. In one example, detection of differential expression of 1 to 3 hemorrhagic stroke-related molecules listed in Tables 2-8 and 15-16 (such as 1 to 3 of IL1R2, haptoglobin, amphiphysin, CD163, granzyme M, Sema4C and TAP2) indicates mild hemorrhagic stroke and differential expression of 5 to 10 hemorrhagic stroke-related molecules listed in Tables 2-8 and 15-16 (such as 5 to 10 that include IL1R2, haptoglobin, amphiphysin, CD163, granzyme M, Sema4C and TAP2) indicates a more severe stroke.

The observed differential expression of the hemorrhagic stroke-related genes (or proteins) can be compared to a reference value, such as values that represent expression levels expected if the hemorrhagic stroke is severe or mild, or expression levels expected if the neurological recovery is good or poor. For example if the subject shows expression levels similar to that expected if the hemorrhagic stroke is severe, then it is predicted that the subject suffered a severe hemorrhagic stroke, and neurological recovery is less likely. If the subject shows expression levels similar to that expected if the hemorrhagic stroke is mild, then it is predicted that the subject suffered a mild hemorrhagic stroke, and neurological recovery is more likely.

In particular examples, the magnitude of the change in expression levels of hemorrhagic stroke-related genes (or proteins) is greater in subjects having suffered a more severe stroke, as compared to those subjects how have suffered a milder stroke. Similarly, the magnitude of the change in expression levels of hemorrhagic stroke-related genes (or proteins) is greater in subjects more likely to suffer permanent neurological damage, as compared to those subjects more likely to suffer permanent neurological damage. For example, a subject having suffered a severe stroke may demonstrate t-values of at least four (such as at least 10 or at least 20) hemorrhagic stroke-related genes (or proteins) listed in Tables 2-8 and 15-16 that are increased (for genes/proteins whose expression is upregulated in response to hemorrhagic stroke) or decreased (for genes/proteins whose expression is downregulated in response to hemorrhagic stroke) at least 2-fold (such as at least 3-fold or at least 4-fold) as compared to a subject having suffered a mild stroke. For example, a subject having suffered a mild stroke may demonstrate a t-value of no more than 5 for the IL1R2, CD163, and amphiphysin genes and a t-statistic value of no less than −5 for TAP2 or Sema4C (for example as compared to a subject who has not suffered a stroke), while a subject having suffered a severe stroke may demonstrate a t-statistic value of at least 10 for the IL1R2, haptoglobin, CD163 and amphiphysin genes and a t-statistic value of less than −6 for TAP2 or Sema4C (for example as compared to a subject who has not suffered a stroke). Subjects indicated to have suffered a more severe hemorrhagic stroke are more likely to suffer permanent neurological damage.

In particular examples, persistence of changes in hemorrhagic stroke-related gene (or protein) expression is used to determine the likely neurological recovery of a subject who has suffered a hemorrhagic stroke. Generally, if the detected changes in hemorrhagic stroke-related gene (or protein) expression persist (for example at least 7 days, at least 14 days, at least 60 days, or at least 90 days after the stroke), it is proposed that processes related to the stroke or a lack of recovery of these processes is occurring, and that such subjects have a worse prognosis. For example, subjects who remain classified as having had a hemorrhagic stroke using the methods provided herein at these later time points are those with the more severe strokes and worse outcomes. For example, subjects demonstrating a change in expression in at least four of the hemorrhagic stroke-related molecules listed in Tables 2-8 and 15-16 at least 7, 14, 60, or 90 days after the intracerebral hemorrhagic stroke are less likely to recover from neurological damage, as these results indicate the subject has suffered a severe stroke. In contrast, subjects who are indicated to not have had a hemorrhagic stroke at least 7, 14, 60, or 90 days after the intracerebral hemorrhagic stroke (using the methods provided herein), indicates that the subject is more likely to recover from neurological damage, as these results indicate the subject has suffered a mild hemorrhagic stroke.

Since the results of this assay are also highly reliable predictors of the hemorrhagic nature of the stroke, the results of the assay can also be used (for example in combination with other clinical evidence and brain scans) to determine whether anti-hemorrhagic therapy, such as therapy designed to reduce high blood pressure or to increase blood clotting, should be administered to the subject. In certain example, anti-hypertensive therapy or clotting therapy (or both) is given to the subject once the results of the differential gene assay are known if the assay provides an indication that the stroke is hemorrhagic in nature.

Moreover, the neurological sequalae of a hemorrhagic event in the central nervous system can have consequences that range from the insignificant to the devastating, and the disclosed assay permits early and accurate stratification of risk of long-lasting neurological impairment. For example, a test performed as early as within the first 24 hours of onset of signs and symptoms of a stroke, and even as late as 7-14 days or even as late as 90 days or more after the event can provide clinical data that is highly predictive of the eventual care needs of the subject.

The disclosed methods are also able to identify subjects who have had a hemorrhagic stroke in the past, for example more than 2 weeks ago, or even more than 90 days ago. The identification of such subjects helps evaluate other clinical data (such as neurological impairment or brain imaging information) to determine whether a hemorrhagic stroke (such as an intracerebral hemorrhagic stroke) has occurred. Subjects identified or evaluated in this manner can then be provided with appropriate treatments, such as clotting agents that would be appropriate for a subject identified as having had a hemorrhagic stroke but not as appropriate for subject who have had an ischemic stroke. It is helpful to be able to classify subject as having had a hemorrhagic stroke, because the treatments for hemorrhagic stroke are often distinct from the treatments for ischemic stroke. In fact, treating a hemorrhagic stroke with a therapy designed for an ischemic stroke (such as a thrombolytic agent) can have devastating clinical consequences. Hence using the results of the disclosed assay to help distinguish ischemic from hemorrhagic stroke offers substantial clinical benefit, and allows subjects to be selected for treatments appropriate to hemorrhagic stroke but not ischemic stroke.

### Example 15

### Arrays for Evaluating a Stroke

This example describes particular arrays that can be used to evaluate a stroke, for example to diagnose an intracerebral hemorrhagic stroke. When describing an array that consists essentially of probes that recognize one or more of the hemorrhagic stroke-related molecules in Tables 2-8 and 15-16, such an array includes probes that recognize at least one of the hemorrhagic stroke-related molecules in Tables 2-8 and 15-16 (for example any sub-combination of molecules listed in Tables 2-8 and 15-16) as well as control probes (for example that can be used to confirm the incubation conditions are sufficient), ischemic probes (such as those in Tables 17-18), stroke probes (such as those in Table 14), but not other probes. Exemplary control probes include GAPDH, actin, and YWHAZ.

In one example, the array includes, consists essentially of, or consists of probes (such as an oligonucleotide or antibody) that can recognize at least one gene (or protein) that is upregulated following hemorrhagic stroke, such as one or more of IL1R2, haptoglobin, amphiphysin, or CD163, or any 1, 2, 3, or 4 of these. For example, the array can include a probe (such as an oligonucleotide or antibody) recognizes IL1R2. In yet another example, the array includes, consists essentially of, or consists of probes (such as an oligonucleotide or antibody) that can recognize at least one gene (or protein) that is down-regulated following hemorrhagic stroke, such as one or more of TAP2, granzyme M and Sema4C. In a particular example, the array includes, consists essentially of, or consists of probes (such as an oligonucleotide or antibody) that can recognize at least one gene (or protein) that is upregulated following a hemorrhagic stroke (such as at least one of IL1R2, haptoglobin, amphiphysin, and CD163) and at least one gene (or protein) that is downregulated following a hemorrhagic stroke (such as one or more of TAP2, Sema 4C or granzyme M).

Other exemplary probes that can be used are listed in Tables 2-8 and 15-16 and are identified by their Affymetrix identification number. The disclosed oligonucleotide probes can further include one or more detectable labels, to permit detection of hybridization signals between the probe and a target sequence.

In one example, the array includes, consists essentially of, or consists of probes (such as an oligonucleotide or antibody) that recognize any combination of at least four different genes (or proteins) listed in Tables 2-8 and 15-16. In particular examples, the array includes, consists essentially of, or consists of probes recognize all 30 genes (or proteins) listed in Table 5, all 316 genes listed in Table 7, all 5 genes listed in Table 15, or all 18 genes listed in Table 16. In some examples, the array includes oligonucleotides, proteins, or antibodies that recognize any combination of at least one gene from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction (such as at least 2 or at least 3 genes from each class).

In another example, the array includes, consists essentially of, or consists of probes (such as an oligonucleotide or antibody) that recognize any combination of at least 150 different genes listed in Tables 2-8 and 15-16, such as all 47 genes listed in Table 2, all 1263 genes listed in Table 3, all 119 genes listed in Table 4, all 30 genes listed in Table 5, all 446 genes listed in Table 6, all 25 genes listed in Table 7, all 316 genes listed in Table 8, all 5 genes listed in Table 15, or all 18 genes listed in Table 16.

Compilation of “loss” and “gain” of hybridization signals will reveal the genetic status of the individual with respect to the hybridization stroke-associated genes listed in Tables 2-8 and 15-16.

### Example 16

### Quantitative Spectroscopic Methods

This example describes quantitative spectroscopic approaches methods, such as SELDI, that can be used to analyze a biological sample to determine if there is differential protein expression of hemorrhagic stroke-related proteins, such as those listed in Tables 2-8 and 15-16.

In one example, surface-enhanced laser desorption-ionization time-of-flight (SELDI-TOF) mass spectrometry is used to detect changes in differential protein expression, for example by using the ProteinChip™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest, such as hemorrhagic stroke-related proteins. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface (such as hemorrhagic stroke-related proteins) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind at least four of the hemorrhagic stroke-related proteins listed in Tables 2-8 and 15-16. In one example, antibodies are immobilized onto the surface using a bacterial Fc binding support. The chromatographic surface is incubated with a sample from the subject, such as a sample that includes PMBC proteins (such as a PBMC lysate). The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

### Example 17

### Nucleic Acid-Based Analysis

The hemorrhagic stroke-related nucleic acid molecules provided herein (such as those disclosed in Tables 2-8 and 15-16) can be used in evaluating a stroke, for example for determining whether a subject has had an intracerebral hemorrhagic stroke, determining the severity or likely neurological recovery of a subject who has had an ICH stroke, and determining a treatment regimen for a subject who has had an ICH stroke. For such procedures, a biological sample of the subject is assayed for an increase or decrease in expression of hemorrhagic stroke-related nucleic acid molecules, such as those listed in Tables 2-8 and 15-16. Suitable biological samples include samples containing genomic DNA or RNA (including mRNA) obtained from cells of a subject, such as those present in peripheral blood, urine, saliva, tissue biopsy, surgical specimen, and autopsy material. In a particular example, the sample includes PBMCs (or components thereof, such as nucleic acids molecules isolated from PBMCs).

The detection in the biological sample of expression four or more hemorrhagic stroke-related nucleic acid molecules, such any combination of four or more molecules listed in Tables 2-8 and 15-16, for example 20 or more molecules listed in Tables 2-8 and 15-16, can be achieved by methods known in the art. In some examples, expression is determined for any combination of at least one gene from each of the following classes, genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction (such as at least 2 or at least 3 genes from each class). In some examples, expression is determined for at least IL1R2, haptoglobin, amphiphysin, and TAP2, and can optionally further include CD163, granzyme M, and Sema4C.

Increased or decreased expression of a hemorrhagic stroke-related molecule also can be detected by measuring the cellular level of hemorrhagic stroke-related nucleic acid molecule-specific mRNA. mRNA can be measured using techniques well known in the art, including for instance Northern analysis, RT-PCR and mRNA in situ hybridization. Details of mRNA analysis procedures can be found, for instance, in provided examples and in Sambrook et al. (ed.), Molecular Cloning: A Laboratory Manual, 2nd ed., vol. 1-3, Cold Spring Harbor Laboratory Press, Cold Spring Harbor, N.Y., 1989.

Oligonucleotides that can specifically hybridize (for example under very high stringency conditions) to hemorrhagic stroke-related sequences (such as those listed in Tables 2-8 and 15-16) can be chemically synthesized using commercially available machines. These oligonucleotides can then be labeled, for example with radioactive isotopes (such as ³²P) or with non-radioactive labels such as biotin (Ward and Langer et al., Proc. Natl. Acad. Sci. USA 78:6633-57, 1981) or a fluorophore, and hybridized to individual DNA samples immobilized on membranes or other solid supports by dot-blot or transfer from gels after electrophoresis. These specific sequences are visualized, for example by methods such as autoradiography or fluorometric (Landegren et al., Science 242:229-37, 1989) or colorimetric reactions (Gebeyehu et al., Nucleic Acids Res. 15:4513-34, 1987).

Nucleic acid molecules isolated from PBMCs can be amplified using routine methods to form nucleic acid amplification products. These nucleic acid amplification products can then be contacted with an oligonucleotide probe that will hybridize under very high stringency conditions with a hemorrhagic stroke-related nucleic acid. The nucleic acid amplification products which hybridize with the probe are then detected and quantified. The sequence of the oligonucleotide probe can hybridize under very high stringency conditions to a nucleic acid molecule represented by the sequences listed in Tables 2-8 and 15-16.

### Example 18

### Protein-Based Analysis

This example describes methods that can be used to detect changes in expression of hemorrhagic stroke-related proteins, such as those listed in Tables 2-8 and 15-16. Hemorrhagic stroke-related protein sequences can be used in methods of evaluating a stroke, for example for determining whether a subject has had an ICH (for example and not an ischemic stroke), determining the severity or likely neurological recovery of a subject who has had an ICH stroke, and determining a treatment regimen for a subject who has had an ICH stroke. For such procedures, a biological sample of the subject is assayed for a change in expression (such as an increase or decrease) of any combination of at least four hemorrhagic stroke-related proteins, such as any combination of at least four of those listed in Table 5 or 8, at least 20 of those listed in Tables 2-8 and 15-16, or at least 100 of those listed in Tables 2-8 and 15-16. In some examples, protein expression is determined for any combination of at least one gene from each of the following classes of genes: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction (such as at least 2 or at least 3 genes from each of the classes). In some examples, protein expression is determined for at least IL1R2, haptoglobin, amphiphysin, and TAP2 and in some examples also CD163, granzyme M, and Sema4C.

Suitable biological samples include samples containing protein obtained from cells of a subject, such as those present in PBMCs. A change in the amount of four or more hemorrhagic stroke-related proteins in a subject, such as an increase or decrease in expression of four or more hemorrhagic stroke-related proteins listed in Tables 2-8 and 15-16, can indicate that the subject has suffered a hemorrhagic stroke, such as an intracerebral hemorrhagic stroke.

The determination of increased or decreased hemorrhagic stroke-related protein levels, in comparison to such expression in a normal subject (such as a subject who has not previously had a hemorrhagic stroke), is an alternative or supplemental approach to the direct determination of the expression level of hemorrhagic stroke-related nucleic acid sequences by the methods outlined above. The availability of antibodies specific to hemorrhagic stroke-related protein(s) will facilitate the detection and quantitation of hemorrhagic stroke-related protein(s) by one of a number of immunoassay methods that are well known in the art, such as those presented in Harlow and Lane (Antibodies, A Laboratory Manual, CSHL, New York, 1988). Methods of constructing such antibodies are known in the art.

Any standard immunoassay format (such as ELISA, Western blot, or RIA assay) can be used to measure hemorrhagic stroke-related protein levels. A comparison to wild-type (normal) hemorrhagic stroke-related protein levels and an increase or decrease in hemorrhagic stroke-related polypeptide levels (such as an increase in any combination of at least 4 proteins listed in Tables 2-4 or 6-7 with a positive t-statistic or a decrease in any combination of at least 4 proteins listed in Tables 2-4 or 6-7 with a negative t-statistic) is indicative of hemorrhagic stroke, particularly ICH. Immunohistochemical techniques can also be utilized for hemorrhagic stroke-related protein detection and quantification. For example, a tissue sample can be obtained from a subject, and a section stained for the presence of a hemorrhagic stroke-related protein using the appropriate hemorrhagic stroke-related protein specific binding agents and any standard detection system (such as one that includes a secondary antibody conjugated to horseradish peroxidase). General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantitating hemorrhagic stroke-related proteins, a biological sample of the subject that includes cellular proteins can be used. Quantitation of a hemorrhagic stroke-related protein can be achieved by immunoassay and the amount compared to levels of the protein found in cells from a subject who has not had a hemorrhagic stroke. A significant increase or decrease in the amount of four or more hemorrhagic stroke-related proteins listed in Tables 2-8 and 15-16 in the cells of a subject compared to the amount of the same hemorrhagic stroke-related protein found in normal human cells is usually at least 2-fold, at least 3-fold, at least 4-fold or greater difference. Substantial over- or under-expression of four or more hemorrhagic stroke-related protein(s) listed in Tables 2-8 and 15-16 can be indicative of a hemorrhagic stroke, particularly an ICH stroke, and can be indicative of a poor prognosis.

An alternative method of evaluating a stroke is to quantitate the level of four or more hemorrhagic stroke-related proteins listed in Tables 2-8 and 15-16 in a subject, for instance in the cells of the subject. This diagnostic tool is useful for detecting reduced or increased levels of hemorrhagic-related proteins, for instance, though specific techniques can be used to detect changes in the size of proteins, for instance. Localization or coordinated expression (temporally or spatially) of hemorrhagic stroke-related proteins can also be examined using well known techniques.

### Example 19

### Kits

Kits are provided for evaluating a stroke, for example for determining whether a subject has had a hemorrhagic stroke (such as an ICH stroke), determining the severity or likely neurological recovery of a subject who has had a hemorrhagic stroke, and determining a treatment regimen for a subject who has had a hemorrhagic stroke (such as kits containing hemorrhagic stroke detection arrays). Kits are also provided that contain the reagents need to detect complexes formed between oligonucleotides on an array and hemorrhagic stroke-related nucleic acid molecules obtained from a subject, or between proteins or antibodies on an array and proteins obtained from a subject suspected of having had (or known to have had) a hemorrhagic stroke. These kits can each include instructions, for instance instructions that provide calibration curves or charts to compare with the determined (such as experimentally measured) values. The disclosed kits can include reagents needed to determine gene copy number (genomic amplification or deletion), such as probes or primers specific for hemorrhagic stroke-related nucleic acid sequences.

Kits are provided to determine the level (or relative level) of expression or of any combination of four or more hemorrhagic stroke-related nucleic acids (such as mRNA) or hemorrhagic stroke-related proteins (such as kits containing nucleic acid probes, proteins, antibodies, or other hemorrhagic stroke-related protein specific binding agents) listed in Tables 2-8 and 15-16. Such kits can also be used to detect expression of ischemic stroke molecules (e.g. Tables 17-18) and stroke diagnostic molecules (e.g. Table 14).

Kits are provided that permit detection of hemorrhagic stroke-related mRNA expression levels (including over- or under-expression, in comparison to the expression level in a control sample). Such kits include an appropriate amount of one or more of the oligonucleotide primers for use in, for instance, reverse transcription PCR reactions, and can also include reagents necessary to carry out RT-PCR or other in vitro amplification reactions, including, for instance, RNA sample preparation reagents (such as an RNAse inhibitor), appropriate buffers (such as polymerase buffer), salts (such as magnesium chloride), and deoxyribonucleotides (dNTPs).

In some examples, kits are provided with the reagents needed to perform quantitative or semi-quantitative Northern analysis of hemorrhagic stroke-related mRNA. Such kits can include at least four hemorrhagic stroke-related sequence-specific oligonucleotides for use as probes. Oligonucleotides can be labeled, for example with a radioactive isotope, enzyme substrate, co-factor, ligand, chemiluminescent or fluorescent agent, hapten, or enzyme.

Kits are provided that permit detection of hemorrhagic stroke-related genomic amplification or deletion. Nucleotide sequences encoding a hemorrhagic stroke-related protein, and fragments thereof, can be supplied in the form of a kit for use in detection of hemorrhagic stroke-related genomic amplification/deletion or diagnosis of a hemorrhagic stroke, progression of a hemorrhagic stroke, or therapy assessment for subjects who have suffered a hemorrhagic stroke. In examples of such a kit, an appropriate amount of one or more oligonucleotide primers specific for a hemorrhagic stroke-related-sequence (such as those listed in Table 8) is provided in one or more containers. The oligonucleotide primers can be provided suspended in an aqueous solution or as a freeze-dried or lyophilized powder, for instance. The container(s) in which the oligonucleotide(s) are supplied can be any conventional container that is capable of holding the supplied form, for instance, microfuge tubes, ampoules, or bottles. In some applications, pairs of primers are provided in pre-measured single use amounts in individual, typically disposable, tubes, or equivalent containers. With such an arrangement, the sample to be tested for the presence of hemorrhagic stroke-related genomic amplification/deletion can be added to the individual tubes and in vitro amplification carried out directly.

The amount of each primer supplied in the kit can be any amount, depending for instance on the market to which the product is directed. For instance, if the kit is adapted for research or clinical use, the amount of each oligonucleotide primer provided is likely an amount sufficient to prime several in vitro amplification reactions. Those of ordinary skill in the art know the amount of oligonucleotide primer that is appropriate for use in a single amplification reaction. General guidelines can be found in Innis et al. (PCR Protocols, A Guide to Methods and Applications, Academic Press, Inc., San Diego, Calif., 1990), Sambrook et al. (In Molecular Cloning: A Laboratory Manual, Cold Spring Harbor, New York, 1989), and Ausubel et al. (In Current Protocols in Molecular Biology, John Wiley & Sons, New York, 1998).

A kit can include more than two primers to facilitate the in vitro amplification of hemorrhagic stroke-related genomic sequences, such as those listed in Tables 2-8 and 15-16, or the 5′ or 3′ flanking region thereof.

In some examples, kits also include the reagents needed to perform in vitro amplification reactions, such as DNA sample preparation reagents, appropriate buffers (for example polymerase buffer), salts (for example magnesium chloride), and deoxyribonucleotides (dNTPs). Written instructions can also be included. Kits can further include labeled or unlabeled oligonucleotide probes to detect the in vitro amplified sequences. The appropriate sequences for such a probe will be any sequence that falls between the annealing sites of two provided oligonucleotide primers, such that the sequence the probe is complementary to is amplified during the in vitro amplification reaction (if it is present in the sample).

One or more control sequences can be included in the kit for use in the in vitro amplification reactions. The design of appropriate positive and negative control sequences is well known to one of ordinary skill in the art.

In particular examples, a kit includes one or more of the hemorrhagic stroke detection arrays disclosed herein (such as those disclosed in Example 15). In one example, the array consists essentially of probes that can detect any combination of at least 4 of the hemorrhagic stroke-related molecules listed in Tables 2-8 and 15-16, and control probes (such as GAPDH, actin, and YWHAZ), ischemic stroke probes (e.g. those specific for molecules listed in Tables 17-18), stroke diagnostic probes (e.g. those specific for molecules listed in Table 14), or combinations thereof. In some examples, the array consists essentially of probes (such as oligonucleotides, proteins, or antibodies) that can recognize any combination of at least one gene (or protein) from each of the following gene classes: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction (such as at least 2 or at least 3 genes (or proteins) from each class), and controls. Probes that recognize hemorrhagic stroke-related and control sequences (such as negative and positive controls) can be on the same array, or on different arrays.

Kits are also provided for the detection of hemorrhagic stroke-related protein expression, for instance increased expression of any combination of at least four proteins listed in Table 5 or 8. Such kits include one or more hemorrhagic stroke-related proteins (full-length, fragments, or fusions) or specific binding agent (such as a polyclonal or monoclonal antibody or antibody fragment), and can include at least one control. The hemorrhagic stroke-related protein specific binding agent and control can be contained in separate containers. The kits can also include agents for detecting hemorrhagic stroke-related protein:agent complexes, for instance the agent can be detectably labeled. If the detectable agent is not labeled, it can be detected by second antibodies or protein A, for example, either of both of which also can be provided in some kits in one or more separate containers. Such techniques are well known.

Additional components in some kits include instructions for carrying out the assay, which can include reference values (e.g. control values). Instructions permit the tester to determine whether hemorrhagic stroke-linked expression levels are elevated, reduced, or unchanged in comparison to a control sample. Reaction vessels and auxiliary reagents such as chromogens, buffers, enzymes, and the like can also be included in the kits.

### Example 20

### Gene Expression Profiles (Fingerprints)

With the disclosure of many hemorrhagic stroke-related molecules (as represented for instance by those listed in Tables 2-8 and 15-16), gene expression profiles that provide information on evaluating a stroke, for example for determining whether a subject has had a hemorrhagic stroke (such as an ICH stroke), determining the severity or likely neurological recovery of a subject who has had a hemorrhagic stroke, and determining a treatment regimen for a subject who has had hemorrhagic stroke, are now enabled.

Hemorrhagic stroke-related expression profiles include the distinct and identifiable pattern of expression (or level) of sets of hemorrhagic stroke-related genes, for instance a pattern of increased and decreased expression of a defined set of genes, or molecules that can be correlated to such genes, such as mRNA levels or protein levels or activities. The set of molecules in a particular profile can include any combination of at least four of the sequences listed in any of Tables 2-8 and 15-16.

Another set of molecules that could be used in a profile include any combination of at least four sequences listed in Tables 2-8 and 15-16, each of which is over- or under-expressed following a hemorrhagic stroke, such as an ICH stroke. For example, a hemorrhagic stroke-related gene expression profile can include one sequence from each of the following classes of genes: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction. In another example, the molecules included in the profile include at least IL1R2, haptoglobin, amphiphysin, and TAP2, or any one of these, and in some examples also CD163, granzyme M, and Sema4C.

Yet another example of a set of molecules that could be used in a profile would include any combination of at least 10 of the sequences listed in Tables 2-8 and 15-16, whose expression is upregulated or downregulated following hemorrhagic stroke. In a particular example, a set of molecules that could be used in a profile would include any combination of at least 100 or at least 200 of the sequences listed in Tables 2-8 and 15-16, whose expression is upregulated or downregulated following hemorrhagic stroke.

Particular profiles can be specific for a particular stage or age of normal tissue (such as PMBCs). Thus, gene expression profiles can be established for a pre-hemorrhagic stroke tissue (such as normal tissue not subjected to a hemorrhagic challenge or preconditioning) or a hemorrhage challenged tissue. Each of these profiles includes information on the expression level of at least four or more genes whose expression is altered following hemorrhagic stroke. Such information can include relative as well as absolute expression levels of specific genes. Likewise, the value measured can be the relative or absolute level of protein expression or protein activity, which can be correlated with a “gene expression level.” Results from the gene expression profiles of an individual subject can be viewed in the context of a test sample compared to a baseline or control sample fingerprint/profile.

The levels of molecules that make up a gene expression profile can be measured in any of various known ways, which may be specific for the type of molecule being measured. Thus, nucleic acid levels (such as direct gene expression levels, such as the level of mRNA expression) can be measured using specific nucleic acid hybridization reactions. Protein levels can be measured using standard protein assays, using immunologic-based assays (such as ELISAs and related techniques), or using activity assays. Examples for measuring nucleic acid and protein levels are provided herein; other methods are well known to those of ordinary skill in the art.

Examples of hemorrhagic-related gene expression profiles can be in array format, such as a nucleotide (such as polynucleotide) or protein array or microarray. The use of arrays to determine the presence and/or level of a collection of biological macromolecules is now well known (see, for example, methods described in published PCT application number WO 99/48916, describing hypoxia-related gene expression arrays). In array-based measurement methods, an array can be contacted with nucleic acid molecules (in the case of a nucleic acid-based array) or peptides (in the case of a protein-based array) from a sample from a subject. The amount or position of binding of the subject's nucleic acids or peptides then can be determined, for instance to produce a gene expression profile for that subject. Such gene expression profile can be compared to another gene expression profile, for instance a control gene expression profile from a subject known to have suffered a stroke (such as ICH), or known to not have suffered a stroke. Such a method could be used to determine whether a subject had a hemorrhagic stroke or determine the prognosis of a subject who had hemorrhagic stroke. In addition, the subject's gene expression profile can be correlated with one or more appropriate treatments, which can be correlated with a control (or set of control) expression profiles for levels of hemorrhage, for instance.

### Example 21

### In Vivo Screening Assay

This example describes particular in vivo methods that can be used to screen test agents for their ability to alter the activity of a hemorrhagic stroke-related molecule. However, the disclosure is not limited to these particular methods. One skilled in the art will appreciate that other in vivo assays could be used (such as other mammals or other means of inducing a hemorrhagic stroke).

As disclosed in the Examples above, expression of the disclosed hemorrhagic stroke-related molecules (such as those listed in Tables 2-8 and 15-16) is increased or decreased following hemorrhagic stroke, such as intracerebral hemorrhagic stroke. Therefore, screening assays can be used to identify and analyze agents that normalize such activity (such as decrease expression/activity of a gene that is increased following a hemorrhagic stroke, increase expression/activity of a gene that is decreased following an hemorrhagic stroke, or combinations thereof), or further enhance the change in activity (such as further decrease expression/activity of a gene that is decreased following hemorrhagic stroke, or further increase expression/activity of a gene that is increased following hemorrhagic stroke). For example, it may be desirable to further enhance the change in activity if such a change provides a beneficial effect to the subject or it may be desirable to neutralize the change in activity if such a change provides a harmful effect to the subject.

A mammal is exposed to conditions that induce a hemorrhagic stroke, such as an ICH stroke. Several methods of inducing hemorrhagic stroke in a mammal are known, and particular examples are provided herein. Mammals of any species, including, but not limited to, mice, rats, rabbits, dogs, guinea pigs, pigs, micro-pigs, goats, and non-human primates, such as baboons, monkeys, and chimpanzees, can be used to generate an animal model of hemorrhagic stroke. Such animal models can also be used to test agents for an ability to ameliorate symptoms associated with hemorrhagic stroke. In addition, such animal models can be used to determine the LD50 and the ED50 in animal subjects, and such data can be used to determine the in vivo efficacy of potential agents.

In a particular example, ICH stroke is induced in a rat by injection of 0.14 U of type IV bacterial collagenase in 10 μL of saline into the basal ganglia, resulting in a small amount of blood collecting in the striatum. In another example, ICH stroke is induced in an adult rat by infusion of 100-200 μl of autologous blood over 15 minutes into the right basal ganglia (such as the striatum), resulting in intraventricular hemorrhage (IVH) and post-hemorrhagic ventricular dilatation. The animal can be under anesthesia (for example 1 mL/kg of a mixture of ketamine (75 mg/mL) and xylazine (5 mg/mL)).

Simultaneous to inducing the hemorrhagic stroke, or at a time later, one or more test agents are administered to the subject under conditions sufficient for the test agent to have the desired effect on the subject. The amount of test agent administered can be determined by skilled practitioners. In some examples, several different doses of the potential therapeutic agent can be administered to different test subjects, to identify optimal dose ranges. Any appropriate method of administration can be used, such as intravenous, intramuscular, or transdermal. In one example, the agent is added at least 30 minutes after the hemorrhagic stroke, such as at least 1 hour, at least 2 hours, at least 6 hours, or at least 24 hours after the hemorrhagic stroke.

Subsequent to the treatment, biological samples from the animals are analyzed to determine expression levels of one or more of the hemorrhagic stroke-related molecules listed in Tables 2-8 and 15-16 using the methods provided herein. Agents that are found to normalize the activity or further enhance the change in activity of one or more of the hemorrhagic stroke-related molecules listed in Tables 2-8 and 15-16 can be selected. Such agents can be useful, for example, in decreasing one or more symptoms associated with hemorrhagic stroke, such as a decrease of at least about 10%, at least about 20%, at least about 50%, or even at least about 90%.

Once identified, test agents found to alter the activity of a hemorrhagic stroke-related molecule can be formulated in therapeutic products (or even prophylactic products) in pharmaceutically acceptable formulations, and used to treat a subject who has had a hemorrhagic stroke.

In particular examples, the method also includes determining a therapeutically effective dose of the selected test agent. For example, a hemorrhagic stroke is induced in the mammal, and one or more test agents identified in the examples above administered. Animals are observed for one or more symptoms associated with hemorrhagic stroke, such as sensory loss, paralysis (such as hemiparesis), pupillary changes, blindness, and ataxia. A decrease in the development of symptoms associated with hemorrhagic stroke in the presence of the test agent provides evidence that the test agent is a therapeutic agent that can be used to decrease or even inhibit hemorrhagic stroke in a subject.

In view of the many possible embodiments to which the principles of the disclosure can be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of evaluating hemorrhagic stroke in a subject, comprising:
detecting differential expression of at least four hemorrhagic stroke-related molecules of the subject, wherein the at least four hemorrhagic stroke-related molecules are represented by any combination of at least four molecules listed in any of Tables 2-8 and 15-16, and wherein the presence of differential expression of at least four hemorrhagic stroke-related molecules indicates that the subject has had a hemorrhagic stroke.

2. The method of claim 1, wherein detecting differential expression comprises detecting differential expression within 24 hours, within 2-5 days, within 7-14 days, or within 90 days of onset of clinical signs and symptoms that indicate a potential stroke.

3. The method of claim 1, wherein the hemorrhagic stroke is an intracerebral hemorrhagic (ICH) stroke.

4. The method of claim 1, wherein the hemorrhagic stroke is not a subarachnoid hemorrhagic stroke.

5-6. (canceled)

7. The method of claim 1, wherein the method comprises determining whether there is an upregulation in any combination of at least IL1R2, haptoglobin, and amphiphysin, and determining whether there is a downregulation in TAP2.

8. The method of claim 7, wherein the method further comprises determining whether there is an upregulation in CD163 and determining whether there is a downregulation in granzyme M or Sema 4C.

9. The method of claim 1, wherein differential expression comprises upregulation and wherein the method comprises determining whether there is an upregulation in any combination of at least four hemorrhagic stroke-related genes listed in Tables 2-4 or 6-7 with a positive t-statistic or Tables 15 and 16 with a positive fold-change (FC) value, wherein the presence of an increase in expression of at least four hemorrhagic stroke-related molecules indicates that the subject has had a hemorrhagic stroke.

10-12. (canceled)

13. The method of claim 1, wherein the method has a sensitivity of at least 75% and accuracy of at least 90%.

14. The method of claim 1, wherein the subject had an onset of clinical signs and symptoms of a hemorrhagic stroke no more than 72 hours prior to determining whether there is differential expression of at least four hemorrhagic stroke-related molecules.

15-21. (canceled)

22. The method of claim 1, wherein the hemorrhagic stroke-related molecules are obtained from peripheral blood mononuclear cells (PBMCs).

23-26. (canceled)

27. The method of claim 1, wherein determining whether there is differential expression of at least four hemorrhagic stroke-related molecules comprises:
measuring a level of at least four hemorrhagic stroke-related nucleic acid molecules in a sample derived from the subject, wherein a difference in the level of the at least four hemorrhagic stroke-related nucleic acid molecules in the sample, relative to a level of the at least four hemorrhagic stroke-related nucleic acid molecules in an analogous sample from a subject not having had an hemorrhagic stroke is differential expression in those at least four hemorrhagic stroke-related molecules.

28-31. (canceled)

32. The method of claim 1, wherein the method comprises determining whether there is an upregulation or downregulation in any combination of at least one gene from each class of genes, wherein the class of genes comprise: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction.

33. The method of claim 1, further comprising:
detecting differential expression of at least four stroke-related molecules listed in Table 14, wherein the presence of increased expression of at least four stroke-related molecules listed in Table 14 indicates that the subject has had a stroke.

34. The method of claim 1, wherein the at least four hemorrhagic stroke-related molecules do not include any of those listed as yes for gender or race in Table 13.

35. The method of claim 1, wherein the at least four hemorrhagic stroke-related molecules include one or more of those listed as yes for draw time in Table 13.

36. The method of claim 1, wherein evaluating the hemorrhagic stroke comprises predicting a likelihood of severity of neurological sequalae of the hemorrhagic stroke.

37-38. (canceled)

39. The method of claim 36, wherein detection of differential expression in at least IL1R2, haptoglobin, amphiphysin, and TAP2 indicates that the subject has a higher risk of long-term adverse neurological sequalae.

40. (canceled)

41. The method of claim 1, further comprising administering to the subject a treatment to avoid or reduce hemorrhagic injury if the presence of differential expression indicates that the subject has had a hemorrhagic stroke.

42. (canceled)

43. A method of evaluating hemorrhagic stroke in a subject, comprising:
applying isolated nucleic acid molecules obtained from PBMCs of the subject to an array, wherein the array consists of oligonucleotides complementary to all 30 genes listed in Table 5;
incubating the isolated nucleic acid molecules with the array for a time sufficient to allow hybridization between the isolated nucleic acid molecules and oligonucleotide probes, thereby forming isolated nucleic acid molecule:oligonucleotide complexes; and
analyzing the isolated nucleic acid molecule:oligonucleotide complexes to determine if expression of the isolated nucleic acid molecules is altered, wherein the presence of differential expression in at least 4 of the 30 genes indicates that the subject has had a hemorrhagic stroke.

44. The method of claim 1, wherein evaluating the hemorrhagic stroke comprises predicting a likelihood of neurological recovery of the subject.

45. (canceled)

46. The method of claim 44, wherein detection of differential expression in at least IL1R2, haptoglobin, amphiphysin, and TAP2 indicates that the subject has a lower likelihood of neurological recovery.

47-50. (canceled)

51. An array consisting essentially of oligonucleotides complementary to hemorrhagic stroke-related gene sequences, wherein the hemorrhagic stroke-related gene sequences comprise any combination of at least four of the genes listed in Tables 2-8 and 15-16.

52-53. (canceled)

54. The array of claim 51, wherein the hemorrhagic stroke-related gene sequences comprise at least one gene from each class of genes, wherein the class of genes comprise: genes involved in acute inflammatory response, genes involved in cell adhesion, genes involved in suppression of the immune response, genes involved in hypoxia, genes involved in hematoma formation or vascular repair, genes involved in the response to the altered cerebral microenvironment, and genes involved in signal transduction.

55. The array of claim 51, wherein the array further consists of 1-50 oligonucleotides complementary to a control sequence, 1-35 oligonucleotides complementary to an ischemic stroke related sequence, 1-18 oligonucleotides complementary to a stroke-related sequence, or combinations thereof.

56. The array of claim 51, wherein the hemorrhagic stroke-related gene sequences consist of all genes listed in any of Tables 2-8 and 15-16.

57-58. (canceled)

59. An array consisting essentially of antibodies that specifically bind to hemorrhagic stroke-related gene sequences, wherein the hemorrhagic stroke-related gene sequences comprise any combination of at least four of the genes listed in Tables 2-8 and 15-16.

60. A kit for evaluating a hemorrhagic stroke in a subject, comprising:
the array of claim 50; and
a buffer solution, in separate packaging.

61. A method of identifying an agent that alters an activity of one or more hemorrhagic stroke-related molecules listed in Tables 2-8 and 15-16, comprising:
administering an agent to a laboratory mammal under conditions sufficient to mimic a hemorrhagic stroke;
administering to the mammal one or more test agents under conditions sufficient for the one or more test agents to alter the activity of one or more hemorrhagic stroke-related molecules;
obtaining a biological sample from the mammal; and
detecting differential expression of the one or more hemorrhagic stroke-related molecules present in the biological sample, wherein the presence of differential expression of the hemorrhagic stroke-related molecule indicates that the test agent alters the activity of an hemorrhagic stroke-related molecule listed in Tables 2-8 and 15-16.

62-64. (canceled)

65. A method of treating a mammal who has had a hemorrhagic stroke, comprising administering the agent identified using the method of claim 61 to the mammal.

66. A method of imaging a mammalian brain in a subject, comprising:
administering to the subject a labeled antibody, wherein the antibody specifically binds one or more of the proteins listed in Tables 2-8 and 15-16; and
detecting the label, thereby permitting imaging of the brain.

67. (canceled)

68. A method of determining whether a subject has suffered a stroke, comprising:
detecting expression of at least four stroke-related molecules of the subject, wherein the at least four stroke-related molecules are represented by any combination of at least four molecules listed in any of Table 14, and wherein the presence of increased expression of at least four hemorrhagic stroke-related molecules indicates that the subject has had a stroke.

69. (canceled)

70. The method of claim 68, further comprising determining whether the stroke was a hemorrhagic stroke or an ischemic stroke.

71. (canceled)
